(19) United States
(12) Patent Application Publication
(10) Pub. No.: US 2022/0354949 A1
(43) Pub. Date: Nov. 10, 2022

(54) NOVEL COMPOUNDS

(71) Applicant: XENIOPRO GmbH, Kelkheim (Taunus) (DE)

(72) Inventors: Viktoria REINMÜLLER, Allschwil (CH); Roman MARTY, Allschwil (CH); Olivier WAGNIÈRES, Lausanne (CH); Jean-Baptiste GUALTIEROTTI, Allschwil (CH); Verena KÜPPERS, Binningen (CH)

(73) Assignee: XENIOPRO GmbH, Kelkheim (Taunus) (DE)

(21) Appl. No.: 17/270,665

(22) PCT Filed: Aug. 23, 2019
(86) PCT No.: PCT/EP2019/072633

§ 371 Date: Feb. 23, 2021

(30) Foreign Application Priority Data
Aug. 24, 2018 (EP) 18190769.2

(51) Int. Cl. A61K 39/39; C07C 311/51; C07D 213/89; C07C 261/04; C07C 259/10; C07C 325/02; C07D 405/04; C07C 217/58; C07C 239/12; C07C 255/43; C07D 309/06; C07D 211/22; C07D 295/092; C07D 453/02; C07D 205/04; C07D 491/107; C07D 401/12; C07D 405/12; C07D 519/00; C07D 407/12; C07D 331/04; C07D 409/12; C07D 211/28; C07D 305/04; C07C 69/78; C07C 47/198; C07D 213/80; C07D 213/643; C07D 237/04; C07D 239/47; C07D 239/52

(52) U.S. Cl. CPC A61K 39/39; A61K 2039/55511; C07D 213/89; C07C 261/04; C07C 259/10; C07C 325/02; C07D 405/04; C07C 217/58; C07C 239/12; C07C 255/43; C07D 309/06; C07D 211/22; C07D 295/092; C07D 453/02; C07D 205/04; C07D 491/107; C07D 401/12; C07D 405/12; C07D 519/00; C07D 407/12; C07D 331/04; C07D 409/12; C07D 211/28; C07D 305/04; C07C 69/78; C07C 47/198; C07D 213/80; C07D 213/643; C07D 237/04; C07D 239/47; C07D 239/52; C07C 311/51

(57) ABSTRACT

The present invention comprises novel aromatic molecules, which can be used in the treatment of pathological conditions, such as cancer, skin diseases, muscle disorders, and immune system-related disorders such as disorders of the haematopoietic system including the haematologic system in human and veterinary medicine.

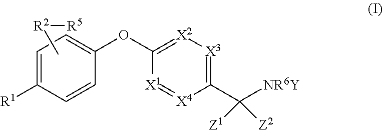

The present invention relates to novel compounds and their use as therapeutic agents in human and veterinary medicine. The compounds of the present invention can be used in the treatment of pathological conditions including cancer, skin disorders, muscle disorders, disorders of the lung, disorders of the haematopoietic system including the haematologic system and immune system-related disorders.

### DESCRIPTION OF THE INVENTION

The present invention covers novel molecules that show remarkable biological activity on human and animal derived cells. According compounds were found to influence the growth and survival of cancer cells and primary non-cancer cells. In particular, molecules were identified that are able to completely or partially inhibit cell growth or result in cell death. Moreover, some of the compounds were found to impact cellular signaling pathways, in particular the Notch signaling pathway. According molecules were found to enhance the Notch signaling pathway.

Thus, the present invention relates to compounds as defined herein that feature antiproliferative activity, which can be used in the treatment of benign and malignant hyperproliferative disorders in human and veterinary medicine. In particular, the present invention relates to compounds as defined herein for the treatment of disorders of the haematopoietic system including the haematologic system and immune system-related disorders, concerning malignancies of both the myeloid lineage and the lymphoid lineage, malignant and non-malignant disorders of the skin and mucosa, e.g. cornification disorders, malignant and non-malignant disorders of the muscle, including hyperproliferative disorders of the muscle, such as muscle hyperplasia and muscle hypertrophy, disorders of the neuroendocrine system, hyperproliferative disorders, cancer and pre-cancerous lesions of the skin and mucosa, such as non-melanoma skin cancer including squamous and basal cell carcinoma, actinic keratosis, hyperproliferative disorders and cancer of the oral cavity and tongue, hyperproliferative disorders and cancer of the neuroendocrine system such as medullary thyroid cancer, hyperproliferative disorders and cancer of the haematopoietic system including the haematologic system such as leukemia and lymphoma, hyperproliferative disorders and cancer of the lung, breast, stomach, genitourinary tract, e.g. cervical cancer and including cancer of the ovaries, in human and veterinary medicine.

The biological activity, e.g. the antiproliferative activity of the claimed compounds can be attributed to but may not be limited to Notch signaling enhancing activity. Thus, the present invention also relates to compounds as defined herein that feature Notch enhancing activity, which can be used in the treatment of pathological conditions that are responsive for Notch-regulation, such as cancer, skin diseases, muscle disorders, disorders of the haematopoietic system including the haematologic system and immune system-related disorders, in human and veterinary medicine.

The compounds of the present invention relate to bisarylether structures composed of two six-membered aromatic cycles, wherein one of the aromatic cycles is an unsubstituted or substituted benzyl ring and the other aromatic cycle is an unsubstituted or substituted aryl ring, which optionally contains N-atoms, thus optionally being a six-membered heteroaromatic cycle.

All such bisarylether structures share the common feature of containing a substituent in both para-positions relative to the ether bond, wherein such substituent on the benzyl ring, which cannot be a heteroaromatic cycle, is preferably selected from apolar residues and/or from sterically demanding residues; and wherein such substituent on the aryl ring which can optionally be a heteroaromatic cycle, is selected from structural units preferably containing a high amount of heteroatoms.

A first aspect of the present invention relates to compounds of general formula (I) and salts and solvates thereof:

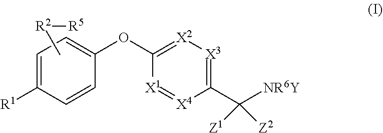

R¹=C₁-C₁₂ preferably C₄-C₁₂ alkyl, C₂-C₁₂ preferably C₄-C₁₂ alkenyl, C₂-C₁₂ preferably C₄-C₁₂ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, —OC₁-C₁₂ preferably —OC₃-C₁₂ alkyl, —OC₂-C₁₂ preferably —OC₃-C₁₂ alkenyl, —OC₂-C₁₂ preferably —OC₃-C₁₂ alkynyl, —OC₃-C₈ cycloalkyl, —OC₅-C₈ cycloalkenyl, —OC₅-C₁₂ bicycloalkyl, —OC₇-C₁₂ bicycloalkenyl, —OC₅-C₁₄ tricycloalkyl, —SC₁-C₁₂ preferably —SC₃-C₁₂ alkyl, —SC₂-C₁₂ preferably —SC₃-C₁₂ alkenyl, —SC₂-C₁₂ preferably —SC₃-C₁₂ alkynyl, —SC₃-C₈ cycloalkyl, —SC₅-C₈ cycloalkenyl, —SC₅-C₁₂ bicycloalkyl, —SC₇-C₁₂ bicycloalkenyl, —SC₅-C₁₄ tricycloalkyl, —NHR⁷ or —NR⁷R⁸ wherein R⁷ and R⁸ are independently from each other selected from: C₁-C₁₂ preferably C₃-C₁₂ alkyl, C₂-C₁₂ preferably C₃-C₁₂ alkenyl, C₂-C₁₂ preferably C₃-C₁₂ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, or wherein R⁷ can form a ring structure together with Re wherein the said ring structure including the N-atom is selected from three to eight membered cyclic structures or five to twelve membered bicyclic structures and wherein all said ring structures can additionally contain one or more heteroatoms independently selected from O, S and N in replacement of a carbon atom contained in the ring structure, and particularly wherein such a replacement results in residues that contain at least twice the number of C atoms than heteroatoms independently selected from O, S and N;

wherein all alkyl, alkenyl and alkynyl residues contained in the definitions of R¹, R⁷ and R⁸ are linear or branched, and are unsubstituted or substituted with one or more substituents independently selected from: —F, —Cl, —Br, —I, —CN, —NCO, —NCS, —OH, —NH₂, —NO₂, ═O, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, linear or branched —OC₁-C₅ alkyl such as —OCH₃, —OC₃-C₅ cycloalkyl such as —O (cyclopropyl), linear or branched —NH(C₁-C₅ alkyl), linear or branched —N(C₁-C₅ alkyl)(C₁-C₅ alkyl), —NH(C₃-C₅ cycloalkyl) such as —NH(cyclopropyl), —N(C₃-C₅ cycloalkyl)(C₃-C₅ cycloalkyl), linear or branched —N(C₁-C₅ alkyl)(C₃-C₅ cycloalkyl);
wherein when an alkyl, alkenyl and alkynyl residue contained in the definitions of R¹, R⁷ and R⁸ is substituted with one or more substituents being ═O, such substitution with ═O cannot result in one of the groups selected from C═O, S═O and N═O directly bound to an aromatic ring; wherein all cyclic structures, bicyclic structures and tricyclic structures including cycloalkyl, cycloalkenyl, bicycloalkyl, bicycloalkenyl and tricycloalkyl residues contained in the definitions of R¹, R⁷ and R⁸ are unsubstituted or substituted with one or more substituents independently selected from: —F, —Cl, —Br, —I, —CN, —NCO, —NCS, —OH, —NH₂, —NO₂, ═O, linear or branched C₁-C₅ alkyl such as —CH₃, linear or branched —OC₁-C₅ alkyl such as —OCH₃, linear or branched —NH(C₁-C₅ alkyl), linear or branched —N(C₁-C₅ alkyl)(C₁-C₅ alkyl), —NH(C₃-C₅ cycloalkyl) such as —NH(cyclopropyl), —N(C₃-C₅ cycloalkyl)(C₃-C₅ cycloalkyl), linear or branched —N(C₁-C₅ alkyl)(C₃-C₅ cycloalkyl);

wherein all alkyl, alkenyl and alkynyl residues contained in the definitions of R¹, R⁷ and R⁸ can contain one or more heteroatoms independently selected from O, S and N in replacement of a carbon atom, and wherein such a replacement results in residues that contain at least twice the number of C atoms than heteroatoms independently selected from O, S and N, and wherein such replacement additionally cannot result in one of the groups selected from C═O, S═O and N═O directly bound to an aromatic ring;

wherein all cycloalkyl, cycloalkenyl, bicycloalkyl, bicycloalkenyl and tricycloalkyl residues contained in the definitions of R¹, R⁷ and R⁸ can contain one or more heteroatoms independently selected from O, S and N in replacement of a carbon atom, and wherein such a replacement results in residues that contain at least the same number of C atoms than heteroatoms independently selected from O, S and N;

wherein all alkyl, alkenyl, alkynyl, cycloalkyl, cycloalkenyl, bicycloalkyl, bicycloalkenyl and tricycloalkyl residues contained in the definitions of R¹, R⁷ and R⁸ can be partially or fully halogenated, particularly fluorinated, more particularly perfluorinated;

wherein bicyclic and tricyclic residues include fused, bridged and spiro systems;

and wherein R¹ is preferably selected from methyl, ethyl, n-propyl, n-butyl, n-pentyl, n-hexyl, iso-propyl, sec-butyl, tert-butyl, tert-pentyl, tert-octyl, 3-pentyl, —CF₃, —CF₂CF₃, —(CF₂)₂CF₃, —CH(CF₃)₂, —CH₂SCH₃, —CH₂CH₂SCH₃, —CH₂SCH₂CH₃, —CH₂CH₂SCH₂CH₃, methoxymethyl, methoxyethyl, methoxypropyl, ethoxymethyl, ethoxyethyl, propoxymethyl, dimethyl-aminomethyl, dimethyl-aminoethyl, diethyl-aminomethyl, ethyl-methyl-aminomethyl, cyclopropyl, methyl-cyclopropyl, ethyl-cyclopropyl, trifluoromethyl-cyclopropyl, perfluoroethyl-cyclopropyl, cyclobutyl, cyclopentyl, cyclohexyl, cycloheptyl, bicyclopentyl, bicyclohexyl, bicycloheptyl preferably norbornyl, bicyclooctyl, bicyclooctenyl, bicyclononyl, methylbicyclononyl, adamantyl, tricyclodecyl, oxiranyl, oxetanyl, tetrahydrofuranyl, methyltetrahydrofuranyl, trimethyltetrahydrofuranyl, tetrahydropyranyl, aziridinyl, N-methylaziridinyl, azetidinyl, N-methylazetidinyl, difluoroazetidinyl, pyrrolidinyl, N-methylpyrrolidinyl, piperidinyl, N-methylpiperidinyl, difluoropiperidinyl, thiiranyl, thietanyl, tetrahydrothiophenyl, tetrahydrothiopyranyl, dioxanyl, piperazinyl, dimethylpiperazinyl, dithianly, morpholinyl, N-methylmorpholinyl, thiomorpholinyl, N-methylthiomorpholinyl, oxa-azaspiroheptyl, N-methyloxa-azaspiroheptyl, azaspiroheptyl, N-methylazaspiroheptyl, thia-azaspiroheptyl, N-methylthia-azaspiroheptyl, difluorothia-azaspiroheptyl, azaspirooctyl, N-methylazaspirooctyl, oxa-azaspirooctyl, N-methyloxa-azaspirooctyl, oxa-azaspirononyl, N-methyloxa-azaspirononyl, azaspirononyl, N-methylazaspirononyl, oxa-azaspirodecyl, N-methyloxa-azaspirodecyl, azaspirodecyl, N-methylazaspirodecyl, dihydro-oxazinyl, N-methyldihydro-oxazinyl, oxazolidinyl, N-methyloxazolidinyl, dioxolanyl, imidazolidinyl, N-methylimidazolidinyl, N,N-dimethylimidazolidinyl, azepanyl, N-methylazepanyl, azaspirohexyl, N-methylazaspirohexyl, oxa-azadispirodecyl, N-methyloxa-azadispirodecyl, azadispirodecyl, N-methylazadispirodecyl, oxa-azabicyclooctyl, N-methyloxa-azabicyclooctyl, azabicyclooctyl, N-methylazabicyclooctyl, azabicycloheptyl, N-methylazabicycloheptyl, azabicyclononyl, N-methylazabicyclononyl, azaadamantyl, —O(adamantyl), oxa-azabicyclononyl, N-methyloxa-azabicyclononyl, oxa-azabicycloheptyl, N-methyloxa-azabicycloheptyl, diazabicyclooctyl, N-methyldiazabicyclooctyl, N,N-dimethyldiazabicyclooctyl, diazabicycloheptyl, N-methyldiazabicycloheptyl, N,N-dimethyldiazabicycloheptyl; 4-oxocyclohexyl; 3-oxocyclopentyl; 2-oxocyclobutyl, 4-oxobicyclo[4.1.0]heptan-1-yl;

and wherein R¹ is even more preferably selected from C₄-C₁₂ alkyl, C₄-C₁₂ alkenyl, C₄-C₁₂ alkynyl, cyclic, bicyclic and tricyclic residues, wherein the alkyl, alkenyl and alkynyl residues are preferably branched, including:

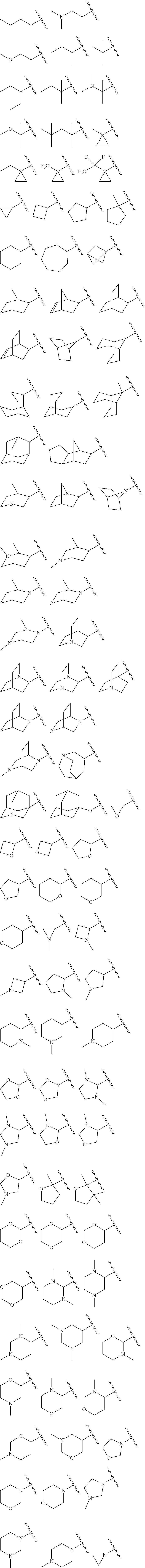

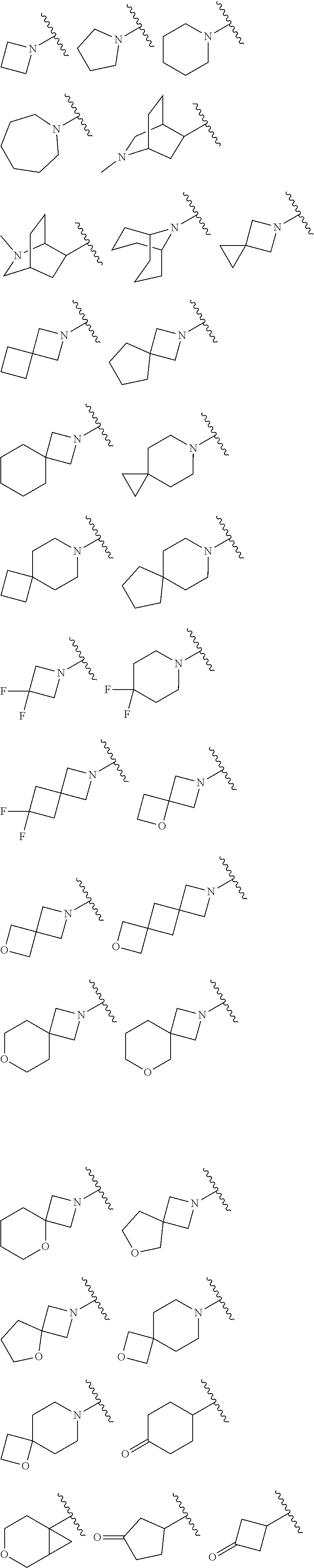

R²-R⁵ are independently from each other selected from —H, —F, —Cl, —Br, —I, —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl); wherein all alkyl, alkenyl, alkynyl and cycloalkyl residues contained in the definitions of R²-R⁵ are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂; wherein all alkyl, alkenyl, alkynyl and cycloalkyl residues contained in the definitions of R²-R⁵ can contain one or more heteroatoms independently selected from O, S and N in replacement of a carbon atom, and wherein such a replacement cannot result in one of the groups selected from C═O and S═O directly bound to an aromatic ring;

wherein R²-R³ each are preferably —H, R⁴ is preferably —H or —F, and R⁵ is preferably —H, —F, —Cl, —Br, —CH₃, —CF₃, —CH═CH₂, —C≡CH, —CH₂OH, —CH₂NHCH₃, —OH, —OCH₃, —OCF₃, cyclopropyl, oxiranyl, —CH₂—N-morpholinyl, —C(CH₃)₃, —CH₂OCH₃, —NO₂, —CN, —NH₂, —N(CH₃)₂, —OCH(CH₃)₂, —CH₂NH₂, —CH₂N(CH₃)₂;

wherein the six-membered aromatic ring, to which substituents R¹ to R⁵ are bound as defined in general formula (I), is preferably selected from:

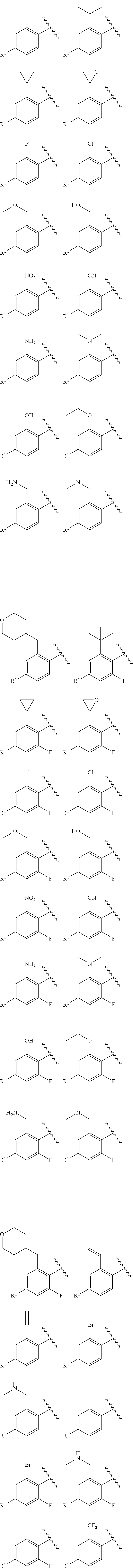

X¹-X⁴ are independently from each other selected from N, CR⁹, CR¹⁰, CR¹¹, CR¹²; R⁹-R¹² are independently from each other selected from —H, —F, —Cl, —Br, —I, —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl and cycloalkyl residues contained in the definitions of R⁹-R¹² are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂;

wherein all alkyl, alkenyl, alkynyl and cycloalkyl residues contained in the definitions of R⁹-R¹² can contain one or more heteroatoms independently selected from O, S and N in replacement of a carbon atom, and wherein such a replacement cannot result in one of the groups selected from C═O and S═O directly bound to an aromatic ring;

wherein R⁹-R¹² are preferably selected from —H, —F, —Cl, —Br, —CH₃, —CF₃, —OH, —OCH₃, —OCF₃, cyclopropyl, oxiranyl, —C(CH₃)₃, —N(CH₃)₂, —NH₂, —CN, —CH₂OCH₃, —OCH(CH₃)₂, —CH₂NH₂, —CH₂N(CH₃)₂, —CH₂OH, —NO₂, —CH₂—N-morpholinyl;

and wherein the six-membered aromatic ring containing X¹-X⁴ as defined in general formula (I) is preferably selected from:

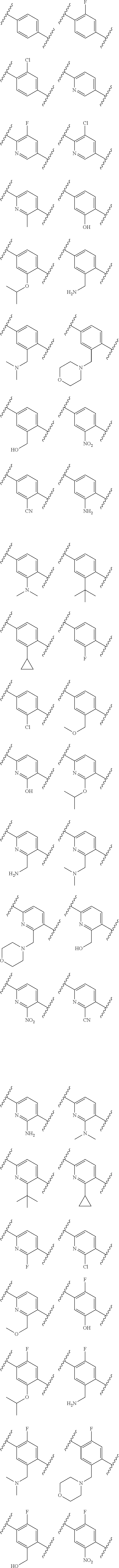

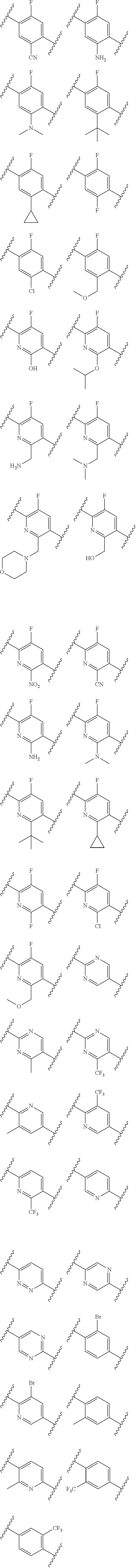

R⁶=—H, C₁-C₈ preferably C₁-C₄ alkyl, C₂-C₈ preferably C₂-C₄ alkenyl, C₂-C₈ preferably C₂-C₄ alkynyl, C₃-C₆ cycloalkyl, C₅-C₆ cycloalkenyl, C₅-C₁₂ bicycloalkyl, C₇-C₁₂ bicycloalkenyl, C₈-C₁₄ tricycloalkyl, and aromatic and heteroaromatic residues preferably six-membered aromatic cycles and five to six membered heteroaromatic cycles;

and wherein bicyclic and tricyclic residues include fused, bridged and spiro systems;

wherein said cycloalkyl, cycloalkenyl bicycloalkyl, bicycloalkenyl, tricycloalkyl, aromatic and heteroaromatic residues contained in the definition of R⁶ can optionally be linked through a C₁ alkylene or a C₂ alkylene or a C₃ alkylene linker to the N to which R⁶ is bound;

wherein all aromatic and heteroaromatic residues contained in the definition of R⁶ are unsubstituted or substituted with one or more substituents independently selected from: —F, —Cl, —Br, —I, —CN, —NCO, —NCS, —OH, —NH₂, —NO₂, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl, cycloalkyl, cycloalkenyl, bicycloalkyl, bicycloalkenyl and tricycloalkyl residues, and alkylene linkers contained in the definition of R⁶ are linear or branched, and are unsubstituted or substituted with one or more substituents independently selected from: —F, —Cl, —Br, —I, —CN, —NCO, —NCS, —OH, —NH₂, ═O, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl, cycloalkyl, cycloalkenyl, bicycloalkyl, bicycloalkenyl, tricycloalkyl and heteroaromatic residues, and alkylene linkers contained in the definition of R⁶ can contain one or more heteroatoms independently selected from O, S and N in replacement of a carbon atom;

wherein R⁶ is preferably —H, —CH₃, —CH₂CH₃, n-propyl, isopropyl, cyclopropyl, —CF₃ and —CF₂CF₃, benzyl, tert-butyl, phenyl, cyclohexyl, 1-phenylethyl, 2,2-dimethyl-1-phenylpropyl, (1-naphtyl)-methyl, 4-methoxybenzyl, 4-trifluoromethylbenzyl, tetrahydropyranyl;

wherein all alkyl, alkenyl, alkynyl, cycloalkyl, cycloalkenyl, bicycloalkyl, bicycloalkenyl, tricycloalkyl, aromatic and heteroaromatic residues contained in the definitions of R²-R⁶ and R⁹-R¹² can be partially or fully halogenated, particularly fluorinated, more particularly perfluorinated;

Y=—H, linear or branched C₁-C₆ alkyl, linear or branched C₂-C₆ alkenyl, linear or branched C₂-C₆ alkynyl, C₃-C₆ cycloalkyl, C₅-C₆ cycloalkenyl, —OH, linear or branched —OC₁-C₆ alkyl, linear or branched —OC₂-C₆ alkenyl, linear or branched —OC₂-C₆ alkynyl, —OC₃-C₆ cycloalkyl, —OC₅-C₆ cycloalkenyl, —CN, aromatic and heteroaromatic residues preferably six-membered aromatic cycles and five- to six-membered heteroaromatic cycles, —S(O)R¹³ and —S(O)₂R¹³ wherein R¹³ is selected from linear or branched C₁-C₆ alkyl, linear or branched C₂-C₆ alkenyl, linear or branched C₂-C₆ alkynyl, C₃-C₆ cycloalkyl, C₅-C₆ cycloalkenyl, —CF₃, and —C₆H₄CH₃;

wherein all cycloalkyl, cycloalkenyl, aromatic and heteroaromatic residues contained in the definition of Y can optionally be linked through a C₁ alkylene, or a C₂ alkylene, or a C₃ alkylene, or an —O—, or an —O—CH₂—, or an —O—CH₂—CH₂— linker to the N to which Y is bound;

wherein all aromatic and heteroaromatic residues contained in the definition of Y are unsubstituted or substituted with one or more substituents independently selected from: —F, —Cl, —Br, —I, —CN, —NCO, —NCS, —OH, —NH₂, —NO₂, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl, cycloalkyl, and cycloalkenyl residues, and alkylene linkers contained in the definition of Y are linear or branched, and are unsubstituted or substituted with one or more substituents independently selected from: —F, —Cl, —Br, —I, —CN, —NCO, —NCS, —OH, —NH₂, ═O, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl, cycloalkyl, cycloalkenyl and heteroaromatic residues, and alkylene linkers contained in the definition of Y can contain one or more heteroatoms independently selected from O, S and N in replacement of a carbon atom;

wherein all alkyl, alkenyl, alkynyl, cycloalkyl, cycloalkenyl, aromatic and heteroaromatic residues and alkylene linkers contained in the definition of Y can be partially or fully halogenated, particularly fluorinated, more particularly perfluorinated;

wherein Y is preferably —H, —CH₃, —CH₂CH₃, n-propyl, isopropyl, cyclopropyl, cyclohexyl, tetrahydropyranyl, —CF₃, —CF₂CF₃, —OH, —OCH₃, —OCH₂CH₃, —OCH₂(cyclopropyl), —CN, —S(O)C(CH₃)₃, —S(O)₂CH₃, —S(O)₂CF₃, —S(O)₂C₆H₄CH₃, —OCH₂C₆H₅ and —OC₆Hs; and for R⁶=—H or —CH₃ or benzyl, then Y is preferably —OH, —OCH₃, —OCH₂CH₃, —OCH₂(cyclopropyl);

wherein Y can form a ring structure together with R⁶, wherein the said ring structure including the N-atom of formula I is selected from three-membered rings, four-membered rings, five-membered rings, six-membered rings, from five- to twelve-membered bicyclic residues, from eight- to fourteen-membered tricyclic residues, and from heteroaromatic residues, wherein all rings, bicyclic, tricyclic and heteroaromatic residues can additionally contain one or more heteroatoms independently selected from O, S and N in replacement of a carbon atom contained in the ring structure, and wherein all rings, bicyclic, tricyclic and heteroaromatic residues are unsubstituted or substituted with one or more substituents independently selected from: —F, —Cl, —Br, —I, —CN, —NCO, —NCS, —OH, —OCH₃, —NH₂, —NHCH₃, —N(CH₃)₂, ═O, —CH₃, —CF₃, morpholinyl;

and wherein bicyclic and tricyclic residues include fused, bridged and spiro systems;

wherein the ring structure of Y together with R⁶ including the N-atom of formula I is preferably selected from aziridinyl, azetidinyl, pyrrolidinyl, piperidinyl, difluoropiperidinyl, morpholinyl, morpholinylazetidinyl, hydroxyazetidinyl, azetidinonyl, azetidinyl, difluoroazetidinyl, azaspirohexyl, azaspiroheptyl, difluoroazaspiroheptyl, hydroxyazaspiroheptyl, methylhydroxyazaspiroheptyl, trifluoromethylhydroxyazaspiroheptyl, azaspirooctyl, azaspirononyl, oxa-azaspiroheptyl, oxa-azaspirooctyl, oxa-azaspirononyl, thia-azaspiroheptyl, oxazolidinyl, tetrahydro-oxazinyl, isoxazolidinyl, oxazinane, isoxazolidine, piperazine;

and wherein the ring structure of Y together with R⁶ including the N-atom of formula I is even more preferably selected from:

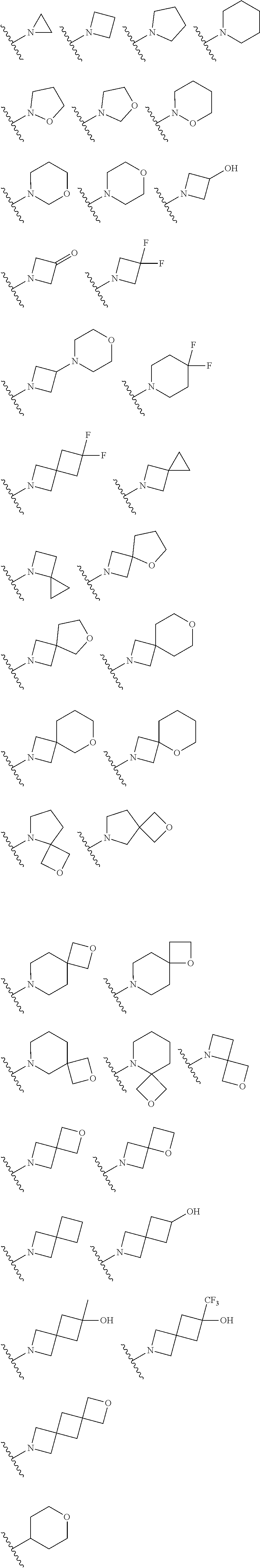

Z¹ and Z² are selected from the following groups:

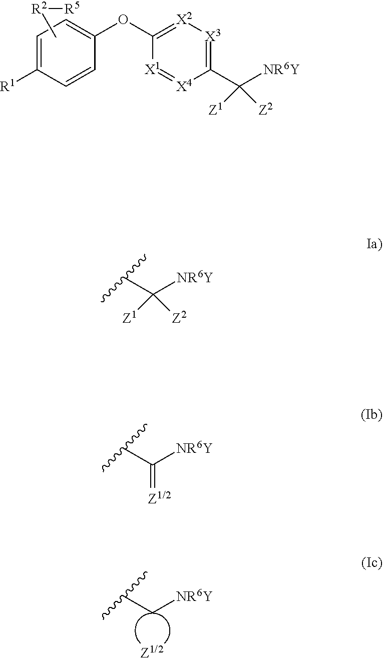

wherein Z¹ is selected from linear or branched C₁-C₃ alkyl preferably —CH₃, cyclopropyl, oxiranyl, N-methyl-aziridinyl, thiiranyl, —CN, —N₃, —CF₃, —CF₂CF₃, and wherein Z² is independently selected from —H and linear or branched C₁-C₃ alkyl preferably —CH₃, —CF₃, —CF₂CF₃ (general formula Ia);

wherein Z¹ is preferably —CH₃, —CF₃, —CN, cyclopropyl; and/or wherein Z² is preferably —H, —CH₃ and —CF₃; e.g.:

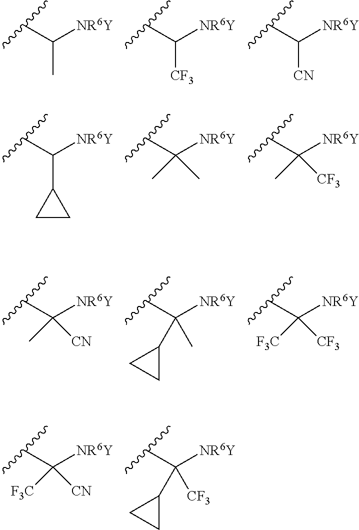

or wherein Z¹ and Z² are together ═O, ═S, ═NR¹⁴ (general formula Ib); wherein R¹⁴ is selected from —H, —OH, —OCH₃, —CN, —S(O)C(CH₃)₃, —S(O)₂CH₃, —S(O)₂CF₃, linear or branched C₁-C₃ alkyl preferably —CH₃, cyclopropyl, —CF₃, —CF₂CF₃, —CH₂CF₃, —C₆Hs, —CH₂C₆Hs;

wherein Z¹ and Z² are together preferably ═O, ═NR¹⁴; wherein R¹⁴ is preferably selected from —H, —CH₃, cyclopropyl, —OH, —OCH₃, —CN:

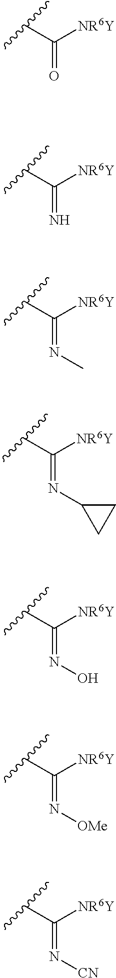

or wherein Z¹ and Z² form together a cyclic residue including the carbon atom to which they are bound (general formula Ic); wherein the cyclic residue is selected from three-membered rings, four-membered rings five-membered rings and six-membered rings, wherein all rings optionally can contain one or more heteroatoms independently selected from O, S and N in replacement of a carbon atom contained in the ring structure; wherein all rings are unsubstituted or substituted with one or more substituents independently selected from: —F, —Cl, —Br, —I, —CN, —NCO, —NCS, —OH, —OCH₃, —NH₂, —NHCH₃, —N(CH₃)₂, ═O, —CH₃ and —CF₃;

wherein Z¹ and Z² form together preferably a three membered or four membered cyclic residue including the carbon atom to which they are bound; wherein this cyclic residue is preferably selected from cyclopropyl, cyclobutyl, oxiranyl, oxetanyl, aziridinyl, azetidinyl and thietanyl; and

wherein this cyclic residue is optionally substituted preferably with —F, —OH, —OCH₃, —NH₂, —NHCH₃, —N(CH₃)₂, ═O, —CH₃ and —CF₃;

and wherein this cyclic residue is even more preferably selected from:

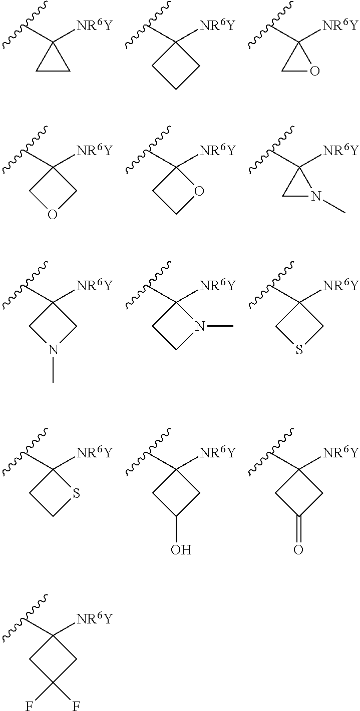

wherein all alkyl and cyclic residues contained in the definitions of Z¹ and Z² can be partially or fully halogenated, particularly fluorinated, more particularly perfluorinated.

Following preferred definitions of R¹-R¹⁴, X¹-X⁴, Z¹, Z² and Y may be optionally independently and/or in combination applied on all aspects including preferred and certain aspects, on all embodiments including preferred and certain embodiments, and on all subgenera as defined in the present invention:
1) R¹ preferably contains four or more preferably six or more and even more preferably seven or more carbon atoms;
2) R¹ is preferably selected from branched alkyl, alkenyl and alkynyl residues;
3) R¹ is preferably selected from cyclic, bicyclic and tricyclic structures, wherein bicyclic and tricyclic residues include fused, bridged and spiro systems;
4) R¹ preferably contains no heteroatom;
5) R¹ is preferably selected from cyclohexyl, norbornyl, bicyclooctyl, bicyclononyl, methylbicyclononyl, tricyclodecyl and most preferably adamantyl, e.g. 1-adamantyl and 2-adamantyl;
6) R¹ preferably contains one or more heteroatoms, preferably one, two or three heteroatoms independently selected from O, S and N in replacement of a carbon atom contained in R¹;
7) R¹ is preferably selected from tetrahydropyranyl, N-methylpiperidinyl, morpholinyl, 4-oxocyclohexyl, azabicycloheptyl, N-methylazabicycloheptyl, oxa-azabicycloheptyl, N-methyldiazabicycloheptyl, azabicyclooctyl, diazabicyclooctyl, N-methyldiazabicyclooctyl, oxa-azabicyclooctyl, azabicyclononyl, azaadamantyl and —O(adamantyl);
8) preferably two, or more preferably three of the substituents independently selected from R²-R⁵ are —H, i.e. preferably two and more preferably one of the substituents independently selected from R²-R⁵ are different from —H;
9) in the case that two of the substituents independently selected from R²-R⁵ are different from —H and are in ortho position relative to the ether bond, these two substituents are preferably different from —F, —Cl, —Br, —I and —NO₂ and more preferably different from each other;
10) the composition of ring atoms as defined by X¹-X⁴ is preferably selected from the cases that all of X¹-X⁴ are independently selected from CR⁹, CR¹⁰, CR¹¹, CR¹², or that one of X¹-X⁴ is N and the other three are independently selected from CR⁹, CR¹⁰, CR¹¹, CR¹², or that two of X¹-X⁴ are N and the other two are independently selected from CR⁹, CR¹⁰, CR¹¹, CR¹²; i.e. the aromatic or heteroaromatic ring is selected from benzene, pyridine, pyrimidine, pyridazine and pyrazine;
11) preferably two, or more preferably three of the substituents independently selected from R⁹-R¹² are —H, i.e. preferably two and more preferably one of the substituents independently selected from R⁹-R¹² are different from —H;
12) in the case that two of the substituents independently selected from R⁹-R¹² are different from —H and are in ortho position relative to the ether bond, these two substituents are preferably different from —F, —Cl, —Br, —I and —NO₂ and more preferably different from each other;
13) Y is preferably selected from residues as contained in the general definition of Y which are bound with an oxygen atom to the N to which Y is bound.

A preferred aspect of the present invention relates to compounds of general formula (Ib) and salts and solvates thereof, wherein Z¹ and Z² are together ═O and wherein at least one of R⁶ and Y is different from H,

and R¹-R⁵, R⁷-R¹³ and X¹-X⁴ are defined as in general formula (I) including their substitutions and preferred definitions.

A further preferred aspect of the present invention relates to compounds of general formula (I) and salts and solvates thereof, wherein Y is selected from residues as contained in the general definition of Y, which are bound with an oxygen atom to the N to which Y is bound,

and wherein Y is even more preferably —OH, —OCH₃, —OCH₂CH₃, —OCH₂(cyclopropyl), —OC₆H₅ and —OCH₂C₆H₅,

and R¹-R¹², R¹⁴, X¹-X⁴, Z¹ and Z² are defined as in general formula (I) including the substitutions and preferred definitions.

A further preferred aspect of the present invention relates to compounds of general formula (I) and salts and solvates thereof, wherein R¹ is selected from residues as contained in the general definition of R¹, which contain four or more preferably six or more and even more preferably seven or more carbon atoms,

and wherein R¹ contains no heteroatom,

and wherein R¹ is even more preferably selected from cyclic, bicyclic and tricyclic structures,

and wherein R¹ is even more preferably selected from cyclohexyl, norbornyl, bicyclooctyl, bicyclononyl, methylbicyclononyl, tricyclodecyl and adamantyl,

and wherein R¹ is most preferably adamantyl,

and R²-R⁶, R⁹-R¹⁴, X¹-X⁴, Z¹, Z² and Y are defined as in general formula (I) including the substitutions and preferred definitions.

A further preferred aspect of the present invention relates to compounds of general formula (I) and salts and solvates thereof, wherein R¹ is selected from residues as contained in the general definition of R¹, which contain four or more, preferably six or more and even more preferably seven or more carbon atoms,

and wherein R¹ contains one or more preferably one to two heteroatoms independently selected from O, S and N in replacement of a carbon atom contained in R¹,

and wherein R¹ is even more preferably selected from cyclic, bicyclic and tricyclic structures, or wherein R¹ is selected from residues containing cyclic, bicyclic and tricyclic structures,

and wherein R¹ is even more preferably selected from tetrahydropyranyl, N-methylpiperidinyl, morpholinyl, 4-oxocyclohexyl, azabicycloheptyl, N-methylazabicycloheptyl, oxa-azabicycloheptyl, N-methyldiazabicycloheptyl, azabicyclooctyl, diazabicyclooctyl, N-methyldiazabicyclooctyl, oxa-azabicyclooctyl, azabicyclononyl, aza-adamantyl and —O(adamantyl),

and wherein R¹ is most preferably tetrahydropyranyl, N-methylpiperidinyl, morpholinyl, 4-oxocyclohexyl, azabicyclooctyl, aza-adamantyl and —O(adamantyl),

and R²-R¹⁴, X¹-X⁴, Z¹, Z² and Y are defined as in general formula (I) including the substitutions and preferred definitions.

A further preferred aspect of the present invention relates to compounds of general formula (I) and salts and solvates thereof, which fall under the scope of the herein defined subgenera:
S.1 If Z¹ and Z² are defined as in general formula (I) including their substitutions and preferred definitions, with the proviso that Z¹ and Z² are different from being together ═O or ═S,
   then R¹-R¹³, X¹-X⁴, and Y are defined as in general formula (I) including their substitutions and preferred definitions.
S.2 If R⁶ is defined as in general formula (I) including the substitutions and preferred definitions, with the proviso that R⁶ is different from —H, or linear unsubstituted or branched unsubstituted C₁-C₆ alkyl,
   then R¹-R⁵, R⁷-R¹⁴, X¹-X⁴, Y, Z¹ and Z² are defined as in general formula (I) including their substitutions and preferred definitions.
S.3 If Y is defined as in general formula (I) including the substitutions and preferred definitions, with the proviso that Y is different from —H, linear unsubstituted or branched unsubstituted C₁-C₆ alkyl, or —OH,
   then R¹-R¹⁴, X¹-X⁴, Z¹ and Z² are defined as in general formula (I) including their substitutions and preferred definitions.
S.4 If Z¹ and Z² are together ═O or ═S, and Y is —OH,
   then R⁶ is defined as in general formula (I) including the substitutions and preferred definitions, with the proviso that R⁶ is different from —H,
   and then R¹-R⁵, R⁷-R¹² and X¹-X⁴ are defined as in general formula (I) including their substitutions and preferred definitions.
S.5 If Z¹ and Z² are together ═O or ═S, and R⁶ is —H, and Y is —H, linear unsubstituted or branched unsubstituted C₁-C₆ alkyl, or —OH,
   or if Z¹ and Z² are together ═O or ═S, and R⁶ is —H, or linear unsubstituted or branched unsubstituted C₁-C₆ alkyl, and Y is —H, linear unsubstituted or branched unsubstituted C₁-C₆ alkyl,
   then R¹=C₁-C₁₂ preferably C₁-C₆ alkyl, C₂-C₁₂ preferably C₂-C₆ alkenyl, C₂-C₁₂ preferably C₂-C₆ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, —OC₁-C₁₂ preferably —OC₁-C₆ alkyl, —OC₂-C₁₂ preferably —OC₂-C₆ alkenyl, —OC₂-C₁₂ preferably —OC₂-C₆ alkynyl, —OC₃-C₈ cycloalkyl, —OC₅-C₈ cycloalkenyl, —OC₅-C₁₂ bicycloalkyl, —OC₇-C₁₂ bicycloalkenyl, —OC₈-C₁₄ tricycloalkyl, —SC₁-C₁₂ preferably —SC₁-C₆ alkyl, —SC₂-C₁₂ preferably —SC₂-C₆ alkenyl, —SC₂-C₁₂ preferably —SC₂-C₆ alkynyl, —SC₃-C₈ cycloalkyl, —SC₅-C₈ cycloalkenyl, —SC₅-C₁₂ bicycloalkyl, —SC₇-C₁₂ bicycloalkenyl, —SC₅-C₁₄ tricycloalkyl, —NHR⁷ or —NR⁷R⁸ wherein R⁷ and R⁸ are independently from each other selected from: C₁-C₁₂ preferably C₁-C₆ alkyl, C₂-C₁₂ preferably C₂-C₆ alkenyl, C₂-C₁₂ preferably C₂-C₆ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, or wherein R⁷ can form a ring structure together with Re wherein the said ring structure including the N-atom is selected from three to eight membered cyclic structures or five to twelve membered bicyclic structures and wherein all said ring structures can additionally contain one or more heteroatoms independently selected from O, S and N in replacement of a carbon atom contained in the ring structure;
   wherein all C₁-C₁₂ alkyl, C₂-C₈ alkenyl, C₂-C₈ alkynyl, C₃-C₈ cycloalkyl, C₅-C₈ cycloalkenyl, norbornyl and adamantyl residues are linear or branched, and are substituted with one or more substituents, here referred to as side-substituents, independently selected from: —OH, —NH₂, —NO₂, ═O, C₃-C₈ cycloalkyl, C₅-C₈ cycloalkenyl, C₅-C₁₂ bicycloalkyl including norbornyl, C₇-C₁₂ bicycloalkenyl, C₈-C₁₄ tricycloalkyl including adamantyl, linear or branched —OC₁-C₅ alkyl such as —OCH₃, —OC₃-C₅ cycloalkyl such as —O (cyclopropyl), linear or branched —NH(C₁-C₅ alkyl), linear or branched —N(C₁-C₅ alkyl)(C₁-C₅ alkyl), —NH(C₃-C₅ cycloalkyl) such as —NH(cyclopropyl), —N(C₃-C₅ cycloalkyl)(C₃-C₅ cycloalkyl), linear or branched —N(C₁-C₅ alkyl)(C₃-C₅ cycloalkyl); and wherein all said C₁-C₁₂ alkyl, C₂-C₈ alkenyl, C₂-C₈ alkynyl, C₃-C₈ cycloalkyl, C₅-C₈ cycloalkenyl, adamantyl or norbornyl residues can optionally contain in addition one or more substituents independently selected from —F, —Cl, —Br, —I, —CN, —NCO, —NCS;
   and all C₉-C₁₂ alkenyl, C₉-C₁₂ alkynyl, —OC₁-C₁₂ alkyl, —OC₂-C₁₂ alkenyl, —OC₂-C₁₂ alkynyl, —SC₁-C₁₂ alkyl, —SC₂-C₁₂ alkenyl, —SC₂-C₁₂ alkynyl, and all alkyl, alkenyl and alkynyl residues contained in the definition of R⁷ and R⁸ are linear or branched, and are unsubstituted or substituted with one or more substituents, here referred to as side-substituents, independently selected from: —F, —Cl, —Br, —I, —CN, —NCO, —NCS, —OH, —NH₂, —NO₂, ═O, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, linear or branched —OC₁-C₅ alkyl such as —OCH₃, —OC₃-C₈ cycloalkyl such as —O (cyclopropyl), linear or branched —NH(C₁-C₅ alkyl), linear or branched —N(C₁-C₅ alkyl)(C₁-C₅ alkyl), —NH(C₃-C₅ cycloalkyl) such as —NH(cyclopropyl), —N(C₃-C₅ cycloalkyl)(C₃-C₅ cycloalkyl), linear or branched —N(C₁-C₅ alkyl)(C₃-C₅ cycloalkyl);
   wherein all —OC₃-C₈ cycloalkyl, —OC₅-C₈ cycloalkenyl, —SC₃-C₈ cycloalkyl, —SC₅-C₈ cycloalkenyl residues, and all cycloalkyl and cycloalkenyl residues contained in the definition of R⁷ and R⁸ and contained in the selection of the named side-substituents, and all bicyclic and tricyclic structures including bicycloalkyl, bicycloalkenyl and tricycloalkyl residues contained in the definitions of R¹, R⁷ and R⁸, with the proviso that they are different from adamantyl and norbornyl, are unsubstituted or substituted with one or more substituents independently selected from: —F, —Cl, —Br, —I, —CN, —NCO, —NCS, —OH, —NH₂, —NO₂, ═O, linear or branched C₁-C₅ alkyl such as —CH₃, linear or branched —OC₁-C₅ alkyl such as —OCH₃, linear or branched —NH(C₁-C₅ alkyl), linear or branched —N(C₁-C₅ alkyl)(C₁-C₅ alkyl), —NH(C₃-C₅ cycloalkyl) such as —NH(cyclopropyl), —N(C₃-C₅ cycloalkyl)(C₃-C₅ cycloalkyl), linear or branched —N(C₁-C₅ alkyl)(C₃-C₅ cycloalkyl);

wherein all alkyl, alkenyl, alkynyl, cycloalkyl, cycloalkenyl, bicycloalkyl, bicycloalkenyl and tricycloalkyl residues contained in the definitions R⁷ and R⁸ can contain one or more heteroatoms independently selected from O, S and N in replacement of a carbon atom;

and wherein all alkyl, alkenyl, alkynyl, cycloalkyl, cycloalkenyl, bicycloalkyl, bicycloalkenyl and tricycloalkyl residues contained in the definition of R¹ can contain one or more heteroatoms independently selected from O, S and N in replacement of a carbon atom, with the optional proviso that the combination of the said heteroatoms in a terminal position is different from the residues —CN, —NCO, —NCS and —N₃ if not explicitly contained in the definition of R¹;

wherein all alkyl, alkenyl, alkynyl, cycloalkyl, cycloalkenyl, bicycloalkyl, bicycloalkenyl and tricycloalkyl residues contained in the definitions of R¹, R⁷ and R⁸ can be partially or fully halogenated, particularly fluorinated, more particularly perfluorinated;
wherein bicyclic and tricyclic residues include fused, bridged and spiro systems;
and then R²-R⁵, R⁹-R¹² and X¹-X⁴ are defined as in general formula (I) including their substitutions and preferred definitions.
S.6 If Z¹ and Z² are together ═O or ═S, and R⁶ is —H, and Y is —H, linear unsubstituted or branched unsubstituted C₁-C₆ alkyl, or —OH,
   or if Z¹ and Z² are together ═O or ═S, and R⁶ is —H, or linear unsubstituted or branched unsubstituted C₁-C₆ alkyl, and Y is —H, linear unsubstituted or branched unsubstituted C₁-C₆ alkyl,
   then R² is selected from —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all C₁-C₄ alkyl, C₂-C₄ alkenyl, C₂-C₄ alkynyl, and C₃-C₄ cycloalkyl residues are substituted with one or more substituents independently selected from —OH, —OCH₃, —OCF₃, —NH₂, —NHCH₃ and —N(CH₃)₂;
   wherein the C₅-C₆ cycloalkyl residues are unsubstituted or substituted with one or more substituents independently selected from —F, —Cl, —Br, —I, —CH₃, —CF₃, —OH, —OCH₃, —OCF₃, —NH₂, —NHCH₃ and —N(CH₃)₂;
   wherein all alkyl, alkenyl, alkynyl and cycloalkyl residues contained in the definition of R² can contain one or more heteroatoms independently selected from O, S and N in replacement of a carbon atom;
   and R³-R⁵ are independently from each other selected from —H, —F, —Cl, —Br, —I, —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all alkyl, alkenyl, alkynyl and cycloalkyl residues contained in the definitions of R³-R⁵ are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂;
   wherein all alkyl, alkenyl, alkynyl and cycloalkyl residues contained in the definitions of R³-R⁵ can contain one or more heteroatoms independently selected from O, S and N in replacement of a carbon atom;
   and then R¹, R⁷-R¹² and X¹-X⁴ are defined as in general formula (I) including their substitutions and preferred definitions.
S.7 If Z¹ and Z² are together ═O or ═S, and R⁶ is —H, and Y is —H, linear unsubstituted or branched unsubstituted C₁-C₆ alkyl, or —OH,
   or if Z¹ and Z² are together ═O or ═S, and R⁶ is —H, or linear unsubstituted or branched unsubstituted C₁-C₆ alkyl, and Y is —H, linear unsubstituted or branched unsubstituted C₁-C₆ alkyl,
   then X¹ is CR⁹
   and R⁹ is selected from —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all C₁-C₄ alkyl, C₂-C₄ alkenyl, C₂-C₄ alkynyl, and C₃-C₄ cycloalkyl residues are substituted with one or more substituents independently selected from —OH, —OCH₃, —OCF₃, —NH₂, —NHCH₃ and —N(CH₃)₂;
   wherein the C₅-C₆ cycloalkyl residues are unsubstituted or substituted with one or more substituents independently selected from —F, —Cl, —Br, —I, —CH₃, —CF₃, —OH, —OCH₃, —OCF₃, —NH₂, —NHCH₃ and —N(CH₃)₂;
   wherein all alkyl, alkenyl, alkynyl and cycloalkyl residues contained in the definition of R⁹ can contain one or more heteroatoms independently selected from O, S and N in replacement of a carbon atom;
   and then R¹-R⁵, R⁷, R⁸, R¹⁰-R¹² and X²-X⁴ are defined as in general formula (I) including their substitutions and preferred definitions.
S.8 If Z¹ and Z² are together ═O or ═S, and R⁶ is —H, and Y is —H, linear unsubstituted or branched unsubstituted C₁-C₆ alkyl, or —OH,
   or if Z¹ and Z² are together ═O or ═S, and R⁶ is —H, or linear unsubstituted or branched unsubstituted C₁-C₆ alkyl, and Y is —H, linear unsubstituted or branched unsubstituted C₁-C₆ alkyl,
   then X² is CR⁹
   and R⁹ is selected from —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all C₁-C₄ alkyl, C₂-C₄ alkenyl, C₂-C₄ alkynyl, and C₃-C₄ cycloalkyl residues are substituted with one or more substituents independently selected from —OH, —OCH₃, —OCF₃, —NH₂, —NHCH₃ and —N(CH₃)₂;
   wherein the C₅-C₆ cycloalkyl residues are unsubstituted or substituted with one or more substituents independently selected from —F, —Cl, —Br, —I, —CH₃, —CF₃, —OH, —OCH₃, —OCF₃, —NH₂, —NHCH₃ and —N(CH₃)₂;
   wherein all alkyl, alkenyl, alkynyl and cycloalkyl residues contained in the definition of R⁹ can contain one or more heteroatoms independently selected from O, S and N in replacement of a carbon atom;
   and then R¹-R⁵, R⁷, R⁸, R¹⁰-R¹², X¹, X³ and X⁴ are defined as in general formula (I) including their substitutions and preferred definitions.
S.9 If Z¹ and Z² are together ═O or ═S, and R⁶ is —H, and Y is —H, linear unsubstituted or branched unsubstituted C₁-C₆ alkyl, or —OH,
   or if Z¹ and Z² are together ═O or ═S, and R⁶ is —H, or linear unsubstituted or branched unsubstituted C₁-C₆ alkyl, and Y is —H, linear unsubstituted or branched unsubstituted C₁-C₆ alkyl,
   then X³ is CR⁹
   and R⁹ is selected from —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all C₁-C₄ alkyl, C₂-C₄ alkenyl, C₂-C₄ alkynyl, and C₃-C₄ cycloalkyl residues are substituted with one or more substituents independently selected from —OH, —OCH₃, —OCF₃, —NH₂, —NHCH₃ and —N(CH₃)₂;
   wherein the C₅-C₆ cycloalkyl residues are unsubstituted or substituted with one or more substituents independently selected from —F, —Cl, —Br, —I, —CH₃, —CF₃, —OH, —OCH₃, —OCF₃, —NH₂, —NHCH₃ and —N(CH₃)₂;
   wherein all alkyl, alkenyl, alkynyl and cycloalkyl residues contained in the definition of R⁹ can contain one or more heteroatoms independently selected from O, S and N in replacement of a carbon atom;
   and then R¹-R⁵, R⁷, R⁸, R¹⁰-R¹², X¹, X² and X⁴ are defined as in general formula (I) including their substitutions and preferred definitions.
S.10 If Z¹ and Z² are together ═O or ═S, and R⁶ is —H, and Y is —H, linear unsubstituted or branched unsubstituted C₁-C₆ alkyl, or —OH,
   or if Z¹ and Z² are together ═O or ═S, and R⁶ is —H, or linear unsubstituted or branched unsubstituted C₁-C₆ alkyl, and Y is —H, linear unsubstituted or branched unsubstituted C₁-C₆ alkyl,
   then X⁴ is CR⁹
   and R⁹ is selected from —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all C₁-C₄ alkyl, C₂-C₄ alkenyl, C₂-C₄ alkynyl, and C₃-C₄ cycloalkyl residues are substituted with one or more substituents independently selected from —OH, —OCH₃, —OCF₃, —NH₂, —NHCH₃ and —N(CH₃)₂;
   wherein the C₅-C₆ cycloalkyl residues are unsubstituted or substituted with one or more substituents independently selected from —F, —Cl, —Br, —I, —CH₃, —CF₃, —OH, —OCH₃, —OCF₃, —NH₂, —NHCH₃ and —N(CH₃)₂;
   wherein all alkyl, alkenyl, alkynyl and cycloalkyl residues contained in the definition of R⁹ can contain one or more heteroatoms independently selected from O, S and N in replacement of a carbon atom;
   and then R¹-R⁵, R⁷, R⁸, R¹⁰-R¹² and X¹-X³ are defined as in general formula (I) including their substitutions and preferred definitions.
S.11 If Z¹ and Z² are together ═O or ═S, and R⁶ is —H, and Y is —H, linear unsubstituted or branched unsubstituted C₁-C₆ alkyl, or —OH,
   or if Z¹ and Z² are together ═O or ═S, and R⁶ is —H, or linear unsubstituted or branched unsubstituted C₁-C₆ alkyl, and Y is —H, linear unsubstituted or branched unsubstituted C₁-C₆ alkyl,
   then X¹, X² and X³ are each N and then R¹-R⁵, R⁷-R¹², and X⁴ are defined as in general formula (I) including their substitutions and preferred definitions.
S.12 If Z¹ and Z² are together ═O or ═S, and R⁶ is —H, and Y is —H, linear unsubstituted or branched unsubstituted C₁-C₆ alkyl, or —OH,
   or if Z¹ and Z² are together ═O or ═S, and R⁶ is —H, or linear unsubstituted or branched unsubstituted C₁-C₆ alkyl, and Y is —H, linear unsubstituted or branched unsubstituted C₁-C₆ alkyl,
   then X¹, X² and X⁴ are each N
   and then R¹-R⁵, R⁷-R¹², and X³ are defined as in general formula (I) including their substitutions and preferred definitions.
S.13 If Z¹ and Z² are together ═O or ═S, and R⁶ is —H, and Y is —H, linear unsubstituted or branched unsubstituted C₁-C₆ alkyl, or —OH,
   or if Z¹ and Z² are together ═O or ═S, and R⁶ is —H, or linear unsubstituted or branched unsubstituted C₁-C₆ alkyl, and Y is —H, linear unsubstituted or branched unsubstituted C₁-C₆ alkyl,
   then X¹, X³ and X⁴ are each N and then R¹-R⁵, R⁷-R¹², and X² are defined as in general formula (I) including their substitutions and preferred definitions.
S.14 If Z¹ and Z² are together ═O or ═S, and R⁶ is —H, and Y is —H, linear unsubstituted or branched unsubstituted C₁-C₆ alkyl, or —OH,
   or if Z¹ and Z² are together ═O or ═S, and R⁶ is —H, or linear unsubstituted or branched unsubstituted C₁-C₆ alkyl, and Y is —H, linear unsubstituted or branched unsubstituted C₁-C₆ alkyl,
   then X², X³ and X⁴ are each N
   and then R¹-R⁵, R⁷-R¹², and X¹ are defined as in general formula (I) including their substitutions and preferred definitions.
S.15 If R¹ is defined as in general formula (I) including the substitutions and preferred definitions, with the proviso that R¹ contains one or more heteroatoms independently selected from O, S and N, with the proviso that the combination of the said heteroatoms in a terminal position is different from the residues —CN, —NCO, —NCS,
   then R²-R¹⁴, X¹-X⁴, Y, Z¹ and Z² are defined as in general formula (I) including their substitutions and preferred definitions.
S.16 If Z¹ and Z² are defined as in general formula (I) including their substitutions and preferred definitions, with the proviso that Z¹ and Z² are different from being together ═O,
   then R¹-R¹⁴, X¹-X⁴, and Y are defined as in general formula (I) including their substitutions and preferred definitions.
S.17 If R⁶ is defined as in general formula (I) including the substitutions and preferred definitions, with the proviso that R⁶ is different from —H, or C₁-C₆ alkyl, or C₃-C₆ cycloalkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then R¹-R⁵, R⁷-R¹⁴, X¹-X⁴, Y, Z¹ and Z² are defined as in general formula (I) including their substitutions and preferred definitions.
S.18 If Y is defined as in general formula (I) including the substitutions and preferred definitions, with the proviso that Y is different from —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then R¹-R¹⁴, X¹-X⁴, Z¹ and Z² are defined as in general formula (I) including their substitutions and preferred definitions.
S.19 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then R¹=C₁-C₁₂ preferably C₁-C₆ alkyl, C₂-C₁₂ preferably C₂-C₆ alkenyl, C₂-C₁₂ preferably C₂-C₆ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, —OC₁-C₁₂ preferably —OC₁-C₆ alkyl, —OC₂-C₁₂ preferably —OC₂-C₆ alkenyl, —OC₂-C₁₂ preferably —OC₂-C₆ alkynyl, —OC₃-C₈ cycloalkyl, —OC₅-C₈ cycloalkenyl, —OC₅-C₁₂ bicycloalkyl, —OC₇-C₁₂ bicycloalkenyl, —OC₅-C₁₄ tricycloalkyl, —SC₁-C₁₂ preferably —SC₁-C₆ alkyl, —SC₂-C₁₂ preferably —SC₂-C₆ alkenyl, —SC₂-C₁₂ preferably —SC₂-C₆ alkynyl, —SC₃-C₈ cycloalkyl, —SC₅-C₈ cycloalkenyl, —SC₅-C₁₂ bicycloalkyl, —SC₇-C₁₂ bicycloalkenyl, —SC₅-C₁₄ tricycloalkyl, —NHR⁷ or —NR⁷R⁸ wherein R⁷ and R⁸ are independently from each other selected from: C₁-C₁₂ preferably C₁-C₆ alkyl, C₂-C₁₂ preferably C₂-C₆ alkenyl, C₂-C₁₂ preferably C₂-C₆ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, or wherein R⁷ can form a ring structure together with R⁸ wherein the said ring structure including the N-atom is selected from three to eight membered cyclic structures or five to twelve membered bicyclic structures and wherein all said ring structures can additionally contain one or more heteroatoms independently selected from O, S and N in replacement of a carbon atom contained in the ring structure;
   wherein all C₁-C₁₂ alkyl, C₂-C₁₂ alkenyl, C₂-C₁₂ alkynyl, C₃-C₈ cycloalkyl, C₅-C₈ cycloalkenyl, C₅-C₁₂ bicycloalkyl, C₇-C₁₂ bicycloalkenyl and C₈-C₁₄ tricycloalkyl residues are linear or branched, and are substituted with one or more substituents, here referred to as side-substituents, independently selected from: —OH, —NH₂, —NO₂, ═O, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, linear or branched —OC₄-C₈ alkyl, —OC₃-C₈ cycloalkyl such as —O(cyclopropyl), linear or branched —NH(C₁-C₅ alkyl), linear or branched —N(C₁-C₅ alkyl)(C₁-C₅ alkyl), —NH(C₃-C₅ cycloalkyl) such as —NH(cyclopropyl), —N(C₃-C₅ cycloalkyl)(C₃-C₅ cycloalkyl), linear or branched —N(C₁-C₅ alkyl)(C₃-C₅ cycloalkyl); and
   wherein all said C₁-C₁₂ alkyl, C₂-C₁₂ alkenyl, C₂-C₁₂ alkynyl, C₃-C₈ cycloalkyl, C₅-C₈ cycloalkenyl, C₅-C₁₂ bicycloalkyl, C₇-C₁₂ bicycloalkenyl and C₈-C₁₄ tricycloalkyl residues can optionally contain in addition one or more substituents independently selected from —F, —Cl, —Br, —I, —CN, —NCO, —NCS;
   and all —OC₁-C₁₂ alkyl, —OC₂-C₁₂ alkenyl, —OC₂-C₁₂ alkynyl, —SC₁-C₁₂ alkyl, —SC₂-C₁₂ alkenyl, —SC₂-C₁₂ alkynyl, and all alkyl, alkenyl and alkynyl residues contained in the definition of R⁷ and R⁸ are linear or branched, and are unsubstituted or substituted with one or more substituents, here referred to as side-substituents, independently selected from: —F, —Cl, —Br, —I, —CN, —NCO, —NCS, —OH, —NH₂, —NO₂, ═O, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, linear or branched —OC₁-C₅ alkyl such as —OCH₃, —OC₃-C₅ cycloalkyl such as —O (cyclopropyl), linear or branched —NH(C₁-C₅ alkyl), linear or branched —N(C₁-C₅ alkyl)(C₁-C₅ alkyl), —NH(C₃-C₅ cycloalkyl) such as —NH(cyclopropyl), —N(C₃-C₅ cycloalkyl)(C₃-C₅ cycloalkyl), linear or branched —N(C₁-C₅ alkyl)(C₃-C₅ cycloalkyl);
   wherein all —OC₃-C₈ cycloalkyl, —OC₅-C₈ cycloalkenyl, —OC₅-C₁₂ bicycloalkyl, —OC₇-C₁₂ bicycloalkenyl, —OC₅-C₁₄ tricycloalkyl, —SC₃-C₈ cycloalkyl, —SC₅-C₈ cycloalkenyl, —SC₅-C₁₂ bicycloalkyl, —SC₇-C₁₂ bicycloalkenyl, —SC₈-C₁₄ tricycloalkyl, residues, and all cycloalkyl, cycloalkenyl, bicycloalkyl, bicycloalkenyl and tricycloalkyl residues contained in the definition of R⁷ and R⁸ and contained in the selection of the named side-substituents, are unsubstituted or substituted with one or more substituents independently selected from: —F, —Cl, —Br, —I, —CN, —NCO, —NCS, —OH, —NH₂, —NO₂, ═O, linear or branched C₁-C₅ alkyl such as —CH₃, linear or branched —OC₁-C₅ alkyl such as —OCH₃, linear or branched —NH(C₁-C₅ alkyl), linear or branched —N(C₁-C₅ alkyl)(C₁-C₅ alkyl), —NH(C₃-C₅ cycloalkyl) such as —NH(cyclopropyl), —N(C₃-C₅ cycloalkyl)(C₃-C₅ cycloalkyl), linear or branched —N(C₁-C₅ alkyl)(C₃-C₅ cycloalkyl);
   wherein all alkyl, alkenyl, alkynyl, cycloalkyl, cycloalkenyl, bicycloalkyl, bicycloalkenyl and tricycloalkyl residues contained in the definitions of R⁷ and R⁸ can contain one or more heteroatoms independently selected from O, S and N in replacement of a carbon atom;
   and wherein all alkyl, alkenyl, alkynyl, cycloalkyl, cycloalkenyl, bicycloalkyl, bicycloalkenyl and tricycloalkyl residues contained in the definition of R¹ can contain one or more heteroatoms independently selected from O, S and N in replacement of a carbon atom, with the proviso that the combination of the said heteroatoms in a terminal position is different from the residues —CN, —NCO, —NCS and —OC₁-C₃ alkyl if not explicitly contained in the definition of R¹;
   wherein bicyclic and tricyclic residues include fused, bridged and spiro systems;
   and then R²-R⁵, R⁹-R¹² and X¹-X⁴ are defined as in general formula (I) including their substitutions and preferred definitions.
S.20 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then R² is selected from —F, —Cl, —Br, —I, —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all alkyl, alkenyl, alkynyl and cycloalkyl residues contained in the definition of R² are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂;
   wherein all alkyl, alkenyl, alkynyl and cycloalkyl residues contained in the definition of R² can contain one or more heteroatoms independently selected from O, S and N in replacement of a carbon atom;
   and then R¹, R³-R⁵, R⁷-R¹² and X¹-X⁴ are defined as in general formula (I) including their substitutions and preferred definitions.
S.21 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then X¹ is CR⁹
   and R⁹ is selected from —Cl, —Br, —I, CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all alkyl, alkenyl, alkynyl and cycloalkyl residues contained in the definition of R⁹ are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂;
   wherein all alkyl, alkenyl, alkynyl and cycloalkyl residues contained in the definition of R⁹ can contain one or more heteroatoms independently selected from O, S and N in replacement of a carbon atom;
   and then R¹-R⁵, R⁷, R⁸, R¹⁰-R¹² and X²-X⁴ are defined as in general formula (I) including their substitutions and preferred definitions.
S.22 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then X² is CR⁹
   and R⁹ is selected from —Cl, —Br, —I, CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all alkyl, alkenyl, alkynyl and cycloalkyl residues contained in the definition of R⁹ are unsubstituted or substituted with one or more substituents independently selected from —F, —Cl, —Br, —I, —CH₃, —CF₃, —OH and —OCH₃, —OCF₃, —NH₂, NHCH₃, —N(CH₃)₂;
   wherein all alkyl, alkenyl, alkynyl and cycloalkyl residues contained in the definition of R⁹ can contain one or more heteroatoms independently selected from O, S and N in replacement of a carbon atom;
   and then R¹-R⁵, R⁷, R⁸, R¹⁰-R¹², X¹, X³ and X⁴ are defined as in general formula (I) including their substitutions and preferred definitions.
S.23 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then X³ is CR⁹
   and R⁹ is selected from —F, —Cl, —Br, —I, CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all alkyl, alkenyl, alkynyl and cycloalkyl residues contained in the definition of R⁹ are unsubstituted or substituted with one or more substituents independently selected from —F, —Cl, —Br, —I, —CH₃, —CF₃, —OH and —OCH₃, —OCF₃, —NH₂, NHCH₃, —N(CH₃)₂;
   wherein all alkyl, alkenyl, alkynyl and cycloalkyl residues contained in the definition of R⁹ can contain one or more heteroatoms independently selected from O, S and N in replacement of a carbon atom;
   and then R¹-R⁵, R⁷, R⁸, R¹⁰-R¹², X¹, X² and X⁴ are defined as in general formula (I) including their substitutions and preferred definitions.
S.24 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then X⁴ is CR⁹
   and R⁹ is selected from —F, —Cl, —Br, —I, CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
   wherein all alkyl, alkenyl, alkynyl and cycloalkyl residues contained in the definition of R⁹ are unsubstituted or substituted with one or more substituents independently selected from —F, —Cl, —Br, —I, —CH₃, —CF₃, —OH and —OCH₃, —OCF₃, —NH₂, NHCH₃, —N(CH₃)₂;
   wherein all alkyl, alkenyl, alkynyl and cycloalkyl residues contained in the definition of R⁹ can contain one or more heteroatoms independently selected from O, S and N in replacement of a carbon atom;
   and then R¹-R⁵, R⁷, R⁸, R¹⁰-R¹² and X¹-X³ are defined as in general formula (I) including their substitutions and preferred definitions.
S.25 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then X³ is N
   and then R¹-R⁵, R⁷-R¹², X¹, X² and X⁴ are defined as in general formula (I) including their substitutions and preferred definitions.
S.26 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then X⁴ is N
   and then R¹-R⁵, R⁷-R¹² and X¹-X³ are defined as in general formula (I) including their substitutions and preferred definitions.
S.27 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then X¹ and X² are each N
   and then R¹-R⁵, R⁷-R¹², X³ and X⁴ are defined as in general formula (I) including their substitutions and preferred definitions.
S.28 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then X¹ and X³ are each N
   and then R¹-R⁵, R⁷-R¹², X² and X⁴ are defined as in general formula (I) including their substitutions and preferred definitions.
S.29 If Z¹ and Z² are together ═O, and R⁶ is —H, or C₁-C₆ alkyl, or C₃-C₆ cycloalkyl, and Y is —H, or C₁-C₆ alkyl, or C₃-C₆ cycloalkyl, or —OH, or —OC₁-C₆ alkyl,
   wherein all said C₁-C₆ alkyl residues are linear or branched, and unsubstituted or substituted with one or more substituents independently selected from —F, —Cl, —Br, —I, C₁-C₃ alkyl and —OC₁-C₃ alkyl,
   and wherein all said C₃-C₆ cycloalkyl residues are unsubstituted or substituted with one or more substituents independently selected from —F, —Cl, —Br, —I, C₁-C₃ alkyl and —OC₁-C₃ alkyl,
   and wherein all said alkyl and cycloalkyl residues can optionally be halogenated or perhalogenated,
   then X¹ and X⁴ are each N
   and then R¹-R⁵, R⁷-R¹², X² and X³ are defined as in general formula (I) including their substitutions and preferred definitions.
S.30 If R¹ is defined as in general formula (I) including the substitutions and preferred definitions, with the proviso that R¹ is different from C₃-C₈ cycloalkyl,
   wherein the said C₃-C₈ cycloalkyl residues are unsubstituted or substituted with one or more substituents independently selected from —F, —Cl, —Br, —I, —CN, —NCO, —NCS, C₁-C₃ alkyl and —OC₁-C₃ alkyl,
   wherein the said C₃-C₈ cycloalkyl residues can optionally be perhalogenated
   and wherein the said C₃-C₈ cycloalkyl residues are substituted at the same carbon atom, which is bound to the phenyl ring as defined in general formula (I), with a substituent selected from C₁-C₁₂ alkyl, C₂-C₁₂ alkenyl, C₂-C₁₂ alkynyl, C₃-C₈ cycloalkyl or C₅-C₈ cycloalkenyl,
   wherein all said alkyl, alkenyl and alkynyl residues are linear or branched, and unsubstituted or substituted with one or more substituents independently selected from —F, —Cl, —Br, —I, —CN, —NCO, —NCS and —OC₁-C₃ alkyl,
   wherein all said cycloalkyl and cycloalkenyl residues are unsubstituted or substituted with one or more substituents independently selected from —F, —Cl, —Br, —I, —CN, —NCO, —NCS, C₁-C₃ alkyl and —OC₁-C₃ alkyl,
   and wherein all said alkyl, alkenyl, alkynyl, cycloalkyl and cycloalkenyl residues can optionally be perhalogenated,
   then R²-R¹⁴, X¹-X⁴, Y, Z¹ and Z² are defined as in general formula (I) including their substitutions and preferred definitions.

In a certain embodiment, the present invention relates to compounds of general formula (I) and salts and solvates thereof, wherein R¹ is adamantyl,

and wherein Z¹ and Z² are defined as in general formula (I), including general formula (Ia), general formula (Ib) and general formula (Ic), including the substitutions and preferred definitions, optionally with the proviso that in the case of general formula (Ib) Z¹ and Z² are together different from ═O,

and wherein R¹⁴ is defined as in general formula (Ib) including the substitutions and preferred definitions,

and wherein Y, R²-R⁶, R⁹-R¹³ and X¹-X⁴ are defined as in general formula (I) including the substitutions and preferred definitions,

and wherein the compounds share the following structure (I-1):

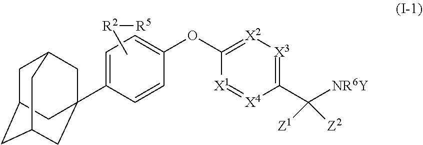

and wherein the compounds of structure (I-1) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-0014, XPW-0028, XPW-0042, XPW-0182, XPW-0924, XPW-3038, XPW-3052, XPW-4633, XPW-4642 and XPW-4643.

In a further certain embodiment, the present invention relates to compounds of general formula (I) and salts and solvates thereof, wherein Y and R⁶ are defined as in general formula (I) including the substitutions and preferred definitions, wherein Y forms a ring structure together with R⁶, and wherein such ring structure contains an O-atom in replacement of one of the ring-C-atoms that is directly linked to the N-atom to which Y and R⁶ are bound,

and wherein Z¹ and Z² are defined as in general formula (I), including general formula (Ia), general formula (Ib) and general formula (Ic), including the substitutions and preferred definitions,

and wherein R¹⁴ is defined as in general formula (Ib) including the substitutions and preferred definitions,

and wherein R¹-R⁵, R⁷-R¹² and X¹-X⁴ are defined as in general formula (I) including the substitutions and preferred definitions,

and wherein the compounds share the following structure (I-2):

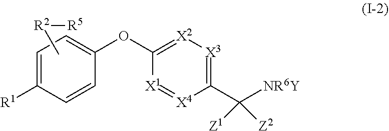

and wherein the compounds of structure (I-2) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, stomach, breast, and cancer of the neuroendocrine system.

Examples are compounds XPW-4637 and XPW-4638.

In a further certain embodiment, the present invention relates to compounds of general formula (I) and salts and solvates thereof, wherein Y is selected from —S(O)R¹³ and —S(O)₂R¹³,

and wherein R¹ is defined as in general formula (I) including the substitutions and preferred definitions, wherein R¹ contains four or more, preferably six or more carbon atoms, which are optionally independently replaced by a heteroatom selected from O, S and N as defined in general formula (I),

and wherein Z¹ and Z² are defined as in general formula (I), including general formula (Ia), general formula (Ib) and general formula (Ic), including the substitutions and preferred definitions,

and wherein R¹⁴ is defined as in general formula (Ib) including the substitutions and preferred definitions,

and wherein R²-R¹³ and X¹-X⁴ are defined as in general formula (I) including the substitutions and preferred definitions,

and wherein the compounds share the following structure (I-3):

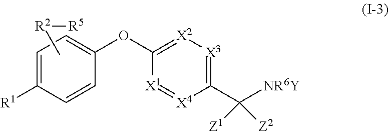

and wherein the compounds of structure (1-3) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-0547, XPW-0548, XPW-0552, XPW-0560, XPW-0566, XPW-0574, XPW-0575, XPW-0576, XPW-0580, XPW-0588, XPW-0603, XPW-0604, XPW-0608, XPW-0616, XPW-2675, XPW-2676, XPW-2688, XPW-2703, XPW-2704, XPW-2708, XPW-2716, XPW-2732, XPW-2744, XPW-4633 and XPW-4642.

In a further certain embodiment, the present invention relates to compounds of general formula (I) and salts and solvates thereof, wherein Y is —OH,

and wherein R⁶ is defined as in general formula (I) including the substitutions and preferred definitions, with the proviso that R⁶ is different from —H,

and wherein Z¹ and Z² are defined as in general formula (I), including general formula (Ia), general formula (Ib) and general formula (Ic), including the substitutions and preferred definitions,

and wherein R¹⁴ is defined as in general formula (Ib) including the substitutions and preferred definitions,

and wherein R¹-R⁵, R⁷-R¹² and X¹-X⁴ are defined as in general formula (I) including the substitutions and preferred definitions,

and wherein the compounds share the following structure (I-4):

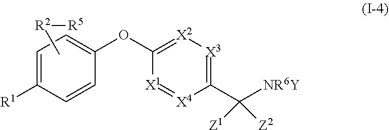

and wherein the compounds of structure (I-4) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-0182, XPW-0674, XPW-0675, XPW-0678, XPW-0679, XPW-0686, XPW-0700, XPW-0734, XPW-0742, XPW-1750, XPW-2805, XPW-2806, XPW-4612, XPW-4614, XPW-4616, XPW-4617, XPW-4618, XPW-4619, XPW-4620, XPW-4621, XPW-4622, XPW-4626, XPW-4631, XPW-4632, XPW-4640, XPW-4644, XPW-4646 and XPW-4647.

In a further certain embodiment, the present invention relates to compounds of general formula (I) and salts and solvates thereof, wherein Y is —OCH₃,

and wherein R¹ is defined as in general formula (I) including the substitutions and preferred definitions, wherein R¹ including any substituent contains no heteroatom selected from O, S, N, optionally with the proviso that R¹ contains two or more carbon atoms,

and wherein Z¹ and Z² are defined as in general formula (I), including general formula (Ia), general formula (Ib) and general formula (Ic), including the substitutions and preferred definitions,

and wherein R¹⁴ is defined as in general formula (Ib) including the substitutions and preferred definitions,

and wherein R²-R⁶, R⁹-R¹² and X¹-X⁴ are defined as in general formula (I) including the substitutions and preferred definitions,

and wherein the compounds share the following structure (I-5):

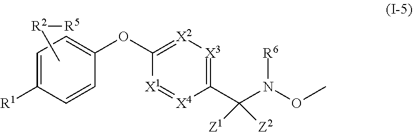

and wherein the compounds of structure (I-5) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast.

Examples are compounds XPW-0702, XPW-0706, XPW-0714, XPW-0716, XPW-0720, XPW-0728, XPW-2833, XPW-2834, XPW-2847, XPW-2848 and XPW-4605.

In a further certain embodiment, the present invention relates to compounds of general formula (I) and salts and solvates thereof, wherein R⁶ is —H and Y is —OCH₃,

and wherein R¹ is defined as in general formula (I) including the substitutions and preferred definitions, with the proviso that R¹ is selected from cyclic, bicyclic and tricyclic structures,

and wherein Z¹ and Z² are defined as in general formula (I), including general formula (Ia), general formula (Ib) and general formula (Ic), including the substitutions and preferred definitions,

and wherein R¹⁴ is defined as in general formula (Ib) including the substitutions and preferred definitions,

and wherein R²-R⁵, R⁷-R¹² and X¹-X⁴ are defined as in general formula (I) including the substitutions and preferred definitions,

and wherein the compounds share the following structure (I-6):

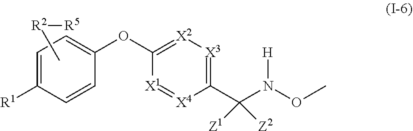

and wherein the compounds of structure (I-6) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast.

Examples are compounds XPW-0706, XPW-0714, XPW-2833 and XPW-2834.

In a further certain embodiment, the present invention relates to compounds of general formula (I) and salts and solvates thereof, wherein Y and R⁶ are defined as in general formula (I) including the substitutions and preferred definitions, wherein Y forms a ring structure together with R⁶,

and wherein R¹ is defined as in general formula (I) including the substitutions and preferred definitions, wherein R¹ is selected from cyclic, bicyclic and tricyclic structures, optionally with the proviso that R¹ including any substituent contains no or one heteroatom selected from O, S, N,

and wherein Z¹, Z² and R¹⁴ are defined as in general formula (Ib), including the substitutions and preferred definitions,

and wherein R²-R⁵, R⁷-R¹², and X¹-X⁴ are defined as in general formula (I) including the substitutions and preferred definitions,

and wherein the compounds share the following structure (I-7):

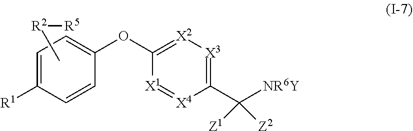

and wherein the compounds of structure (I-7) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, ovaries, and cancer of the neuroendocrine system.

Examples are compounds XPW-0762, XPW-0770, XPW-0776, XPW-0784, XPW-0790, XPW-0798, XPW-0818, XPW-2890, XPW-2898, XPW-2904, XPW-2912, XPW-2918, XPW-2926, XPW-4576, XPW-4577, XPW-4578, XPW-4579, XPW-4580, XPW-4581, XPW-4583, XPW-4586, XPW-4589, XPW-4592 and XPW-4594.

In a further certain embodiment, the present invention relates to compounds of general formula (Ia) and salts and solvates thereof, wherein Z¹ is —CF₃,

and wherein Y is defined as in general formula (I) including the substitutions and preferred definitions, optionally with the proviso that Y is different from —H,

and wherein Z² is defined as in general formula (Ia) including the substitutions and preferred definitions,

and wherein R¹-R¹³ and X¹-X⁴ are defined as in general formula (I) including the substitutions and preferred definitions,

and wherein the compounds share the following structure (Ia-1):

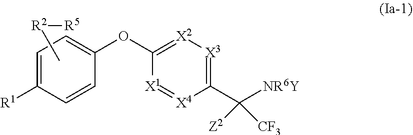

and wherein the compounds of structure (Ia-1) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-0014, XPW-0020, XPW-0028, XPW-0042, XPW-0182, XPW-4633, XPW-4642 and XPW-4643.

In a further certain embodiment, the present invention relates to compounds of general formula (Ia) and salts and solvates thereof, wherein Z¹ is —CF₃,

and wherein R⁶ is defined as in general formula (I) including the substitutions and preferred definitions, optionally with the proviso that R⁶ is different from —H,

and wherein Z² is defined as in general formula (Ia) including the substitutions and preferred definitions,

and wherein R¹-R⁵, R⁷-R¹³, X¹-X⁴ and Y are defined as in general formula (I) including the substitutions and preferred definitions,

and wherein the compounds share the following structure (Ia-2):

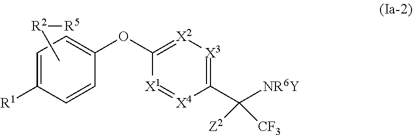

and wherein the compounds of structure (Ia-2) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-0014, XPW-0020, XPW-0028, XPW-0042, XPW-0182, XPW-4633, XPW-4642 and XPW-4643.

In a further certain embodiment, the present invention relates to compounds of general formula (Ia) and salts and solvates thereof, wherein Z¹ is —CF₃, and wherein Y and R⁶ are each —H,

and wherein R¹ is defined as in general formula (I) including the substitutions and preferred definitions, optionally with the proviso that R¹ contains five or more, preferably six or more carbon atoms, which are optionally independently replaced by a heteroatom selected from 0, S and N as defined in general formula (I),

and wherein Z² is defined as in general formula (Ia) including the substitutions and preferred definitions,

and wherein R²-R⁵, R⁷-R¹² and X¹-X⁴ are defined as in general formula (I) including the substitutions and preferred definitions,

and wherein the compounds share the following structure (Ia-3):

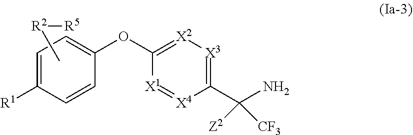

and wherein the compounds of structure (Ia-3) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, ovaries, and cancer of the neuroendocrine system.

An example is compound XPW-0014.

In a further certain embodiment, the present invention relates to compounds of general formula (Ia) and salts and solvates thereof, wherein Z¹ is —CN,

and wherein R¹ is defined as in general formula (I) including the substitutions and preferred definitions, and wherein R¹ contains three or more, preferably six or more carbon atoms, which are optionally independently replaced by a heteroatom selected from O, S and N as defined in general formula (I), optionally with the proviso that R¹ including any substituent contains no heteroatom selected from O, S and N,

and wherein Z² is defined as in general formula (Ia) including the substitutions and preferred definitions,

and wherein R²-R¹³, X¹-X⁴ and Y are defined as in general formula (I) including the substitutions and preferred definitions,

and wherein the compounds share the following structure (Ia-4):

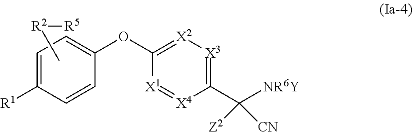

and wherein the compounds of structure (Ia-4) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, hyperproliferative disorders and cancer including cancer of the haematopoietic and haematologic system such as leukemias, and cancer of the skin.

An example is compound XPW-0314.

In a further certain embodiment, the present invention relates to compounds of general formula (Ib) and salts and solvates thereof, wherein R¹ is defined as in general formula (I) including the substitutions and preferred definitions, wherein R¹ contains six or more carbon atoms, which are optionally independently replaced by a heteroatom selected from O, S and N as defined in general formula (I), and wherein R¹ is selected from cyclic, bicyclic and tricyclic structures,

and wherein R⁵ is defined as in general formula (I) including the substitutions and preferred definitions, with the proviso that R⁵ is different from —H,

and wherein Z¹, Z² and R¹⁴ are defined as in general formula (Ib), including the substitutions and preferred definitions,

and wherein R²-R⁴, R⁶-R¹³ and X¹-X⁴ and Y are defined as in general formula (I) including the substitutions and preferred definitions,

and wherein the compounds share the following structure (Ib-1):

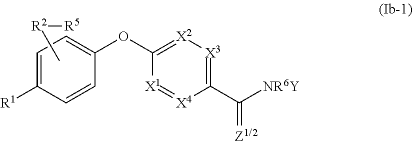

and wherein the compounds of structure (Ib-1) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-4575, XPW-4577, XPW-4578, XPW-4579, XPW-4580, XPW-4581, XPW-4583, XPW-4584, XPW-4585, XPW-4586, XPW-4587, XPW-4588, XPW-4589, XPW-4590, XPW-4591, XPW-4592, XPW-4593, XPW-4594 and XPW-4595.

In a further certain embodiment, the present invention relates to compounds of general formula (Ib) and salts and solvates thereof, wherein X⁴ is N,

and wherein R¹ is defined as in general formula (I) including the substitutions and preferred definitions, wherein R¹ contains six or more carbon atoms, which are optionally independently replaced by a heteroatom selected from O, S and N as defined in general formula (I), and wherein R¹ is selected from cyclic, bicyclic and tricyclic structures,

and wherein Z¹, Z² and R¹⁴ are defined as in general formula (Ib), including the substitutions and preferred definitions,

and wherein R²-R¹³, X¹-X³ and Y are defined as in general formula (I) including the substitutions and preferred definitions,

and wherein the compounds share the following structure (Ib-2):

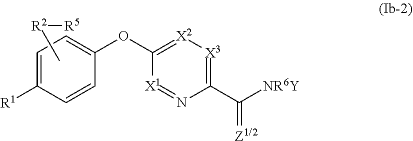

and wherein the compounds of structure (Ib-2) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-4623, XPW-4624, XPW-4628, XPW-4629, XPW-4630, XPW-4631, XPW-4632, XPW-4634, XPW-4635, XPW-4636 and XPW-4644.

In a further certain embodiment, the present invention relates to compounds of general formula (Ib) and salts and solvates thereof, wherein Z¹ and Z² are together ═O, and wherein Y is —OCH₃,

and wherein R¹ is defined as in general formula (I) including the substitutions and preferred definitions, wherein R¹ including any substituent contains no heteroatom selected from O, S, N, optionally with the proviso that R¹ contains two or more carbon atoms,

and wherein R²-R⁶, R⁹-R¹² and X¹-X⁴ are defined as in general formula (I) including the substitutions and preferred definitions,

and wherein the compounds share the following structure (Ib-3):

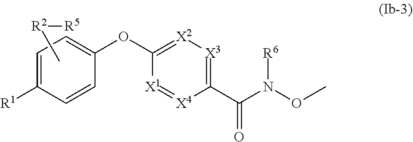

and wherein the compounds of structure (Ib-3) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast.

Examples are compounds XPW-0702, XPW-0706, XPW-0714, XPW-0716, XPW-0720, XPW-0728, XPW-2833, XPW-2834, XPW-2847, XPW-2848 and XPW-4605.

In a further certain embodiment, the present invention relates to compounds of general formula (Ib) and salts and solvates thereof, wherein Y and R⁶ are defined as in general formula (I) including the substitutions and preferred definitions, wherein Y forms a ring structure together with R⁶,

and wherein R¹ is defined as in general formula (I) including the substitutions and preferred definitions, wherein R¹ is selected from cyclic, bicyclic and tricyclic structures, optionally with the proviso that R¹ including any substituent contains no or one heteroatom selected from O, S, N,

and wherein Z¹, Z² and R¹⁴ are defined as in general formula (Ib), including the substitutions and preferred definitions,

and wherein R²-R⁵, R⁷-R¹², and X¹-X⁴ are defined as in general formula (I) including the substitutions and preferred definitions,

and wherein the compounds share the following structure (Ib-4):

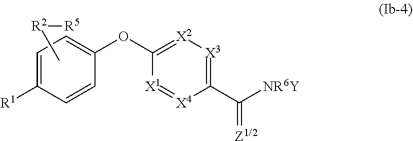

and wherein the compounds of structure (Ib-4) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, ovaries, and cancer of the neuroendocrine system.

Examples are compounds XPW-0762, XPW-0770, XPW-0776, XPW-0784, XPW-0790, XPW-0798, XPW-0818, XPW-2890, XPW-2898, XPW-2904, XPW-2912, XPW-2918, XPW-2926, XPW-4576, XPW-4577, XPW-4578, XPW-4579, XPW-4580, XPW-4581, XPW-4583, XPW-4586, XPW-4589, XPW-4592 and XPW-4594.

In a further certain embodiment, the present invention relates to compounds of general formula (Ib) and salts and solvates thereof, wherein Z¹ and Z² are together ═O, and wherein R¹ is adamantyl,

and wherein R⁵ is defined as in general formula (I) including the substitutions and preferred definitions, with the proviso that R⁵ is different from —H,

and wherein Y, R²-R⁴, R⁶, R⁹-R¹³ and X¹-X⁴ are defined as in general formula (I) including the substitutions and preferred definitions,

and wherein the compounds share the following structure (Ib-5):

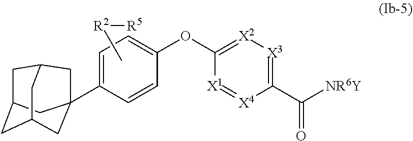

and wherein the compounds of structure (Ib-5) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-4585, XPW-4586, XPW-4587, XPW-4591, XPW-4592 and XPW-4593.

In a further certain embodiment, the present invention relates to compounds of general formula (Ib) and salts and solvates thereof, wherein Z¹ and Z² are together ═O, and wherein R¹ is adamantyl, and wherein X⁴ is N,

and wherein R²-R⁶, R⁹-R¹³, X¹-X³ and Y are defined as in general formula (I) including the substitutions and preferred definitions,

and wherein the compounds share the following structure (Ib-6):

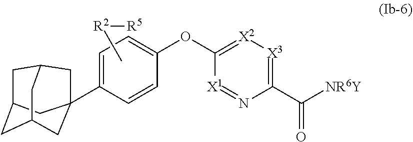

and wherein the compounds of structure (Ib-6) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-4623, XPW-4624, XPW-4628, XPW-4629, XPW-4630, XPW-4631, XPW-4632, XPW-4634, XPW-4635, XPW-4636 and XPW-4644.

In a further certain embodiment, the present invention relates to compounds of general formula (Ib) and salts and solvates thereof, wherein Z¹ and Z² are together ═O, and wherein Y is —OH, and wherein R⁶ is —H,

and wherein R¹ is defined as in general formula (I) including the substitutions and preferred definitions, wherein R¹ contains six or more carbon atoms, which are optionally independently replaced by a heteroatom selected from O, S and N as defined in general formula (I),

and wherein R⁵ is defined as in general formula (I) including the substitutions and preferred definitions, with the proviso that R⁵ is different from —H,

and wherein R²-R⁴, R⁷-R¹² and X¹-X⁴ are defined as in general formula (I) including the substitutions and preferred definitions,

and wherein the compounds share the following structure (Ib-7):

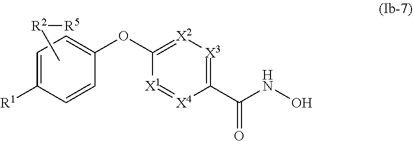

and wherein the compounds of structure (Ib-7) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-4584, XPW-4587, XPW-4590 and XPW-4593.

In a further certain embodiment, the present invention relates to compounds of general formula (Ib) and salts and solvates thereof, wherein Z¹ and Z² are together ═O, and wherein Y is —OH, and wherein R⁶ is —H, and wherein X⁴ is N,

and wherein R¹-R⁵, R⁷-R¹² and X¹-X³ are defined as in general formula (I) including the substitutions and preferred definitions,

and wherein the compounds share the following structure (Ib-8):

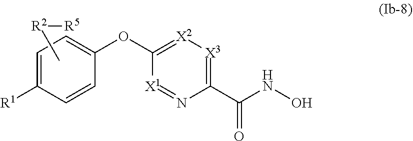

and wherein the compounds of structure (Ib-8) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-4623, XPW-4628, XPW-4630 and XPW-4636.

In a further certain embodiment, the present invention relates to compounds of general formula (Ib) and salts and solvates thereof, wherein Z¹ and Z² are together ═O, and wherein Y is —OH, and wherein R⁶ is —H, and wherein X¹ and X² are each N,

and wherein R¹-R⁵, R⁷-R¹², X³ and X⁴ are defined as in general formula (I) including the substitutions and preferred definitions,

and wherein the compounds share the following structure (Ib-9):

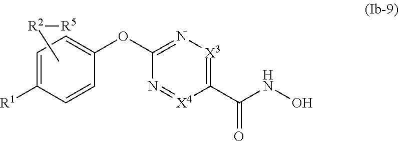

and wherein the compounds of structure (Ib-9) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

An example is compound XPW-4625.

In a further certain embodiment, the present invention relates to compounds of general formula (Ib) and salts and solvates thereof, wherein Z¹ and Z² are together ═O, and wherein Y is —OH, and wherein R⁶ is —H, and wherein X¹ is N, and wherein X⁴ is CR¹⁰, and wherein R¹⁰ is defined as in general formula (I) including the substitutions and preferred definitions, with the proviso that R¹⁰ is different from —H,

and wherein R¹-R⁵, R⁷-R⁹, R¹¹, R¹², X² and X³ are defined as in general formula (I) including the substitutions and preferred definitions,

and wherein the compounds share the following structure (Ib-10):

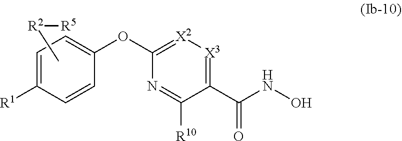

and wherein the compounds of structure (Ib-10) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

An example is compound XPW-4639.

In a further certain embodiment, the present invention relates to compounds of general formula (Ib) and salts and solvates thereof, wherein Z¹ and Z² are together ═O, and wherein Y is —CH₃, and wherein R⁶ is —CH₃,

and wherein R¹ is defined as in general formula (I) including the substitutions and preferred definitions, wherein R¹ contains six or more carbon atoms, which are optionally independently replaced by a heteroatom selected from O, S and N as defined in general formula (I),

and wherein R⁵ is defined as in general formula (I) including the substitutions and preferred definitions, wherein R⁵ is different from —H, optionally with the additional proviso that R⁵ is different from —OCH₃,

and wherein R²-R⁴, R⁷-R¹² and X¹-X⁴ are defined as in general formula (I) including the substitutions and preferred definitions,

and wherein the compounds share the following structure (Ib-11):

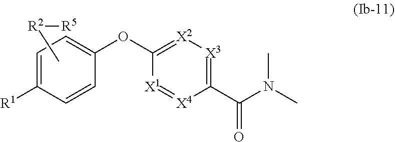

and wherein the compounds of structure (Ib-11) are—particularly without the additional proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, and cancer of the neuroendocrine system.

Examples are compounds XPW-4575, XPW-4585, XPW-4588, XPW-4591 and XPW-4595.

In a further certain embodiment, the present invention relates to compounds of general formula (Ib) and salts and solvates thereof, wherein R¹ is defined as in general formula (I) including the substitutions and preferred definitions, wherein R¹ is selected from unsubstituted and substituted cycloalkyl and cycloalkenyl, wherein such cycle contains four or more, preferably six or more ring carbon atoms that cannot be replaced by a heteroatom selected from O, S and N,

and wherein R⁵ is defined as in general formula (I) including the substitutions and preferred definitions, with the proviso that R⁵ is different from —H,

and wherein Z¹, Z² and R¹⁴ are defined as in general formula (Ib), including the substitutions and preferred definitions,

and wherein R²-R⁴, R⁶-R¹³, X¹-X⁴ and Y are defined as in general formula (I) including the substitutions and preferred definitions,

and wherein the compounds share the following structure (Ib-12):

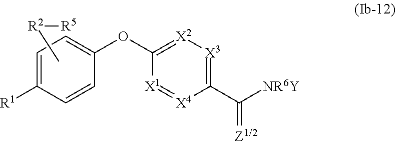

and wherein the compounds of structure (Ib-12) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-4575, XPW-4577, XPW-4578, XPW-4579, XPW-4580, XPW-4581, XPW-4583, XPW-4584, XPW-4588, XPW-4589, XPW-4590, XPW-4594 and XPW-4595.

In a further certain embodiment, the present invention relates to compounds of general formula (Ib) and salts and solvates thereof, wherein Z¹ and Z² are together ═NR¹⁴,

and wherein R¹ is defined as in general formula (I) including the substitutions and preferred definitions, wherein R¹ is selected from cyclic, bicyclic and tricyclic structures, optionally with the proviso that R¹ including any substituent contains no or one heteroatom selected from O, S, N,

and wherein R¹⁴ is defined as in general formula (Ib) including the substitutions and preferred definitions,

and wherein R²-R¹³, X¹-X⁴ and Y are defined as in general formula (I) including the substitutions and preferred definitions,

and wherein the compounds share the following structure (Ib-13):

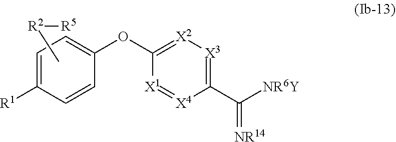

and wherein the compounds of structure (Ib-13) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue and breast.

Examples are compounds XPW-0832 and XPW-4574.

In a further certain embodiment, the present invention relates to compounds of general formula (Ib) and salts and solvates thereof, wherein Z¹ and Z² are together ═O, and wherein Y is —OH, and wherein R⁶ is —H, and wherein X¹ is CR¹¹, X² is CR⁸, X³ is CR⁹ and X⁴ is CR¹⁰,

and wherein R¹ is defined as in general formula (I) including the substitutions and preferred definitions, wherein R¹ contains four or more, preferably six or more carbon atoms, which are optionally independently replaced by a heteroatom selected from O, S and N as defined in general formula (I), with the proviso that R¹ including any substituent contains one or two heteroatoms selected from O, S, N,

and wherein R²-R⁵ and R⁷-R¹² are defined as in general formula (I) including the substitutions and preferred definitions,

and wherein the compounds share the following structure (Ib-14):

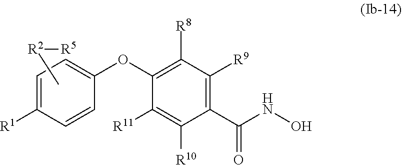

and wherein the compounds of structure (Ib-14) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-0661, XPW-0665, XPW-0667 and XPW-4613.

In a further certain embodiment, the present invention relates to compounds of general formula (Ib) and salts and solvates thereof, wherein Z¹ and Z² are together ═O, and wherein R⁶ is —CH₃,

and wherein X¹ is CR¹¹, X² is CR⁸, X³ is CR⁹ and X⁴ is CR¹⁰,

and wherein R¹ is defined as in general formula (I) including the substitutions and preferred definitions, wherein R¹ is selected from cyclic, bicyclic and tricyclic structures, with the proviso that R¹ including any substituent contains one or two heteroatoms selected from O, S, N,

and wherein R²-R⁵, R⁷-R¹³ and Y are defined as in general formula (I) including the substitutions and preferred definitions,

and wherein the compounds share the following structure (Ib-15):

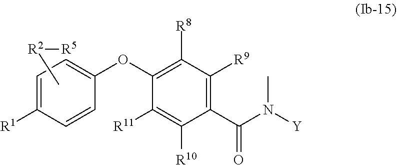

and wherein the compounds of structure (Ib-15) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, breast, ovaries, and cancer of the neuroendocrine system.

Examples are compounds XPW-0539, XPW-0541 and XPW-0679.

In a further certain embodiment, the present invention relates to compounds of general formula (Ic) and salts and solvates thereof, wherein Z¹ and Z² form together a cyclic residue including the carbon atom to which they are bound, and wherein Z¹ and Z² are defined as in general formula (Ic) including the substitutions and preferred definitions,

and wherein R⁶ is defined as in general formula (I) including the substitutions and preferred definitions, optionally with the proviso that R⁶ is different from H,

and wherein R¹-R⁵, R⁷-R¹³, X¹-X⁴ and Y are defined as in general formula (I) including the substitutions and preferred definitions,

and wherein the compounds share the following structure (Ic-1):

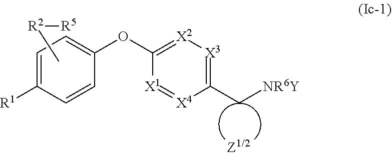

and wherein the compounds of structure (Ic-1) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-0902, XPW-0916, XPW-0924, XPW-0930, XPW-3038 and XPW-3052.

In a further certain embodiment, the present invention relates to compounds of general formula (Ic) and salts and solvates thereof, wherein Z¹ and Z² form together a cyclic residue including the carbon atom to which they are bound, and wherein Z¹ and Z² are defined as in general formula (Ic) including the substitutions and preferred definitions, optionally with the proviso that the said cyclic residue contains one or more heteroatoms independently selected from 0, S and N in replacement of a carbon atom contained in the ring structure, and/or that the said cyclic residue is substituted with one or more substituents as defined in general formula (Ic),

and wherein R¹-R¹³, X¹-X⁴ and Y are defined as in general formula (I) including the substitutions and preferred definitions,

and wherein the compounds share the following structure (Ic-2):

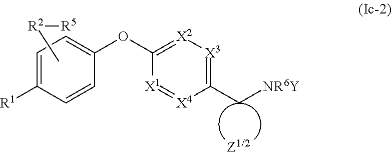

and wherein the compounds of structure (Ic-2) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-0902, XPW-0916, XPW-0924, XPW-0930, XPW-3038 and XPW-3052.

In a further certain embodiment, the present invention relates to compounds of general formula (Ic) and salts and solvates thereof, wherein Z¹ and Z² form together a cyclic residue including the carbon atom to which they are bound, and wherein Z¹ and Z² are defined as in general formula (Ic) including the substitutions and preferred definitions, optionally with the proviso that the said cyclic residue is a four-membered ring,

and wherein R¹-R¹³, X¹-X⁴ and Y are defined as in general formula (I) including the substitutions and preferred definitions,

and wherein the compounds share the following structure (Ic-3):

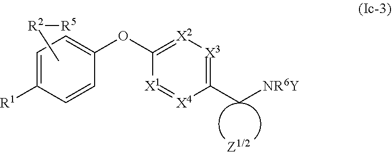

and wherein the compounds of structure (Ic-3) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-0902, XPW-0916, XPW-0924, XPW-0930, XPW-3038 and XPW-3052.

In a further certain embodiment, the present invention relates to compounds of general formula (Ic) and salts and solvates thereof, wherein Z¹ and Z² form together a cyclic residue including the carbon atom to which they are bound, and wherein Z¹ and Z² are defined as in general formula (Ic) including the substitutions and preferred definitions,

and wherein Y and R⁶ are each —H,

and wherein R¹ is defined as in general formula (I) including the substitutions and preferred definitions, optionally with the proviso that R¹ contains five or more, preferably six or more carbon atoms, which are optionally independently replaced by a heteroatom selected from 0, S and N as defined in general formula (I),

and wherein R²-R⁵, R⁷-R¹² and X¹-X⁴ are defined as in general formula (I) including the substitutions and preferred definitions,

and wherein the compounds share the following structure (Ic-4):

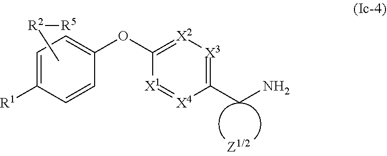

and wherein the compounds of structure (Ic-4) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPW-0916, XPW-0924 and XPW-3052.

In some embodiments, the following compounds shown in Table 1 to Table 3 are explicitly excluded from the scope of the invention:

The compounds of Table 1 specifically indicated by CAS registry numbers have been identified by the inventors as state of the art. In embodiments where these compounds are encompassed by general formula (I) or any subgeneric formula as defined herein, they are explicitly excluded from the 5 scope of the invention with regard to compound protection. To the best of the inventors' knowledge, these compounds are not known for any medical use. Thus, the invention encompasses any medical use for compounds of Table 1.

TABLE 1
CAS

14355-90-9
17353-82-1
55407-08-4
55407-09-5
55407-18-6
59129-54-3
59129-55-4
59129-56-5
69845-77-8
69845-79-0
69845-80-3
69845-81-4
69845-83-6
69845-84-7
69845-85-8
69845-86-9
69845-87-0
69845-88-1
76922-77-5
89446-97-9
102442-07-9
104271-91-2
104271-92-3
115617-06-6
115617-07-7
115617-08-8
115617-09-9
128080-22-8
128080-23-9
129400-83-5
129400-84-6
129400-85-7
129400-86-8
129400-87-9
129400-88-0
129400-92-6
129400-93-7
129400-94-8
129400-95-9
129400-96-0
129400-97-1
129400-98-2
129400-99-3
129401-06-5
129401-07-6
129401-08-7
129401-09-8
129401-10-1
129401-11-2
129475-20-3
129475-21-4
129475-22-5
129475-23-6
129475-24-7
129475-25-8
129475-26-9
129475-27-0
129475-29-2
129475-30-5
129475-31-6
129475-32-7
129475-33-8
129475-34-9
129475-35-0
129475-36-1
129475-37-2
129475-38-3
129475-39-4
129475-40-7
129475-43-0
129475-44-1
129475-51-0
129475-52-1
129480-09-7
129498-86-8
129559-91-7
129590-11-0
130338-67-9
130338-69-1
130338-71-5
130338-73-7
130338-96-4
130338-97-5
132527-22-1
132527-23-2
132527-24-3
132528-57-5
132528-60-0
132528-62-2
132528-63-3
132529-46-5
132529-47-6
132529-60-3
132529-61-4
132529-68-1
147114-90-7
154563-65-2
154606-71-0
171911-63-0
182810-52-2
187282-51-5
189119-36-6
189119-38-8
189119-98-0
189120-00-1
189120-05-6
189120-09-0
189120-10-3
204593-42-0
204593-44-2
213014-15-4
213315-38-9
226989-27-1
253160-52-0
257609-22-6
262862-78-2
282100-97-4
353466-53-2
367912-13-8
391927-63-2
391927-72-3
391927-76-7
500130-25-6
676454-26-5
676492-11-8
676493-30-4
676493-86-0
676493-92-8
676493-93-9
676493-96-2
676494-27-2
676496-49-4
676496-81-4
676496-86-9
676496-94-9
676497-19-1
676497-74-8
676497-75-9
676497-85-1
676498-15-0
676498-16-1
676498-64-9
676498-65-0
676498-76-3
676498-86-5
676498-87-6
676501-02-3
676501-55-6
676501-56-7
676501-57-8
847913-40-0
893751-86-5
915017-68-4
934195-72-9
1000995-45-8
1042448-91-8
1092718-59-6
1120314-64-8
1120314-99-9
1120315-38-9
1120315-78-7
1120316-16-6
1120316-32-6
1120316-56-4
1120316-86-0
1120317-01-2
1120317-24-9
1120317-54-5
1120320-11-7
1120320-44-6
1120320-80-0
1120321-23-4
1120321-67-6
1120321-84-7
1120322-09-9
1120322-40-8
1120322-57-7
1120322-87-3
1120323-18-3
1122598-76-8
1122599-15-8
1122599-60-3
1122600-07-0
1122600-54-7
1122600-71-8
1122600-99-0
1122601-36-8
1122601-59-5
1122601-84-6
1122602-20-3
1122605-24-6
1122605-65-5
1122606-05-6
1122606-53-4
1122607-03-7
1122607-20-8
1122607-45-7
1122608-07-4
1122608-41-6
1122608-87-0
1122609-18-0
1126632-71-0
1126632-93-6
1126632-94-7
1126632-97-0
1136832-69-3
1136832-92-2
1138697-97-8
1138698-02-8
1138698-04-0
1138698-12-0
1169483-46-8
1169483-50-4
1169483-54-8
1169483-80-0
1169483-84-4
1169484-31-4
1169484-35-8
1169484-43-8
1179751-36-0
1201900-79-9
1201900-81-3
1201900-82-4
1201900-84-6
1201900-85-7
1201900-86-8
1201900-87-9
1201900-88-0
1201900-89-1
1201900-91-5
1201900-93-7
1201900-94-8
1201900-95-9
1201900-96-0
1201900-97-1
1201900-98-2
1201900-99-3
1201901-01-0
1201901-02-1
1201901-03-2
1201901-06-5
1201901-07-6
1224590-80-0
1224591-24-5
1224591-71-2
1224592-45-3
1224592-53-3
1224592-54-4
1224592-84-0
1224592-86-2
1230143-02-8
1230144-87-2
1233502-41-4
1238005-63-4
1238010-74-6
1238010-96-2
1242172-37-7
1242172-40-2
1242172-41-3
1242172-56-0
1242172-61-7
1242172-71-9
1242172-74-2
1246663-19-3
1246663-26-2
1246663-34-2
1246663-44-4
1246663-67-1
1274666-36-2
1274666-37-3
1274666-38-4
1274666-39-5
1274666-40-8
1274666-41-9
1274666-42-0
1274666-43-1
1274666-44-2
1274666-45-3
1274666-46-4
1274666-47-5
1274666-48-6
1274666-49-7
1274666-50-0
1274666-51-1
1274666-52-2
1274666-53-3
1274666-54-4
1274666-55-5
1274666-56-6
1274666-57-7
1274666-58-8
1274666-59-9
1274666-60-2
1274666-61-3
1274666-62-4
1274666-63-5
1274666-64-6
1274666-65-7
1274666-66-8
1274666-67-9
1274666-68-0
1274666-69-1
1293368-02-1
1293368-07-6
1319734-41-2
1319734-42-3
1356844-82-0
1356844-83-1
1359735-93-5
1359738-56-9
1393831-69-0
1393831-71-4
1393831-72-5
1393831-73-6
1393831-94-1
1393831-95-2
1393831-96-3
1393831-97-4
1393831-98-5
1393831-99-6
1393832-00-2
1440059-88-0
1440059-91-5
1440059-97-1
1448238-18-3
1448759-73-6
1448766-34-4
1448770-01-1
1448770-02-2
1499185-17-9
1505469-36-2
1505469-39-5
1507265-39-5
1564440-41-0
1580442-00-7
1580442-01-8
1580442-09-6
1580442-10-9
1580442-11-0
1605344-45-3
1605344-59-9
1605344-60-2
1609018-10-1
1609018-11-2
1609018-17-8
1609018-18-9
1609018-19-0
1609018-21-4
1609018-22-5
1609018-23-6
1609018-24-7
1609018-29-2
1609018-30-5
1609018-35-0
1609018-36-1
1609018-37-2
1609018-38-3
1609018-43-0
1609018-44-1
1609018-45-2
1609018-47-4
1609018-53-2
1609019-01-3
1609019-30-8
1609019-31-9
1609019-32-0
1609019-33-1
1609019-34-2
1609019-35-3
1613190-19-4
1622156-68-6
1622156-69-7
1643132-62-0
1643668-51-2
1695558-02-1
1801520-11-5
1801520-13-7
1801520-34-2
1801895-07-7
1802064-68-1
1807453-75-3
1887233-04-6
1998123-43-5
2003250-30-2
2003250-68-6
2093403-97-3
2172931-52-9
2241854-31-7
2242830-97-1
2244556-86-1
2307305-97-9

The compounds of Table 2 specifically indicated by CAS registry numbers have been identified by the inventors as state of the art. In embodiments, where these compounds are encompassed by general formula (I) or any subgeneric formula as defined herein, they are explicitly excluded from the scope of the invention with regard to compound protection. To the best of the inventors' knowledge, these compounds are not known for any medical use as defined in the invention. Thus, the compounds of Table 2 are explicitly included into the scope of the invention with regard to medical use as defined herein, particularly in the treatment of non-malignant or malignant hyperproliferative diseases.

TABLE 2
CAS

51362-92-6
68548-57-2
101586-27-0
132526-69-3
132526-70-6
132528-39-3
132528-40-6
132528-41-7
132528-55-3
132528-56-4
132528-58-6
132528-59-7
132528-61-1
132548-61-9
132548-63-1
173964-51-7
173964-52-8
173964-53-9
173964-55-1
173964-56-2
173964-59-5
175800-05-2
176684-96-1
176684-97-2
180637-08-5
180637-09-6
180637-11-0
180637-13-2
180637-14-3
180637-15-4
180637-17-6
180637-18-7
180637-20-1
180637-26-7
180637-27-8
180637-28-9
180637-56-3
182134-68-5
182135-22-4
182136-38-5
189120-10-3
191657-89-3
191657-90-6
191657-91-7
191657-92-8
191657-95-1
191657-96-2
191657-97-3
191657-98-4
191657-99-5
191658-00-1
191658-03-4
191658-04-5
264927-27-7
264927-29-9
264927-30-2
332008-83-0
332008-84-1
332130-45-7
332130-46-8
332130-49-1
332130-50-4
364322-43-0
364322-45-2
364322-46-3
364322-47-4
364322-49-6
364322-50-9
364322-51-0
364322-53-2
364322-54-3
364322-55-4
364322-56-5
364322-57-6
364322-58-7
364323-14-8
364323-15-9
364323-16-0
364323-17-1
364323-18-2
364323-19-3
364323-20-6
364323-21-7
364323-22-8
364323-23-9
364323-24-0
364323-25-1
364323-26-2
364323-27-3
364323-28-4
364323-29-5
364323-32-0
364323-33-1
364323-34-2
364323-35-3
364324-36-7
400855-58-5
400855-91-6
401467-51-4
401467-69-4
401467-88-7
401467-91-2
401476-77-5
401476-84-4
402910-97-8
402911-01-7
402911-02-8
402911-04-0
402911-05-1
402911-18-6
473255-44-6
500557-07-3
500557-20-0
500557-30-2
500557-33-5
503067-89-8
503067-90-1
503067-91-2
503067-93-4
503067-94-5
586357-05-3
586357-12-2
586357-25-7
586358-31-8
586359-27-5
586359-40-2
586360-37-4
586360-50-1
586361-47-9
586361-60-6
586362-60-9
586362-73-4
676492-00-5
676492-01-6
676492-02-7
676492-03-8
676492-05-0
676492-06-1
676492-10-7
676492-47-0
676492-48-1
676492-49-2
676492-50-5
676492-51-6
676492-52-7
676492-53-8
676492-54-9
676492-61-8
676492-86-7
676492-89-0
676492-92-5
676492-93-6
676492-95-8
676492-97-0
676493-06-4
676493-08-6
676493-09-7
676493-13-3
676493-25-7
676493-27-9
676493-33-7
676493-36-0
676493-38-2
676493-39-3
676493-40-6
676493-46-2
676493-50-8
676493-59-7
676493-60-0
676493-61-1
676493-67-7
676493-71-3
676493-83-7
676493-84-8
676493-88-2
676493-94-0
676493-95-1
676493-97-3
676494-00-1
676494-01-2
676494-05-6
676494-38-5
676494-39-6
676494-40-9
676494-41-0
676494-42-1
676494-43-2
676494-44-3
676494-45-4
676494-49-8
676494-51-2
676494-52-3
676494-63-6
676494-71-6
676494-84-1
676494-92-1
676494-93-2
676494-94-3
676494-95-4
676494-96-5
676494-97-6
676494-98-7
676494-99-8
676495-00-4
676495-01-5
676495-03-7
676495-04-8
676495-05-9
676495-06-0
676495-11-7
676495-23-1
676495-30-0
676495-37-7
676495-43-5
676495-47-9
676495-49-1
676495-51-5
676495-55-9
676495-66-2
676495-81-1
676495-88-8
676496-11-0
676496-12-1
676496-50-7
676496-52-9
676496-55-2
676496-66-5
676496-68-7
676496-70-1
676496-80-3
676496-88-1
676496-93-8
676496-96-1
676496-99-4
676497-01-1
676497-06-6
676497-10-2
676497-12-4
676497-13-5
676497-14-6
676497-15-7
676497-18-0
676497-23-7
676497-24-8
676497-25-9
676497-28-2
676497-29-3
676497-35-1
676497-38-4
676497-41-9
676497-42-0
676497-45-3
676497-67-9
676497-68-0
676497-69-1
676497-86-2
676498-03-6
676498-08-1
676498-13-8
676498-18-3
676498-19-4
676498-20-7
676498-21-8
676498-32-1
676498-33-2
676498-35-4
676498-48-9
676498-53-6
676498-55-8
676498-56-9
676498-59-2
676498-60-5
676498-61-6
676498-66-1
676498-67-2
676498-68-3
676498-71-8
676498-72-9
676498-73-0
676498-77-4
676498-78-5
676498-79-6
676498-81-0
676498-82-1
676498-88-7
676498-89-8
676498-90-1
676498-91-2
676499-89-1
676499-90-4
676499-92-6
676499-93-7
676499-94-8
676499-95-9
676499-96-0
676500-00-8
676500-01-9
676500-02-0
676500-03-1
676500-05-3
676500-07-5
676500-09-7
676500-11-1
676500-13-3
676500-14-4
676500-15-5
676500-16-6
676500-19-9
676500-20-2
676500-23-5
676500-24-6
676500-26-8
676500-28-0
676500-29-1
676500-35-9
676500-37-1
676500-38-2
676500-42-8
676500-45-1
676500-48-4
676500-49-5
676500-65-5
676500-67-7
676500-68-8
676500-71-3
676500-99-5
676501-05-6
676501-06-7
676501-07-8
676501-08-9
676501-09-0
676501-10-3
676501-12-5
676501-13-6
676501-14-7
676501-15-8
676501-19-2
676501-21-6
676501-23-8
676501-25-0
676501-30-7
676501-32-9
676501-33-0
676501-34-1
676501-35-2
676501-36-3
676501-38-5
676501-49-8
676501-61-4
676501-62-5
676501-63-6
676501-67-0
676501-71-6
676501-74-9
676501-77-2
676501-78-3
676501-79-4
676501-81-8
676501-83-0
854207-90-2
854207-91-3
854530-56-6
855244-82-5
859505-28-5
859505-29-6
859505-35-4
859505-38-7
872794-13-3
873995-76-7
873995-77-8
875855-66-6
897036-15-6
916067-65-7
916067-66-8
916067-67-9
916067-68-0
916067-69-1
916067-70-4
916067-71-5
916067-72-6
916067-73-7
916799-08-1
916799-11-6
916799-24-1
916799-26-3
916799-31-0
941010-68-0
941263-10-1
947501-24-8
952433-31-7
1000677-90-6
1000677-99-5
1000678-02-3
1012872-57-9
1012872-71-7
1012872-72-8
1012872-74-0
1012872-76-2
1012872-77-3
1012872-78-4
1012872-79-5
1012872-80-8
1012872-81-9
1012872-82-0
1024033-63-3
1024033-65-5
1024033-69-9
1024033-71-3
1138698-06-2
1166872-27-0
1195173-17-1
1195298-10-2
1240613-51-7
1269055-85-7
1269055-92-6
1293368-03-2
1293368-04-3
1293368-06-5
1293368-08-7
1293368-09-8
1293368-10-1
1293368-13-4
1310048-38-4
1316758-86-7
1316758-87-8
1316758-88-9
1316758-89-0
1316758-90-3
1316758-91-4
1316759-01-9
1316759-02-0
1316759-06-4
1316759-08-6
1316759-10-0
1316759-12-2
1316759-14-4
1316759-16-6
1316759-18-8
1348867-58-2
1354955-46-6
1354955-52-4
1354955-63-7
1354956-13-0
1354956-52-7
1354956-60-7
1354956-94-7
1354956-97-0
1354957-13-3
1354957-35-9
1354957-63-3
1354957-67-7
1354958-07-8
1354958-22-7
1354958-29-4
1354958-33-0
1354958-34-1
1354958-45-4
1354958-53-4
1354958-54-5
1354958-58-9
1354958-59-0
1354958-70-5
1354958-73-8
1354958-94-3
1354958-96-5
1354959-01-5
1354959-18-4
1354959-20-8
1354959-28-6
1354959-60-6
1354959-68-4
1354959-79-7
1354959-87-7
1354960-13-6
1354960-16-9
1354960-33-0
1354960-41-0
1354968-84-5
1354968-85-6
1354968-89-0
1403681-62-8
1403681-84-4
1446444-73-0
1446444-74-1
1446444-76-3
1446444-82-1
1446444-84-3
1446444-91-2
1453854-30-2
1533431-19-4
1533431-20-7
1533431-24-1
1533431-32-1
1533431-55-8
1567711-29-8
1834601-38-5
2019189-07-0
2019189-15-0
2019189-23-0
2019189-24-1
2019189-25-2
2019189-28-5
2019189-34-3
2133003-54-8
2250018-33-6

The compounds of Table 3 specifically indicated by CAS registry numbers have been identified by the inventors as state of the art. In embodiments, where these compounds are encompassed by general formula (I) or any subgeneric formula as defined herein, they are explicitly excluded from the scope of the invention with regard to compound protection. Further, these compounds are, to the best of the inventors' knowledge, known for a medical use, which in some embodiments may be encompassed by a medical use as defined herein. Thus, the compounds of Table 3 may be explicitly excluded from the scope of the invention with regard to compound protection and with regard to certain medical use in some embodiments as defined herein.

TABLE 3

| CAS | Reference |
|---|---|
| 176694-31-8 | EP700899 A1 |
| 176694-32-9 | EP700899 A1 |
| 176694-33-0 | EP700899 A1 |
| 188609-23-6 | WO9707769 A2 |
| 188609-24-7 | WO9707769 A2 |
| 188609-25-8 | WO9707769 A2 |
| 188609-26-9 | WO9707769 A2 |
| 189156-60-3 | WO9920263 A1 |
| 189156-61-4 | WO9920263 A1 |
| 189156-62-5 | WO9920263 A1 |
| 212056-73-0 | WO9835982 A1 |
| 212056-74-1 | WO9835982 A1 |
| 212056-87-6 | WO9835982 A1 |
| 212056-88-7 | WO9835982 A1 |
| 212056-90-1 | WO9835982 A1 |
| 212056-91-2 | WO9835982 A1 |
| 212056-92-3 | WO9835982 A1 |
| 212056-93-4 | WO9835982 A1 |
| 212056-94-5 | WO9835982 A1 |
| 212056-95-6 | WO9835982 A1 |
| 212056-96-7 | WO9835982 A1 |
| 212056-97-8 | WO9835982 A1 |
| 212056-99-0 | WO9835982 A1 |
| 212057-01-7 | WO9835982 A1 |
| 212057-02-8 | WO9835982 A1 |
| 212057-06-2 | WO9835982 A1 |
| 212058-34-9 | WO9835982 A1 |
| 212058-36-1 | WO9835982 A1 |
| 212058-38-3 | WO9835982 A1 |
| 219618-78-7 | 10.1016/j.bmc.2011.10.068 |
| 223466-40-8 | WO9919296 A1 |
| 257605-04-2 | WO2000005198 A1 |
| 257605-05-3 | WO2000005198 A1 |
| 257605-07-5 | WO2000005198 A1 |
| 257605-08-6 | WO2000005198 A1 |
| 257605-09-7 | WO2000005198 A1 |
| 257605-10-0 | WO2000005198 A1 |
| 257609-85-1 | WO2000005198 A1 |
| 257609-86-2 | WO2000005198 A1 |
| 257609-87-3 | WO2000005198 A1 |
| 257610-12-1 | WO2000005198 A1 |
| 257610-13-2 | WO2000005198 A1 |
| 257610-14-3 | WO2000005198 A1 |
| 257610-37-0 | WO2000005198 A1 |
| 262287-94-5 | WO2000017162A1 |
| 311784-70-0 | US20090163545A1 |
| 321521-92-0 | US20090163545A1 |
| 326823-48-7 | WO2001012189 A1 |
| 326823-54-5 | WO2001012189 A1 |
| 342912-73-6 | WO2001040227 A1 |
| 342913-08-0 | WO2001040227 A1 |
| 342913-13-7 | WO2001040227 A1 |
| 342913-61-5 | WO2001040227 A1 |
| 343274-97-5 | WO2001040227 A1 |
| 343275-31-0 | WO2001040227 A1 |
| 343275-39-8 | WO2001040227 A1 |
| 356032-36-5 | WO2001060354 A1 |
| 356032-37-6 | WO2001060354 A1 |
| 420121-86-4 | US20050107414 A1 |
| 420121-95-5 | US20050107414 A1 |
| 420121-97-7 | US20050107414 A1 |
| 428833-44-7 | US20110144043 A1 |
| 461020-11-1 | WO2002074770 A1 |
| 461020-66-6 | WO2002074770 A1 |
| 461020-68-8 | WO2002074770 A1 |
| 461020-82-6 | WO2002074770 A1 |
| 461021-06-7 | WO2002074770 A1 |
| 461021-14-7 | WO2002074770 A1 |
| 461021-42-1 | WO2002074770 A1 |
| 461021-48-7 | WO2002074770 A1 |
| 461024-01-1 | WO2002074770 A1 |
| 461024-54-4 | WO2002074770 A1 |
| 461404-62-6 | WO2002074298 A1 |
| 461441-47-4 | WO2002074770 A1 |
| 461441-66-7 | WO2002074770 A1 |
| 461441-93-0 | WO2002074770 A1 |
| 461441-94-1 | WO2002074770 A1 |
| 461442-01-3 | WO2002074770 A1 |
| 461442-12-6 | WO2002074770 A1 |
| 461442-16-0 | WO2002074770 A1 |
| 461442-28-4 | WO2002074770 A1 |
| 461442-30-8 | WO2002074770 A1 |
| 461442-31-9 | WO2002074770 A1 |
| 461443-89-0 | WO2002074770 A1 |
| 461444-40-6 | WO2002074770 A1 |
| 503067-97-8 | PCT/EP2018/054686 |
| 503067-98-9 | PCT/EP2018/054686 |
| 503068-10-8 | PCT/EP2018/054686 |
| 503068-00-6 | PCT/EP2018/054686 |
| 700358-38-9 | US20110144043 A1 |
| 860457-85-8 | US 20050165049 A1 |
| 866153-56-2 | 10.1016/j.bmcl.2011.05.075 |
| 875671-17-3 | WO2007066784 A2 |
| 875671-18-4 | WO2007066784 A2 |
| 875695-06-0 | WO2007066784 A2 |
| 875701-46-5 | WO2007066784A2 |
| 875701-47-6 | WO2007066784A2 |
| 875705-84-3 | WO2007066784A2 |
| 875705-86-5 | WO2007066784A2 |
| 875709-09-4 | WO2007066784A2 |
| 900015-10-3 | WO2017158190 A1 |
| 918157-10-5 | US20080200521 A1 |
| 918157-12-7 | US20080200521 A1 |
| 918157-13-8 | US20080200521 A1 |
| 918157-16-1 | US20080200521 A1 |
| 918157-17-2 | US20080200521 A1 |
| 918157-20-7 | US20080200521 A1 |
| 918157-24-1 | US20080200521 A1 |
| 918157-25-2 | US20080200521 A1 |
| 918157-26-3 | US20080200521 A1 |
| 918157-27-4 | US20080200521 A1 |
| 918157-38-7 | US20080200521 A1 |
| 918157-39-8 | US20080200521 A1 |
| 918157-40-1 | US20080200521 A1 |
| 918157-47-8 | US20080200521 A1 |
| 918157-48-9 | US20080200521 A1 |
| 918157-49-0 | US20080200521 A1 |
| 918157-50-3 | US20080200521 A1 |
| 918157-51-4 | US20080200521 A1 |
| 918157-52-5 | US20080200521 A1 |
| 918157-53-6 | US20080200521 A1 |
| 918157-54-7 | US20080200521 A1 |
| 918293-77-3 | US20080200521 A1 |
| 918293-78-4 | US20080200521 A1 |
| 921624-35-3 | EP1908466 |
| 921624-36-4 | EP1908466 |
| 934690-71-8 | 10.1016/j.bmcl.2006.12.048 |
| 934690-72-9 | 10.1016/j.bmcl.2006.12.048 |
| 934690-73-0 | 10.1016/j.bmcl.2006.12.048 |
| 941231-94-3 | 10.13822/j.cnki.hxsj.2014.09.002 |
| 1009100-76-8 | WO2008024746 A1 |
| 1009100-84-8 | WO2008024746 A1 |
| 1009100-85-9 | WO2008024746 A1 |
| 1009100-86-0 | WO2008024746 A1 |
| 1009100-87-1 | WO2008024746 A1 |
| 1009100-88-2 | WO2008024746 A1 |
| 1009100-89-3 | WO2008024746 A1 |
| 1009100-90-6 | WO2008024746 A1 |
| 1009100-95-1 | WO2008024746 A1 |
| 1009100-97-3 | WO2008024746 A1 |
| 1009101-19-2 | WO2008024746 A1 |
| 1009101-23-8 | WO2008024746 A1 |
| 1009101-25-0 | WO2008024746 A1 |
| 1009101-27-2 | WO2008024746 A1 |
| 1009101-29-4 | WO2008024746 A1 |
| 1009101-31-8 | WO2008024746 A1 |
| 1009101-33-0 | WO2008024746 A1 |
| 1009101-35-2 | WO2008024746 A1 |
| 1009101-37-4 | WO2008024746 A1 |
| 1009101-39-6 | WO2008024746 A1 |
| 1009101-41-0 | WO2008024746 A1 |
| 1009101-43-2 | WO2008024746 A1 |
| 1009101-45-4 | WO2008024746 A1 |
| 1009101-47-6 | WO2008024746 A1 |
| 1009101-49-8 | WO2008024746 A1 |
| 1009101-51-2 | WO2008024746 A1 |
| 1009101-53-4 | WO2008024746 A1 |
| 1009101-55-6 | WO2008024746 A1 |
| 1009101-61-4 | WO2008024746 A1 |
| 1055361-40-4 | WO 2008109727 A1 |
| 1055361-61-9 | WO 2008109727 A1 |
| 1055361-62-0 | WO 2008109727 A1 |
| 1181576-98-6 | PCT/EP2018/054686 |
| 1260222-07-8 | WO 2004058164 A2 |
| 1260236-81-4 | WO 2004058164 A2 |
| 1260236-88-1 | WO 2004058164 A2 |
| 1316758-92-5 | PCT/EP2018/054686 |
| 1357175-78-0 | 10.1016/j.bmcl.2011.11.034 |
| 1357175-79-1 | 10.1016/j.bmcl.2011.11.034 |
| 1357175-80-4 | 10.1016/j.bmcl.2011.11.034 |
| 1357175-81-5 | 10.1016/j.bmcl.2011.11.034 |
| 1357175-82-6 | 10.1016/j.bmcl.2011.11.034 |
| 1357175-83-7 | 10.1016/j.bmcl.2011.11.034 |
| 1357175-84-8 | 10.1016/j.bmcl.2011.11.034 |
| 1357175-85-9 | 10.1016/j.bmcl.2011.11.034 |
| 1357175-86-0 | 10.1016/j.bmcl.2011.11.034 |
| 1357175-87-1 | 10.1016/j.bmcl.2011.11.034 |
| 1357175-88-2 | 10.1016/j.bmcl.2011.11.034 |
| 1357175-89-3 | 10.1016/j.bmcl.2011.11.034 |
| 1357175-90-6 | 10.1016/j.bmcl.2011.11.034 |
| 1357175-91-7 | 10.1016/j.bmcl.2011.11.034 |
| 1357175-92-8 | 10.1016/j.bmcl.2011.11.034 |
| 1357175-93-9 | 10.1016/j.bmcl.2011.11.034 |
| 1357175-94-0 | 10.1016/j.bmcl.2011.11.034 |
| 1357175-95-1 | 10.1016/j.bmcl.2011.11.034 |
| 1357175-96-2 | 10.1016/j.bmcl.2011.11.034 |
| 1357175-97-3 | 10.1016/j.bmcl.2011.11.034 |
| 1357176-00-1 | 10.1016/j.bmcl.2011.11.034 |
| 1357176-01-2 | 10.1016/j.bmcl.2011.11.034 |
| 1357176-07-8 | 10.1016/j.bmcl.2011.11.034 |
| 1357176-12-5 | 10.1016/j.bmcl.2011.11.034 |
| 1357176-13-6 | 10.1016/j.bmcl.2011.11.034 |
| 1357176-14-7 | 10.1016/j.bmcl.2011.11.034 |
| 1357176-15-8 | 10.1016/j.bmcl.2011.11.034 |
| 1357176-16-9 | 10.1016/j.bmcl.2011.11.034 |
| 1357176-92-1 | 10.1016/j.bmcl.2011.11.034 |
| 1357176-18-1 | 10.1016/j.bmcl.2011.11.034 |
| 1357176-08-9 | 10.1016/j.bmcl.2011.11.034 |
| 1357176-09-0 | 10.1016/j.bmcl.2011.11.034 |
| 1357176-10-3 | 10.1016/j.bmcl.2011.11.034 |
| 1357176-11-4 | 10.1016/j.bmcl.2011.11.034 |
| 1316759-04-2 | 10.1016/j.bmcl.2011.11.034 |
| 1377995-71-5 | WO2012068589A2 |
| 1377996-54-7 | WO2012068589A2 |
| 1377996-60-5 | WO2012068589A2 |
| 1377997-62-0 | WO2012068589A2 |
| 1377997-93-7 | WO2012068589A2 |
| 1393831-70-3 | WO2012110840 A1 |
| 1417988-56-7 | WO 2013004190 A1 |
| 1417989-84-4 | WO 2013004190 A1 |
| 1579998-48-3 | WO 2014043068 A1 |
| 1579998-49-4 | WO 2014043068 A1 |
| 1580439-47-9 | WO 2014043068 A1 |
| 1580439-48-0 | WO 2014043068 A1 |
| 1580439-49-1 | WO 2014043068 A1 |
| 1580439-50-4 | WO 2014043068 A1 |
| 1580439-51-5 | WO 2014043068 A1 |
| 1580439-52-6 | WO 2014043068 A1 |
| 1580439-53-7 | WO 2014043068 A1 |
| 1580439-57-1 | WO 2014043068 A1 |
| 1580439-58-2 | WO 2014043068 A1 |
| 1580439-59-3 | WO 2014043068 A1 |
| 1580439-60-6 | WO 2014043068 A1 |
| 1580439-65-1 | WO 2014043068 A1 |
| 1580439-66-2 | WO 2014043068 A1 |
| 1580439-67-3 | WO 2014043068 A1 |
| 1580439-68-4 | WO 2014043068 A1 |
| 1580439-69-5 | WO 2014043068 A1 |
| 1875062-56-8 | WO 2016016421 A1 |
| 1875062-61-5 | WO 2016016421 A1 |
| 1875062-79-5 | WO 2016016421 A1 |
| 1875063-20-9 | WO 2016016421 A1 |
| 1997336-52-3 | 10.1007/s00044-016-1667-y |
| 2199032-37-4 | PCT/EP2018/054686 |
| 2199032-37-4 | PCT/EP2018/054686 |
| 2199623-41-9 | PCT/EP2018/054686 |
| 2243096-86-6 | PCT/EP2018/054686 |
| 2243096-86-6 | PCT/EP2018/054686 |
| 2243097-09-6 | PCT/EP2018/054686 |
| 2243097-10-9 | PCT/EP2018/054686 |
| 2243097-11-0 | PCT/EP2018/054686 |
| 2243097-12-1 | PCT/EP2018/054686 |
| 2243097-13-2 | PCT/EP2018/054686 |
| 2243097-15-4 | PCT/EP2018/054686 |
| 2243097-16-5 | PCT/EP2018/054686 |
| 2243097-09-6 | PCT/EP2018/054686 |
| 2243097-10-9 | PCT/EP2018/054686 |
| 2243097-11-0 | PCT/EP2018/054686 |
| 2243097-12-1 | PCT/EP2018/054686 |
| 2243097-13-2 | PCT/EP2018/054686 |
| 2243097-15-4 | PCT/EP2018/054686 |
| 2243097-16-5 | PCT/EP2018/054686 |
| 2243097-27-8 | PCT/EP2018/054686 |
| 2243097-29-0 | PCT/EP2018/054686 |
| 2243097-30-3 | PCT/EP2018/054686 |
| 2243097-31-4 | PCT/EP2018/054686 |
| 2243098-06-6 | PCT/EP2018/054686 |
| 2243098-07-7 | PCT/EP2018/054686 |
| 2243098-08-8 | PCT/EP2018/054686 |
| 2243098-09-9 | PCT/EP2018/054686 |
| 2243098-10-2 | PCT/EP2018/054686 |
| 2243098-12-4 | PCT/EP2018/054686 |
| 2243098-14-6 | PCT/EP2018/054686 |
| 2243098-23-7 | PCT/EP2018/054686 |
| 2243098-25-9 | PCT/EP2018/054686 |
| 2243098-26-0 | PCT/EP2018/054686 |
| 2243098-27-1 | PCT/EP2018/054686 |
| 2243100-54-9 | PCT/EP2018/054686 |
| 2243100-55-0 | PCT/EP2018/054686 |
| 2243100-56-1 | PCT/EP2018/054686 |
| 2243100-57-2 | PCT/EP2018/054686 |
| 2243100-58-3 | PCT/EP2018/054686 |
| 2243100-60-7 | PCT/EP2018/054686 |
| 2243100-61-8 | PCT/EP2018/054686 |
| 2243100-63-0 | PCT/EP2018/054686 |
| 2243100-72-1 | PCT/EP2018/054686 |
| 2243100-74-3 | PCT/EP2018/054686 |
| 2243100-75-4 | PCT/EP2018/054686 |
| 2243100-76-5 | PCT/EP2018/054686 |
| 2243101-57-5 | PCT/EP2018/054686 |
| 2243101-58-6 | PCT/EP2018/054686 |
| 2243101-59-7 | PCT/EP2018/054686 |
| 2243101-60-0 | PCT/EP2018/054686 |
| 2243101-61-1 | PCT/EP2018/054686 |
| 2243101-63-3 | PCT/EP2018/054686 |
| 2243101-65-5 | PCT/EP2018/054686 |
| 2243101-67-7 | PCT/EP2018/054686 |
| 2243101-76-8 | PCT/EP2018/054686 |
| 2243101-80-4 | PCT/EP2018/054686 |
| 2243102-98-7 | PCT/EP2018/054686 |
| 2243102-99-8 | PCT/EP2018/054686 |
| 2243103-00-4 | PCT/EP2018/054686 |
| 2243103-01-5 | PCT/EP2018/054686 |
| 2243103-06-0 | PCT/EP2018/054686 |
| 2243103-34-4 | PCT/EP2018/054686 |
| 2243103-35-5 | PCT/EP2018/054686 |
| 2243103-36-6 | PCT/EP2018/054686 |
| 2243103-37-7 | PCT/EP2018/054686 |
| 2243103-42-4 | PCT/EP2018/054686 |
| 2243103-62-8 | PCT/EP2018/054686 |

Specific examples of compounds falling under the scope of compounds contained in pending application PCT/EP2018/054686 have been identified in the present application to have novel medical use, in particular to have growth inhibitory properties on muscle cells, keratinocytes, and cells and malignant cells selected from cervical cancer, cutaneous T-cell lymphoma, acute promyelocytic leukemia, acute myeloid leukemia, oral and tongue squamous cell carcinoma, epidermoid squamous cell carcinoma and lung squamous cell carcinoma cells.

Thus, these compounds as well as salts and solvates thereof are particularly suitable for the treatment of hyperproliferative muscle diseases, hyperproliferative skin diseases as defined herein as well as for the treatment of cervical cancer, cutaneous T-cell lymphoma, acute promyelocytic leukemia, acute myeloid leukemia, epidermoid skin cancer such as non-melanoma skin cancer, cancer of the oral cavity, cancer of the tongue and lung cancer as defined herein.

Specific examples of compounds falling under the scope of compounds contained in pending application PCT/EP2018/054686 have been identified in the present application to have further novel medical use, in particular to have growth inhibitory properties on cells and malignant cells selected from T-cell leukemia, B-cell leukemia, gastric cancer, breast cancer, ovarian cancer and medullary thyroid cancer.

Thus, these compounds as well as salts and solvates thereof are particularly suitable for the treatment of diseases of the haematopoietic system including the haematologic system such as T-cell leukemia, B-cell leukemia, as well as for the treatment of gastric cancer, breast cancer, ovarian cancer and cancer of the neuroendocrine system as defined herein.

The herein identified novel medical use for specific compounds falling under the scope of compounds contained in pending application PCT/EP2018/054686 are shown in Table 4 and Table 5, wherein the medical applications are selected from the treatments of hyperproliferative muscle diseases (A), hyperproliferative skin diseases as defined herein (B), cervical cancer (C), cutaneous T-cell lymphoma (D), acute promyelocytic leukemia (E), acute myeloid leukemia (F), epidermoid skin cancer (G), cancer of the oral cavity (H), cancer of the tongue (I), lung cancer (J), T-cell leukemia (K), B-cell leukemia (L), gastric cancer (M), breast cancer (N), ovarian cancer (0) and cancer of the neuroendocrine system (P).

The following compounds described in PCT/EP2018/054686 are specifically claimed for the indicated medical use.

TABLE 4

| Compound No. | Medical use |
|---|---|
| XPW-0516 | B |
| XPW-0518 | B D E H J |
| XPW-0529 | A B J |
| XPW-0530 | A B J |
| XPW-0532 | B J |
| XPW-0543 | A |
| XPW-0544 | A B J |
| XPW-0546 | A B E G H |
| XPW-0659 | A B D E G H I |
| XPW-0660 | A B C D E F G H I |
| XPW-0663 | A B C D E G H I |
| XPW-0664 | A C D E G I J |
| XPW-0669 | A B C E G H I J |
| XPW-0670 | A C D E G H I J |
| XPW-0672 | A B C D E G I J |
| XPW-1582 | B D F H J |
| XPW-1596 | B |
| XPW-1610 | B D G H J |
| XPW-1727 | A B C D E GH I J |
| XPW-1728 | A C D E G I |
| XPW-1736 | A B C D E G I J |
| XPW-2643 | B |
| XPW-2646 | B |
| XPW-2648 | E |
| XPW-2651 | B |
| XPW-2657 | B |
| XPW-2658 | A D E H |
| XPW-2660 | A B D E H I J |
| XPW-2665 | B D H |
| XPW-2671 | B |
| XPW-2672 | B |
| XPW-2787 | A B C D E G H I J |
| XPW-2788 | A B C D E F G H I J |
| XPW-2791 | A B C D E G H I |
| XPW-2792 | A B C D E G I |
| XPW-2797 | A B C E G H I J |
| XPW-2798 | A D E G H I J |
| XPW-2800 | B C D E G I J |
| XPW-3193 | A B C D E G I |
| XPW-3194 | A B C D E G H I |
| XPW-3196 | B D E |
| XPW-3197 | A C D E G H I J |
| XPW-3199 | B E H |
| XPW-3200 | A B C D E G H I |
| XPW-3201 | A B D E G I J |
| XPW-3202 | A B C D E G H I J |
| XPW-3203 | A B D E G H I |
| XPW-3205 | E |
| XPW-3206 | A B C D E G H I J |
| XPW-3207 | D |
| XPW-3208 | E I |
| XPW-3209 | A B D E G H I |
| XPW-3210 | B D |
| XPW-3211 | B D H |
| XPW-3212 | A B C D E G H I |
| XPW-3213 | A B C D E G H I |
| XPW-3214 | A B D E G H I |
| XPW-3215 | D |
| XPW-3216 | A B D E H I |
| XPW-3217 | A B D E H I |
| XPW-3218 | A B C D E G H I |
| XPW-3219 | A B C D E G I |
| XPW-3221 | A C D E G I J |
| XPW-3223 | A B D E H I J |
| XPW-3224 | B D E G |
| XPW-3225 | A B C D E G H I J |
| XPW-3226 | A B C D E G H I J |
| XPW-3227 | A C D E G H I J |
| XPW-3230 | B |
| XPW-3231 | A B C D E G H I J |
| XPW-3232 | B G H I |
| XPW-3233 | A B C D E H I J |
| XPW-3234 | B G |

The following compounds described in PCT/EP2018/054686 are specifically claimed for the indicated medical use.

TABLE 5

| Compound No. | Medical use |
|---|---|
| XPW-0506 | B |
| XPW-0509 | B |
| XPW-0510 | B D |
| XPW-0515 | I K N |
| XPW-0516 | G N |
| XPW-0518 | K L M N |
| XPW-0520 | A B H |
| XPW-0523 | B |
| XPW-0524 | A D H K L N |
| XPW-0529 | D E G H I K L N |
| XPW-0530 | D E G H K L N P |
| XPW-0532 | D K L |
| XPW-0533 | A B E K L N |
| XPW-0534 | A B K N |
| XPW-0537 | B H K L N |
| XPW-0538 | A B G H K L N |
| XPW-0543 | E G H |
| XPW-0544 | E G H K L N |
| XPW-0546 | K L N |
| XPW-0659 | J K L M N O |
| XPW-0660 | J K L M N O P |
| XPW-0663 | J K L M N O P |
| XPW-0664 | K L M N O |
| XPW-0669 | D K L M N O P |
| XPW-0670 | K L M N O P |
| XPW-0672 | K L M N O |
| XPW-1582 | K L N P |
| XPW-1587 | L N |
| XPW-1588 | K L N |
| XPW-1596 | G H K L M |
| XPW-1601 | B N |
| XPW-1602 | A G I K L N |
| XPW-1610 | E K L N |
| XPW-1727 | K L M N O |
| XPW-1728 | J K L M N O |
| XPW-1736 | K L M N O |
| XPW-2633 | B N |
| XPW-2634 | B K N |
| XPW-2637 | B J L N |
| XPW-2643 | K N |
| XPW-2644 | J |
| XPW-2646 | N |
| XPW-2648 | A B D G H K L N O |
| XPW-2651 | L N |
| XPW-2652 | B K N |
| XPW-2657 | D G H K N |
| XPW-2658 | K L N P |
| XPW-2660 | K L N P |
| XPW-2661 | B E G K N |
| XPW-2662 | B K L N |
| XPW-2665 | K L N |
| XPW-2666 | H I K L N |
| XPW-2671 | K N |
| XPW-2672 | K L N |
| XPW-2674 | D E H I K L N |
| XPW-2787 | K L M N O P |
| XPW-2788 | K L M N O P |
| XPW-2791 | J K L M N O P |
| XPW-2792 | K L M N O |
| XPW-2797 | D K L M N O P |
| XPW-2798 | K L M N O |
| XPW-2800 | K L M N O |
| XPW-3193 | K L M N O |
| XPW-3194 | K L M N O P |
| XPW-3195 | B D K N |
| XPW-3196 | K L N |
| XPW-3197 | K L M N O P |
| XPW-3199 | K N |
| XPW-3200 | K L M N O P |
| XPW-3201 | K L M N O |
| XPW-3202 | K L M N O P |
| XPW-3203 | K L M N O P |
| XPW-3205 | K L N |
| XPW-3206 | K L M N O P |
| XPW-3207 | N |
| XPW-3208 | B K L N |
| XPW-3209 | K L M N O P |
| XPW-3211 | N |
| XPW-3212 | K L M N O P |
| XPW-3213 | J K L M N O P |
| XPW-3214 | K L M N O |
| XPW-3215 | G H I |
| XPW-3216 | K L |
| XPW-3217 | K L M O P |
| XPW-3218 | K L M N O P |
| XPW-3219 | K L M N O P |
| XPW-3221 | K L M N O |
| XPW-3222 | K N |
| XPW-3223 | K L N |
| XPW-3224 | K L N |
| XPW-3225 | K L M N O P |
| XPW-3226 | K L M N O P |
| XPW-3227 | K L M N O P |
| XPW-3228 | N |
| XPW-3229 | L N |
| XPW-3230 | E I K L N |
| XPW-3231 | K L M N O P |
| XPW-3232 | A K N |
| XPW-3233 | G K L M N O P |
| XPW-3234 | A K N |
| XPW-4543 | B |
| XPW-4544 | B N |
| XPW-4545 | A K L N |
| XPW-4546 | B K |
| XPW-4547 | B L N |
| XPW-4548 | B I L N |
| XPW-4549 | B N |
| XPW-4550 | D I N |
| XPW-4551 | B K N |
| XPW-4552 | D |
| XPW-4553 | D N |
| XPW-4554 | N |
| XPW-4555 | B N |
| XPW-4556 | B N |
| XPW-4557 | N |
| XPW-4558 | B |
| XPW-4559 | B |
| XPW-4560 | B J K L N |
| XPW-4561 | K N |
| XPW-4562 | A B K L N |
| XPW-4563 | B N |
| XPW-4564 | B N |
| XPW-4565 | A B G H I L N O |
| XPW-4566 | B N |
| XPW-4567 | A B E G I L N |
| XPW-4568 | B E N |
| XPW-4569 | A B E G H N O |
| XPW-4570 | N |
| XPW-4571 | N |
| XPW-4572 | K N |
| XPW-4573 | K L N |
| XPW-4645 | A B E K L N |
| XPW-4714 | B |
| XPW-4715 | B |
| XPW-4718 | B |
| XPW-4723 | B |
| XPW-4843 | B |

Specific examples of compounds falling under the scope of formula (I) are shown in Table 6 to Table 54. Intermediates are denoted as “XPW-I”.

TABLE 6

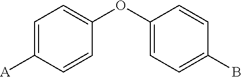

| A\B | 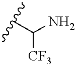 [SMILES: *C(N)C(F)(F)F] | 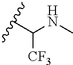 [SMILES: *C(NC)C(F)(F)F] | 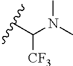 [SMILES: *C(N(C)C)C(F)(F)F] | 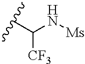 [SMILES: *C(NS(C)(=O)=O)C(F)(F)F] | 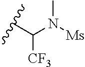 [SMILES: *C(N(C)S(C)(=O)=O)C(F)(F)F] | 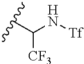 | 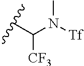 | 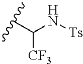 [SMILES: *C(NS(=O)(=O)c1ccc(C)cc1)C(F)(F)F] | 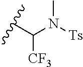 [SMILES: *C(N(C)S(=O)(=O)c1ccc(C)cc1)C(F)(F)F] | 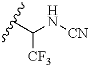 [SMILES: *C(NC#N)C(F)(F)F] | 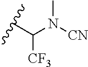 [SMILES: *C(N(C)C#N)C(F)(F)F] |
|---|---|---|---|---|---|---|---|---|---|---|---|
| 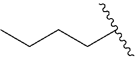 [SMILES: *CCCC] | XPW-0001 | XPW-0015 | XPW-0029 | XPW-0043 | XPW-0057 | XPW-0071 | XPW-0085 | XPW-0099 | XPW-0113 | XPW-0127 | XPW-0141 |
| 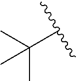 [SMILES: *C(C)(C)C] | XPW-0002 | XPW-0016 | XPW-0030 | XPW-0044 | XPW-0058 | XPW-0072 | XPW-0086 | XPW-0100 | XPW-0114 | XPW-0128 | XPW-0142 |
| 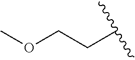 [SMILES: *CCOC] | XPW-0003 | XPW-0017 | XPW-0031 | XPW-0045 | XPW-0059 | XPW-0073 | XPW-0087 | XPW-0101 | XPW-0115 | XPW-0129 | XPW-0143 |
| 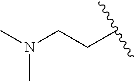 [SMILES: *CCN(C)C] | XPW-0004 | XPW-0018 | XPW-0032 | XPW-0046 | XPW-0060 | XPW-0074 | XPW-0088 | XPW-0102 | XPW-0116 | XPW-0130 | XPW-0144 |
| 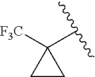 [SMILES: *C1(C(F)(F)F)CC1] | XPW-0005 | XPW-0019 | XPW-0033 | XPW-0047 | XPW-0061 | XPW-0075 | XPW-0089 | XPW-0103 | XPW-0117 | XPW-0131 | XPW-0145 |
| 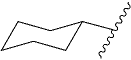 [SMILES: *C1CCCCC1] | XPW-0006 | XPW-0020 | XPW-0034 | XPW-0048 | XPW-0062 | XPW-0076 | XPW-0090 | XPW-0104 | XPW-0118 | XPW-0132 | XPW-0146 |
| 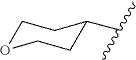 [SMILES: *C1CCOCC1] | XPW-0007 | XPW-0021 | XPW-0035 | XPW-0049 | XPW-0063 | XPW-0077 | XPW-0091 | XPW-0105 | XPW-0119 | XPW-0133 | XPW-0147 |
| 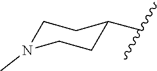 [SMILES: *C1CCN(C)CC1] | XPW-0008 | XPW-0022 | XPW-0036 | XPW-0050 | XPW-0064 | XPW-0078 | XPW-0092 | XPW-0106 | XPW-0120 | XPW-0134 | XPW-0148 |
| 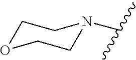 [SMILES: *N1CCOCC1] | XPW-0009 | XPW-0023 | XPW-0037 | XPW-0051 | XPW-0065 | XPW-0079 | XPW-0093 | XPW-0107 | XPW-0121 | XPW-0135 | XPW-0149 |
| 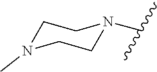 [SMILES: *N1CCN(C)CC1] | XPW-0010 | XPW-0024 | XPW-0038 | XPW-0052 | XPW-0066 | XPW-0080 | XPW-0094 | XPW-0108 | XPW-0122 | XPW-0136 | XPW-0150 |
| 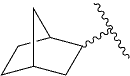 | XPW-0011 | XPW-0025 | XPW-0039 | XPW-0053 | XPW-0067 | XPW-0081 | XPW-0095 | XPW-0109 | XPW-0123 | XPW-0137 | XPW-0151 |
| 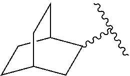 | XPW-0012 | XPW-0026 | XPW-0040 | XPW-0054 | XPW-0068 | XPW-0082 | XPW-0096 | XPW-0110 | XPW-0124 | XPW-0138 | XPW-0152 |
| 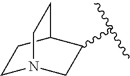 | XPW-0013 | XPW-0027 | XPW-0041 | XPW-0055 | XPW-0069 | XPW-0083 | XPW-0097 | XPW-0111 | XPW-0125 | XPW-0139 | XPW-0153 |
| 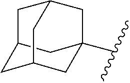 [SMILES: *C12CC3CC(CC(C3)C1)C2] | XPW-0014 | XPW-0028 | XPW-0042 | XPW-0056 | XPW-0070 | XPW-0084 | XPW-0098 | XPW-0112 | XPW-0126 | XPW-0140 | XPW-0154 |

The above table constitutes an individualized description of each of the specifically indicated compounds therein as well as their salts and solvates.

TABLE 7

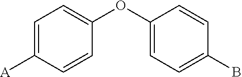

| A\B | 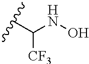 [SMILES: *C(NO)C(F)(F)F] | 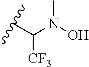 [SMILES: *C(N(C)O)C(F)(F)F] | 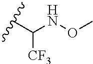 [SMILES: *C(NOC)C(F)(F)F] | 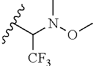 [SMILES: *C(N(C)OC)C(F)(F)F] | 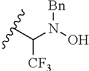 [SMILES: *C(N(O)Cc1ccccc1)C(F)(F)F] | 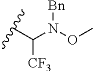 [SMILES: *C(N(Cc1ccccc1)OC)C(F)(F)F] | 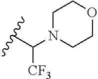 [SMILES: *C(N1CCOCC1)C(F)(F)F] | 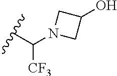 [SMILES: *C(N1CC(O)C1)C(F)(F)F] | 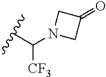 [SMILES: *C(N1CC(=O)C1)C(F)(F)F] |
|---|---|---|---|---|---|---|---|---|---|
| 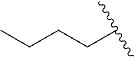 [SMILES: *CCCC] | XPW-0155 | XPW-0169 | XPW-0183 | XPW-0197 | XPW-0211 | XPW-0225 | XPW-0239 | XPW-0253 | XPW-0267 |
| 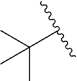 [SMILES: *C(C)(C)C] | XPW-0156 | XPW-0170 | XPW-0184 | XPW-0198 | XPW-0212 | XPW-0226 | XPW-0240 | XPW-0254 | XPW-0268 |
| 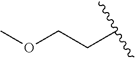 [SMILES: *CCOC] | XPW-0157 | XPW-0171 | XPW-0185 | XPW-0199 | XPW-0213 | XPW-0227 | XPW-0241 | XPW-0255 | XPW-0269 |
| 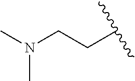 [SMILES: *CCN(C)C] | XPW-0158 | XPW-0172 | XPW-0186 | XPW-0200 | XPW-0214 | XPW-0228 | XPW-0242 | XPW-0256 | XPW-0270 |
| 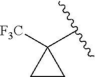 [SMILES: *C1(C(F)(F)F)CC1] | XPW-0159 | XPW-0173 | XPW-0187 | XPW-0201 | XPW-0215 | XPW-0229 | XPW-0243 | XPW-0257 | XPW-0271 |
| 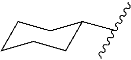 [SMILES: *C1CCCCC1] | XPW-0160 | XPW-0174 | XPW-0188 | XPW-0202 | XPW-0216 | XPW-0230 | XPW-0244 | XPW-0258 | XPW-0272 |
| 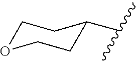 [SMILES: *C1CCOCC1] | XPW-0161 | XPW-0175 | XPW-0189 | XPW-0203 | XPW-0217 | XPW-0231 | XPW-0245 | XPW-0259 | XPW-0273 |
| 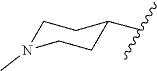 [SMILES: *C1CCN(C)CC1] | XPW-0162 | XPW-0176 | XPW-0190 | XPW-0204 | XPW-0218 | XPW-0232 | XPW-0246 | XPW-0260 | XPW-0274 |
| 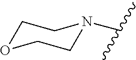 [SMILES: *N1CCOCC1] | XPW-0163 | XPW-0177 | XPW-0191 | XPW-0205 | XPW-0219 | XPW-0233 | XPW-0247 | XPW-0261 | XPW-0275 |
| 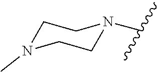 [SMILES: *N1CCN(C)CC1] | XPW-0164 | XPW-0178 | XPW-0192 | XPW-0206 | XPW-0220 | XPW-0234 | XPW-0248 | XPW-0262 | XPW-0276 |
| 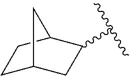 | XPW-0165 | XPW-0179 | XPW-0193 | XPW-0207 | XPW-0221 | XPW-0235 | XPW-0249 | XPW-0263 | XPW-0277 |
| 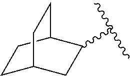 | XPW-0166 | XPW-0180 | XPW-0194 | XPW-0208 | XPW-0222 | XPW-0236 | XPW-0250 | XPW-0264 | XPW-0278 |
| 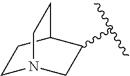 | XPW-0167 | XPW-0181 | XPW-0195 | XPW-0209 | XPW-0223 | XPW-0237 | XPW-0251 | XPW-0265 | XPW-0279 |
| 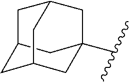 [SMILES: *C12CC3CC(CC(C3)C1)C2] | XPW-0168 | XPW-0182 | XPW-0196 | XPW-0210 | XPW-0224 | XPW-0238 | XPW-0252 | XPW-0266 | XPW-0280 |
| | | | | | | | A\B | 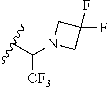 [SMILES: *C(N1CC(F)(F)C1)C(F)(F)F] | 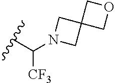 [SMILES: *C(N1CC2(COC2)C1)C(F)(F)F] |
| | | | | | | | 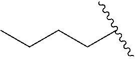 [SMILES: *CCCC] | XPW-0281 | XPW-0295 |
| | | | | | | | 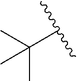 [SMILES: *C(C)(C)C] | XPW-0282 | XPW-0296 |
| | | | | | | | 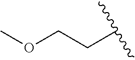 [SMILES: *CCOC] | XPW-0283 | XPW-0297 |
| | | | | | | | 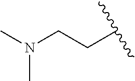 [SMILES: *CCN(C)C] | XPW-0284 | XPW-0298 |
| | | | | | | | 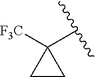 [SMILES: *C1(C(F)(F)F)CC1] | XPW-0285 | XPW-0299 |
| | | | | | | | 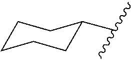 [SMILES: *C1CCCCC1] | XPW-0286 | XPW-0300 |
| | | | | | | | 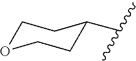 [SMILES: *C1CCOCC1] | XPW-0287 | XPW-0301 |
| | | | | | | | 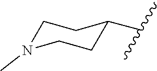 [SMILES: *C1CCN(C)CC1] | XPW-0288 | XPW-0302 |
| | | | | | | | 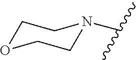 [SMILES: *N1CCOCC1] | XPW-0289 | XPW-0303 |
| | | | | | | | 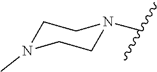 [SMILES: *N1CCN(C)CC1] | XPW-0290 | XPW-0304 |
| | | | | | | | 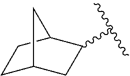 | XPW-0291 | XPW-0305 |
| | | | | | | | 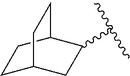 | XPW-0292 | XPW-0306 |
| | | | | | | | 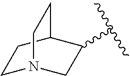 | XPW-0293 | XPW-0307 |
| | | | | | | | 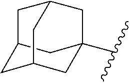 [SMILES: *C12CC3CC(CC(C3)C1)C2] | XPW-0294 | XPW-0308 |

The above table constitutes an individualized description of each of the specifically indicated compounds therein as well as their salts and solvates.

TABLE 8

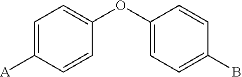

| A\B | 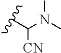 [SMILES: *C(C#N)N(C)C] | 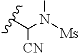 [SMILES: *C(C#N)N(C)S(C)(=O)=O] | 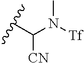 | 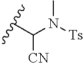 [SMILES: *C(C#N)N(C)S(=O)(=O)c1ccc(C)cc1] | 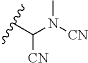 [SMILES: *C(C#N)N(C)C#N] | 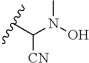 [SMILES: *C(C#N)N(C)O] | 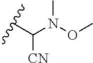 [SMILES: *C(C#N)N(C)OC] | 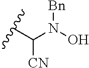 [SMILES: *C(C#N)N(O)Cc1ccccc1] | 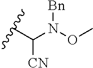 [SMILES: *C(C#N)N(Cc1ccccc1)OC] | 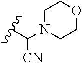 [SMILES: *C(C#N)N1CCOCC1] | 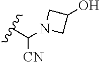 [SMILES: *C(C#N)N1CC(O)C1] |
|---|---|---|---|---|---|---|---|---|---|---|---|
| 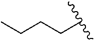 [SMILES: *CCCC] | XPW- 0309 | XPW-0323 | XPW-0337 | XPW-0351 | XPW-0365 | XPW-0379 | XPW-0393 | XPW-0407 | XPW-0421 | XPW-0435 | XPW-0449 |
| 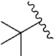 [SMILES: *C(C)(C)C] | XPW- 0310 | XPW-0324 | XPW-0338 | XPW-0352 | XPW-0366 | XPW-0380 | XPW-0394 | XPW-0408 | XPW-0422 | XPW-0436 | XPW-0450 |
| 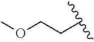 [SMILES: *CCOC] | XPW- 0311 | XPW-0325 | XPW-0339 | XPW-0353 | XPW-0367 | XPW-0381 | XPW-0395 | XPW-0409 | XPW-0423 | XPW-0437 | XPW-0451 |
| 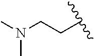 [SMILES: *CCN(C)C] | XPW- 0312 | XPW-0326 | XPW-0340 | XPW-0354 | XPW-0368 | XPW-0382 | XPW-0396 | XPW-0410 | XPW-0424 | XPW-0438 | XPW-0452 |
| 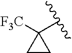 [SMILES: *C1(C(F)(F)F)CC1] | XPW- 0313 | XPW-0327 | XPW-0341 | XPW-0355 | XPW-0369 | XPW-0383 | XPW-0397 | XPW-0411 | XPW-0425 | XPW-0439 | XPW-0453 |
| 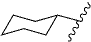 [SMILES: *C1CCCCC1] | XPW- 0314 | XPW-0328 | XPW-0342 | XPW-0356 | XPW-0370 | XPW-0384 | XPW-0398 | XPW-0412 | XPW-0426 | XPW-0440 | XPW-0454 |
| 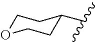 [SMILES: *C1CCOCC1] | XPW- 0315 | XPW-0329 | XPW-0343 | XPW-0357 | XPW-0371 | XPW-0385 | XPW-0399 | XPW-0413 | XPW-0427 | XPW-0441 | XPW-0455 |
| 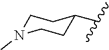 [SMILES: *C1CCN(C)CC1] | XPW- 0316 | XPW-0330 | XPW-0344 | XPW-0358 | XPW-0372 | XPW-0386 | XPW-0400 | XPW-0414 | XPW-0428 | XPW-0442 | XPW-0456 |
| 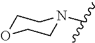 [SMILES: *N1CCOCC1] | XPW- 0317 | XPW-0331 | XPW-0345 | XPW-0359 | XPW-0373 | XPW-0387 | XPW-0401 | XPW-0415 | XPW-0429 | XPW-0443 | XPW-0457 |
| 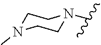 [SMILES: *N1CCN(C)CC1] | XPW- 0318 | XPW-0332 | XPW-0346 | XPW-0360 | XPW-0374 | XPW-0388 | XPW-0402 | XPW-0416 | XPW-0430 | XPW-0444 | XPW-0458 |
| 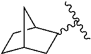 | XPW- 0319 | XPW-0333 | XPW-0347 | XPW-0361 | XPW-0375 | XPW-0389 | XPW-0403 | XPW-0417 | XPW-0431 | XPW-0445 | XPW-0459 |
| 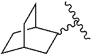 | XPW- 0320 | XPW-0334 | XPW-0348 | XPW-0362 | XPW-0376 | XPW-0390 | XPW-0404 | XPW-0418 | XPW-0432 | XPW-0446 | XPW-0460 |
| 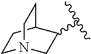 | XPW- 0321 | XPW-0335 | XPW-0349 | XPW-0363 | XPW-0377 | XPW-0391 | XPW-0405 | XPW-0419 | XPW-0433 | XPW-0447 | XPW-0461 |
| 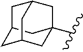 [SMILES: *C12CC3CC(CC(C3)C1)C2] | XPW- 0322 | XPW-0336 | XPW-0350 | XPW-0364 | XPW-0378 | XPW-0392 | XPW-0406 | XPW-0420 | XPW-0434 | XPW-0448 | XPW-0462 |

The above table constitutes an individualized description of each of the specifically indicated compounds therein as well as their salts and solvates.

TABLE 9

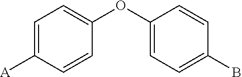

| A\B | 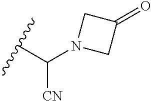 [SMILES: *C(C#N)N1CC(=O)C1] | 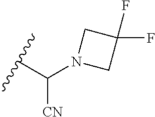 [SMILES: *C(C#N)N1CC(F)(F)C1] | 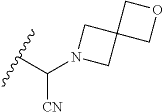 [SMILES: *C(C#N)N1CC2(COC2)C1] | 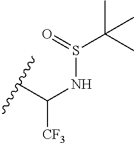 [SMILES: *C(NS(=O)C(C)(C)C)C(F)(F)F] | 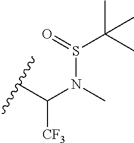 [SMILES: *C(N(C)S(=O)C(C)(C)C)C(F)(F)F] | 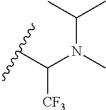 [SMILES: *C(N(C)C(C)C)C(F)(F)F] |
|---|---|---|---|---|---|---|
| 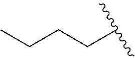 [SMILES: *CCCC] | XPW-0463 | XPW-0477 | XPW-0491 | XPW-4649 | XPW-4662 | XPW-4675 |
| 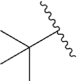 [SMILES: *C(C)(C)C] | XPW-0464 | XPW-0478 | XPW-0492 | XPW-4650 | XPW-4663 | XPW-4676 |
| 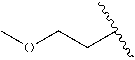 [SMILES: *CCOC] | XPW-0465 | XPW-0479 | XPW-0493 | XPW-4651 | XPW-4664 | XPW-4677 |
| 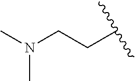 [SMILES: *CCN(C)C] | XPW-0466 | XPW-0480 | XPW-0494 | XPW-4652 | XPW-4665 | XPW-4678 |
| 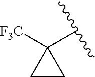 [SMILES: *C1(C(F)(F)F)CC1] | XPW-0467 | XPW-0481 | XPW-0495 | XPW-4653 | XPW-4666 | XPW-4679 |
| 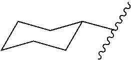 [SMILES: *C1CCCCC1] | XPW-0468 | XPW-0482 | XPW-0496 | XPW-4654 | XPW-4667 | XPW-4680 |
| 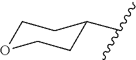 [SMILES: *C1CCOCC1] | XPW-0469 | XPW-0483 | XPW-0497 | XPW-4655 | XPW-4668 | XPW-4681 |
| 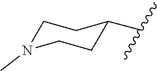 [SMILES: *C1CCN(C)CC1] | XPW-0470 | XPW-0484 | XPW-0498 | XPW-4656 | XPW-4669 | XPW-4682 |
| 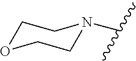 [SMILES: *N1CCOCC1] | XPW-0471 | XPW-0485 | XPW-0499 | XPW-4657 | XPW-4670 | XPW-4683 |
| 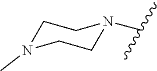 [SMILES: *N1CCN(C)CC1] | XPW-0472 | XPW-0486 | XPW-0500 | XPW-4658 | XPW-4671 | XPW-4684 |
| 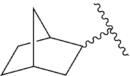 | XPW-0473 | XPW-0487 | XPW-0501 | XPW-4659 | XPW-4672 | XPW-4685 |
| 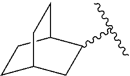 | XPW-0474 | XPW-0488 | XPW-0502 | XPW-4660 | XPW-4673 | XPW-4686 |
| 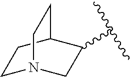 | XPW-0475 | XPW-0489 | XPW-0503 | XPW-4661 | XPW-4674 | XPW-4687 |
| 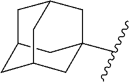 [SMILES: *C12CC3CC(CC(C3)C1)C2] | XPW-0476 | XPW-0490 | XPW-0504 | XPW-4633 | XPW-4642 | XPW-4643 |

The above table constitutes an individualized description of each of the specifically indicated compounds therein as well as their salts and solvates.

TABLE 10

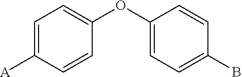

| A\B | 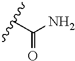 [SMILES: *C(N)=O] | 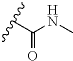 [SMILES: *C(=O)NC] | 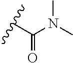 [SMILES: *C(=O)N(C)C] | 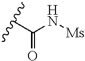 [SMILES: *C(=O)NS(C)(=O)=O] | 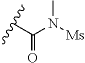 [SMILES: *C(=O)N(C)S(C)(=O)=O] | 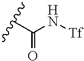 | 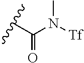 | 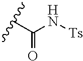 [SMILES: *C(=O)NS(=O)(=O)c1ccc(C)cc1] | 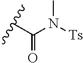 [SMILES: *C(=O)N(C)S(=O)(=O)c1ccc(C)cc1] | 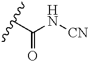 [SMILES: *C(=O)NC#N] | 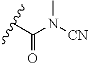 [SMILES: *C(=O)N(C)C#N] |
|---|---|---|---|---|---|---|---|---|---|---|---|
| 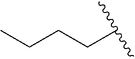 [SMILES: *CCCC] | XPW-0505 | XPW-0519 | XPW-0533 | XPW-0547 | XPW-0561 | XPW-0575 | XPW-0589 | XPW-0603 | XPW-0617 | XPW-0631 | XPW-0645 |
| 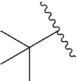 [SMILES: *C(C)(C)C] | XPW-0506 | XPW-0520 | XPW-0534 | XPW-0548 | XPW-0562 | XPW-0576 | XPW-0590 | XPW-0604 | XPW-0618 | XPW-0632 | XPW-0646 |
| 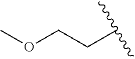 [SMILES: *CCOC] | XPW-0507 | XPW-0521 | XPW-0535 | XPW-0549 | XPW-0563 | XPW-0577 | XPW-0591 | XPW-0605 | XPW-0619 | XPW-0633 | XPW-0647 |
| 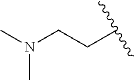 [SMILES: *CCN(C)C] | XPW-0508 | XPW-0522 | XPW-0536 | XPW-0550 | XPW-0564 | XPW-0578 | XPW-0592 | XPW-0606 | XPW-0620 | XPW-0634 | XPW-0648 |
| 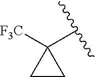 [SMILES: *C1(C(F)(F)F)CC1] | XPW-0509 | XPW-0523 | XPW-0537 | XPW-0551 | XPW-0565 | XPW-0579 | XPW-0593 | XPW-0607 | XPW-0621 | XPW-0635 | XPW-0649 |
| 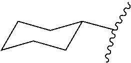 [SMILES: *C1CCCCC1] | XPW-0510 | XPW-0524 | XPW-0538 | XPW-0552 | XPW-0566 | XPW-0580 | XPW-0594 | XPW-0608 | XPW-0622 | XPW-0636 | XPW-0650 |
| 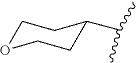 [SMILES: *C1CCOCC1] | XPW-0511 | XPW-0525 | XPW-0539 | XPW-0553 | XPW-0567 | XPW-0581 | XPW-0595 | XPW-0609 | XPW-0623 | XPW-0637 | XPW-0651 |
| 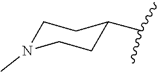 [SMILES: *C1CCN(C)CC1] | XPW-0512 | XPW-0526 | XPW-0540 | XPW-0554 | XPW-0568 | XPW-0582 | XPW-0596 | XPW-0610 | XPW-0624 | XPW-0638 | XPW-0652 |
| 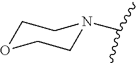 [SMILES: *N1CCOCC1] | XPW-0513 | XPW-0527 | XPW-0541 | XPW-0555 | XPW-0569 | XPW-0583 | XPW-0597 | XPW-0611 | XPW-0625 | XPW-0639 | XPW-0653 |
| 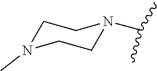 [SMILES: *N1CCN(C)CC1] | XPW-0514 | XPW-0528 | XPW-0542 | XPW-0556 | XPW-0570 | XPW-0584 | XPW-0598 | XPW-0612 | XPW-0626 | XPW-0640 | XPW-0654 |
| 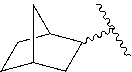 | XPW-0515 | XPW-0529 | XPW-0543 | XPW-0557 | XPW-0571 | XPW-0585 | XPW-0599 | XPW-0613 | XPW-0627 | XPW-0641 | XPW-0655 |
| 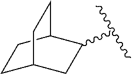 | XPW-0516 | XPW-0530 | XPW-0544 | XPW-0558 | XPW-0572 | XPW-0586 | XPW-0600 | XPW-0614 | XPW-0628 | XPW-0642 | XPW-0656 |
| 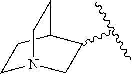 | XPW-0517 | XPW-0531 | XPW-0545 | XPW-0559 | XPW-0573 | XPW-0587 | XPW-0601 | XPW-0615 | XPW-0629 | XPW-0643 | XPW-0657 |
| 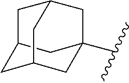 [SMILES: *C12CC3CC(CC(C3)C1)C2] | XPW-0518 | XPW-0532 | XPW-0546 | XPW-0560 | XPW-0574 | XPW-0588 | XPW-0602 | XPW-0616 | XPW-0630 | XPW-0644 | XPW-0658 |

The above table constitutes an individualized description of each of the specifically indicated compounds therein as well as their salts and solvates.

TABLE 11

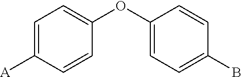

| A\B | 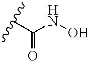 [SMILES: *C(=O)NO] | 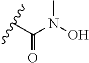 [SMILES: *C(=O)N(C)O] | 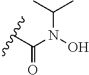 [SMILES: *C(=O)N(O)C(C)C] | 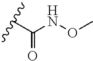 [SMILES: *C(=O)NOC] | 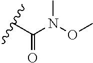 [SMILES: *C(=O)N(C)OC] | 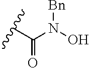 [SMILES: *C(=O)N(O)Cc1ccccc1] | 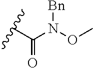 [SMILES: *C(=O)N(Cc1ccccc1)OC] | 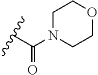 [SMILES: *C(=O)N1CCOCC1] | 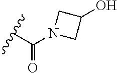 [SMILES: *C(=O)N1CC(O)C1] |
|---|---|---|---|---|---|---|---|---|---|
| 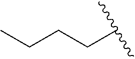 [SMILES: *CCCC] | XPW-0659 | XPW-0673 | XPW-0687 | XPW-0701 | XPW-0715 | XPW-0729 | XPW-0743 | XPW-0757 | XPW-0771 |
| 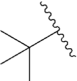 [SMILES: *C(C)(C)C] | XPW-0660 | XPW-0674 | XPW-0688 | XPW-0702 | XPW-0716 | XPW-0730 | XPW-0744 | XPW-0758 | XPW-0772 |
| 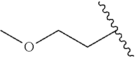 [SMILES: *CCOC] | XPW-0661 | XPW-0675 | XPW-0689 | XPW-0703 | XPW-0717 | XPW-0731 | XPW-0745 | XPW-0759 | XPW-0773 |
| 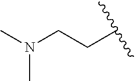 [SMILES: *CCN(C)C] | XPW-0662 | XPW-0676 | XPW-0690 | XPW-0704 | XPW-0718 | XPW-0732 | XPW-0746 | XPW-0760 | XPW-0774 |
| 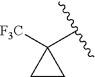 [SMILES: *C1(C(F)(F)F)CC1] | XPW-0663 | XPW-0677 | XPW-0691 | XPW-0705 | XPW-0719 | XPW-0733 | XPW-0747 | XPW-0761 | XPW-0775 |
| 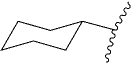 [SMILES: *C1CCCCC1] | XPW-0664 | XPW-0678 | XPW-0692 | XPW-0706 | XPW-0720 | XPW-0734 | XPW-0748 | XPW-0762 | XPW-0776 |
| 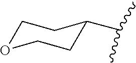 [SMILES: *C1CCOCC1] | XPW-0665 | XPW-0679 | XPW-0693 | XPW-0707 | XPW-0721 | XPW-0735 | XPW-0749 | XPW-0763 | XPW-0777 |
| 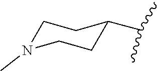 [SMILES: *C1CCN(C)CC1] | XPW-0666 | XPW-0680 | XPW-0694 | XPW-0708 | XPW-0722 | XPW-0736 | XPW-0750 | XPW-0764 | XPW-0778 |
| 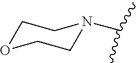 [SMILES: *N1CCOCC1] | XPW-0667 | XPW-0681 | XPW-0695 | XPW-0709 | XPW-0723 | XPW-0737 | XPW-0751 | XPW-0765 | XPW-0779 |
| 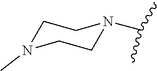 [SMILES: *N1CCN(C)CC1] | XPW-0668 | XPW-0682 | XPW-0696 | XPW-0710 | XPW-0724 | XPW-0738 | XPW-0752 | XPW-0766 | XPW-0780 |
| 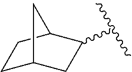 | XPW-0669 | XPW-0683 | XPW-0697 | XPW-0711 | XPW-0725 | XPW-0739 | XPW-0753 | XPW-0767 | XPW-0781 |
| 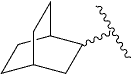 | XPW-0670 | XPW-0684 | XPW-0698 | XPW-0712 | XPW-0726 | XPW-0740 | XPW-0754 | XPW-0768 | XPW-0782 |
| 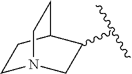 | XPW-0671 | XPW-0685 | XPW-0699 | XPW-0713 | XPW-0727 | XPW-0741 | XPW-0755 | XPW-0769 | XPW-0783 |
| 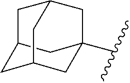 [SMILES: *C12CC3CC(CC(C3)C1)C2] | XPW-0672 | XPW-0686 | XPW-0700 | XPW-0714 | XPW-0728 | XPW-0742 | XPW-0756 | XPW-0770 | XPW-0784 |
| | | | | | | | A\B | 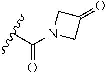 [SMILES: *C(=O)N1CC(=O)C1] | 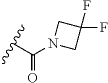 [SMILES: *C(=O)N1CC(F)(F)C1] |
| | | | | | | | 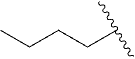 [SMILES: *CCCC] | XPW-0785 | XPW-0799 |
| | | | | | | | 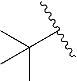 [SMILES: *C(C)(C)C] | XPW-0786 | XPW-0800 |
| | | | | | | | 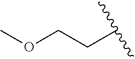 [SMILES: *CCOC] | XPW-0787 | XPW-0801 |
| | | | | | | | 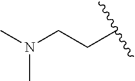 [SMILES: *CCN(C)C] | XPW-0788 | XPW-0802 |
| | | | | | | | 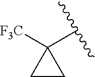 [SMILES: *C1(C(F)(F)F)CC1] | XPW-0789 | XPW-0803 |
| | | | | | | | 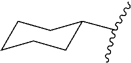 [SMILES: *C1CCCCC1] | XPW-0790 | XPW-0804 |
| | | | | | | | 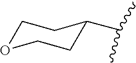 [SMILES: *C1CCOCC1] | XPW-0791 | XPW-0805 |
| | | | | | | | 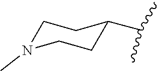 [SMILES: *C1CCN(C)CC1] | XPW-0792 | XPW-0806 |
| | | | | | | | 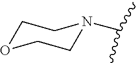 [SMILES: *N1CCOCC1] | XPW-0793 | XPW-0807 |
| | | | | | | | 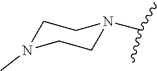 [SMILES: *N1CCN(C)CC1] | XPW-0794 | XPW-0808 |
| | | | | | | | 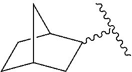 | XPW-0795 | XPW-0809 |
| | | | | | | | 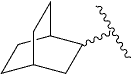 | XPW-0796 | XPW-0810 |
| | | | | | | | 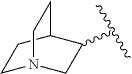 | XPW-0797 | XPW-0811 |
| | | | | | | | 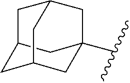 [SMILES: *C12CC3CC(CC(C3)C1)C2] | XPW-0798 | XPW-0812 |

The above table constitutes an individualized description of each of the specifically indicated compounds therein as well as their salts and solvates.

TABLE 12

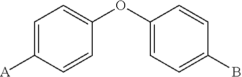

| A\B | 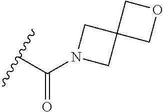 [SMILES: *C(=O)N1CC2(COC2)C1] | 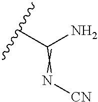 [SMILES: *C(N)=NC#N] | 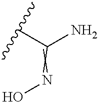 [SMILES: *C(N)=NO] |
|---|---|---|---|
| 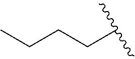 [SMILES: *CCCC] | XPW-0813 | XPW-0827 | XPW-4688 |
| 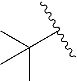 [SMILES: *C(C)(C)C] | XPW-0814 | XPW-0828 | XPW-4689 |
| 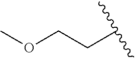 [SMILES: *CCOC] | XPW-0815 | XPW-0829 | XPW-4690 |
| 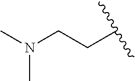 [SMILES: *CCN(C)C] | XPW-0816 | XPW-0830 | XPW-4691 |
| 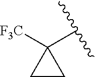 [SMILES: *C1(C(F)(F)F)CC1] | XPW-0817 | XPW-0831 | XPW-4692 |
| 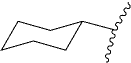 [SMILES: *C1CCCCC1] | XPW-0818 | XPW-0832 | XPW-4574 |
| 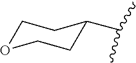 [SMILES: *C1CCOCC1] | XPW-0819 | XPW-0833 | XPW-4693 |
| 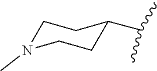 [SMILES: *C1CCN(C)CC1] | XPW-0820 | XPW-0834 | XPW-4694 |
| 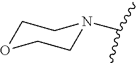 [SMILES: *N1CCOCC1] | XPW-0821 | XPW-0835 | XPW-4695 |
| 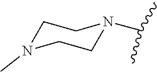 [SMILES: *N1CCN(C)CC1] | XPW-0822 | XPW-0836 | XPW-4696 |
| 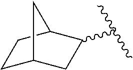 | XPW-0823 | XPW-0837 | XPW-4697 |
| 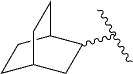 | XPW-0824 | XPW-0838 | XPW-4698 |
| 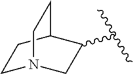 | XPW-0825 | XPW-0839 | XPW-4699 |
| 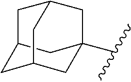 [SMILES: *C12CC3CC(CC(C3)C1)C2] | XPW-0826 | XPW-0840 | XPW-4700 |

The above table constitutes an individualized description of each of the specifically indicated compounds therein as well as their salts and solvates.

TABLE 13

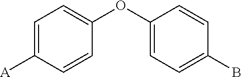

| A\B | 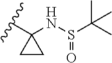 [SMILES: *C1(NS(=O)C(C)(C)C)CC1] | 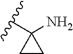 [SMILES: *C1(N)CC1] | 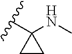 [SMILES: *C1(NC)CC1] | 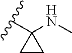 [SMILES: *C1(NC)CC1] | 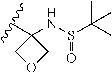 [SMILES: *C1(NS(=O)C(C)(C)C)COC1] | 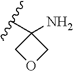 [SMILES: *C1(N)COC1] | 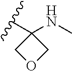 [SMILES: *C1(NC)COC1] | 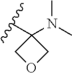 [SMILES: *C1(N(C)C)COC1] | 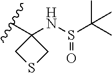 [SMILES: *C1(NS(=O)C(C)(C)C)CSC1] | 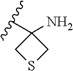 [SMILES: *C1(N)CSC1] | 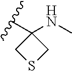 [SMILES: *C1(NC)CSC1] |
|---|---|---|---|---|---|---|---|---|---|---|---|
| 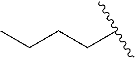 [SMILES: *CCCC] | XPW-0841 | XPW-0855 | XPW-0869 | XPW-0883 | XPW-0897 | XPW-0911 | XPW-0925 | XPW-0939 | XPW-0953 | XPW-0967 | XPW-0981 |
| 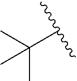 [SMILES: *C(C)(C)C] | XPW-0842 | XPW-0856 | XPW-0870 | XPW-0884 | XPW-0898 | XPW-0912 | XPW-0926 | XPW-0940 | XPW-0954 | XPW-0968 | XPW-0982 |
| 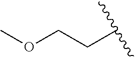 [SMILES: *CCOC] | XPW-0843 | XPW-0857 | XPW-0871 | XPW-0885 | XPW-0899 | XPW-0913 | XPW-0927 | XPW-0941 | XPW-0955 | XPW-0969 | XPW-0983 |
| 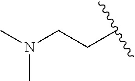 [SMILES: *CCN(C)C] | XPW-0844 | XPW-0858 | XPW-0872 | XPW-0886 | XPW-0900 | XPW-0914 | XPW-0928 | XPW-0942 | XPW-0956 | XPW-0970 | XPW-0984 |
| 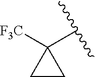 [SMILES: *C1(C(F)(F)F)CC1] | XPW-0845 | XPW-0859 | XPW-0873 | XPW-0887 | XPW-0901 | XPW-0915 | XPW-0929 | XPW-0943 | XPW-0957 | XPW-0971 | XPW-0985 |
| 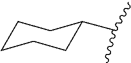 [SMILES: *C1CCCCC1] | XPW-0846 | XPW-0860 | XPW-0874 | XPW-0888 | XPW-0902 | XPW-0916 | XPW-0930 | XPW-0944 | XPW-0958 | XPW-0972 | XPW-0986 |
| 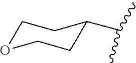 [SMILES: *C1CCOCC1] | XPW-0847 | XPW-0861 | XPW-0875 | XPW-0889 | XPW-0903 | XPW-0917 | XPW-0931 | XPW-0945 | XPW-0959 | XPW-0973 | XPW-0987 |
| 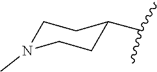 [SMILES: *C1CCN(C)CC1] | XPW-0848 | XPW-0862 | XPW-0876 | XPW-0890 | XPW-0904 | XPW-0918 | XPW-0932 | XPW-0946 | XPW-0960 | XPW-0974 | XPW-0988 |
| 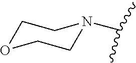 [SMILES: *N1CCOCC1] | XPW-0849 | XPW-0863 | XPW-0877 | XPW-0891 | XPW-0905 | XPW-0919 | XPW-0933 | XPW-0947 | XPW-0961 | XPW-0975 | XPW-0989 |
| 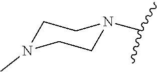 [SMILES: *N1CCN(C)CC1] | XPW-0850 | XPW-0864 | XPW-0878 | XPW-0892 | XPW-0906 | XPW-0920 | XPW-0934 | XPW-0948 | XPW-0962 | XPW-0976 | XPW-0990 |
| 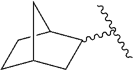 | XPW-0851 | XPW-0865 | XPW-0879 | XPW-0893 | XPW-0907 | XPW-0921 | XPW-0935 | XPW-0949 | XPW-0963 | XPW-0977 | XPW-0991 |
| 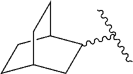 | XPW-0852 | XPW-0866 | XPW-0880 | XPW-0894 | XPW-0908 | XPW-0922 | XPW-0936 | XPW-0950 | XPW-0964 | XPW-0978 | XPW-0992 |
| 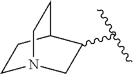 | XPW-0853 | XPW-0867 | XPW-0881 | XPW-0895 | XPW-0909 | XPW-0923 | XPW-0937 | XPW-0951 | XPW-0965 | XPW-0979 | XPW-0993 |
| 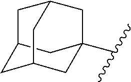 [SMILES: *C12CC3CC(CC(C3)C1)C2] | XPW-0854 | XPW-0868 | XPW-0882 | XPW-0896 | XPW-0910 | XPW-0924 | XPW-0938 | XPW-0952 | XPW-0966 | XPW-0980 | XPW-0994 |

The above table constitutes an individualized description of each of the specifically indicated compounds therein as well as their salts and solvates.

TABLE 14

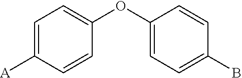

| A\B | 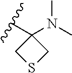 [SMILES: *C1(N(C)C)CSC1] | 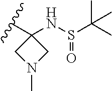 [SMILES: *C1(NS(=O)C(C)(C)C)CN(C)C1] | 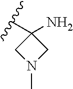 [SMILES: *C1(N)CN(C)C1] | 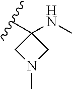 [SMILES: *C1(NC)CN(C)C1] | 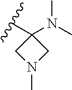 [SMILES: *C1(N(C)C)CN(C)C1] | 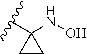 [SMILES: *C1(NO)CC1] | 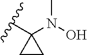 [SMILES: *C1(N(C)O)CC1] | 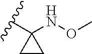 [SMILES: *C1(NOC)CC1] | 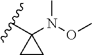 [SMILES: *C1(N(C)OC)CC1] | 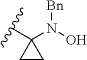 [SMILES: *C1(N(O)Cc2ccccc2)CC1] | 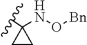 [SMILES: *C1(NOCc2ccccc2)CC1] |
|---|---|---|---|---|---|---|---|---|---|---|---|
| 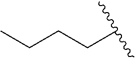 [SMILES: *CCCC] | XPW-0995 | XPW-1009 | XPW-1023 | XPW-1037 | XPW-1051 | XPW-3235 | XPW-3248 | XPW-3262 | XPW-3276 | XPW-3290 | XPW-3304 |
| 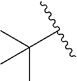 [SMILES: *C(C)(C)C] | XPW-0996 | XPW-1010 | XPW-1024 | XPW-1038 | XPW-1052 | XPW-4542 | XPW-3249 | XPW-3263 | XPW-3277 | XPW-3291 | XPW-3305 |
| 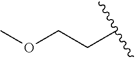 [SMILES: *CCOC] | XPW-0997 | XPW-1011 | XPW-1025 | XPW-1039 | XPW-1053 | XPW-3236 | XPW-3250 | XPW-3264 | XPW-3278 | XPW-3292 | XPW-3306 |
| 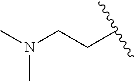 [SMILES: *CCN(C)C] | XPW-0998 | XPW-1012 | XPW-1026 | XPW-1040 | XPW-1054 | XPW-3237 | XPW-3251 | XPW-3265 | XPW-3279 | XPW-3293 | XPW-3307 |
| 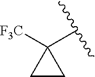 [SMILES: *C1(C(F)(F)F)CC1] | XPW-0999 | XPW-1013 | XPW-1027 | XPW-1041 | XPW-1055 | XPW-3238 | XPW-3252 | XPW-3266 | XPW-3280 | XPW-3294 | XPW-3308 |
| 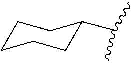 [SMILES: *C1CCCCC1] | XPW-1000 | XPW-1014 | XPW-1028 | XPW-1042 | XPW-1056 | XPW-3239 | XPW-3253 | XPW-3267 | XPW-3281 | XPW-3295 | XPW-3309 |
| 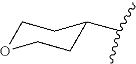 [SMILES: *C1CCOCC1] | XPW-1001 | XPW-1015 | XPW-1029 | XPW-1043 | XPW-1057 | XPW-3240 | XPW-3254 | XPW-3268 | XPW-3282 | XPW-3296 | XPW-3310 |
| 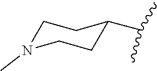 [SMILES: *C1CCN(C)CC1] | XPW-1002 | XPW-1016 | XPW-1030 | XPW-1044 | XPW-1058 | XPW-3241 | XPW-3255 | XPW-3269 | XPW-3283 | XPW-3297 | XPW-3311 |
| 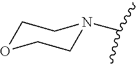 [SMILES: *N1CCOCC1] | XPW-1003 | XPW-1017 | XPW-1031 | XPW-1045 | XPW-1059 | XPW-3242 | XPW-3256 | XPW-3270 | XPW-3284 | XPW-3298 | XPW-3312 |
| 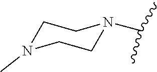 [SMILES: *N1CCN(C)CC1] | XPW-1004 | XPW-1018 | XPW-1032 | XPW-1046 | XPW-1060 | XPW-3243 | XPW-3257 | XPW-3271 | XPW-3285 | XPW-3299 | XPW-3313 |
| 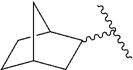 | XPW-1005 | XPW-1019 | XPW-1033 | XPW-1047 | XPW-1061 | XPW-3244 | XPW-3258 | XPW-3272 | XPW-3286 | XPW-3300 | XPW-3314 |
| 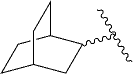 | XPW-1006 | XPW-1020 | XPW-1034 | XPW-1048 | XPW-1062 | XPW-3245 | XPW-3259 | XPW-3273 | XPW-3287 | XPW-3301 | XPW-3315 |
| 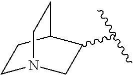 | XPW-1007 | XPW-1021 | XPW-1035 | XPW-1049 | XPW-1063 | XPW-3246 | XPW-3260 | XPW-3274 | XPW-3288 | XPW-3302 | XPW-3316 |
| 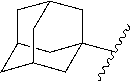 [SMILES: *C12CC3CC(CC(C3)C1)C2] | XPW-1008 | XPW-1022 | XPW-1036 | XPW-1050 | XPW-1064 | XPW-3247 | XPW-3261 | XPW-3275 | XPW-3289 | XPW-3303 | XPW-3317 |

The above table constitutes an individualized description of each of the specifically indicated compounds therein as well as their salts and solvates.

TABLE 15

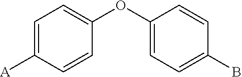

| A\B | 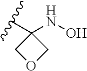 [SMILES: *C1(NO)COC1] | 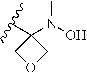 [SMILES: *C1(N(C)O)COC1] | 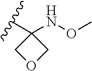 [SMILES: *C1(NOC)COC1] | 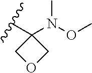 [SMILES: *C1(N(C)OC)COC1] | 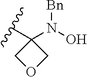 [SMILES: *C1(N(O)Cc2ccccc2)COC1] | 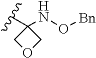 [SMILES: *C1(NOCc2ccccc2)COC1] | 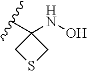 [SMILES: *C1(NO)CSC1] | 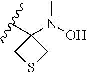 [SMILES: *C1(N(C)O)CSC1] | 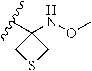 [SMILES: *C1(NOC)CSC1] | 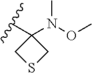 [SMILES: *C1(N(C)OC)CSC1] | 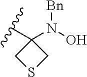 [SMILES: *C1(N(O)Cc2ccccc2)CSC1] |
|---|---|---|---|---|---|---|---|---|---|---|---|
| 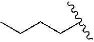 [SMILES: *CCCC] | XPW-3318 | XPW-3332 | XPW-3346 | XPW-3360 | XPW-3374 | XPW-3388 | XPW-3402 | XPW-3416 | XPW-3430 | XPW-3444 | XPW-3458 |
| 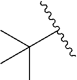 [SMILES: *C(C)(C)C] | XPW-3319 | XPW-3333 | XPW-3347 | XPW-3361 | XPW-3375 | XPW-3389 | XPW-3403 | XPW-3417 | XPW-3431 | XPW-3445 | XPW-3459 |
| 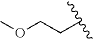 [SMILES: *CCOC] | XPW-3320 | XPW-3334 | XPW-3348 | XPW-3362 | XPW-3376 | XPW-3390 | XPW-3404 | XPW-3418 | XPW-3432 | XPW-3446 | XPW-3460 |
| 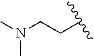 [SMILES: *CCN(C)C] | XPW-3321 | XPW-3335 | XPW-3349 | XPW-3363 | XPW-3377 | XPW-3391 | XPW-3405 | XPW-3419 | XPW-3433 | XPW-3447 | XPW-3461 |
| 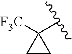 [SMILES: *C1(C(F)(F)F)CC1] | XPW-3322 | XPW-3336 | XPW-3350 | XPW-3364 | XPW-3378 | XPW-3392 | XPW-3406 | XPW-3420 | XPW-3434 | XPW-3448 | XPW-3462 |
| 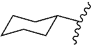 [SMILES: *C1CCCCC1] | XPW-3323 | XPW-3337 | XPW-3351 | XPW-3365 | XPW-3379 | XPW-3393 | XPW-3407 | XPW-3421 | XPW-3435 | XPW-3449 | XPW-3463 |
| 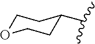 [SMILES: *C1CCOCC1] | XPW-3324 | XPW-3338 | XPW-3352 | XPW-3366 | XPW-3380 | XPW-3394 | XPW-3408 | XPW-3422 | XPW-3436 | XPW-3450 | XPW-3464 |
| 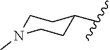 [SMILES: *C1CCN(C)CC1] | XPW-3325 | XPW-3339 | XPW-3353 | XPW-3367 | XPW-3381 | XPW-3395 | XPW-3409 | XPW-3423 | XPW-3437 | XPW-3451 | XPW-3465 |
| 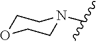 [SMILES: *N1CCOCC1] | XPW-3326 | XPW-3340 | XPW-3354 | XPW-3368 | XPW-3382 | XPW-3396 | XPW-3410 | XPW-3424 | XPW-3438 | XPW-3452 | XPW-3466 |
| 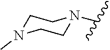 [SMILES: *N1CCN(C)CC1] | XPW-3327 | XPW-3341 | XPW-3355 | XPW-3369 | XPW-3383 | XPW-3397 | XPW-3411 | XPW-3425 | XPW-3439 | XPW-3453 | XPW-3467 |
| 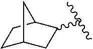 | XPW-3328 | XPW-3342 | XPW-3356 | XPW-3370 | XPW-3384 | XPW-3398 | XPW-3412 | XPW-3426 | XPW-3440 | XPW-3454 | XPW-3468 |
| 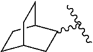 | XPW-3329 | XPW-3343 | XPW-3357 | XPW-3371 | XPW-3385 | XPW-3399 | XPW-3413 | XPW-3427 | XPW-3441 | XPW-3455 | XPW-3469 |
| 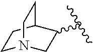 | XPW-3330 | XPW-3344 | XPW-3358 | XPW-3372 | XPW-3386 | XPW-3400 | XPW-3414 | XPW-3428 | XPW-3442 | XPW-3456 | XPW-3470 |
| 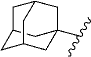 [SMILES: *C12CC3CC(CC(C3)C1)C2] | XPW-3331 | XPW-3345 | XPW-3359 | XPW-3373 | XPW-3387 | XPW-3401 | XPW-3415 | XPW-3429 | XPW-3443 | XPW-3457 | XPW-3471 |

The above table constitutes an individualized description of each of the specifically indicated compounds therein as well as their salts and solvates.

TABLE 16

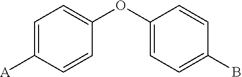

| A\B | 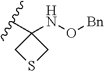 [SMILES: *C1(NOCc2ccccc2)CSC1] | 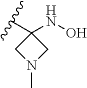 [SMILES: *C1(NO)CN(C)C1] | 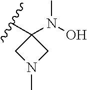 [SMILES: *C1(N(C)O)CN(C)C1] | 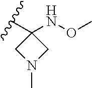 [SMILES: *C1(NOC)CN(C)C1] | 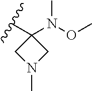 [SMILES: *C1(N(C)OC)CN(C)C1] | 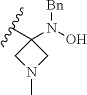 [SMILES: *C1(N(O)Cc2ccccc2)CN(C)C1] | 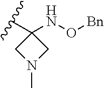 [SMILES: *C1(NOCc2ccccc2)CN(C)C1] | 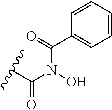 [SMILES: *C(=O)N(O)C(=O)c1ccccc1] |
|---|---|---|---|---|---|---|---|---|
| 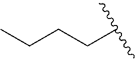 [SMILES: *CCCC] | XPW-3472 | XPW-3486 | XPW-3500 | XPW-3514 | XPW-3528 | XPW-3542 | XPW-3556 | XPW-4701 |
| 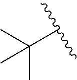 [SMILES: *C(C)(C)C] | XPW-3473 | XPW-3487 | XPW-3501 | XPW-3515 | XPW-3529 | XPW-3543 | XPW-3557 | XPW-4702 |
| 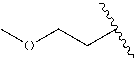 [SMILES: *CCOC] | XPW-3474 | XPW-3488 | XPW-3502 | XPW-3516 | XPW-3530 | XPW-3544 | XPW-3558 | XPW-4703 |
| 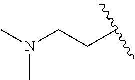 [SMILES: *CCN(C)C] | XPW-3475 | XPW-3489 | XPW-3503 | XPW-3517 | XPW-3531 | XPW-3545 | XPW-3559 | XPW-4704 |
| 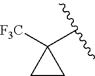 [SMILES: *C1(C(F)(F)F)CC1] | XPW-3476 | XPW-3490 | XPW-3504 | XPW-3518 | XPW-3532 | XPW-3546 | XPW-3560 | XPW-4705 |
| 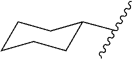 [SMILES: *C1CCCCC1] | XPW-3477 | XPW-3491 | XPW-3505 | XPW-3519 | XPW-3533 | XPW-3547 | XPW-3561 | XPW-4706 |
| 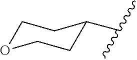 [SMILES: *C1CCOCC1] | XPW-3478 | XPW-3492 | XPW-3506 | XPW-3520 | XPW-3534 | XPW-3548 | XPW-3562 | XPW-4707 |
| 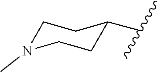 [SMILES: *C1CCN(C)CC1] | XPW-3479 | XPW-3493 | XPW-3507 | XPW-3521 | XPW-3535 | XPW-3549 | XPW-3563 | XPW-4708 |
| 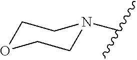 [SMILES: *N1CCOCC1] | XPW-3480 | XPW-3494 | XPW-3508 | XPW-3522 | XPW-3536 | XPW-3550 | XPW-3564 | XPW-4709 |
| 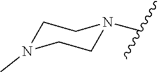 [SMILES: *N1CCN(C)CC1] | XPW-3481 | XPW-3495 | XPW-3509 | XPW-3523 | XPW-3537 | XPW-3551 | XPW-3565 | XPW-4710 |
| 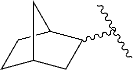 | XPW-3482 | XPW-3496 | XPW-3510 | XPW-3524 | XPW-3538 | XPW-3552 | XPW-3566 | XPW-4711 |
| 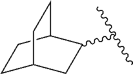 | XPW-3483 | XPW-3497 | XPW-3511 | XPW-3525 | XPW-3539 | XPW-3553 | XPW-3567 | XPW-4712 |
| 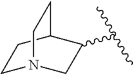 | XPW-3484 | XPW-3498 | XPW-3512 | XPW-3526 | XPW-3540 | XPW-3554 | XPW-3568 | XPW-4713 |
| 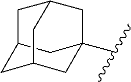 [SMILES: *C12CC3CC(CC(C3)C1)C2] | XPW-3485 | XPW-3499 | XPW-3513 | XPW-3527 | XPW-3541 | XPW-3555 | XPW-3569 | XPW-4647 |

The above table constitutes an individualized description of each of the specifically indicated compounds therein as well as their salts and solvates.

TABLE 17

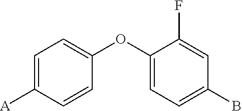

| A\B | 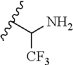 [SMILES: *C(N)C(F)(F)F] | 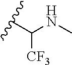 [SMILES: *C(NC)C(F)(F)F] | 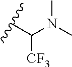 [SMILES: *C(N(C)C)C(F)(F)F] | 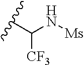 [SMILES: *C(NS(C)(=O)=O)C(F)(F)F] | 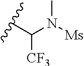 [SMILES: *C(N(C)S(C)(=O)=O)C(F)(F)F] | 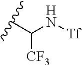 | 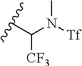 | 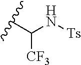 [SMILES: *C(NS(=O)(=O)c1ccc(C)cc1)C(F)(F)F] | 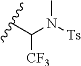 [SMILES: *C(N(C)S(=O)(=O)c1ccc(C)cc1)C(F)(F)F] | 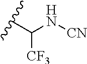 [SMILES: *C(NC#N)C(F)(F)F] | 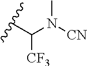 [SMILES: *C(N(C)C#N)C(F)(F)F] |
|---|---|---|---|---|---|---|---|---|---|---|---|
| 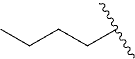 [SMILES: *CCCC] | XPW-1065 | XPW-1079 | XPW-1093 | XPW-1107 | XPW-1121 | XPW-1135 | XPW-1149 | XPW-1163 | XPW-1177 | XPW-1191 | XPW-1205 |
| 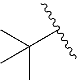 [SMILES: *C(C)(C)C] | XPW-1066 | XPW-1080 | XPW-1094 | XPW-1108 | XPW-1122 | XPW-1136 | XPW-1150 | XPW-1164 | XPW-1178 | XPW-1192 | XPW-1206 |
| 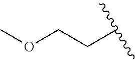 [SMILES: *CCOC] | XPW-1067 | XPW-1081 | XPW-1095 | XPW-1109 | XPW-1123 | XPW-1137 | XPW-1151 | XPW-1165 | XPW-1179 | XPW-1193 | XPW-1207 |
| 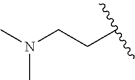 [SMILES: *CCN(C)C] | XPW-1068 | XPW-1082 | XPW-1096 | XPW-1110 | XPW-1124 | XPW-1138 | XPW-1152 | XPW-1166 | XPW-1180 | XPW-1194 | XPW-1208 |
| 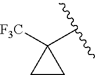 [SMILES: *C1(C(F)(F)F)CC1] | XPW-1069 | XPW-1083 | XPW-1097 | XPW-1111 | XPW-1125 | XPW-1139 | XPW-1153 | XPW-1167 | XPW-1181 | XPW-1195 | XPW-1209 |
| 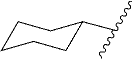 [SMILES: *C1CCCCC1] | XPW-1070 | XPW-1084 | XPW-1098 | XPW-1112 | XPW-1126 | XPW-1140 | XPW-1154 | XPW-1168 | XPW-1182 | XPW-1196 | XPW-1210 |
| 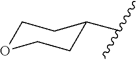 [SMILES: *C1CCOCC1] | XPW-1071 | XPW-1085 | XPW-1099 | XPW-1113 | XPW-1127 | XPW-1141 | XPW-1155 | XPW-1169 | XPW-1183 | XPW-1197 | XPW-1211 |
| 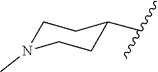 [SMILES: *C1CCN(C)CC1] | XPW-1072 | XPW-1086 | XPW-1100 | XPW-1114 | XPW-1128 | XPW-1142 | XPW-1156 | XPW-1170 | XPW-1184 | XPW-1198 | XPW-1212 |
| 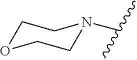 [SMILES: *N1CCOCC1] | XPW-1073 | XPW-1087 | XPW-1101 | XPW-1115 | XPW-1129 | XPW-1143 | XPW-1157 | XPW-1171 | XPW-1185 | XPW-1199 | XPW-1213 |
| 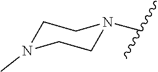 [SMILES: *N1CCN(C)CC1] | XPW-1074 | XPW-1088 | XPW-1102 | XPW-1116 | XPW-1130 | XPW-1144 | XPW-1158 | XPW-1172 | XPW-1186 | XPW-1200 | XPW-1214 |
| 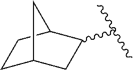 | XPW-1075 | XPW-1089 | XPW-1103 | XPW-1117 | XPW-1131 | XPW-1145 | XPW-1159 | XPW-1173 | XPW-1187 | XPW-1201 | XPW-1215 |
| 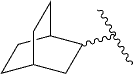 | XPW-1076 | XPW-1090 | XPW-1104 | XPW-1118 | XPW-1132 | XPW-1146 | XPW-1160 | XPW-1174 | XPW-1188 | XPW-1202 | XPW-1216 |
| 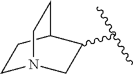 | XPW-1077 | XPW-1091 | XPW-1105 | XPW-1119 | XPW-1133 | XPW-1147 | XPW-1161 | XPW-1175 | XPW-1189 | XPW-1203 | XPW-1217 |
| 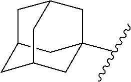 [SMILES: *C12CC3CC(CC(C3)C1)C2] | XPW-1078 | XPW-1092 | XPW-1106 | XPW-1120 | XPW-1134 | XPW-1148 | XPW-1162 | XPW-1176 | XPW-1190 | XPW-1204 | XPW-1218 |

The above table constitutes an individualized description of each of the specifically indicated compounds therein as well as their salts and solvates.

TABLE 18

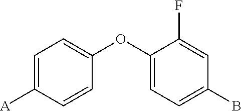

| A\B | 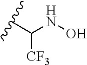 [SMILES: *C(NO)C(F)(F)F] | 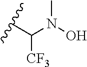 [SMILES: *C(N(C)O)C(F)(F)F] | 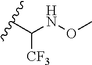 [SMILES: *C(NOC)C(F)(F)F] | 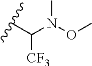 [SMILES: *C(N(C)OC)C(F)(F)F] | 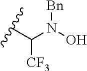 [SMILES: *C(N(O)Cc1ccccc1)C(F)(F)F] | 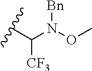 [SMILES: *C(N(Cc1ccccc1)OC)C(F)(F)F] | 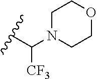 [SMILES: *C(N1CCOCC1)C(F)(F)F] | 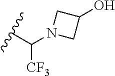 [SMILES: *C(N1CC(O)C1)C(F)(F)F] | 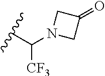 [SMILES: *C(N1CC(=O)C1)C(F)(F)F] |
|---|---|---|---|---|---|---|---|---|---|
| 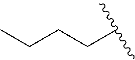 [SMILES: *CCCC] | XPW-1219 | XPW-1233 | XPW-1247 | XPW-1261 | XPW-1275 | XPW-1289 | XPW-1303 | XPW-1317 | XPW-1331 |
| 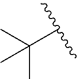 [SMILES: *C(C)(C)C] | XPW-1220 | XPW-1234 | XPW-1248 | XPW-1262 | XPW-1276 | XPW-1290 | XPW-1304 | XPW-1318 | XPW-1332 |
| 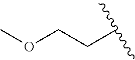 [SMILES: *CCOC] | XPW-1221 | XPW-1235 | XPW-1249 | XPW-1263 | XPW-1277 | XPW-1291 | XPW-1305 | XPW-1319 | XPW-1333 |
| 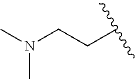 [SMILES: *CCN(C)C] | XPW-1222 | XPW-1236 | XPW-1250 | XPW-1264 | XPW-1278 | XPW-1292 | XPW-1306 | XPW-1320 | XPW-1334 |
| 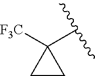 [SMILES: *C1(C(F)(F)F)CC1] | XPW-1223 | XPW-1237 | XPW-1251 | XPW-1265 | XPW-1279 | XPW-1293 | XPW-1307 | XPW-1321 | XPW-1335 |
| 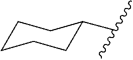 [SMILES: *C1CCCCC1] | XPW-1224 | XPW-1238 | XPW-1252 | XPW-1266 | XPW-1280 | XPW-1294 | XPW-1308 | XPW-1322 | XPW-1336 |
| 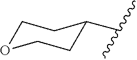 [SMILES: *C1CCOCC1] | XPW-1225 | XPW-1239 | XPW-1253 | XPW-1267 | XPW-1281 | XPW-1295 | XPW-1309 | XPW-1323 | XPW-1337 |
| 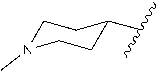 [SMILES: *C1CCN(C)CC1] | XPW-1226 | XPW-1240 | XPW-1254 | XPW-1268 | XPW-1282 | XPW-1296 | XPW-1310 | XPW-1324 | XPW-1338 |
| 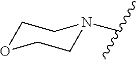 [SMILES: *N1CCOCC1] | XPW-1227 | XPW-1241 | XPW-1255 | XPW-1269 | XPW-1283 | XPW-1297 | XPW-1311 | XPW-1325 | XPW-1339 |
| 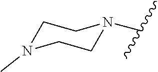 [SMILES: *N1CCN(C)CC1] | XPW-1228 | XPW-1242 | XPW-1256 | XPW-1270 | XPW-1284 | XPW-1298 | XPW-1312 | XPW-1326 | XPW-1340 |
| 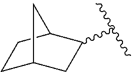 | XPW-1229 | XPW-1243 | XPW-1257 | XPW-1271 | XPW-1285 | XPW-1299 | XPW-1313 | XPW-1327 | XPW-1341 |
| 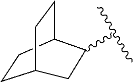 | XPW-1230 | XPW-1244 | XPW-1258 | XPW-1272 | XPW-1286 | XPW-1300 | XPW-1314 | XPW-1328 | XPW-1342 |
| 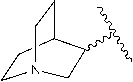 | XPW-1231 | XPW-1245 | XPW-1259 | XPW-1273 | XPW-1287 | XPW-1301 | XPW-1315 | XPW-1329 | XPW-1343 |
| 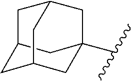 [SMILES: *C12CC3CC(CC(C3)C1)C2] | XPW-1232 | XPW-1246 | XPW-1260 | XPW-1274 | XPW-1288 | XPW-1302 | XPW-1316 | XPW-1330 | XPW-1344 |
| | | | | | | | A\B | 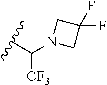 [SMILES: *C(N1CC(F)(F)C1)C(F)(F)F] | 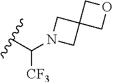 [SMILES: *C(N1CC2(COC2)C1)C(F)(F)F] |
| | | | | | | | 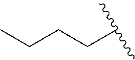 [SMILES: *CCCC] | XPW-1345 | XPW-1359 |
| | | | | | | | 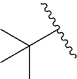 [SMILES: *C(C)(C)C] | XPW-1346 | XPW-1360 |
| | | | | | | | 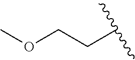 [SMILES: *CCOC] | XPW-1347 | XPW-1361 |
| | | | | | | | 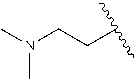 [SMILES: *CCN(C)C] | XPW-1348 | XPW-1362 |
| | | | | | | | 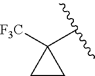 [SMILES: *C1(C(F)(F)F)CC1] | XPW-1349 | XPW-1363 |
| | | | | | | | 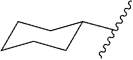 [SMILES: *C1CCCCC1] | XPW-1350 | XPW-1364 |
| | | | | | | | 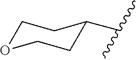 [SMILES: *C1CCOCC1] | XPW-1351 | XPW-1365 |
| | | | | | | | 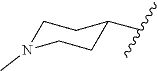 [SMILES: *C1CCN(C)CC1] | XPW-1352 | XPW-1366 |
| | | | | | | | 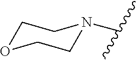 [SMILES: *N1CCOCC1] | XPW-1353 | XPW-1367 |
| | | | | | | | 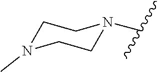 [SMILES: *N1CCN(C)CC1] | XPW-1354 | XPW-1368 |
| | | | | | | | 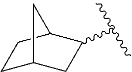 | XPW-1355 | XPW-1369 |
| | | | | | | | 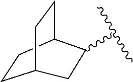 | XPW-1356 | XPW-1370 |
| | | | | | | | 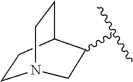 | XPW-1357 | XPW-1371 |
| | | | | | | | 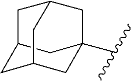 [SMILES: *C12CC3CC(CC(C3)C1)C2] | XPW-1358 | XPW-1372 |

The above table constitutes an individualized description of each of the specifically indicated compounds therein as well as their salts and solvates.

TABLE 19

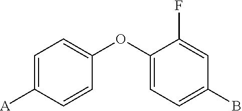

| A\B | 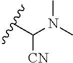 [SMILES: *C(C#N)N(C)C] | 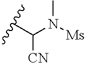 [SMILES: *C(C#N)N(C)S(C)(=O)=O] | 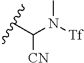 | 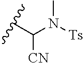 [SMILES: *C(C#N)N(C)S(=O)(=O)c1ccc(C)cc1] | 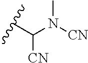 [SMILES: *C(C#N)N(C)C#N] | 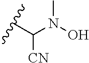 [SMILES: *C(C#N)N(C)O] | 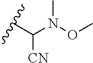 [SMILES: *C(C#N)N(C)OC] | 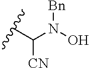 [SMILES: *C(C#N)N(O)Cc1ccccc1] | 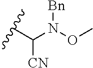 [SMILES: *C(C#N)N(Cc1ccccc1)OC] |
|---|---|---|---|---|---|---|---|---|---|
| 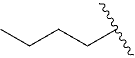 [SMILES: *CCCC] | XPW-1373 | XPW-1387 | XPW-1401 | XPW-1415 | XPW-1429 | XPW-1443 | XPW-1457 | XPW-1471 | XPW-1485 |
| 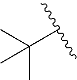 [SMILES: *C(C)(C)C] | XPW-1374 | XPW-1388 | XPW-1402 | XPW-1416 | XPW-1430 | XPW-1444 | XPW-1458 | XPW-1472 | XPW-1486 |
| 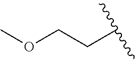 [SMILES: *CCOC] | XPW-1375 | XPW-1389 | XPW-1403 | XPW-1417 | XPW-1431 | XPW-1445 | XPW-1459 | XPW-1473 | XPW-1487 |
| 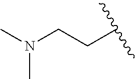 [SMILES: *CCN(C)C] | XPW-1376 | XPW-1390 | XPW-1404 | XPW-1418 | XPW-1432 | XPW-1446 | XPW-1460 | XPW-1474 | XPW-1488 |
| 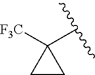 [SMILES: *C1(C(F)(F)F)CC1] | XPW-1377 | XPW-1391 | XPW-1405 | XPW-1419 | XPW-1433 | XPW-1447 | XPW-1461 | XPW-1475 | XPW-1489 |
| 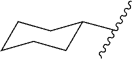 [SMILES: *C1CCCCC1] | XPW-1378 | XPW-1392 | XPW-1406 | XPW-1420 | XPW-1434 | XPW-1448 | XPW-1462 | XPW-1476 | XPW-1490 |
| 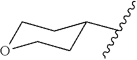 [SMILES: *C1CCOCC1] | XPW-1379 | XPW-1393 | XPW-1407 | XPW-1421 | XPW-1435 | XPW-1449 | XPW-1463 | XPW-1477 | XPW-1491 |
| 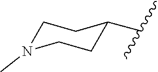 [SMILES: *C1CCN(C)CC1] | XPW-1380 | XPW-1394 | XPW-1408 | XPW-1422 | XPW-1436 | XPW-1450 | XPW-1464 | XPW-1478 | XPW-1492 |
| 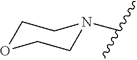 [SMILES: *N1CCOCC1] | XPW-1381 | XPW-1395 | XPW-1409 | XPW-1423 | XPW-1437 | XPW-1451 | XPW-1465 | XPW-1479 | XPW-1493 |
| 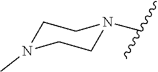 [SMILES: *N1CCN(C)CC1] | XPW-1382 | XPW-1396 | XPW-1410 | XPW-1424 | XPW-1438 | XPW-1452 | XPW-1466 | XPW-1480 | XPW-1494 |
| 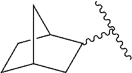 | XPW-1383 | XPW-1397 | XPW-1411 | XPW-1425 | XPW-1439 | XPW-1453 | XPW-1467 | XPW-1481 | XPW-1495 |
| 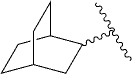 | XPW-1384 | XPW-1398 | XPW-1412 | XPW-1426 | XPW-1440 | XPW-1454 | XPW-1468 | XPW-1482 | XPW-1496 |
| 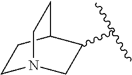 | XPW-1385 | XPW-1399 | XPW-1413 | XPW-1427 | XPW-1441 | XPW-1455 | XPW-1469 | XPW-1483 | XPW-1497 |
| 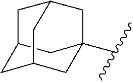 [SMILES: *C12CC3CC(CC(C3)C1)C2] | XPW-1386 | XPW-1400 | XPW-1414 | XPW-1428 | XPW-1442 | XPW-1456 | XPW-1470 | XPW-1484 | XPW-1498 |
| | | | | | | | A\B | 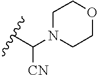 [SMILES: *C(C#N)N1CCOCC1] | 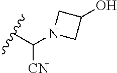 [SMILES: *C(C#N)N1CC(O)C1] |
| | | | | | | | 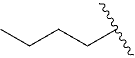 [SMILES: *CCCC] | XPW-1499 | XPW-1513 |
| | | | | | | | 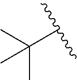 [SMILES: *C(C)(C)C] | XPW-1500 | XPW-1514 |
| | | | | | | | 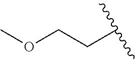 [SMILES: *CCOC] | XPW-1501 | XPW-1515 |
| | | | | | | | 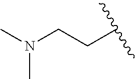 [SMILES: *CCN(C)C] | XPW-1502 | XPW-1516 |
| | | | | | | | 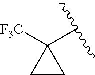 [SMILES: *C1(C(F)(F)F)CC1] | XPW-1503 | XPW-1517 |
| | | | | | | | 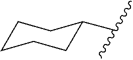 [SMILES: *C1CCCCC1] | XPW-1504 | XPW-1518 |
| | | | | | | | 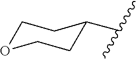 [SMILES: *C1CCOCC1] | XPW-1505 | XPW-1519 |
| | | | | | | | 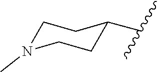 [SMILES: *C1CCN(C)CC1] | XPW-1506 | XPW-1520 |
| | | | | | | | 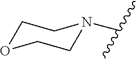 [SMILES: *N1CCOCC1] | XPW-1507 | XPW-1521 |
| | | | | | | | 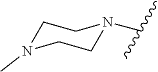 [SMILES: *N1CCN(C)CC1] | XPW-1508 | XPW-1522 |
| | | | | | | | 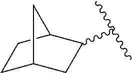 | XPW-1509 | XPW-1523 |
| | | | | | | | 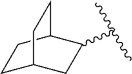 | XPW-1510 | XPW-1524 |
| | | | | | | | 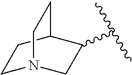 | XPW-1511 | XPW-1525 |
| | | | | | | | 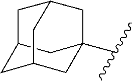 [SMILES: *C12CC3CC(CC(C3)C1)C2] | XPW-1512 | XPW-1526 |

The above table constitutes an individualized description of each of the specifically indicated compounds therein as well as their salts and solvates.

TABLE 20

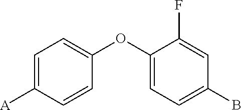

| A\B | 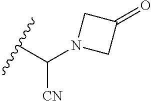 [SMILES: *C(C#N)N1CC(=O)C1] | 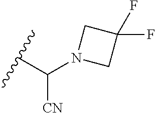 [SMILES: *C(C#N)N1CC(F)(F)C1] | 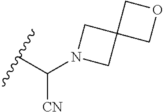 [SMILES: *C(C#N)N1CC2(COC2)C1] | 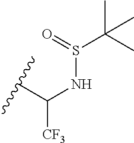 [SMILES: *C(NS(=O)C(C)(C)C)C(F)(F)F] | 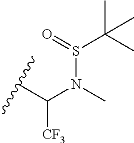 [SMILES: *C(N(C)S(=O)C(C)(C)C)C(F)(F)F] | 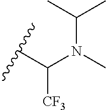 [SMILES: *C(N(C)C(C)C)C(F)(F)F] |
|---|---|---|---|---|---|---|
| 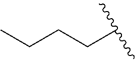 [SMILES: *CCCC] | XPW-1527 | XPW-1541 | XPW-1555 | XPW-4902 | XPW-4916 | XPW-4930 |
| 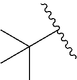 [SMILES: *C(C)(C)C] | XPW-1528 | XPW-1542 | XPW-1556 | XPW-4903 | XPW-4917 | XPW-4931 |
| 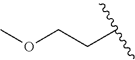 [SMILES: *CCOC] | XPW-1529 | XPW-1543 | XPW-1557 | XPW-4904 | XPW-4918 | XPW-4932 |
| 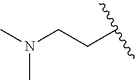 [SMILES: *CCN(C)C] | XPW-1530 | XPW-1544 | XPW-1558 | XPW-4905 | XPW-4919 | XPW-4933 |
| 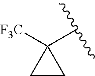 [SMILES: *C1(C(F)(F)F)CC1] | XPW-1531 | XPW-1545 | XPW-1559 | XPW-4906 | XPW-4920 | XPW-4934 |
| 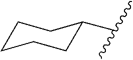 [SMILES: *C1CCCCC1] | XPW-1532 | XPW-1546 | XPW-1560 | XPW-4907 | XPW-4921 | XPW-4935 |
| 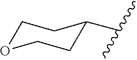 [SMILES: *C1CCOCC1] | XPW-1533 | XPW-1547 | XPW-1561 | XPW-4908 | XPW-4922 | XPW-4936 |
| 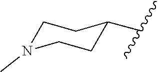 [SMILES: *C1CCN(C)CC1] | XPW-1534 | XPW-1548 | XPW-1562 | XPW-4909 | XPW-4923 | XPW-4937 |
| 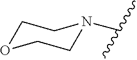 [SMILES: *N1CCOCC1] | XPW-1535 | XPW-1549 | XPW-1563 | XPW-4910 | XPW-4924 | XPW-4938 |
| 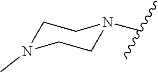 [SMILES: *N1CCN(C)CC1] | XPW-1536 | XPW-1S50 | XPW-1564 | XPW-4911 | XPW-4925 | XPW-4939 |
| 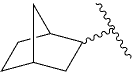 | XPW-1537 | XPW-1551 | XPW-1565 | XPW-4912 | XPW-4926 | XPW-4940 |
| 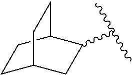 | XPW-1538 | XPW-1552 | XPW-1566 | XPW-4913 | XPW-4927 | XPW-4941 |
| 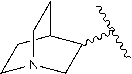 | XPW-1539 | XPW-1S53 | XPW-1567 | XPW-4914 | XPW-4928 | XPW-4942 |
| 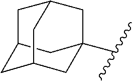 [SMILES: *C12CC3CC(CC(C3)C1)C2] | XPW-1540 | XPW-1554 | XPW-1568 | XPW-4915 | XPW-4929 | XPW-4943 |

The above table constitutes an individualized description of each of the specifically indicated compounds therein as well as their salts and solvates.

TABLE 21

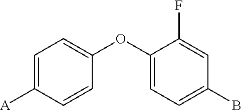

| A\B | 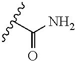 [SMILES: *C(N)=O] | 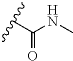 [SMILES: *C(=O)NC] | 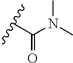 [SMILES: *C(=O)N(C)C] | 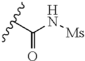 [SMILES: *C(=O)NS(C)(=O)=O] | 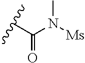 [SMILES: *C(=O)N(C)S(C)(=O)=O] | 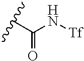 | 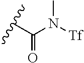 | 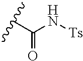 [SMILES: *C(=O)NS(=O)(=O)c1ccc(C)cc1] | 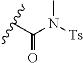 [SMILES: *C(=O)N(C)S(=O)(=O)c1ccc(C)cc1] | 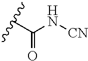 [SMILES: *C(=O)NC#N] | 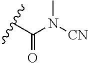 [SMILES: *C(=O)N(C)C#N] |
|---|---|---|---|---|---|---|---|---|---|---|---|
| 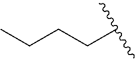 [SMILES: *CCCC] | XPW-1569 | XPW-1583 | XPW-1597 | XPW-1611 | XPW-1625 | XPW-1639 | XPW-1653 | XPW-1667 | XPW-1681 | XPW-1695 | XPW-1709 |
| 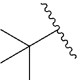 [SMILES: *C(C)(C)C] | XPW-1570 | XPW-1584 | XPW-1598 | XPW-1612 | XPW-1626 | XPW-1640 | XPW-1654 | XPW-1668 | XPW-1682 | XPW-1696 | XPW-1710 |
| 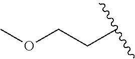 [SMILES: *CCOC] | XPW-1571 | XPW-1585 | XPW-1599 | XPW-1613 | XPW-1627 | XPW-1641 | XPW-1655 | XPW-1669 | XPW-1683 | XPW-1697 | XPW-1711 |
| 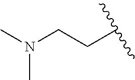 [SMILES: *CCN(C)C] | XPW-1572 | XPW-1586 | XPW-1600 | XPW-1614 | XPW-1628 | XPW-1642 | XPW-1656 | XPW-1670 | XPW-1684 | XPW-1698 | XPW-1712 |
| 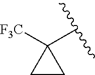 [SMILES: *C1(C(F)(F)F)CC1] | XPW-1573 | XPW-1587 | XPW-1601 | XPW-1615 | XPW-1629 | XPW-1643 | XPW-1657 | XPW-1671 | XPW-1685 | XPW-1699 | XPW-1713 |
| 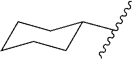 [SMILES: *C1CCCCC1] | XPW-1574 | XPW-1588 | XPW-1602 | XPW-1616 | XPW-1630 | XPW-1644 | XPW-1658 | XPW-1672 | XPW-1686 | XPW-1700 | XPW-1714 |
| 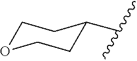 [SMILES: *C1CCOCC1] | XPW-1575 | XPW-1589 | XPW-1603 | XPW-1617 | XPW-1631 | XPW-1645 | XPW-1659 | XPW-1673 | XPW-1687 | XPW-1701 | XPW-1715 |
| 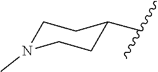 [SMILES: *C1CCN(C)CC1] | XPW-1576 | XPW-1590 | XPW-1604 | XPW-1618 | XPW-1632 | XPW-1646 | XPW-1660 | XPW-1674 | XPW-1688 | XPW-1702 | XPW-1716 |
| 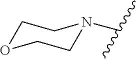 [SMILES: *N1CCOCC1] | XPW-1577 | XPW-1591 | XPW-1605 | XPW-1619 | XPW-1633 | XPW-1647 | XPW-1661 | XPW-1675 | XPW-1689 | XPW-1703 | XPW-1717 |
| 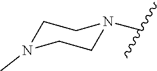 [SMILES: *N1CCN(C)CC1] | XPW-1578 | XPW-1S92 | XPW-1606 | XPW-1620 | XPW-1634 | XPW-1648 | XPW-1662 | XPW-1676 | XPW-1690 | XPW-1704 | XPW-1718 |
| 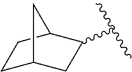 | XPW-1579 | XPW-1593 | XPW-1607 | XPW-1621 | XPW-1635 | XPW-1649 | XPW-1663 | XPW-1677 | XPW-1691 | XPW-1705 | XPW-1719 |
| 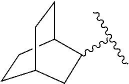 | XPW-1580 | XPW-1594 | XPW-1608 | XPW-1622 | XPW-1636 | XPW-1650 | XPW-1664 | XPW-1678 | XPW-1692 | XPW-1706 | XPW-1720 |
| 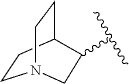 | XPW-1581 | XPW-1S95 | XPW-1609 | XPW-1623 | XPW-1637 | XPW-1651 | XPW-1665 | XPW-1679 | XPW-1693 | XPW-1707 | XPW-1721 |
| 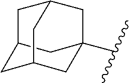 [SMILES: *C12CC3CC(CC(C3)C1)C2] | XPW-1582 | XPW-1596 | XPW-1610 | XPW-1624 | XPW-1638 | XPW-1652 | XPW-1666 | XPW-1680 | XPW-1694 | XPW-1708 | XPW-1722 |

The above table constitutes an individualized description of each of the specifically indicated compounds therein as well as their salts and solvates.

TABLE 22

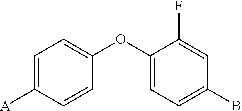

| A\B | 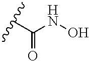 [SMILES: *C(=O)NO] | 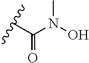 [SMILES: *C(=O)N(C)O] | 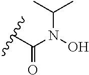 [SMILES: *C(=O)N(O)C(C)C] | 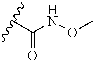 [SMILES: *C(=O)NOC] | 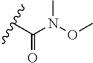 [SMILES: *C(=O)N(C)OC] | 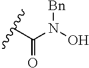 [SMILES: *C(=O)N(O)Cc1ccccc1] | 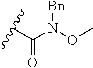 [SMILES: *C(=O)N(Cc1ccccc1)OC] | 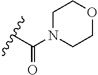 [SMILES: *C(=O)N1CCOCC1] | 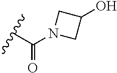 [SMILES: *C(=O)N1CC(O)C1] |
|---|---|---|---|---|---|---|---|---|---|
| 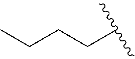 [SMILES: *CCCC] | XPW-1723 | XPW-1737 | XPW-1751 | XPW-1765 | XPW-1779 | XPW-1793 | XPW-1807 | XPW-1821 | XPW-1835 |
| 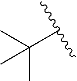 [SMILES: *C(C)(C)C] | XPW-1724 | XPW-1738 | XPW-1752 | XPW-1766 | XPW-1780 | XPW-1794 | XPW-1808 | XPW-1822 | XPW-1836 |
| 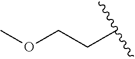 [SMILES: *CCOC] | XPW-1725 | XPW-1739 | XPW-1753 | XPW-1767 | XPW-1781 | XPW-1795 | XPW-1809 | XPW-1823 | XPW-1837 |
| 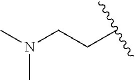 [SMILES: *CCN(C)C] | XPW-1726 | XPW-1740 | XPW-1754 | XPW-1768 | XPW-1782 | XPW-1796 | XPW-1810 | XPW-1824 | XPW-1838 |
| 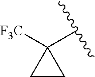 [SMILES: *C1(C(F)(F)F)CC1] | XPW-1727 | XPW-1741 | XPW-1755 | XPW-1769 | XPW-1783 | XPW-1797 | XPW-1811 | XPW-1825 | XPW-1839 |
| 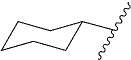 [SMILES: *C1CCCCC1] | XPW-1728 | XPW-1742 | XPW-1756 | XPW-1770 | XPW-1784 | XPW-1798 | XPW-1812 | XPW-1826 | XPW-1840 |
| 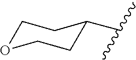 [SMILES: *C1CCOCC1] | XPW-1729 | XPW-1743 | XPW-1757 | XPW-1771 | XPW-1785 | XPW-1799 | XPW-1813 | XPW-1827 | XPW-1841 |
| 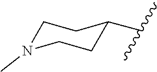 [SMILES: *C1CCN(C)CC1] | XPW-1730 | XPW-1744 | XPW-1758 | XPW-1772 | XPW-1786 | XPW-1800 | XPW-1814 | XPW-1828 | XPW-1842 |
| 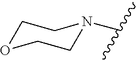 [SMILES: *N1CCOCC1] | XPW-1731 | XPW-1745 | XPW-1759 | XPW-1773 | XPW-1787 | XPW-1801 | XPW-1815 | XPW-1829 | XPW-1843 |
| 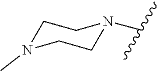 [SMILES: *N1CCN(C)CC1] | XPW-1732 | XPW-1746 | XPW-1760 | XPW-1774 | XPW-1788 | XPW-1802 | XPW-1816 | XPW-1830 | XPW-1844 |
| 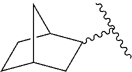 | XPW-1733 | XPW-1747 | XPW-1761 | XPW-1775 | XPW-1789 | XPW-1803 | XPW-1817 | XPW-1831 | XPW-1845 |
| 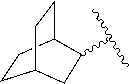 | XPW-1734 | XPW-1748 | XPW-1762 | XPW-1776 | XPW-1790 | XPW-1804 | XPW-1818 | XPW-1832 | XPW-1846 |
| 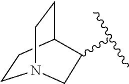 | XPW-1735 | XPW-1749 | XPW-1763 | XPW-1777 | XPW-1791 | XPW-1805 | XPW-1819 | XPW-1833 | XPW-1847 |
| 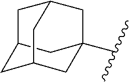 [SMILES: *C12CC3CC(CC(C3)C1)C2] | XPW-1736 | XPW-1750 | XPW-1764 | XPW-1778 | XPW-1792 | XPW-1806 | XPW-1820 | XPW-1834 | XPW-1848 |
| | | | | | | | A\B | 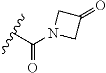 [SMILES: *C(=O)N1CC(=O)C1] | 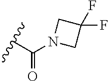 [SMILES: *C(=O)N1CC(F)(F)C1] |
| | | | | | | | 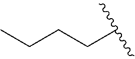 [SMILES: *CCCC] | XPW-1849 | XPW-1863 |
| | | | | | | | 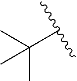 [SMILES: *C(C)(C)C] | XPW-1850 | XPW-1864 |
| | | | | | | | 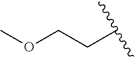 [SMILES: *CCOC] | XPW-1851 | XPW-1865 |
| | | | | | | | 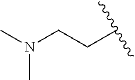 [SMILES: *CCN(C)C] | XPW-1852 | XPW-1866 |
| | | | | | | | 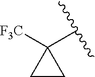 [SMILES: *C1(C(F)(F)F)CC1] | XPW-1853 | XPW-1867 |
| | | | | | | | 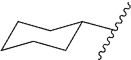 [SMILES: *C1CCCCC1] | XPW-1854 | XPW-1868 |
| | | | | | | | 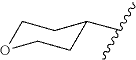 [SMILES: *C1CCOCC1] | XPW-1855 | XPW-1869 |
| | | | | | | | 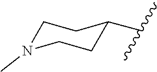 [SMILES: *C1CCN(C)CC1] | XPW-1856 | XPW-1870 |
| | | | | | | | 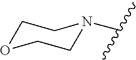 [SMILES: *N1CCOCC1] | XPW-1857 | XPW-1871 |
| | | | | | | | 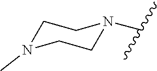 [SMILES: *N1CCN(C)CC1] | XPW-1858 | XPW-1872 |
| | | | | | | | 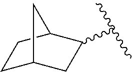 | XPW-1859 | XPW-1873 |
| | | | | | | | 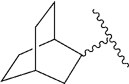 | XPW-1860 | XPW-1874 |
| | | | | | | | 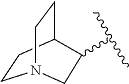 | XPW-1861 | XPW-1875 |
| | | | | | | | 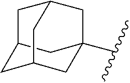 [SMILES: *C12CC3CC(CC(C3)C1)C2] | XPW-1862 | XPW-1876 |

The above table constitutes an individualized description of each of the specifically indicated compounds therein as well as their salts and solvates.

TABLE 23

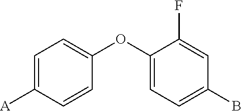

| A\B | 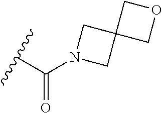 [SMILES: *C(=O)N1CC2(COC2)C1] | 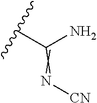 [SMILES: *C(N)=NC#N] | 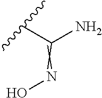 [SMILES: *C(N)=NO] |
|---|---|---|---|
| 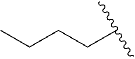 [SMILES: *CCCC] | XPW-1877 | XPW-1891 | XPW-4944 |
| 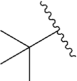 [SMILES: *C(C)(C)C] | XPW-1878 | XPW-1892 | XPW-4945 |
| 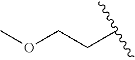 [SMILES: *CCOC] | XPW-1879 | XPW-1893 | XPW-4946 |
| 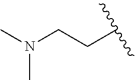 [SMILES: *CCN(C)C] | XPW-1880 | XPW-1894 | XPW-4947 |
| 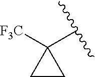 [SMILES: *C1(C(F)(F)F)CC1] | XPW-1881 | XPW-1895 | XPW-4948 |
| 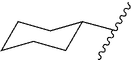 [SMILES: *C1CCCCC1] | XPW-1882 | XPW-1896 | XPW-4949 |
| 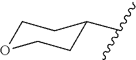 [SMILES: *C1CCOCC1] | XPW-1883 | XPW-1897 | XPW-4950 |
| 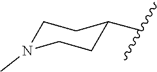 [SMILES: *C1CCN(C)CC1] | XPW-1884 | XPW-1898 | XPW-4951 |
| 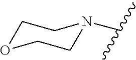 [SMILES: *N1CCOCC1] | XPW-1885 | XPW-1899 | XPW-4952 |
| 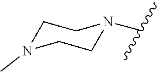 [SMILES: *N1CCN(C)CC1] | XPW-1886 | XPW-1900 | XPW-4953 |
| 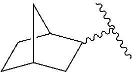 | XPW-1887 | XPW-1901 | XPW-4954 |
| 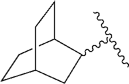 | XPW-1888 | XPW-1902 | XPW-4955 |
| 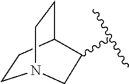 | XPW-1889 | XPW-1903 | XPW-4956 |
| 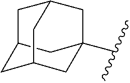 [SMILES: *C12CC3CC(CC(C3)C1)C2] | XPW-1890 | XPW-1904 | XPW-4957 |

The above table constitutes an individualized description of each of the specifically indicated compounds therein as well as their salts and solvates.

TABLE 24

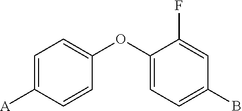

| A\B | 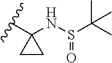 [SMILES: *C1(NS(=O)C(C)(C)C)CC1] | 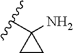 [SMILES: *C1(N)CC1] | 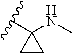 [SMILES: *C1(NC)CC1] | 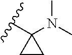 [SMILES: *C1(N(C)C)CC1] | 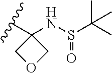 [SMILES: *C1(NS(=O)C(C)(C)C)COC1] | 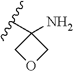 [SMILES: *C1(N)COC1] | 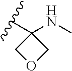 [SMILES: *C1(NC)COC1] | 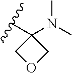 [SMILES: *C1(N(C)C)COC1] | 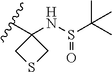 [SMILES: *C1(NS(=O)C(C)(C)C)CSC1] | 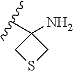 [SMILES: *C1(N)CSC1] | 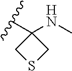 [SMILES: *C1(NC)CSC1] |
|---|---|---|---|---|---|---|---|---|---|---|---|
| 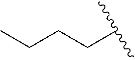 [SMILES: *CCCC] | XPW-1905 | XPW-1919 | XPW-1933 | XPW-1947 | XPW-1961 | XPW-1975 | XPW-1989 | XPW-2003 | XPW-2017 | XPW-2031 | XPW-2045 |
| 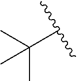 [SMILES: *C(C)(C)C] | XPW-1906 | XPW-1920 | XPW-1934 | XPW-1948 | XPW-1962 | XPW-1976 | XPW-1990 | XPW-2004 | XPW-2018 | XPW-2032 | XPW-2046 |
| 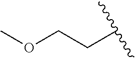 [SMILES: *CCOC] | XPW-1907 | XPW-1921 | XPW-1935 | XPW-1949 | XPW-1963 | XPW-1977 | XPW-1991 | XPW-2005 | XPW-2019 | XPW-2033 | XPW-2047 |
| 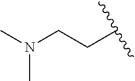 [SMILES: *CCN(C)C] | XPW-1908 | XPW-1922 | XPW-1936 | XPW-1950 | XPW-1964 | XPW-1978 | XPW-1992 | XPW-2006 | XPW-2020 | XPW-2034 | XPW-2048 |
| 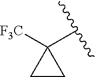 [SMILES: *C1(C(F)(F)F)CC1] | XPW-1909 | XPW-1923 | XPW-1937 | XPW-1951 | XPW-1965 | XPW-1979 | XPW-1993 | XPW-2007 | XPW-2021 | XPW-2035 | XPW-2049 |
| 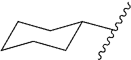 [SMILES: *C1CCCCC1] | XPW-1910 | XPW-1924 | XPW-1938 | XPW-1952 | XPW-1966 | XPW-1980 | XPW-1994 | XPW-2008 | XPW-2022 | XPW-2036 | XPW-2050 |
| 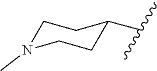 [SMILES: *C1CCN(C)CC1] | XPW-1911 | XPW-1925 | XPW-1939 | XPW-1953 | XPW-1967 | XPW-1981 | XPW-1995 | XPW-2009 | XPW-2023 | XPW-2037 | XPW-2051 |
| 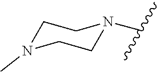 [SMILES: *N1CCN(C)CC1] | XPW-1912 | XPW-1926 | XPW-1940 | XPW-1954 | XPW-1968 | XPW-1982 | XPW-1996 | XPW-2010 | XPW-2024 | XPW-2038 | XPW-2052 |
| 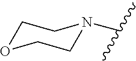 [SMILES: *N1CCOCC1] | XPW-1913 | XPW-1927 | XPW-1941 | XPW-1955 | XPW-1969 | XPW-1983 | XPW-1997 | XPW-2011 | XPW-2025 | XPW-2039 | XPW-2053 |
| 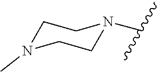 [SMILES: *N1CCN(C)CC1] | XPW-1914 | XPW-1928 | XPW-1942 | XPW-1956 | XPW-1970 | XPW-1984 | XPW-1998 | XPW-2012 | XPW-2026 | XPW-2040 | XPW-2054 |
| 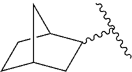 | XPW-1915 | XPW-1929 | XPW-1943 | XPW-1957 | XPW-1971 | XPW-1985 | XPW-1999 | XPW-2013 | XPW-2027 | XPW-2041 | XPW-2055 |
| 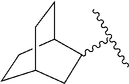 | XPW-1916 | XPW-1930 | XPW-1944 | XPW-1958 | XPW-1972 | XPW-1986 | XPW-2000 | XPW-2014 | XPW-2028 | XPW-2042 | XPW-2056 |
| 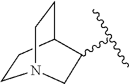 | XPW-1917 | XPW-1931 | XPW-1945 | XPW-1959 | XPW-1973 | XPW-1987 | XPW-2001 | XPW-2015 | XPW-2029 | XPW-2043 | XPW-2057 |
| 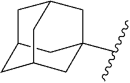 [SMILES: *C12CC3CC(CC(C3)C1)C2] | XPW-1918 | XPW-1932 | XPW-1946 | XPW-1960 | XPW-1974 | XPW-1988 | XPW-2002 | XPW-2016 | XPW-2030 | XPW-2044 | XPW-2058 |

The above table constitutes an individualized description of each of the specifically indicated compounds therein as well as their salts and solvates.

TABLE 25

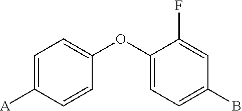

| A\B | 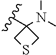 [SMILES: *C1(N(C)C)CSC1] | 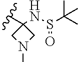 [SMILES: *C1(NS(=O)C(C)(C)C)CN(C)C1] | 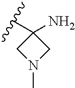 [SMILES: *C1(N)CN(C)C1] | 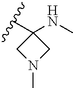 [SMILES: *C1(NC)CN(C)C1] | 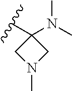 [SMILES: *C1(N(C)C)CN(C)C1] | 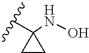 [SMILES: *C1(NO)CC1] | 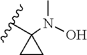 [SMILES: *C1(N(C)O)CC1] | 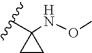 [SMILES: *C1(NOC)CC1] | 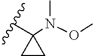 [SMILES: *C1(N(C)OC)CC1] | 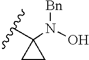 [SMILES: *C1(N(O)Cc2ccccc2)CC1] | 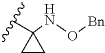 [SMILES: *C1(NOCc2ccccc2)CC1] |
|---|---|---|---|---|---|---|---|---|---|---|---|
| 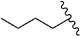 [SMILES: *CCCC] | XPW- 2059 | XPW-2073 | XPW-2087 | XPW-2101 | XPW-2115 | XPW-3570 | XPW-3584 | XPW-3598 | XPW-3612 | XPW-3626 | XPW-3640 |
| 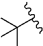 [SMILES: *C(C)(C)C] | XPW- 2060 | XPW-2074 | XPW-2088 | XPW-2102 | XPW-2116 | XPW-3571 | XPW-3585 | XPW-3599 | XPW-3613 | XPW-3627 | XPW-3641 |
| 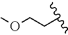 [SMILES: *CCOC] | XPW- 2061 | XPW-2075 | XPW-2089 | XPW-2103 | XPW-2117 | XPW-3572 | XPW-3586 | XPW-3600 | XPW-3614 | XPW-3628 | XPW-3642 |
| 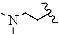 [SMILES: *CCN(C)C] | XPW- 2062 | XPW-2076 | XPW-2090 | XPW-2104 | XPW-2118 | XPW-3573 | XPW-3587 | XPW-3601 | XPW-3615 | XPW-3629 | XPW-3643 |
| 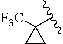 [SMILES: *C1(C(F)(F)F)CC1] | XPW- 2063 | XPW-2077 | XPW-2091 | XPW-2105 | XPW-2119 | XPW-3574 | XPW-3588 | XPW-3602 | XPW-3616 | XPW-3630 | XPW-3644 |
| 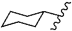 [SMILES: *C1CCCCC1] | XPW- 2064 | XPW-2078 | XPW-2092 | XPW-2106 | XPW-2120 | XPW-3575 | XPW-3589 | XPW-3603 | XPW-3617 | XPW-3631 | XPW-3645 |
| 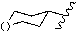 [SMILES: *C1CCOCC1] | XPW- 2065 | XPW-2079 | XPW-2093 | XPW-2107 | XPW-2121 | XPW-3576 | XPW-3590 | XPW-3604 | XPW-3618 | XPW-3632 | XPW-3646 |
| 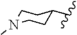 [SMILES: *C1CCN(C)CC1] | XPW- 2066 | XPW-2080 | XPW-2094 | XPW-2108 | XPW-2122 | XPW-3577 | XPW-3591 | XPW-3605 | XPW-3619 | XPW-3633 | XPW-3647 |
| 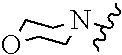 [SMILES: *N1CCOCC1] | XPW- 2067 | XPW-2081 | XPW-2095 | XPW-2109 | XPW-2123 | XPW-3578 | XPW-3592 | XPW-3606 | XPW-3620 | XPW-3634 | XPW-3648 |
| 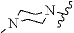 [SMILES: *N1CCN(C)CC1] | XPW- 2068 | XPW-2082 | XPW-2096 | XPW-2110 | XPW-2124 | XPW-3579 | XPW-3593 | XPW-3607 | XPW-3621 | XPW-3635 | XPW-3649 |
| 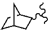 | XPW- 2069 | XPW-2083 | XPW-2097 | XPW-2111 | XPW-2125 | XPW-3580 | XPW-3594 | XPW-3608 | XPW-3622 | XPW-3636 | XPW-3650 |
| 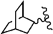 | XPW- 2070 | XPW-2084 | XPW-2098 | XPW-2112 | XPW-2126 | XPW-3581 | XPW-3595 | XPW-3609 | XPW-3623 | XPW-3637 | XPW-3651 |
| 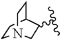 | XPW- 2071 | XPW-2085 | XPW-2099 | XPW-2113 | XPW-2127 | XPW-3582 | XPW-3596 | XPW-3610 | XPW-3624 | XPW-3638 | XPW-3652 |
| 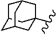 [SMILES: *C12CC3CC(CC(C3)C1)C2] | XPW- 2072 | XPW-2086 | XPW-2100 | XPW-2114 | XPW-2128 | XPW-3583 | XPW-3597 | XPW-3611 | XPW-3625 | XPW-3639 | XPW-3653 |

The above table constitutes an individualized description of each of the specifically indicated compounds therein as well as their salts and solvates.

TABLE 26

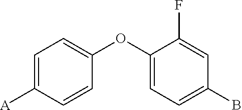

| A\B | 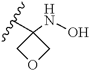 [SMILES: *C1(NO)COC1] | 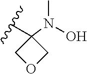 [SMILES: *C1(N(C)O)COC1] | 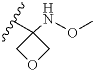 [SMILES: *C1(NOC)COC1] | 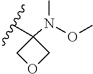 [SMILES: *C1(N(C)OC)COC1] | 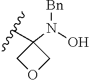 [SMILES: *C1(N(O)Cc2ccccc2)COC1] | 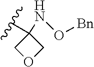 [SMILES: *C1(NOCc2ccccc2)COC1] | 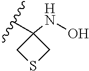 [SMILES: *C1(NO)CSC1] | 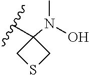 [SMILES: *C1(N(C)O)CSC1] | 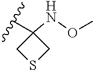 [SMILES: *C1(NOC)CSC1] | 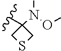 [SMILES: *C1(N(C)OC)CSC1] | 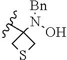 [SMILES: *C1(N(O)Cc2ccccc2)CSC1] |
|---|---|---|---|---|---|---|---|---|---|---|---|
| 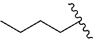 [SMILES: *CCCC] | XPW-3654 | XPW-3668 | XPW-3682 | XPW-3696 | XPW-3710 | XPW-3724 | XPW-3738 | XPW-3752 | XPW-3766 | XPW- 3780 | XPW- 3794 |
| 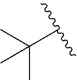 [SMILES: *C(C)(C)C] | XPW-3655 | XPW-3669 | XPW-3683 | XPW-3697 | XPW-3711 | XPW-3725 | XPW-3739 | XPW-3753 | XPW-3767 | XPW- 3781 | XPW- 3795 |
| 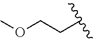 [SMILES: *CCOC] | XPW-3656 | XPW-3670 | XPW-3684 | XPW-3698 | XPW-3712 | XPW-3726 | XPW-3740 | XPW-3754 | XPW-3768 | XPW- 3782 | XPW- 3796 |
| 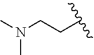 [SMILES: *CCN(C)C] | XPW-3657 | XPW-3671 | XPW-3685 | XPW-3699 | XPW-3713 | XPW-3727 | XPW-3741 | XPW-3755 | XPW-3769 | XPW- 3783 | XPW- 3797 |
| 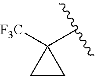 [SMILES: *C1(C(F)(F)F)CC1] | XPW-3658 | XPW-3672 | XPW-3686 | XPW-3700 | XPW-3714 | XPW-3728 | XPW-3742 | XPW-3756 | XPW-3770 | XPW- 3784 | XPW- 3798 |
| 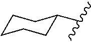 [SMILES: *C1CCCCC1] | XPW-3659 | XPW-3673 | XPW-3687 | XPW-3701 | XPW-3715 | XPW-3729 | XPW-3743 | XPW-3757 | XPW-3771 | XPW- 3785 | XPW- 3799 |
| 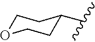 [SMILES: *C1CCOCC1] | XPW-3660 | XPW-3674 | XPW-3688 | XPW-3702 | XPW-3716 | XPW-3730 | XPW-3744 | XPW-3758 | XPW-3772 | XPW- 3786 | XPW- 3800 |
| 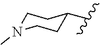 [SMILES: *C1CCN(C)CC1] | XPW-3661 | XPW-3675 | XPW-3689 | XPW-3703 | XPW-3717 | XPW-3731 | XPW-3745 | XPW-3759 | XPW-3773 | XPW- 3787 | XPW- 3801 |
| 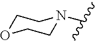 [SMILES: *N1CCOCC1] | XPW-3662 | XPW-3676 | XPW-3690 | XPW-3704 | XPW-3718 | XPW-3732 | XPW-3746 | XPW-3760 | XPW-3774 | XPW- 3788 | XPW- 3802 |
| 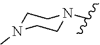 [SMILES: *N1CCN(C)CC1] | XPW-3663 | XPW-3677 | XPW-3691 | XPW-3705 | XPW-3719 | XPW-3733 | XPW-3747 | XPW-3761 | XPW-3775 | XPW- 3789 | XPW- 3803 |
| 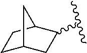 | XPW-3664 | XPW-3678 | XPW-3692 | XPW-3706 | XPW-3720 | XPW-3734 | XPW-3748 | XPW-3762 | XPW-3776 | XPW- 3790 | XPW- 3804 |
| 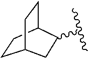 | XPW-3665 | XPW-3679 | XPW-3693 | XPW-3707 | XPW-3721 | XPW-3735 | XPW-3749 | XPW-3763 | XPW-3777 | XPW- 3791 | XPW- 3805 |
| 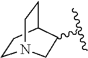 | XPW-3666 | XPW-3680 | XPW-3694 | XPW-3708 | XPW-3722 | XPW-3736 | XPW-3750 | XPW-3764 | XPW-3778 | XPW- 3792 | XPW- 3806 |
| 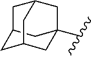 [SMILES: *C12CC3CC(CC(C3)C1)C2] | XPW-3667 | XPW-3681 | XPW-3695 | XPW-3709 | XPW-3723 | XPW-3737 | XPW-3751 | XPW-3765 | XPW-3779 | XPW- 3793 | XPW- 3807 |

The above table constitutes an individualized description of each of the specifically indicated compounds therein as well as their salts and solvates.

TABLE 27

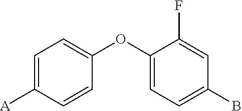

| A\B | 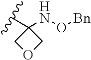 [SMILES: *C1(NOCc2ccccc2)COC1] | 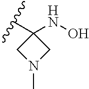 [SMILES: *C1(NO)CN(C)C1] | 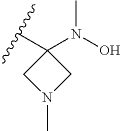 [SMILES: *C1(N(C)O)CN(C)C1] | 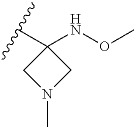 [SMILES: *C1(NOC)CN(C)C1] | 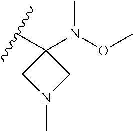 [SMILES: *C1(N(C)OC)CN(C)C1] | 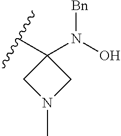 [SMILES: *C1(N(O)Cc2ccccc2)CN(C)C1] | 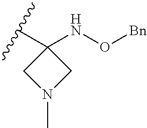 [SMILES: *C1(NOCc2ccccc2)CN(C)C1] | 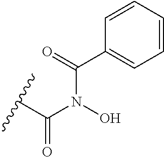 [SMILES: *C(=O)N(O)C(=O)c1ccccc1] |
|---|---|---|---|---|---|---|---|---|
| 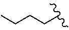 [SMILES: *CCCC] | XPW-3808 | XPW-3822 | XPW-3836 | XPW-3850 | XPW-3864 | XPW-3878 | XPW-3892 | XPW-4958 |
| 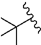 [SMILES: *C(C)(C)C] | XPW-3809 | XPW-3823 | XPW-3837 | XPW-3851 | XPW-3865 | XPW-3879 | XPW-3893 | XPW-4959 |
| 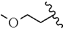 [SMILES: *CCOC] | XPW-3810 | XPW-3824 | XPW-3838 | XPW-3852 | XPW-3866 | XPW-3880 | XPW-3894 | XPW-4960 |
| 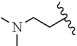 [SMILES: *CCN(C)C] | XPW-3811 | XPW-3825 | XPW-3839 | XPW-3853 | XPW-3867 | XPW-3881 | XPW-3895 | XPW-4961 |
| 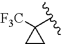 [SMILES: *C1(C(F)(F)F)CC1] | XPW-3812 | XPW-3826 | XPW-3840 | XPW-3854 | XPW-3868 | XPW-3882 | XPW-3896 | XPW-4962 |
| 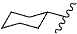 [SMILES: *C1CCCCC1] | XPW-3813 | XPW-3827 | XPW-3841 | XPW-3855 | XPW-3869 | XPW-3883 | XPW-3897 | XPW-4963 |
| 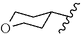 [SMILES: *C1CCOCC1] | XPW-3814 | XPW-3828 | XPW-3842 | XPW-3856 | XPW-3870 | XPW-3884 | XPW-3898 | XPW-4964 |
| 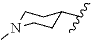 [SMILES: *C1CCN(C)CC1] | XPW-3815 | XPW-3829 | XPW-3843 | XPW-3857 | XPW-3871 | XPW-3885 | XPW-3899 | XPW-4965 |
| 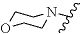 [SMILES: *N1CCOCC1] | XPW-3816 | XPW-3830 | XPW-3844 | XPW-3858 | XPW-3872 | XPW-3886 | XPW-3900 | XPW-4966 |
| 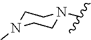 [SMILES: *N1CCN(C)CC1] | XPW-3817 | XPW-3831 | XPW-3845 | XPW-3859 | XPW-3873 | XPW-3887 | XPW-3901 | XPW-4967 |
| 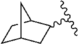 | XPW-3818 | XPW-3832 | XPW-3846 | XPW-3860 | XPW-3874 | XPW-3888 | XPW-3902 | XPW-4968 |
| 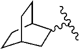 | XPW-3819 | XPW-3833 | XPW-3847 | XPW-3861 | XPW-3875 | XPW-3889 | XPW-3903 | XPW-4969 |
| 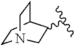 | XPW-3820 | XPW-3834 | XPW-3848 | XPW-3862 | XPW-3876 | XPW-3890 | XPW-3904 | XPW-4970 |
| 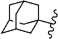 [SMILES: *C12CC3CC(CC(C3)C1)C2] | XPW-3821 | XPW-3835 | XPW-3849 | XPW-3863 | XPW-3877 | XPW-3891 | XPW-3905 | XPW-4971 |

The above table constitutes an individualized description of each of the specifically indicated compounds therein as well as their salts and solvates.

TABLE 28

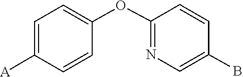

| A\B | 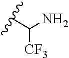 [SMILES: *C(N)C(F)(F)F] | 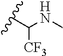 [SMILES: *C(NC)C(F)(F)F] | 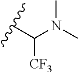 [SMILES: *C(N(C)C)C(F)(F)F] | 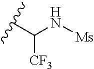 [SMILES: *C(NS(C)(=O)=O)C(F)(F)F] | 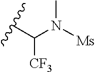 [SMILES: *C(N(C)S(C)(=O)=O)C(F)(F)F] | 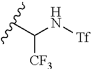 | 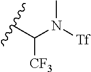 | 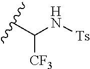 [SMILES: *C(NS(=O)(=O)c1ccc(C)cc1)C(F)(F)F] | 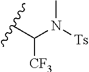 [SMILES: *C(N(C)S(=O)(=O)c1ccc(C)cc1)C(F)(F)F] | 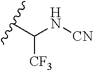 [SMILES: *C(NC#N)C(F)(F)F] | 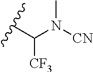 [SMILES: *C(N(C)C#N)C(F)(F)F] |
|---|---|---|---|---|---|---|---|---|---|---|---|
| 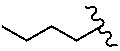 [SMILES: *CCCC] | XPW-2129 | XPW-2143 | XPW-2157 | XPW-2171 | XPW-2185 | XPW-2199 | XPW-2213 | XPW-2227 | XPW-2241 | XPW-2255 | XPW-2269 |
| 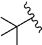 [SMILES: *C(C)(C)C] | XPW-2130 | XPW-2144 | XPW-2158 | XPW-2172 | XPW-2186 | XPW-2200 | XPW-2214 | XPW-2228 | XPW-2242 | XPW-2256 | XPW-2270 |
| 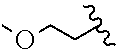 [SMILES: *CCOC] | XPW-2131 | XPW-2145 | XPW-2159 | XPW-2173 | XPW-2187 | XPW-2201 | XPW-2215 | XPW-2229 | XPW-2243 | XPW-2257 | XPW-2271 |
| 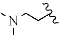 [SMILES: *CCN(C)C] | XPW-2132 | XPW-2146 | XPW-2160 | XPW-2174 | XPW-2188 | XPW-2202 | XPW-2216 | XPW-2230 | XPW-2244 | XPW-2258 | XPW-2272 |
| 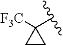 [SMILES: *C1(C(F)(F)F)CC1] | XPW-2133 | XPW-2147 | XPW-2161 | XPW-2175 | XPW-2189 | XPW-2203 | XPW-2217 | XPW-2231 | XPW-2245 | XPW-2259 | XPW-2273 |
| 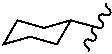 [SMILES: *C1CCCCC1] | XPW-2134 | XPW-2148 | XPW-2162 | XPW-2176 | XPW-2190 | XPW-2204 | XPW-2218 | XPW-2232 | XPW-2246 | XPW-2260 | XPW-2274 |
| 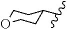 [SMILES: *C1CCOCC1] | XPW-2135 | XPW-2149 | XPW-2163 | XPW-2177 | XPW-2191 | XPW-2205 | XPW-2219 | XPW-2233 | XPW-2247 | XPW-2261 | XPW-2275 |
| 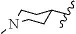 [SMILES: *C1CCN(C)CC1] | XPW-2136 | XPW-2150 | XPW-2164 | XPW-2178 | XPW-2192 | XPW-2206 | XPW-2220 | XPW-2234 | XPW-2248 | XPW-2262 | XPW-2276 |
| 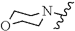 [SMILES: *N1CCOCC1] | XPW-2137 | XPW-2151 | XPW-2165 | XPW-2179 | XPW-2193 | XPW-2207 | XPW-2221 | XPW-2235 | XPW-2249 | XPW-2263 | XPW-2277 |
| 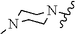 [SMILES: *N1CCN(C)CC1] | XPW-2138 | XPW-2152 | XPW-2166 | XPW-2180 | XPW-2194 | XPW-2208 | XPW-2222 | XPW-2236 | XPW-2250 | XPW-2264 | XPW-2278 |
| 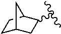 | XPW-2139 | XPW-2153 | XPW-2167 | XPW-2181 | XPW-2195 | XPW-2209 | XPW-2223 | XPW-2237 | XPW-2251 | XPW-2265 | XPW-2279 |
| 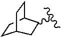 | XPW-2140 | XPW-2154 | XPW-2168 | XPW-2182 | XPW-2196 | XPW-2210 | XPW-2224 | XPW-2238 | XPW-2252 | XPW-2266 | XPW-2280 |
| 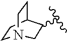 | XPW-2141 | XPW-2155 | XPW-2169 | XPW-2183 | XPW-2197 | XPW-2211 | XPW-2225 | XPW-2239 | XPW-2253 | XPW-2267 | XPW-2281 |
| 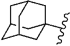 [SMILES: *C12CC3CC(CC(C3)C1)C2] | XPW-2142 | XPW-2156 | XPW-2170 | XPW-2184 | XPW-2198 | XPW-2212 | XPW-2226 | XPW-2240 | XPW-2254 | XPW-2268 | XPW-2282 |

The above table constitutes an individualized description of each of the specifically indicated compounds therein as well as their salts and solvates.

TABLE 29

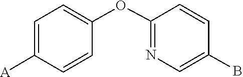

| A\B | 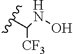 [SMILES: *C(NO)C(F)(F)F] | 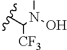 [SMILES: *C(N(C)O)C(F)(F)F] | 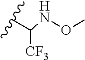 [SMILES: *C(NOC)C(F)(F)F] | 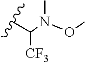 [SMILES: *C(N(C)OC)C(F)(F)F] | 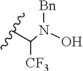 [SMILES: *C(N(O)Cc1ccccc1)C(F)(F)F] | 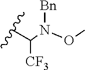 [SMILES: *C(N(Cc1ccccc1)OC)C(F)(F)F] | 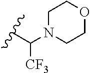 [SMILES: *C(N1CCOCC1)C(F)(F)F] | 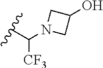 [SMILES: *C(N1CC(O)C1)C(F)(F)F] | 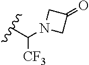 [SMILES: *C(N1CC(=O)C1)C(F)(F)F] | 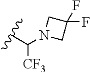 [SMILES: *C(N1CC(F)(F)C1)C(F)(F)F] | 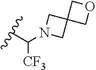 [SMILES: *C(N1CC2(COC2)C1)C(F)(F)F] |
|---|---|---|---|---|---|---|---|---|---|---|---|
| 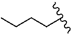 [SMILES: *CCCC] | XPW-2283 | XPW-2297 | XPW-2311 | XPW-2325 | XPW-2339 | XPW-2353 | XPW-2367 | XPW-2381 | XPW-2395 | XPW-2409 | XPW-2423 |
| 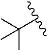 [SMILES: *C(C)(C)C] | XPW-2284 | XPW-2298 | XPW-2312 | XPW-2326 | XPW-2340 | XPW-2354 | XPW-2368 | XPW-2382 | XPW-2396 | XPW-2410 | XPW-2424 |
| 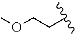 [SMILES: *CCOC] | XPW-2285 | XPW-2299 | XPW-2313 | XPW-2327 | XPW-2341 | XPW-2355 | XPW-2369 | XPW-2383 | XPW-2397 | XPW-2411 | XPW-2425 |
| 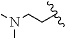 [SMILES: *CCN(C)C] | XPW-2286 | XPW-2300 | XPW-2314 | XPW-2328 | XPW-2342 | XPW-2356 | XPW-2370 | XPW-2384 | XPW-2398 | XPW-2412 | XPW-2426 |
| 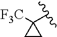 [SMILES: *C1(C(F)(F)F)CC1] | XPW-2287 | XPW-2301 | XPW-2315 | XPW-2329 | XPW-2343 | XPW-2357 | XPW-2371 | XPW-2385 | XPW-2399 | XPW-2413 | XPW-2427 |
| 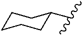 [SMILES: *C1CCCCC1] | XPW-2288 | XPW-2302 | XPW-2316 | XPW-2330 | XPW-2344 | XPW-2358 | XPW-2372 | XPW-2386 | XPW-2400 | XPW-2414 | XPW-2428 |
| 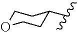 [SMILES: *C1CCOCC1] | XPW-2289 | XPW-2303 | XPW-2317 | XPW-2331 | XPW-2345 | XPW-2359 | XPW-2373 | XPW-2387 | XPW-2401 | XPW-2415 | XPW-2429 |
| 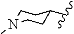 [SMILES: *C1CCN(C)CC1] | XPW-2290 | XPW-2304 | XPW-2318 | XPW-2332 | XPW-2346 | XPW-2360 | XPW-2374 | XPW-2388 | XPW-2402 | XPW-2416 | XPW-2430 |
| 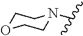 [SMILES: *N1CCOCC1] | XPW-2291 | XPW-2305 | XPW-2319 | XPW-2333 | XPW-2347 | XPW-2361 | XPW-2375 | XPW-2389 | XPW-2403 | XPW-2417 | XPW-2431 |
| 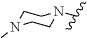 [SMILES: *N1CCN(C)CC1] | XPW-2292 | XPW-2306 | XPW-2320 | XPW-2334 | XPW-2348 | XPW-2362 | XPW-2376 | XPW-2390 | XPW-2404 | XPW-2418 | XPW-2432 |
| 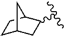 | XPW-2293 | XPW-2307 | XPW-2321 | XPW-2335 | XPW-2349 | XPW-2363 | XPW-2377 | XPW-2391 | XPW-2405 | XPW-2419 | XPW-2433 |
| 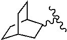 | XPW-2294 | XPW-2308 | XPW-2322 | XPW-2336 | XPW-2350 | XPW-2364 | XPW-2378 | XPW-2392 | XPW-2406 | XPW-2420 | XPW-2434 |
| 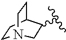 | XPW-2295 | XPW-2309 | XPW-2323 | XPW-2337 | XPW-2351 | XPW-2365 | XPW-2379 | XPW-2393 | XPW-2407 | XPW-2421 | XPW-2435 |
| 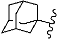 [SMILES: *C12CC3CC(CC(C3)C1)C2] | XPW-2296 | XPW-2310 | XPW-2324 | XPW-2338 | XPW-2352 | XPW-2366 | XPW-2380 | XPW-2394 | XPW-2408 | XPW-2422 | XPW-2436 |

The above table constitutes an individualized description of each of the specifically indicated compounds therein as well as their salts and solvates.

TABLE 30

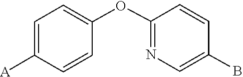

| A\B | 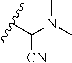 [SMILES: *C(C#N)N(C)C] | 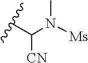 [SMILES: *C(C#N)N(C)S(C)(=O)=O] | 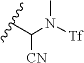 | 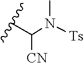 [SMILES: *C(C#N)N(C)S(=O)(=O)c1ccc(C)cc1] | 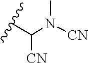 [SMILES: *C(C#N)N(C)C#N] | 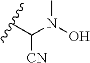 [SMILES: *C(C#N)N(C)O] | 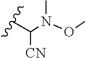 [SMILES: *C(C#N)N(C)OC] | 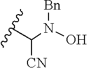 [SMILES: *C(C#N)N(O)Cc1ccccc1] | 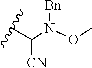 [SMILES: *C(C#N)N(Cc1ccccc1)OC] | 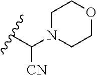 [SMILES: *C(C#N)N1CCOCC1] | 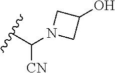 [SMILES: *C(C#N)N1CC(O)C1] |
|---|---|---|---|---|---|---|---|---|---|---|---|
| 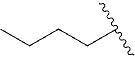 [SMILES: *CCCC] | XPW-2437 | XPW-2451 | XPW-2465 | XPW-2479 | XPW-2493 | XPW-2507 | XPW-2521 | XPW-2535 | XPW-2549 | XPW-2563 | XPW-2577 |
| 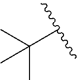 [SMILES: *C(C)(C)C] | XPW-2438 | XPW-2452 | XPW-2466 | XPW-2480 | XPW-2494 | XPW-2508 | XPW-2522 | XPW-2536 | XPW-2550 | XPW-2564 | XPW-2578 |
| 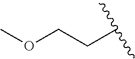 [SMILES: *CCOC] | XPW-2439 | XPW-2453 | XPW-2467 | XPW-2481 | XPW-2495 | XPW-2509 | XPW-2523 | XPW-2537 | XPW-2551 | XPW-2565 | XPW-2579 |
| 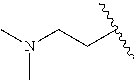 [SMILES: *CCN(C)C] | XPW-2440 | XPW-2454 | XPW-2468 | XPW-2482 | XPW-2496 | XPW-2510 | XPW-2524 | XPW-2538 | XPW-2552 | XPW-2566 | XPW-2580 |
| 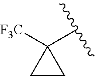 [SMILES: *C1(C(F)(F)F)CC1] | XPW-2441 | XPW-2455 | XPW-2469 | XPW-2483 | XPW-2497 | XPW-2511 | XPW-2525 | XPW-2539 | XPW-2553 | XPW-2567 | XPW-2581 |
| 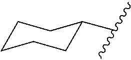 [SMILES: *C1CCCCC1] | XPW-2442 | XPW-2456 | XPW-2470 | XPW-2484 | XPW-2498 | XPW-2512 | XPW-2526 | XPW-2540 | XPW-2554 | XPW-2568 | XPW-2582 |
| 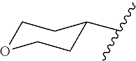 [SMILES: *C1CCOCC1] | XPW-2443 | XPW-2457 | XPW-2471 | XPW-2485 | XPW-2499 | XPW-2513 | XPW-2527 | XPW-2541 | XPW-2555 | XPW-2569 | XPW-2583 |
| 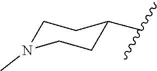 [SMILES: *C1CCN(C)CC1] | XPW-2444 | XPW-2458 | XPW-2472 | XPW-2486 | XPW-2500 | XPW-2514 | XPW-2528 | XPW-2542 | XPW-2556 | XPW-2570 | XPW-2584 |
| 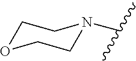 [SMILES: *N1CCOCC1] | XPW-2445 | XPW-2459 | XPW-2473 | XPW-2487 | XPW-2501 | XPW-2515 | XPW-2529 | XPW-2543 | XPW-2557 | XPW-2571 | XPW-2585 |
| 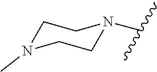 [SMILES: *N1CCN(C)CC1] | XPW-2446 | XPW-2460 | XPW-2474 | XPW-2488 | XPW-2502 | XPW-2516 | XPW-2530 | XPW-2544 | XPW-2558 | XPW-2572 | XPW-2586 |
| 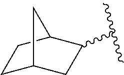 | XPW-2447 | XPW-2461 | XPW-2475 | XPW-2489 | XPW-2503 | XPW-2517 | XPW-2531 | XPW-2545 | XPW-2559 | XPW-2573 | XPW-2587 |
| 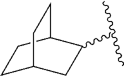 | XPW-2448 | XPW-2462 | XPW-2476 | XPW-2490 | XPW-2504 | XPW-2518 | XPW-2532 | XPW-2546 | XPW-2560 | XPW-2574 | XPW-2588 |
| 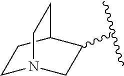 | XPW-2449 | XPW-2463 | XPW-2477 | XPW-2491 | XPW-2505 | XPW-2519 | XPW-2533 | XPW-2547 | XPW-2561 | XPW-2575 | XPW-2589 |
| 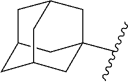 [SMILES: *C12CC3CC(CC(C3)C1)C2] | XPW-2450 | XPW-2464 | XPW-2478 | XPW-2492 | XPW-2506 | XPW-2520 | XPW-2534 | XPW-2548 | XPW-2562 | XPW-2576 | XPW-2590 |

The above table constitutes an individualized description of each of the specifically indicated compounds therein as well as their salts and solvates.

TABLE 31

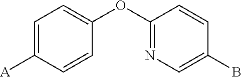

| A\B | 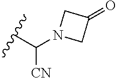 [SMILES: *C(C#N)N1CC(=O)C1] | 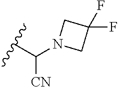 [SMILES: *C(C#N)N1CC(F)(F)C1] | 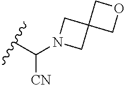 [SMILES: *C(C#N)N1CC2(COC2)C1] | 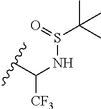 [SMILES: *C(NS(=O)C(C)(C)C)C(F)(F)F] | 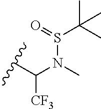 [SMILES: *C(N(C)S(=O)C(C)(C)C)C(F)(F)F] | 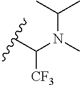 [SMILES: *C(N(C)C(C)C)C(F)(F)F] |
|---|---|---|---|---|---|---|
| 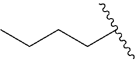 [SMILES: *CCCC] | XPW-2591 | XPW-2605 | XPW-2619 | XPW-4972 | XPW-4986 | XPW-5000 |
| 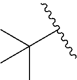 [SMILES: *C(C)(C)C] | XPW-2592 | XPW-2606 | XPW-2620 | XPW-4973 | XPW-4987 | XPW-5001 |
| 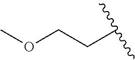 [SMILES: *CCOC] | XPW-2593 | XPW-2607 | XPW-2621 | XPW-4974 | XPW-4988 | XPW-5002 |
| 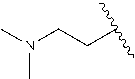 [SMILES: *CCN(C)C] | XPW-2594 | XPW-2608 | XPW-2622 | XPW-4975 | XPW-4989 | XPW-5003 |
| 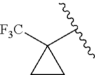 [SMILES: *C1(C(F)(F)F)CC1] | XPW-2595 | XPW-2609 | XPW-2623 | XPW-4976 | XPW-4990 | XPW-5004 |
| 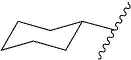 [SMILES: *C1CCCCC1] | XPW-2596 | XPW-2610 | XPW-2624 | XPW-4977 | XPW-4991 | XPW-5005 |
| 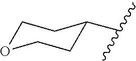 [SMILES: *C1CCOCC1] | XPW-2597 | XPW-2611 | XPW-2625 | XPW-4978 | XPW-4992 | XPW-5006 |
| 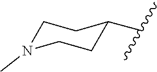 [SMILES: *C1CCN(C)CC1] | XPW-2598 | XPW-2612 | XPW-2626 | XPW-4979 | XPW-4993 | XPW-5007 |
| 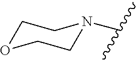 [SMILES: *N1CCOCC1] | XPW-2599 | XPW-2613 | XPW-2627 | XPW-4980 | XPW-4994 | XPW-5008 |
| 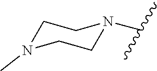 [SMILES: *N1CCN(C)CC1] | XPW-2600 | XPW-2614 | XPW-2628 | XPW-4981 | XPW-4995 | XPW-5009 |
| 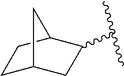 | XPW-2601 | XPW-2615 | XPW-2629 | XPW-4982 | XPW-4996 | XPW-5010 |
| 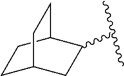 | XPW-2602 | XPW-2616 | XPW-2630 | XPW-4983 | XPW-4997 | XPW-5011 |
| 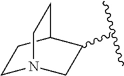 | XPW-2603 | XPW-2617 | XPW-2631 | XPW-4984 | XPW-4998 | XPW-5012 |
| 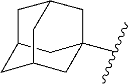 [SMILES: *C12CC3CC(CC(C3)C1)C2] | XPW-2604 | XPW-2618 | XPW-2632 | XPW-4985 | XPW-4999 | XPW-5013 |

TABLE 32

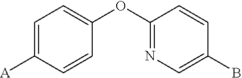

| A\B | 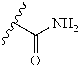 [SMILES: *C(N)=O] | 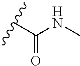 [SMILES: *C(=O)NC] | 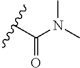 [SMILES: *C(=O)N(C)C] | 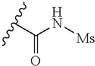 [SMILES: *C(=O)NS(C)(=O)=O] | 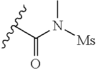 [SMILES: *C(=O)N(C)S(C)(=O)=O] | 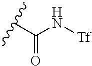 | 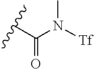 | 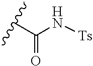 [SMILES: *C(=O)NS(=O)(=O)c1ccc(C)cc1] | 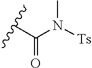 [SMILES: *C(=O)N(C)S(=O)(=O)c1ccc(C)cc1] | 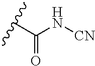 [SMILES: *C(=O)NC#N] | 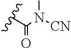 [SMILES: *C(=O)N(C)C#N] |
|---|---|---|---|---|---|---|---|---|---|---|---|
| 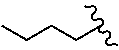 [SMILES: *CCCC] | XPW-2633 | XPW-2647 | XPW-2661 | XPW-2675 | XPW-2689 | XPW-2703 | XPW-2717 | XPW-2731 | XPW-2745 | XPW-2759 | XPW- 2773 |
| 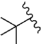 [SMILES: *C(C)(C)C] | XPW-2634 | XPW-2648 | XPW-2662 | XPW-2676 | XPW-2690 | XPW-2704 | XPW-2718 | XPW-2732 | XPW-2746 | XPW-2760 | XPW- 2774 |
| 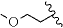 [SMILES: *CCOC] | XPW-2635 | XPW-2649 | XPW-2663 | XPW-2677 | XPW-2691 | XPW-2705 | XPW-2719 | XPW-2733 | XPW-2747 | XPW-2761 | XPW- 2775 |
| 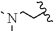 [SMILES: *CCN(C)C] | XPW-2636 | XPW-2650 | XPW-2664 | XPW-2678 | XPW-2692 | XPW-2706 | XPW-2720 | XPW-2734 | XPW-2748 | XPW-2762 | XPW- 2776 |
| 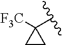 [SMILES: *C1(C(F)(F)F)CC1] | XPW-2637 | XPW-2651 | XPW-2665 | XPW-2679 | XPW-2693 | XPW-2707 | XPW-2721 | XPW-2735 | XPW-2749 | XPW-2763 | XPW- 2777 |
| 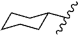 [SMILES: *C1CCCCC1] | XPW-2638 | XPW-2652 | XPW-2666 | XPW-2680 | XPW-2694 | XPW-2708 | XPW-2722 | XPW-2736 | XPW-2750 | XPW-2764 | XPW- 2778 |
| 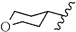 [SMILES: *C1CCOCC1] | XPW-2639 | XPW-2653 | XPW-2667 | XPW-2681 | XPW-2695 | XPW-2709 | XPW-2723 | XPW-2737 | XPW-2751 | XPW-2765 | XPW- 2779 |
| 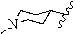 [SMILES: *C1CCN(C)CC1] | XPW-2640 | XPW-2654 | XPW-2668 | XPW-2682 | XPW-2696 | XPW-2710 | XPW-2724 | XPW-2738 | XPW-2752 | XPW-2766 | XPW- 2780 |
| 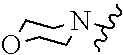 [SMILES: *N1CCOCC1] | XPW-2641 | XPW-2655 | XPW-2669 | XPW-2683 | XPW-2697 | XPW-2711 | XPW-2725 | XPW-2739 | XPW-2753 | XPW-2767 | XPW- 2781 |
| 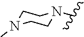 [SMILES: *N1CCN(C)CC1] | XPW-2642 | XPW-2656 | XPW-2670 | XPW-2684 | XPW-2698 | XPW-2712 | XPW-2726 | XPW-2740 | XPW-2754 | XPW-2768 | XPW- 2782 |
| 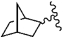 | XPW-2643 | XPW-2657 | XPW-2671 | XPW-2685 | XPW-2699 | XPW-2713 | XPW-2727 | XPW-2741 | XPW-2755 | XPW-2769 | XPW- 2783 |
| 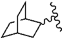 | XPW-2644 | XPW-2658 | XPW-2672 | XPW-2686 | XPW-2700 | XPW-2714 | XPW-2728 | XPW-2742 | XPW-2756 | XPW-2770 | XPW- 2784 |
| 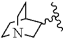 | XPW-2645 | XPW-2659 | XPW-2673 | XPW-2687 | XPW-2701 | XPW-2715 | XPW-2729 | XPW-2743 | XPW-2757 | XPW-2771 | XPW- 2785 |
| 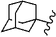 [SMILES: *C12CC3CC(CC(C3)C1)C2] | XPW-2646 | XPW-2660 | XPW-2674 | XPW-2688 | XPW-2702 | XPW-2716 | XPW-2730 | XPW-2744 | XPW-2758 | XPW-2772 | XPW- 2786 |

The above table constitutes an individualized description of each of the specifically indicated compounds therein as well as their salts and solvates.

TABLE 33

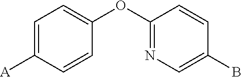

| A\B | 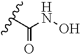 [SMILES: *C(=O)NO] | 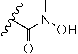 [SMILES: *C(=O)N(C)O] | 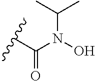 [SMILES: *C(=O)N(O)C(C)C] | 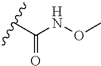 [SMILES: *C(=O)NOC] | 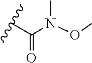 [SMILES: *C(=O)N(C)OC] | 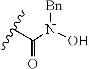 [SMILES: *C(=O)N(O)Cc1ccccc1] | 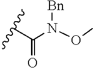 [SMILES: *C(=O)N(Cc1ccccc1)OC] | 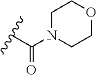 [SMILES: *C(=O)N1CCOCC1] | 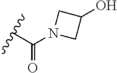 [SMILES: *C(=O)N1CC(O)C1] | 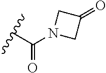 [SMILES: *C(=O)N1CC(=O)C1] | 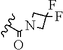 [SMILES: *C(=O)N1CC(F)(F)C1] |
|---|---|---|---|---|---|---|---|---|---|---|---|
| 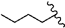 [SMILES: *CCCC] | XPW-2787 | XPW-2801 | XPW-2815 | XPW-2829 | XPW-2843 | XPW-2857 | XPW-2871 | XPW-2885 | XPW-2899 | XPW-2913 | XPW- 2927 |
| 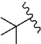 [SMILES: *C(C)(C)C] | XPW-2788 | XPW-2802 | XPW-2816 | XPW-2830 | XPW-2844 | XPW-2858 | XPW-2872 | XPW-2886 | XPW-2900 | XPW-2914 | XPW- 2928 |
| 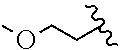 [SMILES: *CCOC] | XPW-2789 | XPW-2803 | XPW-2817 | XPW-2831 | XPW-2845 | XPW-2859 | XPW-2873 | XPW-2887 | XPW-2901 | XPW-2915 | XPW- 2929 |
| 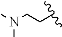 [SMILES: *CCN(C)C] | XPW-2790 | XPW-2804 | XPW-2818 | XPW-2832 | XPW-2846 | XPW-2860 | XPW-2874 | XPW-2888 | XPW-2902 | XPW-2916 | XPW- 2930 |
| 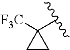 [SMILES: *C1(C(F)(F)F)CC1] | XPW-2791 | XPW-2805 | XPW-2819 | XPW-2833 | XPW-2847 | XPW-2861 | XPW-2875 | XPW-2889 | XPW-2903 | XPW-2917 | XPW- 2931 |
| 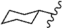 [SMILES: *C1CCCCC1] | XPW-2792 | XPW-2806 | XPW-2820 | XPW-2834 | XPW-2848 | XPW-2862 | XPW-2876 | XPW-2890 | XPW-2904 | XPW-2918 | XPW- 2932 |
| 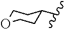 [SMILES: *C1CCOCC1] | XPW-2793 | XPW-2807 | XPW-2821 | XPW-2835 | XPW-2849 | XPW-2863 | XPW-2877 | XPW-2891 | XPW-2905 | XPW-2919 | XPW- 2933 |
| 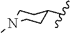 [SMILES: *C1CCN(C)CC1] | XPW-2794 | XPW-2808 | XPW-2822 | XPW-2836 | XPW-2850 | XPW-2864 | XPW-2878 | XPW-2892 | XPW-2906 | XPW-2920 | XPW- 2934 |
| 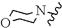 [SMILES: *N1CCOCC1] | XPW-2795 | XPW-2809 | XPW-2823 | XPW-2837 | XPW-2851 | XPW-2865 | XPW-2879 | XPW-2893 | XPW-2907 | XPW-2921 | XPW- 2935 |
| 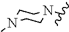 [SMILES: *N1CCN(C)CC1] | XPW-2796 | XPW-2810 | XPW-2824 | XPW-2838 | XPW-2852 | XPW-2866 | XPW-2880 | XPW-2894 | XPW-2908 | XPW-2922 | XPW- 2936 |
| 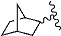 | XPW-2797 | XPW-2811 | XPW-2825 | XPW-2839 | XPW-2853 | XPW-2867 | XPW-2881 | XPW-2895 | XPW-2909 | XPW-2923 | XPW- 2937 |
| 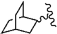 | XPW-2798 | XPW-2812 | XPW-2826 | XPW-2840 | XPW-2854 | XPW-2868 | XPW-2882 | XPW-2896 | XPW-2910 | XPW-2924 | XPW- 2938 |
| 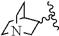 | XPW-2799 | XPW-2813 | XPW-2827 | XPW-2841 | XPW-2855 | XPW-2869 | XPW-2883 | XPW-2897 | XPW-2911 | XPW-2925 | XPW- 2939 |
| 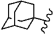 [SMILES: *C12CC3CC(CC(C3)C1)C2] | XPW-2800 | XPW-2814 | XPW-2828 | XPW-2842 | XPW-2856 | XPW-2870 | XPW-2884 | XPW-2898 | XPW-2912 | XPW-2926 | XPW- 2940 |

The above table constitutes an individualized description of each of the specifically indicated compounds therein as well as their salts and solvates.

TABLE 34

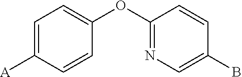

| A/B | 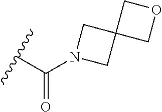 [SMILES: *C(=O)N1CC2(COC2)C1] | 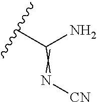 [SMILES: *C(N)=NC#N] | 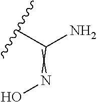 [SMILES: *C(N)=NO] |
|---|---|---|---|
| 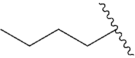 [SMILES: *CCCC] | XPW-2941 | XPW-2955 | XPW-5014 |
| 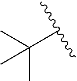 [SMILES: *C(C)(C)C] | XPW-2942 | XPW-2956 | XPW-5015 |
| 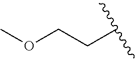 [SMILES: *CCOC] | XPW-2943 | XPW-2957 | XPW-5016 |
| 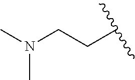 [SMILES: *CCN(C)C] | XPW-2944 | XPW-2958 | XPW-5017 |
| 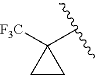 [SMILES: *C1(C(F)(F)F)CC1] | XPW-2945 | XPW-2959 | XPW-5018 |
| 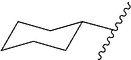 [SMILES: *C1CCCCC1] | XPW-2946 | XPW-2960 | XPW-5019 |
| 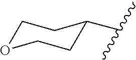 [SMILES: *C1CCOCC1] | XPW-2947 | XPW-2961 | XPW-5020 |
| 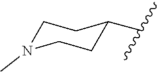 [SMILES: *C1CCN(C)CC1] | XPW-2948 | XPW-2962 | XPW-5021 |
| 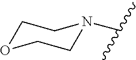 [SMILES: *N1CCOCC1] | XPW-2949 | XPW-2963 | XPW-5022 |
| 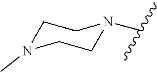 [SMILES: *N1CCN(C)CC1] | XPW-2950 | XPW-2964 | XPW-5023 |
| 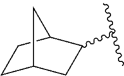 | XPW-2951 | XPW-2965 | XPW-5024 |
| 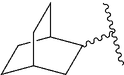 | XPW-2952 | XPW-2966 | XPW-5025 |
| 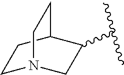 | XPW-2953 | XPW-2967 | XPW-5026 |
| 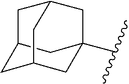 [SMILES: *C12CC3CC(CC(C3)C1)C2] | XPW-2954 | XPW-2968 | XPW-5027 |

TABLE 35

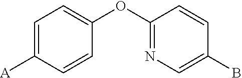

| A\B | 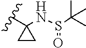 [SMILES: *C1(NS(=O)C(C)(C)C)CC1] | 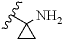 [SMILES: *C1(N)CC1] | 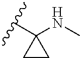 [SMILES: *C1(NC)CC1] | 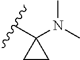 [SMILES: *C1(N(C)C)CC1] | 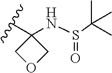 [SMILES: *C1(NS(=O)C(C)(C)C)COC1] | 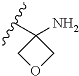 [SMILES: *C1(N)COC1] | 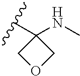 [SMILES: *C1(NC)COC1] | 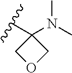 [SMILES: *C1(N(C)C)COC1] | 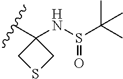 [SMILES: *C1(NS(=O)C(C)(C)C)CSC1] | 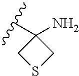 [SMILES: *C1(N)CSC1] | 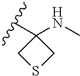 [SMILES: *C1(NC)CSC1] |
|---|---|---|---|---|---|---|---|---|---|---|---|
| 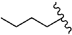 [SMILES: *CCCC] | XPW-2969 | XPW-2983 | XPW-2997 | XPW-3011 | XPW-3025 | XPW-3039 | XPW-3053 | XPW-3067 | XPW-3081 | XPW-3095 | XPW-3109 |
| 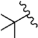 [SMILES: *C(C)(C)C] | XPW-2970 | XPW-2984 | XPW-2998 | XPW-3012 | XPW-3026 | XPW-3040 | XPW-3054 | XPW-3068 | XPW-3082 | XPW-3096 | XPW-3110 |
| 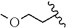 [SMILES: *CCOC] | XPW-2971 | XPW-2985 | XPW-2999 | XPW-3013 | XPW-3027 | XPW-3041 | XPW-3055 | XPW-3069 | XPW-3083 | XPW-3097 | XPW-3111 |
| 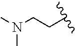 [SMILES: *CCN(C)C] | XPW-2972 | XPW-2986 | XPW-3000 | XPW-3014 | XPW-3028 | XPW-3042 | XPW-3056 | XPW-3070 | XPW-3084 | XPW-3098 | XPW-3112 |
| 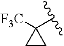 [SMILES: *C1(C(F)(F)F)CC1] | XPW-2973 | XPW-2987 | XPW-3001 | XPW-3015 | XPW-3029 | XPW-3043 | XPW-3057 | XPW-3071 | XPW-3085 | XPW-3099 | XPW-3113 |
| 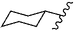 [SMILES: *C1CCCCC1] | XPW-2974 | XPW-2988 | XPW-3002 | XPW-3016 | XPW-3030 | XPW-3044 | XPW-3058 | XPW-3072 | XPW-3086 | XPW-3100 | XPW-3114 |
| 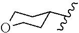 [SMILES: *C1CCOCC1] | XPW-2975 | XPW-2989 | XPW-3003 | XPW-3017 | XPW-3031 | XPW-3045 | XPW-3059 | XPW-3073 | XPW-3087 | XPW-3101 | XPW-3115 |
| 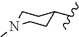 [SMILES: *C1CCN(C)CC1] | XPW-2976 | XPW-2990 | XPW-3004 | XPW-3018 | XPW-3032 | XPW-3046 | XPW-3060 | XPW-3074 | XPW-3088 | XPW-3102 | XPW-3116 |
| 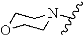 [SMILES: *N1CCOCC1] | XPW-2977 | XPW-2991 | XPW-3005 | XPW-3019 | XPW-3033 | XPW-3047 | XPW-3061 | XPW-3075 | XPW-3089 | XPW-3103 | XPW-3117 |
| 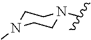 [SMILES: *N1CCN(C)CC1] | XPW-2978 | XPW-2992 | XPW-3006 | XPW-3020 | XPW-3034 | XPW-3048 | XPW-3062 | XPW-3076 | XPW-3090 | XPW-3104 | XPW-3118 |
| 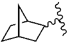 | XPW-2979 | XPW-2993 | XPW-3007 | XPW-3021 | XPW-3035 | XPW-3049 | XPW-3063 | XPW-3077 | XPW-3091 | XPW-3105 | XPW-3119 |
| 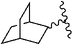 | XPW-2980 | XPW-2994 | XPW-3008 | XPW-3022 | XPW-3036 | XPW-3050 | XPW-3064 | XPW-3078 | XPW-3092 | XPW-3106 | XPW-3120 |
| 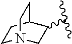 | XPW-2981 | XPW-2995 | XPW-3009 | XPW-3023 | XPW-3037 | XPW-3051 | XPW-3065 | XPW-3079 | XPW-3093 | XPW-3107 | XPW-3121 |
| 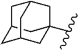 [SMILES: *C12CC3CC(CC(C3)C1)C2] | XPW-2982 | XPW-2996 | XPW-3010 | XPW-3024 | XPW-3038 | XPW-3052 | XPW-3066 | XPW-3080 | XPW-3094 | XPW-3108 | XPW-3122 |

The above table constitutes an individualized description of each of the specifically indicated compounds therein as well as their salts and solvates.

TABLE 36

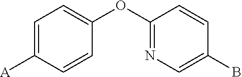

| A\B | 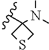 [SMILES: *C1(N(C)C)CSC1] | 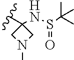 [SMILES: *C1(NS(=O)C(C)(C)C)CN(C)C1] | 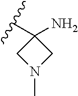 [SMILES: *C1(N)CN(C)C1] | 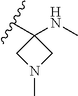 [SMILES: *C1(NC)CN(C)C1] | 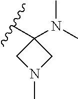 [SMILES: *C1(N(C)C)CN(C)C1] | 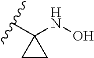 [SMILES: *C1(NO)CC1] | 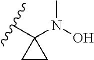 [SMILES: *C1(N(C)O)CC1] | 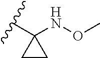 [SMILES: *C1(NOC)CC1] | 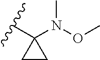 [SMILES: *C1(N(C)OC)CC1] | 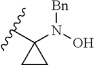 [SMILES: *C1(N(O)Cc2ccccc2)CC1] | 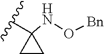 [SMILES: *C1(NOCc2ccccc2)CC1] |
|---|---|---|---|---|---|---|---|---|---|---|---|
| 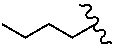 [SMILES: *CCCC] | XPW- 3123 | XPW-3137 | XPW-3151 | XPW-3165 | XPW-3179 | XPW-3906 | XPW-3920 | XPW-3934 | XPW-3948 | XPW-3962 | XPW-3976 |
| 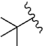 [SMILES: *C(C)(C)C] | XPW- 3124 | XPW-3138 | XPW-3152 | XPW-3166 | XPW-3180 | XPW-3907 | XPW-3921 | XPW-3935 | XPW-3949 | XPW-3963 | XPW-3977 |
| 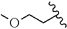 [SMILES: *CCOC] | XPW- 3125 | XPW-3139 | XPW-3153 | XPW-3167 | XPW-3181 | XPW-3908 | XPW-3922 | XPW-3936 | XPW-3950 | XPW-3964 | XPW-3978 |
| 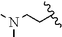 [SMILES: *CCN(C)C] | XPW- 3126 | XPW-3140 | XPW-3154 | XPW-3168 | XPW-3182 | XPW-3909 | XPW-3923 | XPW-3937 | XPW-3951 | XPW-3965 | XPW-3979 |
| 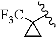 [SMILES: *C1(C(F)(F)F)CC1] | XPW- 3127 | XPW-3141 | XPW-3155 | XPW-3169 | XPW-3183 | XPW-3910 | XPW-3924 | XPW-3938 | XPW-3952 | XPW-3966 | XPW-3980 |
| 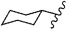 [SMILES: *C1CCCCC1] | XPW- 3128 | XPW-3142 | XPW-3156 | XPW-3170 | XPW-3184 | XPW-3911 | XPW-3925 | XPW-3939 | XPW-3953 | XPW-3967 | XPW-3981 |
| 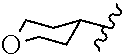 [SMILES: *C1CCOCC1] | XPW- 3129 | XPW-3143 | XPW-3157 | XPW-3171 | XPW-3185 | XPW-3912 | XPW-3926 | XPW-3940 | XPW-3954 | XPW-3968 | XPW-3982 |
| 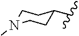 [SMILES: *C1CCN(C)CC1] | XPW- 3130 | XPW-3144 | XPW-3158 | XPW-3172 | XPW-3186 | XPW-3913 | XPW-3927 | XPW-3941 | XPW-3955 | XPW-3969 | XPW-3983 |
| 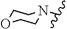 [SMILES: *N1CCOCC1] | XPW- 3131 | XPW-3145 | XPW-3159 | XPW-3173 | XPW-3187 | XPW-3914 | XPW-3928 | XPW-3942 | XPW-3956 | XPW-3970 | XPW-3984 |
| 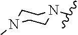 [SMILES: *N1CCN(C)CC1] | XPW- 3132 | XPW-3146 | XPW-3160 | XPW-3174 | XPW-3188 | XPW-3915 | XPW-3929 | XPW-3943 | XPW-3957 | XPW-3971 | XPW-3985 |
| 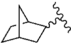 | XPW- 3133 | XPW-3147 | XPW-3161 | XPW-3175 | XPW-3189 | XPW-3916 | XPW-3930 | XPW-3944 | XPW-3958 | XPW-3972 | XPW-3986 |
| 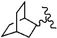 | XPW- 3134 | XPW-3148 | XPW-3162 | XPW-3176 | XPW-3190 | XPW-3917 | XPW-3931 | XPW-3945 | XPW-3959 | XPW-3973 | XPW-3987 |
| 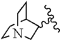 | XPW- 3135 | XPW-3149 | XPW-3163 | XPW-3177 | XPW-3191 | XPW-3918 | XPW-3932 | XPW-3946 | XPW-3960 | XPW-3974 | XPW-3988 |
| 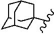 [SMILES: *C12CC3CC(CC(C3)C1)C2] | XPW- 3136 | XPW-3150 | XPW-3164 | XPW-3178 | XPW-3192 | XPW-3919 | XPW-3933 | XPW-3947 | XPW-3961 | XPW-3975 | XPW-3989 |

The above table constitutes an individualized description of each of the specifically indicated compounds therein as well as their salts and solvates.

TABLE 37

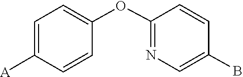

| A\B | 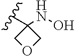 [SMILES: *C1(NO)COC1] | 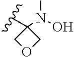 [SMILES: *C1(N(C)O)COC1] | 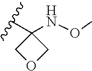 [SMILES: *C1(NOC)COC1] | 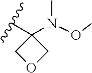 [SMILES: *C1(N(C)OC)COC1] | 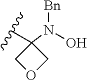 [SMILES: *C1(N(O)Cc2ccccc2)COC1] | 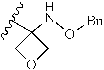 [SMILES: *C1(NOCc2ccccc2)COC1] | 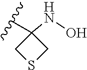 [SMILES: *C1(NO)CSC1] | 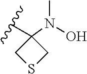 [SMILES: *C1(N(C)O)CSC1] | 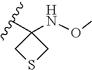 [SMILES: *C1(NOC)CSC1] | 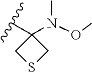 [SMILES: *C1(N(C)OC)CSC1] | 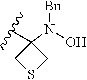 [SMILES: *C1(N(O)Cc2ccccc2)CSC1] |
|---|---|---|---|---|---|---|---|---|---|---|---|
| 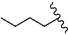 [SMILES: *CCCC] | XPW-3990 | XPW-4004 | XPW-4018 | XPW-4032 | XPW-4046 | XPW-4060 | XPW-4074 | XPW-4088 | XPW-4102 | XPW-4116 | XPW-4130 |
| 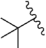 [SMILES: *C(C)(C)C] | XPW-3991 | XPW-4005 | XPW-4019 | XPW-4033 | XPW-4047 | XPW-4061 | XPW-4075 | XPW-4089 | XPW-4103 | XPW-4117 | XPW-4131 |
| 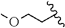 [SMILES: *CCOC] | XPW-3992 | XPW-4006 | XPW-4020 | XPW-4034 | XPW-4048 | XPW-4062 | XPW-4076 | XPW-4090 | XPW-4104 | XPW-4118 | XPW-4132 |
| 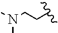 [SMILES: *CCN(C)C] | XPW-3993 | XPW-4007 | XPW-4021 | XPW-4035 | XPW-4049 | XPW-4063 | XPW-4077 | XPW-4091 | XPW-4105 | XPW-4119 | XPW-4133 |
| 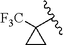 [SMILES: *C1(C(F)(F)F)CC1] | XPW-3994 | XPW-4008 | XPW-4022 | XPW-4036 | XPW-4050 | XPW-4064 | XPW-4078 | XPW-4092 | XPW-4106 | XPW-4120 | XPW-4134 |
| 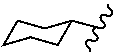 [SMILES: *C1CCCCC1] | XPW-3995 | XPW-4009 | XPW-4023 | XPW-4037 | XPW-4051 | XPW-4065 | XPW-4079 | XPW-4093 | XPW-4107 | XPW-4121 | XPW-4135 |
| 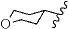 [SMILES: *C1CCOCC1] | XPW-3996 | XPW-4010 | XPW-4024 | XPW-4038 | XPW-4052 | XPW-4066 | XPW-4080 | XPW-4094 | XPW-4108 | XPW-4122 | XPW-4136 |
| 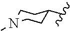 [SMILES: *C1CCN(C)CC1] | XPW-3997 | XPW-4011 | XPW-4025 | XPW-4039 | XPW-4053 | XPW-4067 | XPW-4081 | XPW-4095 | XPW-4109 | XPW-4123 | XPW-4137 |
| 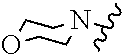 [SMILES: *N1CCOCC1] | XPW-3998 | XPW-4012 | XPW-4026 | XPW-4040 | XPW-4054 | XPW-4068 | XPW-4082 | XPW-4096 | XPW-4110 | XPW-4124 | XPW-4138 |
| 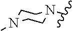 [SMILES: *N1CCN(C)CC1] | XPW-3999 | XPW-4013 | XPW-4027 | XPW-4041 | XPW-4055 | XPW-4069 | XPW-4083 | XPW-4097 | XPW-4111 | XPW-4125 | XPW-4139 |
| 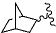 | XPW-4000 | XPW-4014 | XPW-4028 | XPW-4042 | XPW-4056 | XPW-4070 | XPW-4084 | XPW-4098 | XPW-4112 | XPW-4126 | XPW-4140 |
| 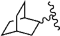 | XPW-4001 | XPW-4015 | XPW-4029 | XPW-4043 | XPW-4057 | XPW-4071 | XPW-4085 | XPW-4099 | XPW-4113 | XPW-4127 | XPW-4141 |
| 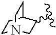 | XPW-4002 | XPW-4016 | XPW-4030 | XPW-4044 | XPW-4058 | XPW-4072 | XPW-4086 | XPW-4100 | XPW-4114 | XPW-4128 | XPW-4142 |
| 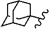 [SMILES: *C12CC3CC(CC(C3)C1)C2] | XPW-4003 | XPW-4017 | XPW-4031 | XPW-4045 | XPW-4059 | XPW-4073 | XPW-4087 | XPW-4101 | XPW-4115 | XPW-4129 | XPW-4143 |

The above table constitutes an individualized description of each of the specifically indicated compounds therein as well as their salts and solvates.

TABLE 38

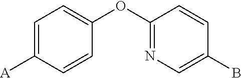

| A\B | 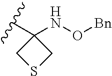 [SMILES: *C1(NOCc2ccccc2)CSC1] | 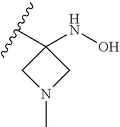 [SMILES: *C1(NO)CN(C)C1] | 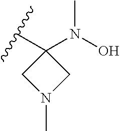 [SMILES: *C1(N(C)O)CN(C)C1] | 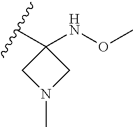 [SMILES: *C1(NOC)CN(C)C1] | 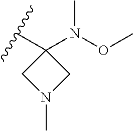 [SMILES: *C1(N(C)OC)CN(C)C1] | 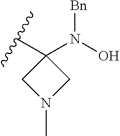 [SMILES: *C1(N(O)Cc2ccccc2)CN(C)C1] | 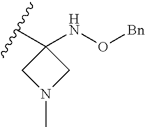 [SMILES: *C1(NOCc2ccccc2)CN(C)C1] | 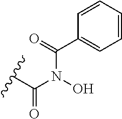 [SMILES: *C(=O)N(O)C(=O)c1ccccc1] |
|---|---|---|---|---|---|---|---|---|
| 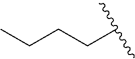 [SMILES: *CCCC] | XPW-4144 | XPW-4158 | XPW-4172 | XPW-4186 | XPW-4500 | XPW-4514 | XPW-4528 | XPW-5028 |
| 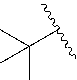 [SMILES: *C(C)(C)C] | XPW-4145 | XPW-4159 | XPW-4173 | XPW-4187 | XPW-4501 | XPW-4515 | XPW-4529 | XPW-5029 |
| 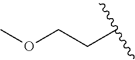 [SMILES: *CCOC] | XPW-4146 | XPW-4160 | XPW-4174 | XPW-4188 | XPW-4502 | XPW-4516 | XPW-4530 | XPW-5030 |
| 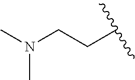 [SMILES: *CCN(C)C] | XPW-4147 | XPW-4161 | XPW-4175 | XPW-4189 | XPW-4503 | XPW-4517 | XPW-4531 | XPW-5031 |
| 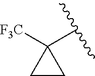 [SMILES: *C1(C(F)(F)F)CC1] | XPW-4148 | XPW-4162 | XPW-4176 | XPW-4190 | XPW-4504 | XPW-4518 | XPW-4532 | XPW-5032 |
| 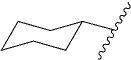 [SMILES: *C1CCCCC1] | XPW-4149 | XPW-4163 | XPW-4177 | XPW-4191 | XPW-4505 | XPW-4519 | XPW-4533 | XPW-5033 |
| 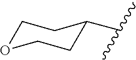 [SMILES: *C1CCOCC1] | XPW-4150 | XPW-4164 | XPW-4178 | XPW-4192 | XPW-4506 | XPW-4520 | XPW-4534 | XPW-5034 |
| 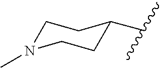 [SMILES: *C1CCN(C)CC1] | XPW-4151 | XPW-4165 | XPW-4179 | XPW-4193 | XPW-4507 | XPW-4521 | XPW-4535 | XPW-5035 |
| 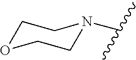 [SMILES: *N1CCOCC1] | XPW-4152 | XPW-4166 | XPW-4180 | XPW-4194 | XPW-4508 | XPW-4522 | XPW-4536 | XPW-5036 |
| 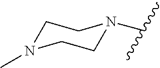 [SMILES: *N1CCN(C)CC1] | XPW-4153 | XPW-4167 | XPW-4181 | XPW-4195 | XPW-4509 | XPW-4523 | XPW-4537 | XPW-5037 |
| 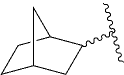 | XPW-4154 | XPW-4168 | XPW-4182 | XPW-4196 | XPW-4510 | XPW-4524 | XPW-4538 | XPW-5038 |
| 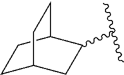 | XPW-4155 | XPW-4169 | XPW-4183 | XPW-4197 | XPW-4511 | XPW-4525 | XPW-4539 | XPW-5039 |
| 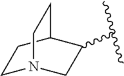 | XPW-4156 | XPW-4170 | XPW-4184 | XPW-4198 | XPW-4512 | XPW-4526 | XPW-4540 | XPW-5040 |
| 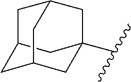 [SMILES: *C12CC3CC(CC(C3)C1)C2] | XPW-4157 | XPW-4171 | XPW-4185 | XPW-4199 | XPW-4513 | XPW-4527 | XPW-4541 | XPW-5041 |

The above table constitutes an individualized description of each of the specifically indicated compounds therein as well as their salts and solvates.

TABLE 39

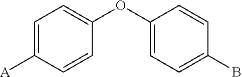

| A\B | 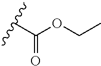 [SMILES: *C(=O)OCC] | 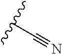 [SMILES: *C#N] | 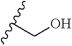 [SMILES: *CO] | 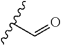 [SMILES: *C=O] | 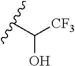 [SMILES: *C(O)C(F)(F)F] | 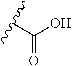 [SMILES: *C(=O)O] | 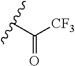 [SMILES: *C(=O)C(F)(F)F] | 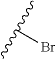 [SMILES: *Br] |
|---|---|---|---|---|---|---|---|---|
| 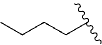 [SMILES: *CCCC] | XPW-I-0003 | | | | | XPW-I-0023 | | |
| 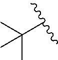 [SMILES: *C(C)(C)C] | XPW-I-0001 | | | | | XPW-I-0024 | | |
| 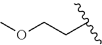 [SMILES: *CCOC] | XPW-I-0011 | | | | | XPW-I-0014 | | |
| 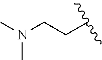 [SMILES: *CCN(C)C] | XPW-I-0012 | | | | | XPW-I-0015 | | |
| 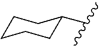 [SMILES: *C1CCCCC1] | XPW-I-0002 | XPW-I-0007 | XPW-I-0008 | XPW-I-0009 | XPW-I-0010 | XPW-I-0025 | XPW-I-0017 | XPW-I-0021 |
| 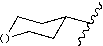 [SMILES: *C1CCOCC1] | XPW-I-0019 | | | | | XPW-I-0022 | | |
| 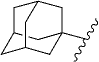 [SMILES: *C12CC3CC(CC(C3)C1)C2] | XPW-I-0004 | | | | | XPW-I-0030 | | XPW-I-0018 |
| 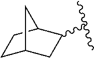 | XPW-I-0026 | | | | | XPW-I-0027 | | |
| 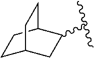 | XPW-I-0028 | | | | | XPW-I-0029 | | |
| 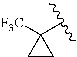 [SMILES: *C1(C(F)(F)F)CC1] | XPW-I-0032 | | | | | XPW-I-0031 | | |

The above table constitutes an individualized description of each of the specifically indicated intermediates used for the synthesis of XPW-0001 to XPW-5062 as well as their salts and solvates. Intermediates as such as well as their salts and solvates are also part of the invention, also in the frame of the process of generating the final compounds.

TABLE 40

| | 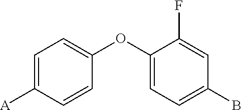 | | 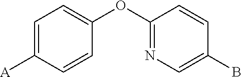 | | |
|---|---|---|---|---|---|
| A\B | 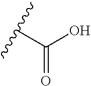 [SMILES: *C(=O)O] | 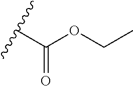 [SMILES: *C(=O)OCC] | 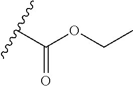 [SMILES: *C(=O)OCC] | 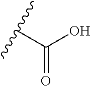 [SMILES: *C(=O)O] | 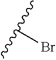 [SMILES: *Br] |
| 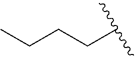 [SMILES: *CCCC] | XPW-I-0034 | XPW-I-0033 | XPW-I-0035 | XPW-I-0036 | |
| 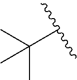 [SMILES: *C(C)(C)C] | XPW-I-0050 | XPW-I-0049 | XPW-I-0037 | XPW-I-0038 | |
| 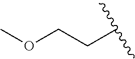 [SMILES: *CCOC] | | | XPW-I-0013 | XPW-I-0016 | |
| 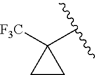 [SMILES: *C1(C(F)(F)F)CC1] | XPW-I-0006 | XPW-I-0054 | XPW-I-0047 | XPW-I-0048 | |
| 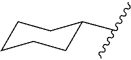 [SMILES: *C1CCCCC1] | XPW-I-0052 | XPW-I-0051 | XPW-I-0039 | XPW-I-0040 | |
| 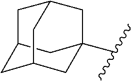 [SMILES: *C12CC3CC(CC(C3)C1)C2] | XPW-I-0005 | XPW-I-0053 | XPW-I-0045 | XPW-I-0046 | XPW-I-0020 |
| 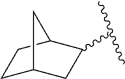 | | | XPW-I-0041 | XPW-I-0042 | |
| 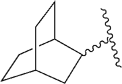 | | | XPW-I-0043 | XPW-I-0044 | |

The above table constitutes an individualized description of each of the specifically indicated intermediates used for the synthesis of XPW-0001 to XPW-5062 as well as their salts and solvates. Intermediates as such as well as their salts and solvates are also part of the invention, also in the frame of the process of generating the final compounds.

TABLE 41

| | 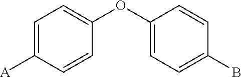 | | | | | 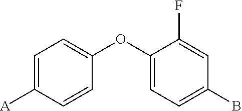 | | | |
|---|---|---|---|---|---|---|---|---|---|
| A\B | 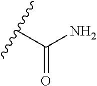 [SMILES: *C(N)=O] | 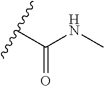 [SMILES: *C(=O)NC] | 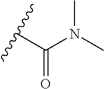 [SMILES: *C(=O)N(C)C] | 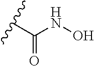 [SMILES: *C(=O)NO] | 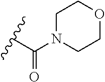 [SMILES: *C(=O)N1CCOCC1] | 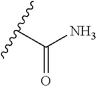 [SMILES: *C(N)=O] | 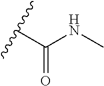 [SMILES: *C(=O)NC] | 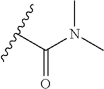 [SMILES: *C(=O)N(C)C] | 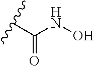 [SMILES: *C(=O)NO] |
| 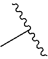 | XPW-4714 | XPW-4565 | XPW-4723 | XPW-3217 | XPW-4726 | XPW-4736 | XPW-4744 | XPW-4752 | XPW-4761 |
| 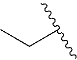 [SMILES: *CC] | XPW-4715 | XPW-3215 | XPW-4645 | XPW-3216 | XPW-4603 | XPW-4737 | XPW-4745 | XPW-4753 | XPW-4762 |
| 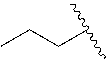 [SMILES: *CCC] | XPW-4716 | XPW-4563 | XPW-4564 | XPW-3214 | XPW-4730 | XPW-4738 | XPW-4746 | XPW-4754 | XPW-4763 |
| 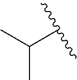 [SMILES: *C(C)C] | XPW-3210 | XPW-3211 | XPW-4560 | XPW-3212 | XPW-4731 | XPW-4739 | XPW-4747 | XPW-4755 | XPW-4764 |
| 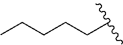 [SMILES: *CCCCC] | XPW-4717 | XPW-4561 | XPW-4562 | XPW-3213 | XPW-4732 | XPW-4740 | XPW-4748 | XPW-4756 | XPW-4765 |
| 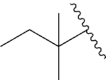 [SMILES: *C(C)(C)CC] | XPW-4718 | XPW-4544 | XPW-4543 | XPW-3193 | XPW-4733 | XPW-3198 | XPW-3199 | XPW-4757 | XPW-3200 |
| 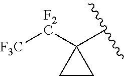 [SMILES: *C1(C(F)(F)C(F)(F)F)CC1] | XPW-4719 | XPW-4721 | XPW-3232 | XPW-3233 | XPW-4734 | XPW-4741 | XPW-4749 | XPW-4758 | XPW-4766 |
| 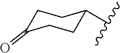 [SMILES: *C1CCC(=O)CC1] | XPW-4720 | XPW-4722 | XPW-4724 | XPW-4725 | XPW-4576 | XPW-4742 | XPW-4750 | XPW-4759 | XPW-4767 |
| 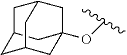 [SMILES: *OC12CC3CC(CC(C3)C1)C2] | XPW-4727 | XPW-4728 | XPW-4729 | XPW-4613 | XPW-4735 | XPW-4743 | XPW-4751 | XPW-4760 | XPW-4768 |

The above table constitutes an individualized description of each of the specifically indicated compounds therein as well as their salts and solvates.

TABLE 42

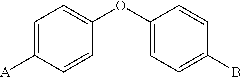

| A\B | 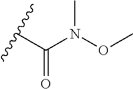 [SMILES: *C(=O)N(C)OC] |
|---|---|
| 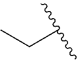 [SMILES: *CC] | XPW-4605 |
| 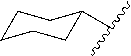 [SMILES: *C1CCCCC1] | XPW-0720 |
| 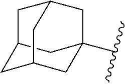 [SMILES: *C12CC3CC(CC(C3)C1)C2] | XPW-4787 |

The above table constitutes an individualized description of each of the specifically indicated compounds therein as well as their salts and solvates.

TABLE 43

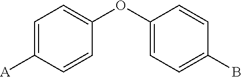

| A\B | 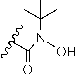 [SMILES: *C(=O)N(O)C(C)(C)C] | 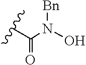 [SMILES: *C(=O)N(O)Cc1ccccc1] | 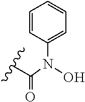 [SMILES: *C(=O)N(O)c1ccccc1] | 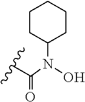 [SMILES: *C(=O)N(O)C1CCCCC1] | 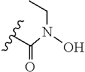 [SMILES: *C(=O)N(O)CC] | 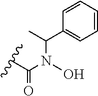 [SMILES: *C(=O)N(O)C(C)c1ccccc1] |
|---|---|---|---|---|---|---|
| 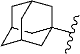 [SMILES: *C12CC3CC(CC(C3)C1)C2] | XPW-4612 | XPW-4802 | XPW-4616 | XPW-4617 | XPW-4618 | XPW-4619 |
| 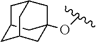 [SMILES: *OC12CC3CC(CC(C3)C1)C2] | XPW-4804 | XPW-4614 | XPW-4805 | XPW-4806 | XPW-4807 | XPW-4808 |
| 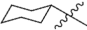 [SMILES: *C1CCCCC1] | XPW-4815 | XPW-4816 | XPW-4817 | XPW-4818 | XPW-4819 | XPW-4820 |
| A\B | 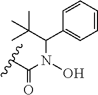 [SMILES: *C(=O)N(O)C(c1ccccc1)C(C)(C)C] | 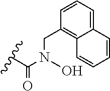 [SMILES: *C(=O)N(O)Cc1cccc2ccccc12] | 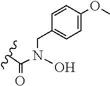 [SMILES: *C(=O)N(O)Cc1ccc(OC)cc1] | 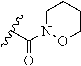 [SMILES: *C(=O)N1CCCCO1] | 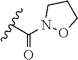 [SMILES: *C(=O)N1CCCO1] | 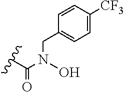 [SMILES: *C(=O)N(O)Cc1ccc(C(F)(F)F)cc1] |
| 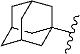 [SMILES: *C12CC3CC(CC(C3)C1)C2] | XPW-4620 | XPW-4621 | XPW-4622 | XPW-4637 | XPW-4803 | XPW-4646 |
| 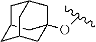 [SMILES: *OC12CC3CC(CC(C3)C1)C2] | XPW-4809 | XPW-4810 | XPW-4811 | XPW-4812 | XPW-4813 | XPW-4814 |
| 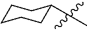 [SMILES: *C1CCCCC1] | XPW-4821 | XPW-4822 | XPW-4823 | XPW-4824 | XPW-4638 | XPW-4825 |

The above table constitutes an individualized description of each of the specifically indicated compounds therein as well as their salts and solvates.

TABLE 44

| | 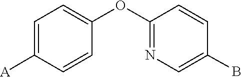 | | | | 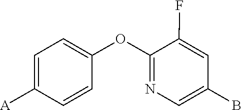 | | | |
|---|---|---|---|---|---|---|---|---|
| A\B | 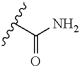 [SMILES: *C(N)=O] | 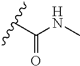 [SMILES: *C(=O)NC] | 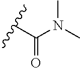 [SMILES: *C(=O)N(C)C] | 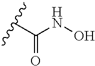 [SMILES: *C(=O)NO] | 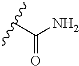 [SMILES: *C(N)=O] | 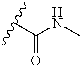 [SMILES: *C(=O)NC] | 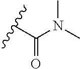 [SMILES: *C(=O)N(C)C] | 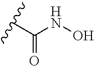 [SMILES: *C(=O)NO] |
| 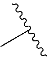 | XPW-4552 | XPW-4553 | XPW-4554 | XPW-3202 | XPW-4838 | XPW-4846 | XPW-4855 | XPW-4862 |
| 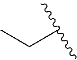 [SMILES: *CC] | XPW-4555 | XPW-4556 | XPW-4557 | XPW-3203 | XPW-4839 | XPW-4847 | XPW-4856 | XPW-4863 |
| 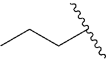 [SMILES: *CCC] | XPW-4558 | XPW-3204 | XPW-3205 | XPW-3206 | XPW-4840 | XPW-4848 | XPW-4857 | XPW-4864 |
| 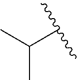 [SMILES: *C(C)C] | XPW-4547 | XPW-4548 | XPW-4549 | XPW-3201 | XPW-4841 | XPW-4849 | XPW-4858 | XPW-4865 |
| 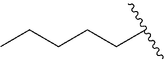 [SMILES: *CCCCC] | XPW-4559 | XPW-3207 | XPW-3208 | XPW-3209 | XPW-4842 | XPW-4850 | XPW-4859 | XPW-4866 |
| 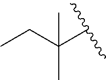 [SMILES: *C(C)(C)CC] | XPW-3195 | XPW-3196 | XPW-4545 | XPW-3197 | XPW-4843 | XPW-4851 | XPW-4546 | XPW-3219 |
| 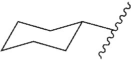 [SMILES: *C1CCCCC1] | XPW-2638 | XPW-2652 | XPW-2666 | XPW-2792 | XPW-3220 | XPW-4550 | XPW-4551 | XPW-3221 |
| 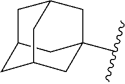 [SMILES: *C12CC3CC(CC(C3)C1)C2] | XPW-2646 | XPW-2660 | XPW-2674 | XPW-2800 | XPW-3222 | XPW-3223 | XPW-3224 | XPW-3225 |
| 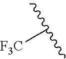 [SMILES: *C(F)(F)F] | XPW-4568 | XPW-4569 | XPW-4570 | XPW-3218 | XPW-4844 | XPW-4852 | XPW-4860 | XPW-4867 |
| 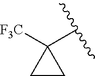 [SMILES: *C1(C(F)(F)F)CC1] | XPW-4828 | XPW-4831 | XPW-4834 | XPW-4837 | XPW-4566 | XPW-4853 | XPW-4567 | XPW-3226 |
| 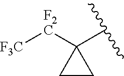 [SMILES: *C1(C(F)(F)C(F)(F)F)CC1] | XPW-3228 | XPW-3229 | XPW-3230 | XPW-3231 | XPW-4845 | XPW-4854 | XPW-4861 | XPW-4868 |

The above table constitutes an individualized description of each of the specifically indicated compounds therein as well as their salts and solvates.

TABLE 45

| | 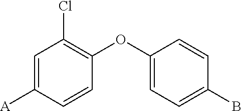 | | | | |
|---|---|---|---|---|---|
| A\B | 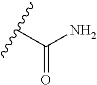 [SMILES: *C(N)=O] | 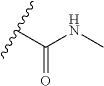 [SMILES: *C(=O)NC] | 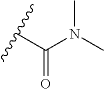 [SMILES: *C(=O)N(C)C] | 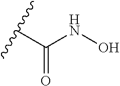 [SMILES: *C(=O)NO] | 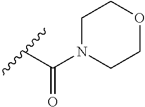 [SMILES: *C(=O)N1CCOCC1] |
| 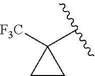 [SMILES: *C1(C(F)(F)F)CC1] | XPW-4869 | XPW-4871 | XPW-3234 | XPW-3194 | XPW-4874 |
| 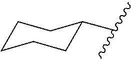 [SMILES: *C1CCCCC1] | XPW-4869 | XPW-4872 | XPW-4575 | XPW-4584 | XPW-4583 |
| 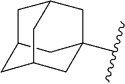 [SMILES: *C12CC3CC(CC(C3)C1)C2] | XPW-4870 | XPW-4873 | XPW-4585 | XPW-4587 | XPW-4586 |
| | | 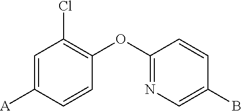 | | | |
| | A/B | 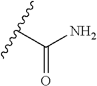 [SMILES: *C(N)=O] | 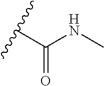 [SMILES: *C(=O)NC] | 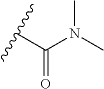 [SMILES: *C(=O)N(C)C] | 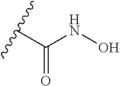 [SMILES: *C(=O)NO] |
| | 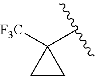 [SMILES: *C1(C(F)(F)F)CC1] | XPW-4571 | XPW-4572 | XPW-4573 | XPW-3227 |
| | 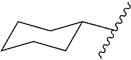 [SMILES: *C1CCCCC1] | XPW-4875 | XPW-4877 | XPW-4879 | XPW-4881 |
| | 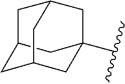 [SMILES: *C12CC3CC(CC(C3)C1)C2] | XPW-4876 | XPW-4878 | XPW-4880 | XPW-4882 |

The above table constitutes an individualized description of each of the specifically indicated compounds therein as well as their salts and solvates.

TABLE 46

| | 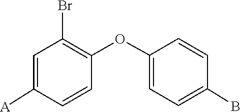 | | | | |
|---|---|---|---|---|---|
| A\B | 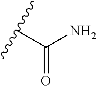 [SMILES: *C(N)=O] | 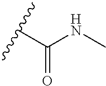 [SMILES: *C(=O)NC] | 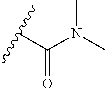 [SMILES: *C(=O)N(C)C] | 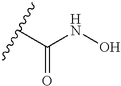 [SMILES: *C(=O)NO] | 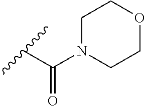 [SMILES: *C(=O)N1CCOCC1] |
| 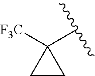 [SMILES: *C1(C(F)(F)F)CC1] | XPW-4883 | XPW-4886 | XPW-4889 | XPW-4890 | XPW-4891 |
| 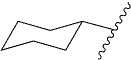 [SMILES: *C1CCCCC1] | XPW-4884 | XPW-4887 | XPW-4588 | XPW-4590 | XPW-4589 |
| 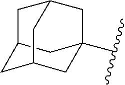 [SMILES: *C12CC3CC(CC(C3)C1)C2] | XPW-4885 | XPW-4888 | XPW-4591 | XPW-4593 | XPW-4592 |
| | | 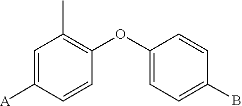 | | | |
| | A\B | 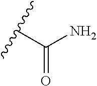 [SMILES: *C(N)=O] | 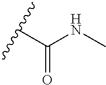 [SMILES: *C(=O)NC] | 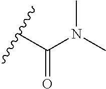 [SMILES: *C(=O)N(C)C] | 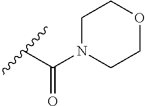 [SMILES: *C(=O)N1CCOCC1] |
| | 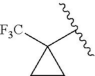 [SMILES: *C1(C(F)(F)F)CC1] | XPW-4892 | XPW-4895 | XPW-4898 | XPW-4900 |
| | 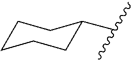 [SMILES: *C1CCCCC1] | XPW-4893 | XPW-4896 | XPW-4595 | XPW-4594 |
| | 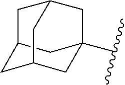 [SMILES: *C12CC3CC(CC(C3)C1)C2] | XPW-4894 | XPW-4897 | XPW-4899 | XPW-4901 |

The above table constitutes an individualized description of each of the specifically indicated compounds therein as well as their salts and solvates.

TABLE 47

| | 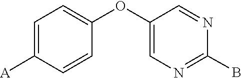 | | | | 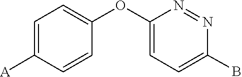 | | | | |
|---|---|---|---|---|---|---|---|---|---|
| A\B | 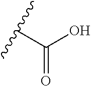 [SMILES: *C(=O)O] | 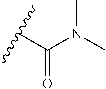 [SMILES: *C(=O)N(C)C] | 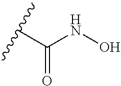 [SMILES: *C(=O)NO] | 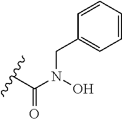 [SMILES: *C(=O)N(O)Cc1ccccc1] | 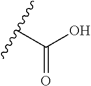 [SMILES: *C(=O)O] | 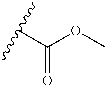 [SMILES: *C(=O)OC] | 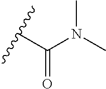 [SMILES: *C(=O)N(C)C] | 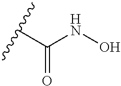 [SMILES: *C(=O)NO] | 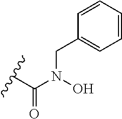 [SMILES: *C(=O)N(O)Cc1ccccc1] |
| 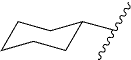 [SMILES: *C1CCCCC1] | | XPW-5043 | XPW-5044 | XPW-5045 | | | XPW-5048 | XPW-5049 | XPW-5050 |
| 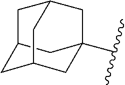 [SMILES: *C12CC3CC(CC(C3)C1)C2] | XPW-I-0139 | XPW-4634 | XPW-4636 | XPW-4644 | XPW-I-0143 | XPW-I-0140 | XPW-4624 | XPW-4623 | XPW-4632 |

The above table constitutes an individualized description of each of the specifically indicated compounds therein as well as their salts and solvates used as intermediates for the synthesis. Intermediates as such as well as their salts and solvates are also part of the invention, also in the frame of the process of generating the final compounds.

TABLE 48

| | 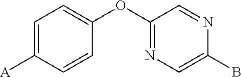 | | | | | 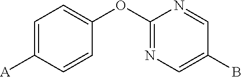 | | | | |
|---|---|---|---|---|---|---|---|---|---|---|
| A\B | 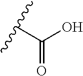 [SMILES: *C(=O)O] | 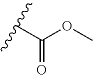 [SMILES: *C(=O)OC] | 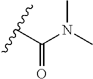 [SMILES: *C(=O)N(C)C] | 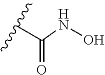 [SMILES: *C(=O)NO] | 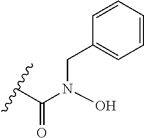 [SMILES: *C(=O)N(O)Cc1ccccc1] | 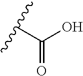 [SMILES: *C(=O)O] | 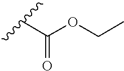 [SMILES: *C(=O)OCC] | 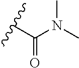 [SMILES: *C(=O)N(C)C] | 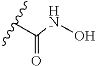 [SMILES: *C(=O)NO] | 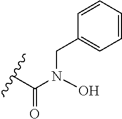 [SMILES: *C(=O)N(O)Cc1ccccc1] |
| 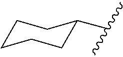 [SMILES: *C1CCCCC1] | | | XPW-5051 | XPW-5052 | XPW-5053 | | | XPW-5054 | XPW-5055 | XPW-5056 |
| 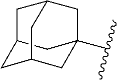 [SMILES: *C12CC3CC(CC(C3)C1)C2] | XPW-I-0145 | XPW-I-0141 | XPW-4629 | XPW-4628 | XPW-4909 | XPW-I-0147 | XPW-I-0143 | XPW-4627 | XPW-4625 | XPW-4626 |

The above table constitutes an individualized description of each of the specifically indicated compounds therein as well as their salts and solvates used as intermediates for the synthesis. Intermediates as such as well as their salts and solvates are also part of the invention, also in the frame of the process of generating the final compounds.

TABLE 49

| | 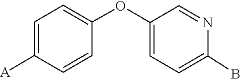 | | | | | 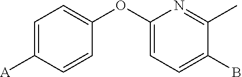 | | | | |
|---|---|---|---|---|---|---|---|---|---|---|
| A\B | 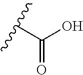 [SMILES: *C(=O)O] | 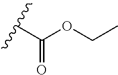 [SMILES: *C(=O)OCC] | 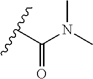 [SMILES: *C(=O)N(C)C] | 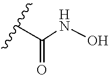 [SMILES: *C(=O)NO] | 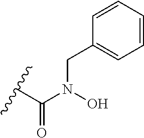 [SMILES: *C(=O)N(O)Cc1ccccc1] | 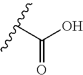 [SMILES: *C(=O)O] | 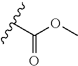 [SMILES: *C(=O)OC] | 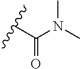 [SMILES: *C(=O)N(C)C] | 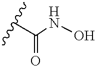 [SMILES: *C(=O)NO] | 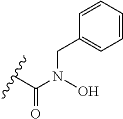 [SMILES: *C(=O)N(O)Cc1ccccc1] |
| 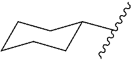 [SMILES: *C1CCCCC1] | | | XPW-5057 | XPW-5058 | XPW-5059 | | | XPW-5060 | XPW-5061 | XPW-5062 |
| 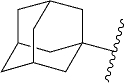 [SMILES: *C12CC3CC(CC(C3)C1)C2] | XPW-I-0146 | XPW-I-0142 | XPW-4635 | XPW-4630 | XPW-4631 | XPW-I-0149 | XPW-I-0148 | XPW-4641 | XPW-4639 | XPW-4640 |

The above table constitutes an individualized description of each of the specifically indicated compounds therein as well as their salts and solvates used as intermediates for the synthesis. Intermediates as such as well as their salts and solvates are also part of the invention, also in the frame of the process of generating the final compounds.

TABLE 50

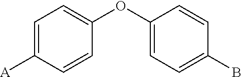

| A\B | 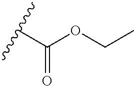 [SMILES: *C(=O)OCC] | 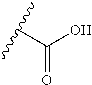 [SMILES: *C(=O)O] |
|---|---|---|
| 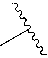 | XPW-I-0064 | XPW-I-0087 |
| 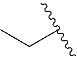 [SMILES: *CC] | XPW-I-0065 | XPW-I-0066 |
| 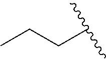 [SMILES: *CCC] | XPW-I-0067 | XPW-I-0068 |
| 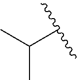 [SMILES: *C(C)C] | XPW-I-0069 | XPW-I-0070 |
| 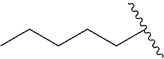 [SMILES: *CCCCC] | XPW-I-0062 | XPW-I-0063 |
| 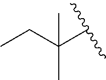 [SMILES: *C(C)(C)CC] | XPW-I-0071 | XPW-I-0072 |
| 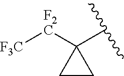 [SMILES: *C1(C(F)(F)C(F)(F)F)CC1] | XPW-I-0100 | XPW-I-0102 |
| 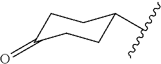 [SMILES: *C1CCC(=O)CC1] | XPW-I-0120 | XPW-I-0121 |
| 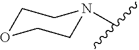 [SMILES: *N1CCOCC1] | XPW-I-0128 | XPW-I-0130 |
| 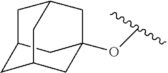 [SMILES: *OC12CC3CC(CC(C3)C1)C2] | XPW-I-0135 | XPW-I-0136 |

The above table constitutes an individualized description of each of the specifically indicated compounds therein as well as their salts and solvates used as intermediates for the synthesis. Intermediates as such as well as their salts and solvates are also part of the invention, also in the frame of the process of generating the final compounds.

TABLE 51

| | 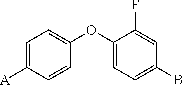 | | 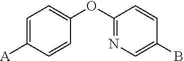 | | 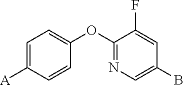 | |
|---|---|---|---|---|---|---|
| A\B | 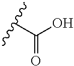 [SMILES: *C(=O)O] | 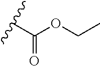 [SMILES: *C(=O)OCC] | 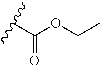 [SMILES: *C(=O)OCC] | 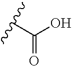 [SMILES: *C(=O)O] | 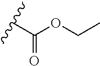 [SMILES: *C(=O)OCC] | 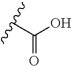 [SMILES: *C(=O)O] |
| 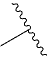 | XPW-I-0057 | XPW-I-0104 | XPW-I-0073 | XPW-I-0074 | XPW-I-0114 | XPW-I-0118 |
| 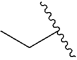 [SMILES: *CC] | XPW-I-0056 | XPW-I-0105 | XPW-I-0075 | XPW-I-0076 | | |
| 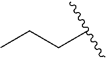 [SMILES: *CCC] | XPW-I-0111 | XPW-I-0109 | XPW-I-0077 | XPW-I-0078 | | |
| 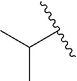 [SMILES: *C(C)C] | XPW-I-0058 | XPW-I-0106 | XPW-I-0081 | XPW-I-0082 | XPW-I-0112 | XPW-I-0116 |
| 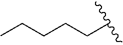 [SMILES: *CCCCC] | XPW-I-0110 | XPW-I-0108 | XPW-I-0079 | XPW-I-0080 | | |
| 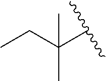 [SMILES: *C(C)(C)CC] | XPW-I-0055 | XPW-I-0086 | XPW-I-0083 | XPW-I-0060 | XPW-I-0088 | XPW-I-0089 |
| 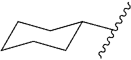 [SMILES: *C1CCCCC1] | | | | | XPW-I-0090 | XPW-I-0091 |
| 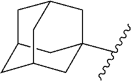 [SMILES: *C12CC3CC(CC(C3)C1)C2] | | | | | XPW-I-0092 | XPW-I-0093 |
| 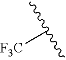 [SMILES: *C(F)(F)F] | XPW-I-0059 | XPW-I-0107 | XPW-I-0084 | XPW-I-0085 | XPW-I-0115 | XPW-I-0119 |
| 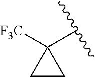 [SMILES: *C1(C(F)(F)F)CC1] | | | | | XPW-I-0094 | XPW-I-0095 |
| 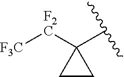 [SMILES: *C1(C(F)(F)C(F)(F)F)CC1] | | | XPW-I-0101 | XPW-I-0103 | | |
| 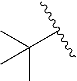 [SMILES: *C(C)(C)C] | | | | | XPW-I-0113 | XPW-I-0117 |
| 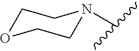 [SMILES: *N1CCOCC1] | | | XPW-I-0129 | XPW-I-0151 | | |

The above table constitutes an individualized description of each of the specifically indicated compounds therein as well as their salts and solvates used as intermediates for the synthesis. Intermediates as such as well as their salts and solvates are also part of the invention, also in the frame of the process of generating the final compounds.

TABLE 52

| | 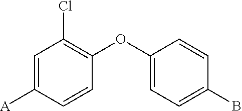 | | 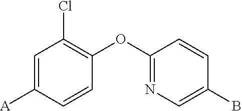 | |
|---|---|---|---|---|
| A\B | 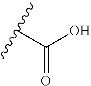 [SMILES: *C(=O)O] | 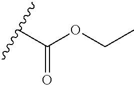 [SMILES: *C(=O)OCC] | 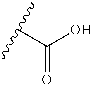 [SMILES: *C(=O)O] | 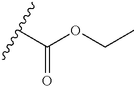 [SMILES: *C(=O)OCC] |
| 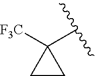 [SMILES: *C1(C(F)(F)F)CC1] | XPW-I-0096 | XPW-I-0097 | XPW-I-0099 | XPW-I-0098 |
| 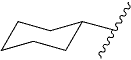 [SMILES: *C1CCCCC1] | XPW-I-0131 | XPW-I-0124 | | |
| 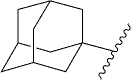 [SMILES: *C12CC3CC(CC(C3)C1)C2] | XPW-I-0132 | XPW-I-0125 | | |

The above table constitutes an individualized description of each of the specifically indicated compounds therein as well as their salts and solvates used as intermediates for the synthesis. Intermediates as such as well as their salts and solvates are also part of the invention, also in the frame of the process of generating the final compounds.

TABLE 53

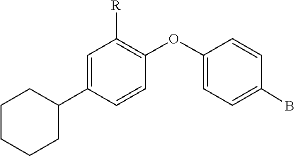

| B\R | 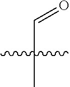 [SMILES: *C=O] | 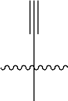 [SMILES: *C#C] | 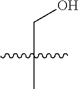 [SMILES: *CO] | 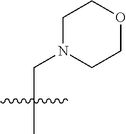 [SMILES: *CN1CCOCC1] | 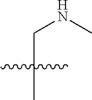 [SMILES: *CNC] | 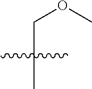 [SMILES: *COC] |
|---|---|---|---|---|---|---|
| 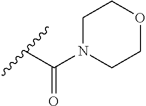 [SMILES: *C(=O)N1CCOCC1] | XPW-I-0150 | XPW-4577 | XPW-4578 | XPW-4579 | XPW-4580 | XPW-4581 |
| 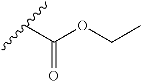 [SMILES: *C(=O)OCC] | XPW-I-0122 | | | | | |
| 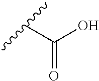 [SMILES: *C(=O)O] | XPW-I-0123 | | | | | |

The above table constitutes an individualized description of each of the specifically indicated compounds therein as well as their salts and solvates used as intermediates for the synthesis. Intermediates as such as well as their salts and solvates are also part of the invention, also in the frame of the process of generating the final compounds.

TABLE 54

| | 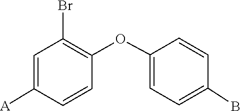 | | 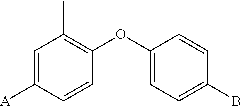 | |
|---|---|---|---|---|
| A\B | 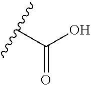 [SMILES: *C(=O)O] | 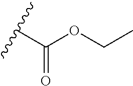 [SMILES: *C(=O)OCC] | 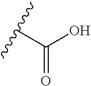 [SMILES: *C(=O)O] | 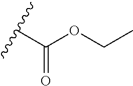 [SMILES: *C(=O)OCC] |
| 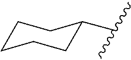 [SMILES: *C1CCCCC1] | XPW-I- 0133 | XPW-I- 0126 | XPW-I- 0138 | XPW-I- 0137 |
| 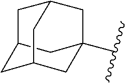 [SMILES: *C12CC3CC(CC(C3)C1)C2] | XPW-I- 0134 | XPW-I- 0127 | | |

The above table constitutes an individualized description of each of the specifically indicated compounds therein as well as their salts and solvates used as intermediates for the synthesis. Intermediates as such as well as their salts and solvates are also part of the invention, also in the frame of the process of generating the final compounds.

Also included are isomers, e.g. enantiomers or diastereomers or mixtures of isomers, salts, particularly pharmaceutically acceptable salts, and solvates of the compounds listed above.

### Further Definitions

The term “C₁-C₁₂ alkyl” comprises all isomers of the corresponding saturated aliphatic hydrocarbon groups containing one to twelve carbon atoms; this includes methyl, ethyl, n-propyl, iso-propyl, n-butyl, sec-butyl, iso-butyl, tert-butyl, n-pentyl, sec-pentyl, 3-pentyl, 2-methylbutyl, iso-pentyl, 2-methylbut-2-yl, 3-methylbut-2-yl, all hexyl-isomers, all heptyl-isomers, all octyl-isomers, all nonyl-isomers, all decyl-isomers, all undecyl-isomers and all dodecyl-isomers.

The term “C₂-C₁₂ alkenyl” comprises all isomers of the corresponding unsaturated olefinic hydrocarbon groups containing two to twelve carbon atoms linked by (i.e. comprising) one or more double bonds; this includes vinyl, all propenyl-isomers, all butenyl-isomers, all pentenyl-isomers, all hexenyl-isomers, all heptenyl-isomers, all octenyl-isomers, all nonenyl-isomers, all decenyl-isomers, all undecenyl-isomers and all dodecenyl-isomers.

The term “C₂-C₁₂ alkynyl” comprises all isomers of the corresponding unsaturated acetylenic hydrocarbon groups containing two to twelve carbon atoms linked by (i.e. comprising) one or more triple bonds; this includes ethynyl, all propynyl-isomers, all butynyl-isomers, all pentynyl-isomers, all hexynyl-isomers, all heptynyl-isomers, all octynyl-isomers, all nonynyl-isomers, all decynyl-isomers, all undecynyl-isomers and all dodecynyl-isomers. The term “alkynyl” also includes compounds having one or more triple bonds and one or more double bonds.

The term “C₃-C₈ cycloalkyl” comprises the corresponding saturated hydrocarbon groups containing three to eight carbon atoms arranged in a monocyclic ring structure; this includes cyclopropyl, cyclobutyl, cyclopentyl, cyclohexyl, cycloheptyl, cyclooctyl.

The term “C₅-C₈ cycloalkenyl” comprises the corresponding unsaturated non-aromatic and non-heteroaromatic hydrocarbon groups containing five to eight carbon atoms, of which at least one is sp³-hybridized, and which are arranged in a monocyclic ring structure and linked by (i.e. comprising) one or more double bonds; this includes all cyclopentenyl-isomers, all cyclohexenyl-isomers, all cycloheptenyl-isomers, all cyclooctenyl-isomers.

The term “C₅-C₁₂ bicycloalkyl” comprises the corresponding saturated hydrocarbon groups containing five to twelve carbon atoms arranged in a bicyclic ring structure; wherein these bicyclic ring structures include fused, bridged and spiro systems;

The term “C₇-C₁₂ bicycloalkenyl” comprises the corresponding unsaturated non-aromatic and non-heteroaromatic hydrocarbon groups containing seven to twelve carbon atoms arranged in a bicyclic ring structure and linked by (i.e. comprising) one or more double bonds; wherein these bicyclic ring structures include fused, bridged and spiro systems;

The term “C₈-C₁₄ tricycloalkyl” comprises the corresponding saturated hydrocarbon groups containing eight to fourteen carbon atoms arranged in a tricyclic ring structure; wherein these tricyclic ring structures include fused, bridged and spiro systems;

The terms “cyclic”, “bicyclic”, “tricyclic”, “cycloalkyl”, “cycloalkenyl”, “bicycloalkyl”, “bicycloalkenyl” and “tricycloalkyl” for R¹ mean that such cyclic, bicyclic or tricyclic residue is directly linked by a chemical bond to the aromatic ring to which R¹ is bound, and wherein the terms “cyclic”, “bicyclic”, “tricyclic”, “cycloalkyl”, “cycloalkenyl”, “bicycloalkyl”, “bicycloalkenyl” and “tricycloalkyl” for a substituent of R¹ mean that such cyclic, bicyclic or tricyclic residue is directly linked by a chemical bond to one of the C-atoms or N-atoms or O-atoms or S-atoms contained in R¹; e.g. “R¹ is cyclohexyl” means that the cyclohexyl residue is linked to the aromatic ring to which R¹ is bound; and “R¹ is methyl and R¹ is substituted with cyclohexyl” means that the resulting —CH₂(cyclohexyl) residue is linked to the aromatic ring to which R¹ is bound.

In case a carbon atom is replaced by a heteroatom selected from O, N, or S, the number of substituents on the respective heteroatom is adapted according to its valency, e.g. a —CR₂— group may be replaced by a —NR—, —NR^{2′}—, —O— or —S— group.

The term “perhalogenated” relates to the exhaustive halogenation of the carbon scaffold; according residues comprise the corresponding perfluorinated, perchlorinated, perbrominated and periodinated groups. Preferably, the term “perhalogenated” relates to perfluorinated or perchlorinated groups, more preferably to perfluorinated groups.

The following contains definitions of terms used in this specification. The initial definition provided for a group or term herein applies to that group or term throughout the present specification, individually or as part of another group, unless otherwise indicated.

The compounds of the present invention may form salts, which are also within the scope of this invention. Reference to a compound of the invention herein is understood to include reference to salts thereof, unless otherwise indicated. The term “salt(s)”, as employed herein, denotes acidic and/or basic salts formed with inorganic and/or organic acids and bases. Zwitterions (internal or inner salts) are included within the term “salt(s)” as used herein (and may be formed, for example, where the substituents comprise an acid moiety such as a carboxyl group and an amino group). Also included herein are quaternary ammonium salts such as alkylammonium salts. Salts of the compounds may be formed, for example, by reacting a compound with an amount of acid or base, such as an equivalent amount, in a medium such as one in which the salt precipitates or in an aqueous medium followed by lyophilization.

Exemplary salts resulting from the addition of an acid include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, chlorates, bromates, iodates, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary salts resulting from the addition of a base (formed, for example, where the substituents comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, tert-butyl amines, and salts with amino acids such as arginine, lysine and the like. The basic nitrogen-containing groups may be quaternized with agents such as lower alkyl halides (e.g., methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science 1977, 66 (2), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Furthermore, in the case of the compounds of the invention which contain an asymmetric carbon atom or an atropoisomeric bond, the invention relates to the D form, the L form and D,L mixtures and also, where more than one asymmetric carbon atom or atropoisomeric bond is present, to the diastereomeric forms. Those compounds of the invention which contain asymmetric carbon atoms or atropoisomeric bonds, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Compounds of the invention can also include all isotopes of atoms occurring in the intermediates or final compounds. Isotopes include those atoms having the same atomic number but different mass numbers. For example, isotopes of hydrogen include tritium and deuterium.

Also included are solvates and hydrates of the compounds of the invention and solvates and hydrates of their pharmaceutically acceptable salts.

The term “compound” as used herein is meant to include all stereoisomers, geometric isomers, tautomers, rotamers, and isotopes of the structures depicted, unless otherwise indicated.

In some embodiments, the compound can be provided as a prodrug. The term “prodrug”, as employed herein, denotes a compound, which, upon administration to a subject, undergoes chemical conversion by metabolic or chemical processes to yield a compound of the invention, or a salt and/or solvate thereof.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof.

Pharmaceutical Methods

The compounds according to the invention have been found to have pharmacologically important properties, which can be used therapeutically. The compounds of the invention can be used alone, in combination with each other or in combination with other active compounds.

In certain embodiments, compounds of the present invention may exhibit growth inhibiting properties in hyperproliferative processes.

The antiproliferative activities of compounds falling under formula (Ia), (Ib) and (Ic), respectively, were investigated on cells or cell lines originating from a disorder of the haematopoietic system, including the myeloid cell compartment and the lymphoid cell compartment (T-cells and B-cells), the neuroendocrine system, the cervix, the breast, the ovaries, the lung, the gastrointestinal tract, and the mucosal epithelium, as well as from the skin epithelium and from the muscle. To this end, HL-60 cells, NB-4 cells, HH cells, RPMI-8402 cells, TANOUE cells, TT cells, HeLa cells, MDA-MB-231 cells, FU-OV-1 cells, LOU-NH91 cells, 23132/87 cells, CAL-27 cells, BHY cells, SCC-25 cells, A-431 cells, human primary epidermal keratinocytes (HPEK), and C2C12 cells were seeded into 96-well plates suitable for fluorescence assays (CORNING #3598) at following initial cell numbers: 1000 cells per well for HL-60; 1000 cells per well for NB-4; 5000 cells per well for HH; 5000 cells per well for RPMI-8402; 1500 cells per well for TANOUE; 9000 cells per well for TT; 2000 cells per well for HeLa; 3000 cells per well for MDA-MB-231; 3000 cells per well for FU-OV-1; 4000 cells per well for LOU-NH91; 2000 cells per well for 23132/87; 2000 cells per well for CAL-27; 1500 cells per well for BHY; 1500 cells per well for SCC-25; 700 cells per well for A-431; 1000 cells per well for HPEK; 500 cells per well for C2C12. The cells were treated with compounds at indicated final concentrations (diluted from the 1000× stock-solutions in DMSO to a final DMSO concentration of 0.1% v/v in H₂O (Water For Injection, WFI, Fisherscientific #10378939)) or with the empty carrier DMSO at 0.1% v/v as control for 5 days. At day 5 after starting the treatments the cells were subjected to the alamarBlue® Proliferation Assay (Bio-Rad Serotec GmbH, BUF012B) according to the protocol of the manufacturer. The readout was taken with a multi-well plate-reader in the fluorescence mode with applying a filter for excitation at 560 nm (band width 10 nm) and for emission at 590 nm (band width 10 nm). Control treatments for growth inhibition with commercial compounds such as Methotrexate (MTREX) and Resveratrol (RES) were included on every plate. Some of the test compounds of the present invention were obtained and applied as their salts. According cases are indicated in the column “Specification” in Table 55 to Table 92 and by their sum formula in Table 93.

The assays were performed in duplicate or more replicates of independent single experiments each containing a six-fold replicate for every condition. For every individual plate, the measured fluorescence intensity values of the conditions with compound treatment were normalized against the corresponding equally weighted arithmetic mean of the fluorescence intensity values of the six DMSO treated control wells in order to obtain the relative values to a baseline level of 1.0.

Two independent outlier analyses were performed according to the methods by Peirce and Chauvenet (Ross, Journal of Engineering Technology 2003, 1-12). Outliers confirmed by at least one of the methods were excluded from the calculations but not more than one value out of six per compound within a single experiment. The weighted arithmetic mean (here abbreviated as AVE_{w}) for each compound was calculated from the normalized values over all independent replicates of the single experiments comprising the six replicates each. The corresponding standard deviation for the weighted arithmetic mean was calculated according to the method described by Bronstein et al. (Bronstein, Semendjajew, Musiol, Müihlig, Taschenbuch der Mathematik, 5^{th} edition 2001 (German), publisher: Verlag Harri Deutsch, Frankfurt am Main and Thun) and was combined with the Gauß' error propagation associated with the performed calculation for the normalization. The resulting standard deviation is herein referred to as “combined standard deviation”.

In cases with considerable variation in the normalized equally weighted arithmetic means derived from two independent replicates, the number of independent replicates was increased to three or more. In the cases of four or more independent replicates, a second-line outlier analysis was applied on all normalized equally weighted arithmetic means according to the methods by Peirce and Chauvenet as described above.

In certain embodiments, the compounds of the present invention may be growth inhibitors in hyperproliferative processes, including malignant and non-malignant hyperproliferative processes.

In one embodiment, several compounds of the invention were found to inhibit the growth of HL-60 cells (human acute myeloid leukemia cells) obtainable from the Deutsche Sammlung von Mikroorganismen und Zellkulturen GmbH (DSMZ) under the accession number ACC 3. HL-60 cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of HL-60 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of HL-60 cells. The so far identified HL-60 growth inhibitors relate to the compounds listed in Table 55 and Table 56. The entries of Table 55 and Table 56 are categorized by the corresponding weighted arithmetic means of the compounds without consideration of the respective standard deviations, hence falling into the activity ranges as indicated.

TABLE 55
Proliferation assay with HL-60 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-2890 | |
| | 3 | XPW-2898 | |
| | 4 | XPW-3038 | |
| | 5 | XPW-3052 | |
| | 6 | XPW-4610 | |
| 0.7 < AVE_{w} ≤ 0.8 | 7 | XPW-0818 | |
| | 8 | XPW-2912 | |
| | 9 | XPW-4578 | |
| | 10 | XPW-4634 | |
| 0.6 < AVE_{w} ≤ 0.7 | 11 | XPW-0042 | |
| | 12 | XPW-0706 | |
| | 13 | XPW-4580 | |
| | 14 | XPW-4624 | |
| 0.4 < AVE_{w} ≤ 0.6 | 15 | XPW-0182 | |
| | 16 | XPW-0314 | |
| | 17 | XPW-0539 | |
| | 18 | XPW-0667 | |
| | 19 | XPW-0762 | |
| | 20 | XPW-0902 | |
| | 21 | XPW-2833 | |
| | 22 | XPW-2847 | |
| | 23 | XPW-4575 | |
| | 24 | XPW-4583 | |
| | 25 | XPW-4591 | |
| | 26 | XPW-4592 | |
| | 27 | XPW-4629 | |
| | 28 | XPW-4641 | |
| | 29 | XPW-4642 | |
| 0.4 ± 0.1 | 30 | RES | Control at 20 μM |
| 0.2 < AVE_{w} ≤ 0.4 | 31 | XPW-0014 | |
| | 32 | XPW-0028 | Used as HCl salt |
| | 33 | XPW-0720 | |
| | 34 | XPW-0776 | |
| | 35 | XPW-2904 | |
| | 36 | XPW-2918 | |
| | 37 | XPW-2926 | |
| | 38 | XPW-4585 | |
| | 39 | XPW-4588 | |
| | 40 | XPW-4589 | |
| | 41 | XPW-4594 | |
| | 42 | XPW-4595 | |
| | 43 | XPW-4635 | |
| | 44 | XPW-4637 | |
| | 45 | XPW-4638 | |
| 0.2 ± 0.1 | 46 | RES | Control at 40 μM |
| 0.1 ± 0.1 | 47 | MTREX | Control at 20 μM |
| 0.0 < AVE_{w} ≤ 0.2 | 48 | XPW-0574 | |
| | 49 | XPW-0661 | |
| | 50 | XPW-0665 | |
| | 51 | XPW-0675 | |
| | 52 | XPW-0679 | |
| | 53 | XPW-0686 | |
| | 54 | XPW-0700 | |
| | 55 | XPW-0714 | |
| | 56 | XPW-0728 | |
| | 57 | XPW-0734 | |
| | 58 | XPW-0742 | |
| | 59 | XPW-0770 | |
| | 60 | XPW-0784 | |
| | 61 | XPW-0790 | |
| | 62 | XPW-0798 | |
| | 63 | XPW-0916 | |
| | 64 | XPW-0924 | |
| | 65 | XPW-0930 | |
| | 66 | XPW-1750 | |
| | 67 | XPW-2744 | |
| | 68 | XPW-2805 | |
| | 69 | XPW-4584 | |
| | 70 | XPW-4586 | |
| | 71 | XPW-4587 | |
| | 72 | XPW-4590 | |
| | 73 | XPW-4593 | |
| | 74 | XPW-4612 | |
| | 75 | XPW-4613 | |
| | 76 | XPW-4614 | |
| | 77 | XPW-4616 | |
| | 78 | XPW-4617 | |
| | 79 | XPW-4618 | |
| | 80 | XPW-4619 | |
| | 81 | XPW-4620 | |
| | 82 | XPW-4621 | |
| | 83 | XPW-4622 | |
| | 84 | XPW-4623 | |
| | 85 | XPW-4625 | |
| | 86 | XPW-4626 | |
| | 87 | XPW-4628 | |
| | 88 | XPW-4630 | |
| | 89 | XPW-4631 | |
| | 90 | XPW-4632 | |
| | 91 | XPW-4633 | |
| | 92 | XPW-4636 | |
| | 93 | XPW-4639 | |
| | 94 | XPW-4640 | |
| | 95 | XPW-4644 | |
| | 96 | XPW-4646 | |
| | 97 | XPW-4647 | |

TABLE 56
Proliferation assay with HL-60 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-1582 | |
| 0.4 ± 0.1 | 3 | RES | Control at 20 μM |
| 0.2 ± 0.1 | 7 | RES | Control at 40 μM |
| 0.0 < AVE_{w} ≤ 0.2 | 4 | XPW-0660 | |
| | 5 | XPW-2788 | |
| 0.1 ± 0.1 | 6 | MTREX | Control at 20 μM |

The data in Table 55 relate to novel compounds, wherein the data in Table 56 relate to a novel medical use of compounds disclosed in PCT/EP2018/054686.

In one embodiment, several compounds of the invention were found to inhibit the growth of NB-4 cells (human acute promyelocytic leukemia cells) obtainable from the Deutsche Sammlung von Mikroorganismen und Zellkulturen GmbH (DSMZ) under the accession number ACC 207. NB-4 cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of NB-4 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of NB-4 cells. The so far identified NB-4 growth inhibitors relate to the compounds listed in Table 57 and Table 58. The entries of Table 57 and Table 58 are categorized by the corresponding weighted arithmetic means of the compounds without consideration of the respective standard deviations, hence falling into the activity ranges as indicated.

TABLE 57
Proliferation assay with NB-4 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0539 | |
| | 3 | XPW-2806 | |
| | 4 | XPW-4624 | |
| 0.7 < AVE_{w} ≤ 0.8 | 5 | XPW-0720 | |
| | 6 | XPW-0832 | |
| 0.6 < AVE_{w} ≤ 0.7 | 7 | XPW-2795 | |
| | 8 | XPW-4595 | |
| 0.4 < AVE_{w} ≤ 0.6 | 9 | XPW-0014 | |
| | 10 | XPW-0667 | |
| | 11 | XPW-0902 | |
| | 12 | XPW-4580 | |
| | 13 | XPW-4583 | |
| | 14 | XPW-4588 | |
| | 15 | XPW-4635 | |
| 0.2 < AVE_{w} ≤ 0.4 | 16 | XPW-2926 | |
| | 17 | XPW-4585 | |
| | 18 | XPW-4589 | |
| | 19 | XPW-4594 | |
| | 20 | XPW-4629 | |
| 0.1 ± 0.0 | 21 | MTREX | Control at 20 μM |
| 0.1 ± 0.0 | 22 | RES | Control at 20 μM |
| 0.0 < AVE_{w} ≤ 0.2 | 23 | XPW-0574 | |
| | 24 | XPW-0661 | |
| | 25 | XPW-0665 | |
| | 26 | XPW-0674 | |
| | 27 | XPW-0675 | |
| | 28 | XPW-0678 | |
| | 29 | XPW-0679 | |
| | 30 | XPW-0686 | |
| | 31 | XPW-0700 | |
| | 32 | XPW-0706 | |
| | 33 | XPW-0714 | |
| | 34 | XPW-0728 | |
| | 35 | XPW-0734 | |
| | 36 | XPW-0742 | |
| | 37 | XPW-0770 | |
| | 38 | XPW-0784 | |
| | 39 | XPW-0790 | |
| | 40 | XPW-0798 | |
| | 41 | XPW-0916 | |
| | 42 | XPW-0924 | |
| | 43 | XPW-0930 | |
| | 44 | XPW-1750 | |
| | 45 | XPW-2744 | |
| | 46 | XPW-2805 | |
| | 47 | XPW-4584 | |
| | 48 | XPW-4586 | |
| | 49 | XPW-4587 | |
| | 50 | XPW-4590 | |
| | 51 | XPW-4593 | |
| | 52 | XPW-4612 | |
| | 53 | XPW-4613 | |
| | 54 | XPW-4614 | |
| | 55 | XPW-4616 | |
| | 56 | XPW-4617 | |
| | 57 | XPW-4618 | |
| | 58 | XPW-4619 | |
| | 59 | XPW-4620 | |
| | 60 | XPW-4621 | |
| | 61 | XPW-4622 | |
| | 62 | XPW-4623 | |
| | 63 | XPW-4625 | |
| | 64 | XPW-4626 | |
| | 65 | XPW-4628 | |
| | 66 | XPW-4630 | |
| | 67 | XPW-4631 | |
| | 68 | XPW-4632 | |
| | 69 | XPW-4633 | |
| | 70 | XPW-4636 | |
| | 71 | XPW-4637 | |
| | 72 | XPW-4638 | |
| | 73 | XPW-4639 | |
| | 74 | XPW-4640 | |
| | 75 | XPW-4644 | |
| | 76 | XPW-4646 | |
| | 77 | XPW-4647 | |
| 0.0 ± 0.0 | 78 | RES | Control at 40 μM |

TABLE 58
Proliferation assay with NB-4 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-2648 | |
| | 3 | XPW-2661 | |
| | 4 | XPW-3199 | |
| | 5 | XPW-3205 | |
| | 6 | XPW-3208 | |
| | 7 | XPW-3230 | |
| | 8 | XPW-4568 | |
| 0.7 < AVE_{w} ≤ 0.8 | 9 | XPW-0543 | |
| | 10 | XPW-3196 | |
| 0.6 < AVE_{w} ≤ 0.7 | 11 | XPW-0518 | |
| | 12 | XPW-0529 | |
| | 13 | XPW-2660 | |
| | 14 | XPW-2674 | |
| 0.4 < AVE_{w} ≤ 0.6 | 15 | XPW-0533 | |
| | 16 | XPW-0546 | |
| | 17 | XPW-1610 | |
| | 18 | XPW-2658 | |
| | 19 | XPW-3216 | |
| | 20 | XPW-3224 | |
| | 21 | XPW-4567 | |
| | 22 | XPW-4569 | |
| 0.2 < AVE_{w} ≤ 0.4 | 23 | XPW-0530 | |
| | 24 | XPW-3223 | |
| 0.1 ± 0.0 | 25 | MTREX | Control at 20 μM |
| 0.1 ± 0.0 | 26 | RES | Control at 20 μM |
| 0.0 < AVE_{w} ≤ 0.2 | 27 | XPW-0544 | |
| | 28 | XPW-0659 | |
| | 29 | XPW-0660 | |
| | 30 | XPW-0663 | |
| | 31 | XPW-0664 | |
| | 32 | XPW-0669 | |
| | 33 | XPW-0670 | |
| | 34 | XPW-0672 | |
| | 35 | XPW-1727 | |
| | 36 | XPW-1728 | |
| | 37 | XPW-1736 | |
| | 38 | XPW-2787 | |
| | 39 | XPW-2788 | |
| | 40 | XPW-2791 | |
| | 41 | XPW-2792 | |
| | 42 | XPW-2797 | |
| | 43 | XPW-2798 | |
| | 44 | XPW-2800 | |
| | 45 | XPW-3193 | |
| | 46 | XPW-3194 | |
| | 47 | XPW-3197 | |
| | 48 | XPW-3200 | |
| | 49 | XPW-3201 | |
| | 50 | XPW-3202 | |
| | 51 | XPW-3203 | |
| | 52 | XPW-3206 | |
| | 53 | XPW-3209 | |
| | 54 | XPW-3212 | |
| | 55 | XPW-3213 | |
| | 56 | XPW-3214 | |
| | 57 | XPW-3217 | |
| | 58 | XPW-3218 | |
| | 59 | XPW-3219 | |
| | 60 | XPW-3221 | |
| | 61 | XPW-3225 | |
| | 62 | XPW-3226 | |
| | 63 | XPW-3227 | |
| | 64 | XPW-3231 | |
| | 65 | XPW-3233 | |
| | 66 | XPW-4645 | |
| 0.0 ± 0.0 | 67 | RES | Control at 40 μM |

The data in Table 57 relate to novel compounds, wherein the data in Table 58 relate to a novel medical use of compounds disclosed in PCT/EP2018/054686.

In one embodiment, several compounds of the invention were found to inhibit the growth of HH cells (human cutaneous T-cell lymphoma cells) obtainable from the Deutsche Sammlung von Mikroorganismen und Zellkulturen GmbH (DSMZ) under the accession number ACC 707. HH cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of HH cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of HH cells. The so far identified HH growth inhibitors relate to the compounds listed in Table 59 and Table 60. The entries of Table 59 and Table 60 are categorized by the corresponding weighted arithmetic means of the compounds without consideration of the respective standard deviations, hence falling into the activity ranges as indicated.

TABLE 59
Proliferation assay with HH cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0028 | Used as HCl salt |
| | 3 | XPW-0718 | Used as HCl salt |
| | 4 | XPW-0720 | |
| | 5 | XPW-0728 | |
| | 6 | XPW-0776 | |
| | 7 | XPW-0916 | |
| | 8 | XPW-2912 | |
| | 9 | XPW-4586 | |
| | 10 | XPW-4589 | |
| | 11 | XPW-4637 | |
| 0.7 < AVE_{w} ≤ 0.8 | 12 | XPW-0014 | |
| | 13 | XPW-0182 | |
| | 14 | XPW-0770 | |
| | 15 | XPW-0784 | |
| | 16 | XPW-0832 | |
| | 17 | XPW-2795 | |
| | 18 | XPW-2926 | |
| | 19 | XPW-3038 | |
| 0.6 < AVE_{w} ≤ 0.7 | 20 | XPW-0574 | |
| | 21 | XPW-0667 | |
| | 22 | XPW-0674 | |
| | 23 | XPW-0679 | |
| | 24 | XPW-0706 | |
| | 25 | XPW-2806 | |
| | 26 | XPW-4580 | |
| 0.6 ± 0.1 | 27 | RES | Control at 20 μM |
| 0.4 < AVE_{w} ≤ 0.6 | 28 | XPW-0665 | |
| | 29 | XPW-0675 | |
| | 30 | XPW-0714 | |
| | 31 | XPW-0924 | |
| | 32 | XPW-0930 | |
| | 33 | XPW-1750 | |
| | 34 | XPW-2805 | |
| | 35 | XPW-4626 | |
| 0.4 ± 0.1 | 36 | MTREX | Control at 20 μM |
| 0.4 ± 0.1 | 37 | RES | Control at 40 μM |
| 0.2 < AVE_{w} ≤ 0.4 | 38 | XPW-0661 | |
| | 39 | XPW-0678 | |
| | 40 | XPW-0686 | |
| | 41 | XPW-0700 | |
| | 42 | XPW-0734 | |
| | 43 | XPW-0742 | |
| | 44 | XPW-2744 | |
| | 45 | XPW-4584 | |
| | 46 | XPW-4591 | |
| | 47 | XPW-4612 | |
| | 48 | XPW-4613 | |
| | 49 | XPW-4614 | |
| | 50 | XPW-4616 | |
| | 51 | XPW-4617 | |
| | 52 | XPW-4618 | |
| | 53 | XPW-4619 | |
| | 54 | XPW-4620 | |
| | 55 | XPW-4621 | |
| | 56 | XPW-4622 | |
| | 57 | XPW-4628 | |
| | 58 | XPW-4633 | |
| | 59 | XPW-4636 | |
| | 60 | XPW-4646 | |
| 0.0 < AVE_{w} ≤ 0.2 | 61 | XPW-4587 | |
| | 62 | XPW-4590 | |
| | 63 | XPW-4593 | |
| | 64 | XPW-4623 | |
| | 65 | XPW-4625 | |
| | 66 | XPW-4630 | |
| | 67 | XPW-4631 | |
| | 68 | XPW-4632 | |
| | 69 | XPW-4639 | |
| | 70 | XPW-4640 | |
| | 71 | XPW-4644 | |
| | 72 | XPW-4647 | |

TABLE 60
Proliferation assay with HH cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0510 | |
| | 3 | XPW-2648 | |
| | 4 | XPW-2657 | |
| | 5 | XPW-2674 | |
| | 6 | XPW-3195 | |
| | 7 | XPW-3196 | |
| | 8 | XPW-3207 | |
| | 9 | XPW-3211 | |
| | 10 | XPW-3215 | |
| | 11 | XPW-3216 | |
| | 12 | XPW-4550 | |
| | 13 | XPW-4552 | |
| | 14 | XPW-4553 | |
| 0.7 < AVE_{w} ≤ 0.8 | 15 | XPW-0524 | |
| | 16 | XPW-0529 | |
| | 17 | XPW-0532 | |
| | 18 | XPW-1582 | |
| | 19 | XPW-1610 | |
| | 20 | XPW-2665 | |
| | 21 | XPW-3210 | |
| 0.6 < AVE_{w} ≤ 0.7 | 22 | XPW-0518 | |
| | 23 | XPW-2658 | |
| | 24 | XPW-3214 | |
| | 25 | XPW-3217 | |
| | 26 | XPW-3224 | |
| 0.6 ± 0.1 | 27 | PES | Control at 20 μM |
| 0.4 < AVE_{w} ≤ 0.6 | 28 | XPW-0530 | |
| | 29 | XPW-2660 | |
| | 30 | XPW-3223 | |
| 0.4 ± 0.1 | 31 | MTREX | Control at 20 μM |
| 0.4 ± 0.1 | 32 | RES | Control at 40 μM |
| 0.2 < AVE_{w} ≤ 0.4 | 33 | XPW-0659 | |
| | 34 | XPW-0664 | |
| | 35 | XPW-0669 | |
| | 36 | XPW-0670 | |
| | 37 | XPW-1727 | |
| | 38 | XPW-1728 | |
| | 39 | XPW-1736 | |
| | 40 | XPW-2788 | |
| | 41 | XPW-2792 | |
| | 42 | XPW-2797 | |
| | 43 | XPW-2800 | |
| | 44 | XPW-3193 | |
| | 45 | XPW-3194 | |
| | 46 | XPW-3197 | |
| | 47 | XPW-3200 | |
| | 48 | XPW-3201 | |
| | 49 | XPW-3202 | |
| | 50 | XPW-3209 | |
| | 51 | XPW-3212 | |
| | 52 | XPW-3218 | |
| | 53 | XPW-3219 | |
| | 54 | XPW-3221 | |
| | 55 | XPW-3227 | |
| 0.0 < AVE_{w} ≤ 0.2 | 56 | XPW-0660 | |
| | 57 | XPW-0663 | |
| | 58 | XPW-0672 | |
| | 59 | XPW-2787 | |
| | 60 | XPW-2791 | |
| | 61 | XPW-2798 | |
| | 62 | XPW-3203 | |
| | 63 | XPW-3206 | |
| | 64 | XPW-3213 | |
| | 65 | XPW-3225 | |
| | 66 | XPW-3226 | |
| | 67 | XPW-3231 | |
| | 68 | XPW-3233 | |

The data in Table 59 relate to novel compounds, wherein the data in Table 60 relate to a novel medical use of compounds disclosed in PCT/EP2018/054686.

In one embodiment, several compounds of the invention were found to inhibit the growth of RPMI-8402 cells (human T cell acute lymphoblastic leukemia cells) obtainable from the Deutsche Sammlung von Mikroorganismen und Zellkulturen GmbH (DSMZ) under the accession number ACC 290. RPMI-8402 cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of RPMI-8402 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds.

The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of RPMI-8402 cells. The so far identified RPMI-8402 growth inhibitors relate to the compounds listed in Table 61 and Table 62. The entries of Table 61 and Table 62 are categorized by the corresponding weighted arithmetic means of the compounds without consideration of the respective standard deviations, hence falling into the activity ranges as indicated.

TABLE 61
Proliferation assay with RPMI-8402 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0536 | |
| | 3 | XPW-0616 | |
| | 4 | XPW-0716 | |
| | 5 | XPW-2847 | |
| | 6 | XPW-2890 | |
| | 7 | XPW-2898 | |
| | 8 | XPW-4579 | |
| | 9 | XPW-4603 | |
| | 10 | XPW-4605 | |
| | 11 | XPW-4627 | |
| 0.7 < AVE_{w} ≤ 0.8 | 12 | XPW-0535 | |
| | 13 | XPW-2806 | |
| | 14 | XPW-3052 | |
| | 15 | XPW-4610 | |
| 0.6 < AVE_{w} ≤ 0.7 | 16 | XPW-0541 | |
| | 17 | XPW-0703 | |
| | 18 | XPW-0717 | |
| | 19 | XPW-0832 | |
| | 20 | XPW-2833 | |
| | 21 | XPW-2918 | |
| | 22 | XPW-3038 | |
| | 23 | XPW-4581 | |
| 0.6 ± 0.0 | 24 | RES | Control at 20 μM |
| 0.4 < AVE_{w} ≤ 0.6 | 25 | XPW-0042 | |
| | 26 | XPW-0314 | |
| | 27 | XPW-0539 | |
| | 28 | XPW-0674 | |
| | 29 | XPW-0706 | |
| | 30 | XPW-0720 | |
| | 31 | XPW-0762 | |
| | 32 | XPW-0818 | |
| | 33 | XPW-0902 | |
| | 34 | XPW-2795 | |
| | 35 | XPW-2904 | |
| | 36 | XPW-4575 | |
| | 37 | XPW-4583 | |
| | 38 | XPW-4588 | |
| | 39 | XPW-4589 | |
| | 40 | XPW-4591 | |
| | 41 | XPW-4592 | |
| | 42 | XPW-4595 | |
| | 43 | XPW-4624 | |
| | 44 | XPW-4629 | |
| | 45 | XPW-4634 | |
| | 46 | XPW-4635 | |
| | 47 | XPW-4641 | |
| | 48 | XPW-4642 | |
| 0.2 < AVE_{w} ≤ 0.4 | 49 | XPW-0014 | |
| | 50 | XPW-0028 | Used as HCl salt |
| | 51 | XPW-0182 | |
| | 52 | XPW-0574 | |
| | 53 | XPW-0728 | |
| | 54 | XPW-0776 | |
| | 55 | XPW-0790 | |
| | 56 | XPW-2805 | |
| | 57 | XPW-4578 | |
| | 58 | XPW-4580 | |
| | 59 | XPW-4585 | |
| | 60 | XPW-4586 | |
| | 61 | XPW-4594 | |
| | 62 | XPW-4637 | |
| | 63 | XPW-4638 | |
| 0.1 ± 0.0 | 64 | MTREX | Control at 20 μM |
| 0.1 ± 0.0 | 65 | RES | Control at 40 μM |
| 0.0 < AVE_{w} ≤ 0.2 | 66 | XPW-0661 | |
| | 67 | XPW-0665 | |
| | 68 | XPW-0667 | |
| | 69 | XPW-0675 | |
| | 70 | XPW-0678 | |
| | 71 | XPW-0679 | |
| | 72 | XPW-0686 | |
| | 73 | XPW-0700 | |
| | 74 | XPW-0714 | |
| | 75 | XPW-0734 | |
| | 76 | XPW-0742 | |
| | 77 | XPW-0770 | |
| | 78 | XPW-0784 | |
| | 79 | XPW-0798 | |
| | 80 | XPW-0916 | |
| | 81 | XPW-0924 | |
| | 82 | XPW-0930 | |
| | 83 | XPW-1750 | |
| | 84 | XPW-2744 | |
| | 85 | XPW-2926 | |
| | 86 | XPW-4574 | |
| | 87 | XPW-4584 | |
| | 88 | XPW-4587 | |
| | 89 | XPW-4590 | |
| | 90 | XPW-4593 | |
| | 91 | XPW-4612 | |
| | 92 | XPW-4613 | |
| | 93 | XPW-4614 | |
| | 94 | XPW-4616 | |
| | 95 | XPW-4617 | |
| | 96 | XPW-4618 | |
| | 97 | XPW-4619 | |
| | 98 | XPW-4620 | |
| | 99 | XPW-4621 | |
| | 100 | XPW-4622 | |
| | 101 | XPW-4623 | |
| | 102 | XPW-4625 | |
| | 103 | XPW-4626 | |
| | 104 | XPW-4628 | |
| | 105 | XPW-4630 | |
| | 106 | XPW-4631 | |
| | 107 | XPW-4632 | |
| | 108 | XPW-4633 | |
| | 109 | XPW-4636 | |
| | 110 | XPW-4639 | |
| | 111 | XPW-4640 | |
| | 112 | XPW-4644 | |
| | 113 | XPW-4646 | |
| | 114 | XPW-4647 | |

TABLE 62
Proliferation assay with RPMI-8402 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0533 | |
| | 3 | XPW-1588 | |
| | 4 | XPW-2634 | |
| | 5 | XPW-2643 | |
| | 6 | XPW-2652 | |
| | 7 | XPW-2672 | |
| | 8 | XPW-3195 | |
| | 9 | XPW-3196 | |
| | 10 | XPW-3199 | |
| | 11 | XPW-3222 | |
| | 12 | XPW-3230 | |
| | 13 | XPW-3232 | |
| | 14 | XPW-3234 | |
| | 15 | XPW-4546 | |
| | 16 | XPW-4551 | |
| | 17 | XPW-4561 | |
| | 18 | XPW-4562 | |
| | 19 | XPW-4572 | |
| 0.7 < AVE_{w} ≤ 0.8 | 20 | XPW-0524 | |
| | 21 | XPW-1596 | Measured at 10 μM |
| | 22 | XPW-2648 | |
| | 23 | XPW-2657 | |
| | 24 | XPW-2658 | |
| | 25 | XPW-2661 | |
| | 26 | XPW-2662 | |
| | 27 | XPW-2665 | |
| | 28 | XPW-2671 | |
| | 29 | XPW-3205 | |
| | 30 | XPW-4560 | |
| | 31 | XPW-4645 | |
| 0.6 < AVE_{w} ≤ 0.7 | 32 | XPW-0534 | |
| | 33 | XPW-1582 | |
| | 34 | XPW-3208 | |
| | 35 | XPW-4545 | |
| | 36 | XPW-4573 | |
| 0.6 ± 0.0 | 37 | RES | Control at 20 μM |
| 0.4 < AVE_{w} ≤ 0.6 | 38 | XPW-0515 | |
| | 39 | XPW-0518 | |
| | 40 | XPW-0532 | |
| | 41 | XPW-0537 | |
| | 42 | XPW-0538 | |
| | 43 | XPW-0544 | |
| | 44 | XPW-0546 | |
| | 45 | XPW-1602 | |
| | 46 | XPW-1610 | |
| | 47 | XPW-2660 | |
| | 48 | XPW-2666 | |
| | 49 | XPW-3224 | |
| 0.2 < AVE_{w} ≤ 0.4 | 50 | XPW-0529 | |
| | 51 | XPW-0530 | |
| | 52 | XPW-2674 | |
| | 53 | XPW-3216 | |
| | 54 | XPW-3223 | |
| 0.1 ± 0.0 | 55 | MTREX | Control at 20 μM |
| 0.1 ± 0.0 | 56 | RES | Control at 40 μM |
| 0.0 < AVE_{w} ≤ 0.2 | 57 | XPW-0659 | |
| | 58 | XPW-0660 | |
| | 59 | XPW-0663 | |
| | 60 | XPW-0664 | |
| | 61 | XPW-0669 | |
| | 62 | XPW-0670 | |
| | 63 | XPW-0672 | |
| | 64 | XPW-1727 | |
| | 65 | XPW-1728 | |
| | 66 | XPW-1736 | |
| | 67 | XPW-2787 | |
| | 68 | XPW-2788 | |
| | 69 | XPW-2791 | |
| | 70 | XPW-2792 | |
| | 71 | XPW-2797 | |
| | 72 | XPW-2798 | |
| | 73 | XPW-2800 | |
| | 74 | XPW-3193 | |
| | 75 | XPW-3194 | |
| | 76 | XPW-3197 | |
| | 77 | XPW-3200 | |
| | 78 | XPW-3201 | |
| | 79 | XPW-3202 | |
| | 80 | XPW-3203 | |
| | 81 | XPW-3206 | |
| | 82 | XPW-3209 | |
| | 83 | XPW-3212 | |
| | 84 | XPW-3213 | |
| | 85 | XPW-3214 | |
| | 86 | XPW-3217 | |
| | 87 | XPW-3218 | |
| | 88 | XPW-3219 | |
| | 89 | XPW-3221 | |
| | 90 | XPW-3225 | |
| | 91 | XPW-3226 | |
| | 92 | XPW-3227 | |
| | 93 | XPW-3231 | |
| | 94 | XPW-3233 | |

The data in Table 61 relate to novel compounds, wherein the data in Table 62 relate to a novel medical use of compounds disclosed in PCT/EP2018/054686.

In one embodiment, several compounds of the invention were found to inhibit the growth of TANOUE cells (human B cell leukemia cells) obtainable from the Deutsche Sammlung von Mikroorganismen und Zellkulturen GmbH (DSMZ) under the accession number ACC 399. TANOUE cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of TANOUE cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of TANOUE cells. The so far identified TANOUE growth inhibitors relate to the compounds listed in Table 63 and Table 64. The entries of Table 63 and Table 64 are categorized by the corresponding weighted arithmetic means of the compounds without consideration of the respective standard deviations, hence falling into the activity ranges as indicated.

TABLE 63
Proliferation assay with TANOUE cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0042 | |
| | 3 | XPW-0535 | |
| | 4 | XPW-0541 | |
| | 5 | XPW-0566 | |
| | 6 | XPW-0616 | |
| | 7 | XPW-0703 | |
| | 8 | XPW-0717 | |
| | 9 | XPW-2675 | |
| | 10 | XPW-2688 | |
| | 11 | XPW-2731 | |
| | 12 | XPW-2847 | |
| | 13 | XPW-2912 | |
| | 14 | XPW-4603 | |
| 0.7 < AVE_{w} ≤ 0.8 | 15 | XPW-0674 | |
| | 16 | XPW-0832 | |
| | 17 | XPW-2795 | |
| | 18 | XPW-2806 | |
| | 19 | XPW-2833 | |
| | 20 | XPW-2890 | |
| | 21 | XPW-3052 | |
| | 22 | XPW-4624 | |
| | 23 | XPW-4634 | |
| 0.6 < AVE_{w} ≤ 0.7 | 24 | XPW-0020 | Used as HCl salt |
| 0.4 < AVE_{w} ≤ 0.6 | 25 | XPW-0028 | Used as HCl salt |
| | 26 | XPW-0182 | |
| | 27 | XPW-0314 | |
| | 28 | XPW-0539 | |
| | 29 | XPW-0675 | |
| | 30 | XPW-0720 | |
| | 31 | XPW-0762 | |
| | 32 | XPW-2904 | |
| | 33 | XPW-2918 | |
| | 34 | XPW-4578 | |
| | 35 | XPW-4591 | |
| | 36 | XPW-4592 | |
| | 37 | XPW-4595 | |
| | 38 | XPW-4629 | |
| | 39 | XPW-4641 | |
| 0.2 < AVE_{w} ≤ 0.4 | 40 | XPW-0706 | |
| | 41 | XPW-0770 | |
| | 42 | XPW-0790 | |
| | 43 | XPW-2805 | |
| | 44 | XPW-2926 | |
| | 45 | XPW-4575 | |
| | 46 | XPW-4580 | |
| | 47 | XPW-4583 | |
| | 48 | XPW-4589 | |
| | 49 | XPW-4594 | |
| 0.1 ± 0.0 | 50 | MTREX | Control at 20 μM |
| 0.1 ± 0.0 | 51 | RES | Control at 20 μM |
| 0.0 < AVE_{w} ≤ 0.2 | 52 | XPW-0014 | |
| | 53 | XPW-0574 | |
| | 54 | XPW-0661 | |
| | 55 | XPW-0665 | |
| | 56 | XPW-0667 | |
| | 57 | XPW-0678 | |
| | 58 | XPW-0679 | |
| | 59 | XPW-0686 | |
| | 60 | XPW-0700 | |
| | 61 | XPW-0714 | |
| | 62 | XPW-0728 | |
| | 63 | XPW-0734 | |
| | 64 | XPW-0742 | |
| | 65 | XPW-0776 | |
| | 66 | XPW-0784 | |
| | 67 | XPW-0798 | |
| | 68 | XPW-0902 | |
| | 69 | XPW-0916 | |
| | 70 | XPW-0924 | |
| | 71 | XPW-0930 | |
| | 72 | XPW-1750 | |
| | 73 | XPW-2744 | |
| | 74 | XPW-4584 | |
| | 75 | XPW-4585 | |
| | 76 | XPW-4586 | |
| | 77 | XPW-4587 | |
| | 78 | XPW-4588 | |
| | 79 | XPW-4590 | |
| | 80 | XPW-4593 | |
| | 81 | XPW-4612 | |
| | 82 | XPW-4613 | |
| | 83 | XPW-4614 | |
| | 84 | XPW-4616 | |
| | 85 | XPW-4617 | |
| | 86 | XPW-4618 | |
| | 87 | XPW-4619 | |
| | 88 | XPW-4620 | |
| | 89 | XPW-4621 | |
| | 90 | XPW-4622 | |
| | 91 | XPW-4623 | |
| | 92 | XPW-4625 | |
| | 93 | XPW-4626 | |
| | 94 | XPW-4628 | |
| | 95 | XPW-4630 | |
| | 96 | XPW-4631 | |
| | 97 | XPW-4632 | |
| | 98 | XPW-4633 | |
| | 99 | XPW-4635 | |
| | 100 | XPW-4636 | |
| | 101 | XPW-4637 | |
| | 102 | XPW-4638 | |
| | 103 | XPW-4639 | |
| | 104 | XPW-4640 | |
| | 105 | XPW-4644 | |
| | 106 | XPW-4646 | |
| | 107 | XPW-4647 | |
| 0.0 ± 0.0 | 108 | RES | Control at 40 μM |

TABLE 64
Proliferation assay with TANOUE cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0533 | |
| | 3 | XPW-0537 | |
| | 4 | XPW-1587 | |
| | 5 | XPW-1588 | |
| | 6 | XPW-2637 | |
| | 7 | XPW-2651 | |
| | 8 | XPW-2666 | |
| | 9 | XPW-2672 | |
| | 10 | XPW-3196 | |
| | 11 | XPW-3205 | |
| | 12 | XPW-3229 | |
| | 13 | XPW-3230 | |
| | 14 | XPW-4545 | |
| | 15 | XPW-4548 | |
| | 16 | XPW-4560 | |
| | 17 | XPW-4562 | |
| | 18 | XPW-4565 | |
| | 19 | XPW-4567 | |
| | 20 | XPW-4573 | |
| 0.7 < AVE_{w} ≤ 0.8 | 21 | XPW-0524 | |
| | 22 | XPW-2648 | |
| | 23 | XPW-2662 | |
| | 24 | XPW-2665 | |
| | 25 | XPW-3208 | |
| | 26 | XPW-4547 | |
| | 27 | XPW-4645 | |
| 0.6 < AVE_{w} ≤ 0.7 | 28 | XPW-1602 | |
| 0.4 < AVE_{w} ≤ 0.6 | 29 | XPW-0529 | |
| | 30 | XPW-0538 | |
| | 31 | XPW-1582 | |
| | 32 | XPW-1610 | |
| | 33 | XPW-2658 | |
| | 34 | XPW-2674 | |
| 0.2 < AVE_{w} ≤ 0.4 | 35 | XPW-0518 | |
| | 36 | XPW-0544 | |
| | 37 | XPW-3216 | |
| | 38 | XPW-3217 | |
| | 39 | XPW-3223 | |
| | 40 | XPW-3224 | |
| 0.1 ± 0.0 | 41 | MTREX | Control at 20 μM |
| 0.1 ± 0.0 | 42 | RES | Control at 20 μM |
| 0.0 < AVE_{w} ≤ 0.2 | 43 | XPW-0530 | |
| | 44 | XPW-0532 | |
| | 45 | XPW-0546 | |
| | 46 | XPW-0659 | |
| | 47 | XPW-0660 | |
| | 48 | XPW-0663 | |
| | 49 | XPW-0664 | |
| | 50 | XPW-0669 | |
| | 51 | XPW-0670 | |
| | 52 | XPW-0672 | |
| | 53 | XPW-1596 | Measured at 10 μM |
| | 54 | XPW-1727 | |
| | 55 | XPW-1728 | |
| | 56 | XPW-1736 | |
| | 57 | XPW-2660 | |
| | 58 | XPW-2787 | |
| | 59 | XPW-2788 | |
| | 60 | XPW-2791 | |
| | 61 | XPW-2792 | |
| | 62 | XPW-2797 | |
| | 63 | XPW-2798 | |
| | 64 | XPW-2800 | |
| | 65 | XPW-3193 | |
| | 66 | XPW-3194 | |
| | 67 | XPW-3197 | |
| | 68 | XPW-3200 | |
| | 69 | XPW-3201 | |
| | 70 | XPW-3202 | |
| | 71 | XPW-3203 | |
| | 72 | XPW-3206 | |
| | 73 | XPW-3209 | |
| | 74 | XPW-3212 | |
| | 75 | XPW-3213 | |
| | 76 | XPW-3214 | |
| | 77 | XPW-3218 | |
| | 78 | XPW-3219 | |
| | 79 | XPW-3221 | |
| | 80 | XPW-3225 | |
| | 81 | XPW-3226 | |
| | 82 | XPW-3227 | |
| | 83 | XPW-3231 | |
| | 84 | XPW-3233 | |
| 0.0 ± 0.0 | 85 | RES | Control at 40 μM |

The data in Table 63 relate to novel compounds, wherein the data in Table 64 relate to a novel medical use of compounds disclosed in PCT/EP2018/054686.

In one embodiment, several compounds of the invention were found to inhibit the growth of TT cells (human medullary thyroid carcinoma cells) obtainable from the American Type Culture Collection (ATCC) under the accession number ATCC-CRL-1803. TT cells were cultivated in F-12K medium (Fisherscientific, #11580556, or ATCC, #ATCC-30-2004) containing 10% fetal bovine serum (Fisherscientific, #15517589) at 37° C. and 5% CO₂.

A compound is considered as a growth inhibitor of TT cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of TT cells. The so far identified TT growth inhibitors relate to the compounds listed in Table 65 and Table 66. The entries of Table 65 and Table 66 are categorized by the corresponding weighted arithmetic means of the compounds without consideration of the respective standard deviations, hence falling into the activity ranges as indicated.

TABLE 65
Proliferation assay with TT cells at 20 μM

| Activity Range | Entry | Compound No. | Specification |
|---|---|---|---|
| 1.0 ± 0.0 | 1 | DMSO | Baseline control |
| 1.0 ± 0.0 | 2 | MTREX | Control at 20 μM |
| 0.9 ± 0.0 | 3 | RES | Control at 20 μM |
| 0.8 < AVE_{w} ≤ 0.9 | 4 | XPW-0028 | Used as HCl salt |
| | 5 | XPW-0042 | |
| | 6 | XPW-0539 | |
| | 7 | XPW-0790 | |
| | 8 | XPW-2805 | |
| | 9 | XPW-2904 | |
| | 10 | XPW-2918 | |
| | 11 | XPW-4626 | |
| | 12 | XPW-4629 | |
| 0.7 < AVE_{w} ≤ 0.8 | 13 | XPW-0182 | |
| | 14 | XPW-0924 | |
| | 15 | XPW-2795 | |
| | 16 | XPW-4580 | |
| | 17 | XPW-4583 | |
| | 18 | XPW-4585 | |
| | 19 | XPW-4589 | |
| | 20 | XPW-4594 | |
| 0.7 ± 0.0 | 21 | RES | Control at 40 μM |
| 0.6 < AVE_{w} ≤ 0.7 | 22 | XPW-0667 | |
| | 23 | XPW-0784 | |
| | 24 | XPW-0916 | |
| | 25 | XPW-0930 | |
| | 26 | XPW-4578 | |
| | 27 | XPW-4616 | |
| 0.4 < AVE_{w} ≤ 0.6 | 28 | XPW-0014 | |
| | 29 | XPW-0700 | |
| | 30 | XPW-0734 | |
| | 31 | XPW-0770 | |
| | 32 | XPW-4586 | |
| | 33 | XPW-4614 | |
| | 34 | XPW-4620 | |
| | 35 | XPW-4625 | |
| | 36 | XPW-4636 | |
| | 37 | XPW-4637 | |
| | 38 | XPW-4638 | |
| | 39 | XPW-4644 | |
| 0.2 < AVE_{w} ≤ 0.4 | 40 | XPW-0661 | |
| | 41 | XPW-0665 | |
| | 42 | XPW-0686 | |
| | 43 | XPW-0742 | |
| | 44 | XPW-1750 | |
| | 45 | XPW-4584 | |
| | 46 | XPW-4587 | |
| | 47 | XPW-4590 | |
| | 48 | XPW-4613 | |
| | 49 | XPW-4617 | |
| | 50 | XPW-4618 | |
| | 51 | XPW-4619 | |
| | 52 | XPW-4621 | |
| | 53 | XPW-4622 | |
| | 54 | XPW-4631 | |
| | 55 | XPW-4632 | |
| | 56 | XPW-4633 | |
| | 57 | XPW-4646 | |
| | 58 | XPW-4647 | |
| 0.0 < AVE_{w} ≤ 0.2 | 59 | XPW-4593 | |
| | 60 | XPW-4612 | |
| | 61 | XPW-4623 | |
| | 62 | XPW-4630 | |
| | 63 | XPW-4639 | |
| | 64 | XPW-4640 | |

TABLE 66
Proliferation assay with TT cells at 20 μM

| Activity Range | Entry | Compound No. | Specification |
|---|---|---|---|
| 1 ± 0.0 | 1 | MTREX | Control at 20 μM |
| 1.0 ± 0.0 | 2 | DMSO | Baseline control |
| 0.9 ± 0.0 | 3 | RES | Control at 20 μM |
| 0.8 < AVE_{w} ≤ 0.9 | 4 | XPW-2658 | |
| | 5 | XPW-2660 | |
| 0.7 ± 0.0 | 6 | RES | Control at 40 μM |
| 0.7 < AVE_{w} ≤ 0.8 | 7 | XPW-0530 | |
| | 8 | XPW-1582 | |
| | 9 | XPW-3217 | |
| 0.4 < AVE_{w} ≤ 0.6 | 10 | XPW-0663 | |
| | 11 | XPW-0669 | |
| | 12 | XPW-3194 | |
| | 13 | XPW-3200 | |
| | 14 | XPW-3213 | |
| | 15 | XPW-3233 | |
| 0.2 < AVE_{w} ≤ 0.4 | 16 | XPW-0660 | |
| | 17 | XPW-0670 | |
| | 18 | XPW-2797 | |
| | 19 | XPW-3202 | |
| | 20 | XPW-3212 | |
| | 21 | XPW-3225 | |
| | 22 | XPW-3227 | |
| | 23 | XPW-3231 | |
| 0.0 < AVE_{w} ≤ 0.2 | 24 | XPW-2787 | |
| | 25 | XPW-2788 | |
| | 26 | XPW-2791 | |
| | 27 | XPW-3197 | |
| | 28 | XPW-3203 | |
| | 29 | XPW-3206 | |
| | 30 | XPW-3209 | |
| | 31 | XPW-3218 | |
| | 32 | XPW-3219 | |
| | 33 | XPW-3226 | |

The data in Table 65 relate to novel compounds, wherein the data in Table 66 relate to a novel medical use of compounds disclosed in PCT/EP2018/054686.

In one embodiment, several compounds of the invention were found to inhibit the growth of HeLa cells (human cervical adenocarcinoma cells) obtainable from the American Type Culture Collection (ATCC) under the accession number ATCC-CCL-2. HeLa cells were cultivated in DMEM medium (Fisherscientific, #11584456) containing 10% fetal bovine serum (Fisherscientific, #15517589) at 37° C. and 5% CO₂.

A compound is considered as a growth inhibitor of HeLa cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of HeLa cells. The so far identified HeLa growth inhibitors relate to the compounds listed in Table 67 and Table 68. The entries of Table 67 and Table 68 are categorized by the corresponding weighted arithmetic means of the compounds without consideration of the respective standard deviations, hence falling into the activity ranges as indicated.

TABLE 67
Proliferation assay with HeLa cells at 20 μM

| Activity Range | Entry | Compound No. | Specification |
|---|---|---|---|
| 1.0 ± 0.0 | 1 | DMSO | Baseline control |
| 0.9 ± 0.0 | 2 | RES | Control at 20 μM |
| 0.7 < AVE_{w} ≤ 0.8 | 3 | XPW-0661 | |
| 0.6 < AVE_{w} ≤ 0.7 | 4 | XPW-4633 | |
| 0.4 < AVE_{w} ≤ 0.6 | 5 | XPW-4626 | |
| 0.4 ± 0.1 | 6 | RES | Control at 40 μM |
| 0.4 ± 0.0 | 7 | MTREX | Control at 20 μM |
| 0.2 < AVE_{w} ≤ 0.4 | 8 | XPW-0674 | |
| | 9 | XPW-4613 | |
| | 10 | XPW-4614 | |
| | 11 | XPW-4618 | |
| | 12 | XPW-4620 | |
| 0.0 < AVE_{w} ≤ 0.2 | 13 | XPW-0678 | |
| | 14 | XPW-0686 | |
| | 15 | XPW-0700 | |
| | 16 | XPW-0734 | |
| | 17 | XPW-0742 | |
| | 18 | XPW-0924 | |
| | 19 | XPW-1750 | |
| | 20 | XPW-4584 | |
| | 21 | XPW-4587 | |
| | 22 | XPW-4590 | |
| | 23 | XPW-4593 | |
| | 24 | XPW-4616 | |
| | 25 | XPW-4617 | |
| | 26 | XPW-4619 | |
| | 27 | XPW-4621 | |
| | 28 | XPW-4622 | |
| | 29 | XPW-4623 | |
| | 30 | XPW-4625 | |
| | 31 | XPW-4630 | |
| | 32 | XPW-4631 | |
| | 33 | XPW-4632 | |
| | 34 | XPW-4639 | |
| | 35 | XPW-4640 | |
| | 36 | XPW-4644 | |
| | 37 | XPW-4646 | |
| | 38 | XPW-4647 | |

TABLE 68
Proliferation assay with HeLa cells at 20 μM

| Activity Range | Entry | Compound No. | Specification |
|---|---|---|---|
| 1.0 ± 0.0 | 1 | DMSO | Baseline control |
| 0.9 ± 0.0 | 2 | RES | Control at 20 μM |
| 0.8 < AVE_{w} ≤ 0.9 | 3 | XPW-3202 | |
| 0.6 < AVE_{w} ≤ 0.7 | 4 | XPW-1727 | |
| | 5 | XPW-3218 | |
| 0.4 < AVE_{w} ≤ 0.6 | 6 | XPW-3193 | |
| | 7 | XPW-3206 | |
| | 8 | XPW-3212 | |
| | 9 | XPW-3221 | |
| 0.4 ± 0.0 | 10 | MTREX | Control at 20 μM |
| 0.4 ± 0.1 | 11 | RES | Control at 40 μM |
| 0.2 < AVE_{w} ≤ 0.4 | 12 | XPW-2787 | |
| | 13 | XPW-2788 | |
| | 14 | XPW-2791 | |
| | 15 | XPW-2792 | |
| | 16 | XPW-3194 | |
| | 17 | XPW-3213 | |
| | 18 | XPW-3226 | |
| | 19 | XPW-3227 | |
| 0.0 ≤ AVE_{w} ≤ 0.2 | 20 | XPW-0660 | |
| | 21 | XPW-0663 | |
| | 22 | XPW-0664 | |
| | 23 | XPW-0669 | |
| | 24 | XPW-0670 | |
| | 25 | XPW-0672 | |
| | 26 | XPW-1728 | |
| | 27 | XPW-1736 | |
| | 28 | XPW-2797 | |
| | 29 | XPW-2800 | |
| | 30 | XPW-3197 | |
| | 31 | XPW-3200 | |
| | 32 | XPW-3219 | |
| | 33 | XPW-3225 | |
| | 34 | XPW-3231 | |
| | 35 | XPW-3233 | |

The data in Table 67 relate to novel compounds, wherein the data in Table 68 relate to a novel medical use of compounds disclosed in PCT/EP2018/054686.

In one embodiment, several compounds of the invention were found to inhibit the growth of MDA-MB-231 cells (human breast carcinoma cells) obtainable from the Deutsche Sammlung von Mikroorganismen und Zellkulturen GmbH (DSMZ) under the accession number ACC 732. MDA-MB-231 cells were cultivated in Leibovitz's L-15 (no phenol red) medium (Fisherscientific, #11540556) containing 10% fetal bovine serum (Fisherscientific, #15517589) at 37° C. and 0% CO₂.

A compound is considered as a growth inhibitor of MDA-MB-231 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of MDA-MB-231 cells. The so far identified MDA-MB-231 growth inhibitors relate to the compounds listed in Table 69 and Table 70. The entries of Table 69 and Table 70 are categorized by the corresponding weighted arithmetic means of the compounds without consideration of the respective standard deviations, hence falling into the activity ranges as indicated.

TABLE 69
Proliferation assay with MDA-MB-231 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0042 | |
| | 3 | XPW-0616 | |
| | 4 | XPW-0667 | |
| | 5 | XPW-0704 | Used as HCl salt |
| | 6 | XPW-0716 | |
| | 7 | XPW-0718 | |
| | 8 | XPW-2731 | |
| | 9 | XPW-2806 | |
| | 10 | XPW-2834 | |
| | 11 | XPW-2848 | |
| | 12 | XPW-4627 | |
| | 13 | XPW-4636 | |
| 0.7 < AVE_{w} ≤ 0.8 | 14 | XPW-0832 | |
| | 15 | XPW-2847 | |
| | 16 | XPW-3038 | |
| | 17 | XPW-4579 | |
| | 18 | XPW-4603 | |
| | 19 | XPW-4624 | |
| 0.6 < AVE_{w} ≤ 0.7 | 20 | XPW-2744 | |
| | 21 | XPW-2805 | |
| | 22 | XPW-2833 | |
| | 23 | XPW-3052 | |
| | 24 | XPW-4634 | |
| | 25 | XPW-4642 | |
| 0.6 ± 0.0 | 26 | MTREX | Control at 20 μM |
| 0.6 ± 0.0 | 27 | RES | Control at 20 μM |
| 0.4 < AVE_{w} ≤ 0.6 | 28 | XPW-0028 | Used as HCl salt |
| | 29 | XPW-0182 | |
| | 30 | XPW-0535 | |
| | 31 | XPW-0541 | |
| | 32 | XPW-0675 | |
| | 33 | XPW-0678 | |
| | 34 | XPW-0703 | |
| | 35 | XPW-0717 | |
| | 36 | XPW-0798 | |
| | 37 | XPW-2890 | |
| | 38 | XPW-2898 | |
| | 39 | XPW-2926 | |
| | 40 | XPW-4581 | |
| | 41 | XPW-4641 | |
| 0.2 < AVE_{w} ≤ 0.4 | 42 | XPW-0574 | |
| | 43 | XPW-0661 | |
| | 44 | XPW-0665 | |
| | 45 | XPW-0679 | |
| | 46 | XPW-0790 | |
| | 47 | XPW-0818 | |
| | 48 | XPW-2918 | |
| | 49 | XPW-4629 | |
| | 50 | XPW-4635 | |
| | 51 | XPW-4640 | |
| 0.0 < AVE_{w} ≤ 0.2 | 52 | XPW-0014 | |
| | 53 | XPW-0539 | |
| | 54 | XPW-0686 | |
| | 55 | XPW-0700 | |
| | 56 | XPW-0706 | |
| | 57 | XPW-0714 | |
| | 58 | XPW-0720 | |
| | 59 | XPW-0728 | |
| | 60 | XPW-0734 | |
| | 61 | XPW-0742 | |
| | 62 | XPW-0762 | |
| | 63 | XPW-0770 | |
| | 64 | XPW-0776 | |
| | 65 | XPW-0784 | |
| | 66 | XPW-0902 | |
| | 67 | XPW-0916 | |
| | 68 | XPW-0924 | |
| | 69 | XPW-0930 | |
| | 70 | XPW-1750 | |
| | 71 | XPW-2904 | |
| | 72 | XPW-4575 | |
| | 73 | XPW-4578 | |
| | 74 | XPW-4580 | |
| | 75 | XPW-4583 | |
| | 76 | XPW-4584 | |
| | 77 | XPW-4585 | |
| | 78 | XPW-4586 | |
| | 79 | XPW-4587 | |
| | 80 | XPW-4588 | |
| | 81 | XPW-4589 | |
| | 82 | XPW-4590 | |
| | 83 | XPW-4591 | |
| | 84 | XPW-4592 | |
| | 85 | XPW-4593 | |
| | 86 | XPW-4594 | |
| | 87 | XPW-4595 | |
| | 88 | XPW-4612 | |
| | 89 | XPW-4613 | |
| | 90 | XPW-4614 | |
| | 91 | XPW-4616 | |
| | 92 | XPW-4617 | |
| | 93 | XPW-4618 | |
| | 94 | XPW-4619 | |
| | 95 | XPW-4620 | |
| | 96 | XPW-4621 | |
| | 97 | XPW-4622 | |
| | 98 | XPW-4623 | |
| | 99 | XPW-4625 | |
| | 100 | XPW-4626 | |
| | 101 | XPW-4630 | |
| | 102 | XPW-4631 | |
| | 103 | XPW-4632 | |
| | 104 | XPW-4633 | |
| | 105 | XPW-4637 | |
| | 106 | XPW-4638 | |
| | 107 | XPW-4639 | |
| | 108 | XPW-4644 | |
| | 109 | XPW-4646 | |

TABLE 70
Proliferation assay with MDA-MB-231 cells at 20 μM

| Activity Range | Entry | Compound No. | Specification |
|---|---|---|---|
| 1.0 ± 0.0 | 1 | BMSO | Baseline control |
| 0.8 < AVE_{w} ≤ 0.9 | 2 | XPW-0533 | |
| | 3 | XPW-2633 | |
| | 4 | XPW-2634 | |
| | 5 | XPW-2637 | |
| | 6 | XPW-2646 | |
| | 7 | XPW-3195 | |
| | 8 | XPW-3207 | |
| | 9 | XPW-3211 | |
| | 10 | XPW-3228 | |
| | 11 | XPW-4547 | |
| | 12 | XPW-4548 | |
| | 13 | XPW-4550 | |
| | 14 | XPW-4553 | |
| | 15 | XPW-4554 | |
| | 16 | XPW-4555 | |
| | 17 | XPW-4557 | |
| | 18 | XPW-4563 | |
| | 19 | XPW-4566 | |
| | 20 | XPW-4568 | |
| | 21 | XPW-4570 | |
| | 22 | XPW-4571 | |
| 0.7 < AVE_{w} ≤ 0.8 | 23 | XPW-0516 | |
| | 24 | XPW-0534 | |
| | 25 | XPW-1582 | |
| | 26 | XPW-1588 | |
| | 27 | XPW-1601 | |
| | 28 | XPW-2643 | |
| | 29 | XPW-2652 | |
| | 30 | XPW-3199 | |
| | 31 | XPW-3202 | |
| | 32 | XPW-3205 | |
| | 33 | XPW-3222 | |
| | 34 | XPW-3229 | |
| | 35 | XPW-3230 | |
| | 36 | XPW-3232 | |
| | 37 | XPW-3234 | |
| | 38 | XPW-4544 | |
| | 39 | XPW-4549 | |
| | 40 | XPW-4551 | |
| | 41 | XPW-4556 | |
| | 42 | XPW-4560 | |
| | 43 | XPW-4561 | |
| | 44 | XPW-4564 | |
| | 45 | XPW-4565 | |
| | 46 | XPW-4567 | |
| | 47 | XPW-4572 | |
| | 48 | XPW-4645 | |
| 0.6 < AVE_{w} ≤ 0.7 | 49 | XPW-1587 | |
| | 50 | XPW-2651 | |
| | 51 | XPW-2662 | |
| | 52 | XPW-2672 | |
| | 53 | XPW-2798 | |
| | 54 | XPW-3196 | |
| | 55 | XPW-3203 | |
| | 56 | XPW-4569 | |
| 0.6 ± 0.0 | 57 | MTREX | Control at 20 μM |
| 0.6 ± 0.0 | 58 | RES | Control at 20 μM |
| 0.4 < AVE_{w} ≤ 0.6 | 59 | XPW-0515 | |
| | 60 | XPW-0524 | |
| | 61 | XPW-2648 | |
| | 62 | XPW-2658 | |
| | 63 | XPW-2661 | |
| | 64 | XPW-2665 | |
| | 65 | XPW-2671 | |
| | 66 | XPW-3208 | |
| | 67 | XPW-3209 | |
| | 68 | XPW-4545 | |
| | 69 | XPW-4562 | |
| | 70 | XPW-4573 | |
| 0.2 < AVE_{w} ≤ 0.4 | 71 | XPW-0518 | |
| | 72 | XPW-0537 | |
| | 73 | XPW-0660 | |
| | 74 | XPW-1602 | |
| | 75 | XPW-1727 | |
| | 76 | XPW-2657 | |
| | 77 | XPW-2666 | |
| | 78 | XPW-2788 | |
| | 79 | XPW-3194 | |
| | 80 | XPW-3201 | |
| | 81 | XPW-3206 | |
| | 82 | XPW-3212 | |
| | 83 | XPW-3213 | |
| | 84 | XPW-3214 | |
| | 85 | XPW-3218 | |
| | 86 | XPW-3223 | |
| | 87 | XPW-3224 | |
| | 88 | XPW-3233 | |
| 0.0 < AVE_{w} ≤ 0.2 | 89 | XPW-0529 | |
| | 90 | XPW-0530 | |
| | 91 | XPW-0538 | |
| | 92 | XPW-0544 | |
| | 93 | XPW-0546 | |
| | 94 | XPW-0659 | |
| | 95 | XPW-0663 | |
| | 96 | XPW-0664 | |
| | 97 | XPW-0669 | |
| | 98 | XPW-0670 | |
| | 99 | XPW-0672 | |
| | 100 | XPW-1610 | |
| | 101 | XPW-1728 | |
| | 102 | XPW-1736 | |
| | 103 | XPW-2660 | |
| | 104 | XPW-2674 | |
| | 105 | XPW-2787 | |
| | 106 | XPW-2791 | |
| | 107 | XPW-2792 | |
| | 108 | XPW-2797 | |
| | 109 | XPW-2800 | |
| | 110 | XPW-3193 | |
| | 111 | XPW-3197 | |
| | 112 | XPW-3200 | |
| | 113 | XPW-3219 | |
| | 114 | XPW-3221 | |
| | 115 | XPW-3225 | |
| | 116 | XPW-3226 | |
| | 117 | XPW-3227 | |
| | 118 | XPW-3231 | |

The data in Table 69 relate to novel compounds, wherein the data in Table 70 relate to a novel medical use of compounds disclosed in PCT/EP2018/054686.

In one embodiment, several compounds of the invention were found to inhibit the growth of FU-OV-1 cells (human ovarian carcinoma cells) obtainable from the Deutsche Sammlung von Mikroorganismen und Zellkulturen GmbH (DSMZ) under the accession number ACC 444. FU-OV-1 cells were cultivated in Ham's F-12/DMEM (1:1) medium (Fisherscientific, #11514436) containing 10% fetal bovine serum (Fisherscientific, #15517589) and 1 mM sodium pyruvate (Fisherscientific, #11501871) at 37° C. and 5% CO₂.

A compound is considered as a growth inhibitor of FU-OV-1 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ib) have been identified as growth inhibitors of FU-OV-1 cells. The so far identified FU-OV-1 growth inhibitors relate to the compounds listed in Table 71 and Table 72. The entries of Table 71 and Table 72 are categorized by the corresponding weighted arithmetic means of the compounds without consideration of the respective standard deviations, hence falling into the activity ranges as indicated.

TABLE 71
Proliferation assay with FU-OV-1 cells at 20 μM

| Activity Range | Entry | Compound No. | Specification |
|---|---|---|---|
| 1.0 ± 0.0 | 1 | DMSO | Baseline control |
| 1.0 ± 0.0 | 2 | MTREX | Control at 20 μM |
| 0.8 < AVE_{w} ≤ 0.9 | 3 | XPW-0548 | |
| | 4 | XPW-4580 | |
| 0.7 < AVE_{w} ≤ 0.8 | 5 | XPW-0667 | |
| | 6 | XPW-0674 | |
| | 7 | XPW-0679 | |
| | 8 | XPW-2744 | |
| | 9 | XPW-2805 | |
| 0.7 ± 0.0 | 10 | RES | Control at 20 μM |
| 0.6 < AVE_{w} ≤ 0.7 | 11 | XPW-0675 | |
| | 12 | XPW-0678 | |
| | 13 | XPW-4626 | |
| 0.4 < AVE_{w} ≤ 0.6 | 14 | XPW-0665 | |
| | 15 | XPW-0734 | |
| | 16 | XPW-4614 | |
| | 17 | XPW-4618 | |
| | 18 | XPW-4620 | |
| | 19 | XPW-4622 | |
| | 20 | XPW-4625 | |
| | 21 | XPW-4631 | |
| 0.4 ± 0.0 | 22 | RES | Control at 40 μM |
| 0.2 < AVE_{w} ≤ 0.4 | 23 | XPW-0661 | |
| | 24 | XPW-0686 | |
| | 25 | XPW-0700 | |
| | 26 | XPW-0742 | |
| | 27 | XPW-1750 | |
| | 28 | XPW-4584 | |
| | 29 | XPW-4590 | |
| | 30 | XPW-4612 | |
| | 31 | XPW-4613 | |
| | 32 | XPW-4616 | |
| | 33 | XPW-4617 | |
| | 34 | XPW-4619 | |
| | 35 | XPW-4621 | |
| | 36 | XPW-4632 | |
| | 37 | XPW-4636 | |
| | 38 | XPW-4640 | |
| | 39 | XPW-4644 | |
| 0.0 < AVE_{w} ≤ 0.2 | 40 | XPW-4587 | |
| | 41 | XPW-4593 | |
| | 42 | XPW-4623 | |
| | 43 | XPW-4630 | |
| | 44 | XPW-4639 | |
| | 45 | XPW-4646 | |
| | 46 | XPW-4647 | |

TABLE 72
Proliferation assay with FU-OV-1 cells at 20 μM

| Activity Range | Entry | Compound No. | Specification |
|---|---|---|---|
| 1.0 ± 0.0 | 1 | DMSO | Baseline control |
| 1.0 ± 0.0 | 2 | MTREX | Control at 20 μM |
| 0.8 < AVE_{w} ≤ 0.9 | 3 | XPW-4569 | |
| 0.7 < AVE_{w} ≤ 0.8 | 4 | XPW-2648 | |
| | 5 | XPW-3214 | |
| | 6 | XPW-3217 | |
| | 7 | XPW-4565 | |
| 0.7 ± 0.0 | 8 | RES | Control at 20 μM |
| 0.4 < AVE_{w} ≤ 0.6 | 9 | XPW-0659 | |
| | 10 | XPW-1727 | |
| | 11 | XPW-2798 | |
| | 12 | XPW-3194 | |
| | 13 | XPW-3202 | |
| | 14 | XPW-3203 | |
| | 15 | XPW-3209 | |
| | 16 | XPW-3212 | |
| | 17 | XPW-3218 | |
| | 18 | XPW-3219 | |
| | 19 | XPW-3227 | |
| 0.4 ± 0.0 | 20 | RES | Control at 40 μM |
| 0.2 < AVE_{w} ≤ 0.4 | 21 | XPW-0660 | |
| | 22 | XPW-0663 | |
| | 23 | XPW-2787 | |
| | 24 | XPW-2788 | |
| | 25 | XPW-2791 | |
| | 26 | XPW-2792 | |
| | 27 | XPW-2797 | |
| | 28 | XPW-2800 | |
| | 29 | XPW-3197 | |
| | 30 | XPW-3201 | |
| | 31 | XPW-3206 | |
| | 32 | XPW-3213 | |
| | 33 | XPW-3221 | |
| | 34 | XPW-3226 | |
| | 35 | XPW-3231 | |
| | 36 | XPW-3233 | |
| 0.0 < AVE_{w} ≤ 0.2 | 37 | XPW-0664 | |
| | 38 | XPW-0669 | |
| | 39 | XPW-0670 | |
| | 40 | XPW-0672 | |
| | 41 | XPW-1728 | |
| | 42 | XPW-1736 | |
| | 43 | XPW-3193 | |
| | 44 | XPW-3200 | |
| | 45 | XPW-3225 | |

The data in Table 71 relate to novel compounds, wherein the data in Table 72 relate to a novel medical use of compounds disclosed in PCT/EP2018/054686.

In one embodiment, several compounds of the invention were found to inhibit the growth of LOU-NH91 cells (human lung squamous cell carcinoma cells) obtainable from the Deutsche Sammlung von Mikroorganismen und Zellkulturen GmbH (DSMZ) under the accession number ACC 393. LOU-NH91 cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of LOU-NH91 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of LOU-NH91 cells. The so far identified LOU-NH91 growth inhibitors relate to the compounds listed in Table 73 and Table 74. The entries of Table 73 and Table 74 are categorized by the corresponding weighted arithmetic means of the compounds without consideration of the respective standard deviations, hence falling into the activity ranges as indicated.

TABLE 73
Proliferation assay with LOU-NH91 cells at 20 μM

| Activity Range | Entry | Compound No. | Specification |
|---|---|---|---|
| 1.0 ± 0.0 | 1 | DMSO | Baseline control |
| 0.9 ± 0.1 | 2 | RES | Control at 20 μM |
| 0.8 < AVE_{w} ≤ 0.9 | 3 | XPW-0028 | Used as HCl salt |
| | 4 | XPW-0667 | |
| | 5 | XPW-0728 | |
| | 6 | XPW-4575 | |
| | 7 | XPW-4594 | |
| | 8 | XPW-4628 | |
| 0.7 < AVE_{w} ≤ 0.8 | 9 | XPW-0014 | |
| | 10 | XPW-0182 | |
| | 11 | XPW-2847 | |
| | 12 | XPW-4586 | |
| | 13 | XPW-4589 | |
| | 14 | XPW-4612 | |
| 0.7 ± 0.0 | 15 | RES | Control at 40 μM |
| 0.6 < AVE_{w} ≤ 0.7 | 16 | XPW-0665 | |
| | 17 | XPW-0714 | |
| | 18 | XPW-2805 | |
| | 19 | XPW-2833 | |
| | 20 | XPW-4636 | |
| 0.5 ± 0.1 | 21 | MTREX | Control at 20 μM |
| 0.4 < AVE_{w} ≤ 0.6 | 22 | XPW-0661 | |
| | 23 | XPW-0678 | |
| | 24 | XPW-4626 | |
| 0.2 < AVE_{w} ≤ 0.4 | 25 | XPW-0734 | |
| | 26 | XPW-0742 | |
| | 27 | XPW-4613 | |
| | 28 | XPW-4614 | |
| | 29 | XPW-4633 | |
| 0.0 < AVE_{w} ≤ 0.2 | 30 | XPW-0686 | |
| | 31 | XPW-0700 | |
| | 32 | XPW-0924 | |
| | 33 | XPW-0930 | |
| | 34 | XPW-1750 | |
| | 35 | XPW-4584 | |
| | 36 | XPW-4587 | |
| | 37 | XPW-4590 | |
| | 38 | XPW-4593 | |
| | 39 | XPW-4616 | |
| | 40 | XPW-4617 | |
| | 41 | XPW-4618 | |
| | 42 | XPW-4619 | |
| | 43 | XPW-4620 | |
| | 44 | XPW-4621 | |
| | 45 | XPW-4622 | |
| | 46 | XPW-4623 | |
| | 47 | XPW-4625 | |
| | 48 | XPW-4630 | |
| | 49 | XPW-4631 | |
| | 50 | XPW-4632 | |
| | 51 | XPW-4639 | |
| | 52 | XPW-4640 | |
| | 53 | XPW-4644 | |
| | 54 | XPW-4646 | |
| | 55 | XPW-4647 | |

TABLE 74
Proliferation assay with LOU-NH91 cells at 20 μM

| Activity Range | Entry | Compound No. | Specification |
|---|---|---|---|
| 1.0 ± 0.0 | 1 | DMSO | Baseline control |
| 0.9 ± 0.1 | 2 | RES | Control at 20 μM |
| 0.8 < AVE_{w} ≤ 0.9 | 3 | XPW-0518 | |
| | 4 | XPW-0544 | |
| | 5 | XPW-0659 | |
| | 6 | XPW-1610 | |
| | 7 | XPW-2637 | |
| | 8 | XPW-2644 | |
| | 9 | XPW-3223 | |
| | 10 | XPW-4560 | |
| 0.7 < AVE_{w} ≤ 0.8 | 11 | XPW-0529 | |
| | 12 | XPW-0532 | |
| | 13 | XPW-1582 | |
| | 14 | XPW-1727 | |
| | 15 | XPW-2660 | |
| | 16 | XPW-3202 | |
| | 17 | XPW-3206 | |
| 0.7 ± 0.0 | 18 | RES | Control at 40 μM |
| 0.6 < AVE_{w} ≤ 0.7 | 19 | XPW-0530 | |
| | 20 | XPW-3213 | |
| 0.5 ± 0.1 | 21 | MTREX | Control at 20 μM |
| 0.4 < AVE_{w} ≤ 0.6 | 22 | XPW-3197 | |
| | 23 | XPW-3201 | |
| 0.2 < AVE_{w} ≤ 0.4 | 24 | XPW-0660 | |
| | 25 | XPW-0663 | |
| | 26 | XPW-2787 | |
| | 27 | XPW-2788 | |
| | 28 | XPW-2791 | |
| | 29 | XPW-2798 | |
| | 30 | XPW-3221 | |
| 0.0 < AVE_{w} ≤ 0.2 | 31 | XPW-0664 | |
| | 32 | XPW-0669 | |
| | 33 | XPW-0670 | |
| | 34 | XPW-0672 | |
| | 35 | XPW-1728 | |
| | 36 | XPW-1736 | |
| | 37 | XPW-2797 | |
| | 38 | XPW-2800 | |
| | 39 | XPW-3225 | |
| | 40 | XPW-3226 | |
| | 41 | XPW-3227 | |
| | 42 | XPW-3231 | |
| | 43 | XPW-3233 | |

The data in Table 73 relate to novel compounds, wherein the data in Table 74 relate to a novel medical use of compounds disclosed in PCT/EP2018/054686.

In one embodiment, several compounds of the invention were found to inhibit the growth of 23132/87 cells (human gastric adenocarcinoma cells) obtainable from the Deutsche Sammlung von Mikroorganismen und Zellkulturen GmbH (DSMZ) under the accession number ACC 201. 23132/87 cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of 23132/87 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of 23132/87 cells. The so far identified 23132/87 growth inhibitors relate to the compounds listed in Table 75 and Table 76. The entries of Table 75 and Table 76 are categorized by the corresponding weighted arithmetic means of the compounds without consideration of the respective standard deviations, hence falling into the activity ranges as indicated.

TABLE 75
Proliferation assay with 23132/87 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0776 | |
| | 3 | XPW-0790 | |
| | 4 | XPW-2795 | |
| | 5 | XPW-2805 | |
| | 6 | XPW-4638 | |
| 0.7 < AVE_{w} ≤ 0.8 | 7 | XPW-0014 | |
| | 8 | XPW-0667 | |
| | 9 | XPW-4583 | |
| | 10 | XPW-4589 | |
| | 11 | XPW-4610 | |
| | 12 | XPW-4612 | |
| 0.6 < AVE_{w} ≤ 0.7 | 13 | XPW-0728 | |
| | 14 | XPW-0784 | |
| | 15 | XPW-0916 | |
| | 16 | XPW-2744 | |
| | 17 | XPW-4585 | |
| | 18 | XPW-4637 | |
| 0.5 ± 0.1 | 19 | RES | Control at 20 μM |
| 0.4 < AVE_{w} ≤ 0.6 | 20 | XPW-0574 | |
| | 21 | XPW-0714 | |
| | 22 | XPW-4580 | |
| | 23 | XPW-4586 | |
| 0.3 ± 0.0 | 24 | MTREX | Control at 20 μM |
| 0.2 < AVE_{w} ≤ 0.4 | 25 | XPW-0661 | |
| | 26 | XPW-4626 | |
| | 27 | XPW-4636 | |
| 0.2 ± 0.0 | 28 | RES | Control at 40 μM |
| 0.0 < AVE_{w} ≤ 0.2 | 29 | XPW-0665 | |
| | 30 | XPW-0678 | |
| | 31 | XPW-0686 | |
| | 32 | XPW-0700 | |
| | 33 | XPW-0734 | |
| | 34 | XPW-0742 | |
| | 35 | XPW-0924 | |
| | 36 | XPW-0930 | |
| | 37 | XPW-1750 | |
| | 38 | XPW-4584 | |
| | 39 | XPW-4587 | |
| | 40 | XPW-4590 | |
| | 41 | XPW-4593 | |
| | 42 | XPW-4613 | |
| | 43 | XPW-4614 | |
| | 44 | XPW-4616 | |
| | 45 | XPW-4617 | |
| | 46 | XPW-4618 | |
| | 47 | XPW-4619 | |
| | 48 | XPW-4620 | |
| | 49 | XPW-4621 | |
| | 50 | XPW-4622 | |
| | 51 | XPW-4623 | |
| | 52 | XPW-4625 | |
| | 53 | XPW-4628 | |
| | 54 | XPW-4630 | |
| | 55 | XPW-4631 | |
| | 56 | XPW-4632 | |
| | 57 | XPW-4633 | |
| | 58 | XPW-4639 | |
| | 59 | XPW-4640 | |
| | 60 | XPW-4644 | |
| | 61 | XPW-4646 | |
| | 62 | XPW-4647 | |

TABLE 76
Proliferation assay with 23132/87 cells at 20 uM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0518 | |
| 0.7 < AVE_{w} ≤ 0.8 | 3 | XPW-1596 | Measured at 10 μM |
| | 4 | XPW-3217 | |
| 0.5 ± 0.1 | 5 | RES | Control at 20 μM |
| 0.4 < AVE_{w} ≤ 0.6 | 6 | XPW-3202 | |
| | 7 | XPW-3214 | |
| 0.3 ± 0.0 | 8 | MTREX | Control at 20 μM |
| 0.2 ± 0.0 | 9 | RES | Control at 40 μM |
| 0.0 < AVE_{w} ≤ 0.2 | 10 | XPW-0659 | |
| | 11 | XPW-0660 | |
| | 12 | XPW-0663 | |
| | 13 | XPW-0664 | |
| | 14 | XPW-0669 | |
| | 15 | XPW-0670 | |
| | 16 | XPW-0672 | |
| | 17 | XPW-1727 | |
| | 18 | XPW-1728 | |
| | 19 | XPW-1736 | |
| | 20 | XPW-2787 | |
| | 21 | XPW-2788 | |
| | 22 | XPW-2791 | |
| | 23 | XPW-2792 | |
| | 24 | XPW-2797 | |
| | 25 | XPW-2798 | |
| | 26 | XPW-2800 | |
| | 27 | XPW-3193 | |
| | 28 | XPW-3194 | |
| | 29 | XPW-3197 | |
| | 30 | XPW-3200 | |
| | 31 | XPW-3201 | |
| | 32 | XPW-3203 | |
| | 33 | XPW-3206 | |
| | 34 | XPW-3209 | |
| | 35 | XPW-3212 | |
| | 36 | XPW-3213 | |
| | 37 | XPW-3218 | |
| | 38 | XPW-3219 | |
| | 39 | XPW-3221 | |
| | 40 | XPW-3225 | |
| | 41 | XPW-3226 | |
| | 42 | XPW-3227 | |
| | 43 | XPW-3231 | |
| | 44 | XPW-3233 | |

The data in Table 75 relate to novel compounds, wherein the data in Table 76 relate to a novel medical use of compounds disclosed in PCT/EP2018/054686.

In one embodiment, several compounds of the invention were found to inhibit the growth of CAL-27 cells (human tongue squamous cell carcinoma cells) obtainable from the Deutsche Sammlung von Mikroorganismen und Zellkulturen GmbH (DSMZ) under the accession number ACC 446. CAL-27 cells were cultivated in DMEM medium (Fisherscientific, #11584456) containing 10% fetal bovine serum (Fisherscientific, #15517589) at 37° C. and 5% CO₂.

A compound is considered as a growth inhibitor of CAL-27 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of CAL-27 cells. The so far identified CAL-27 growth inhibitors relate to the compounds listed in Table 77 and Table 78. The entries of Table 77 and Table 78 are categorized by the corresponding weighted arithmetic means of the compounds without consideration of the respective standard deviations, hence falling into the activity ranges as indicated.

TABLE 77
Proliferation assay with CAL-27 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0702 | |
| | 3 | XPW-0716 | |
| | 4 | XPW-0790 | |
| | 5 | XPW-0818 | |
| | 6 | XPW-2912 | |
| | 7 | XPW-4610 | |
| | 8 | XPW-4636 | |
| 0.8 ± 0.1 | 9 | RES | Control at 20 μM |
| 0.7 < AVE_{w} ≤ 0.8 | 10 | XPW-0675 | |
| | 11 | XPW-0832 | |
| | 12 | XPW-2795 | |
| | 13 | XPW-2805 | |
| | 14 | XPW-2890 | |
| | 15 | XPW-2918 | |
| | 16 | XPW-4591 | |
| 0.6 < AVE_{w} ≤ 0.7 | 17 | XPW-0667 | |
| | 18 | XPW-4580 | |
| 0.4 < AVE_{w} ≤ 0.6 | 19 | XPW-0930 | |
| | 20 | XPW-2744 | |
| | 21 | XPW-2806 | |
| | 22 | XPW-2904 | |
| | 23 | XPW-4578 | |
| | 24 | XPW-4588 | |
| | 25 | XPW-4595 | |
| | 26 | XPW-4626 | |
| | 27 | XPW-4635 | |
| 0.4 ± 0.3 | 28 | RES | Control at 40 μM |
| 0.2 < AVE_{w} ≤ 0.4 | 29 | XPW-0014 | |
| | 30 | XPW-0539 | |
| | 31 | XPW-0574 | |
| | 32 | XPW-0679 | |
| | 33 | XPW-0720 | |
| | 34 | XPW-0762 | |
| | 35 | XPW-0916 | |
| | 36 | XPW-4575 | |
| | 37 | XPW-4583 | |
| | 38 | XPW-4585 | |
| | 39 | XPW-4589 | |
| | 40 | XPW-4594 | |
| 0.1 ± 0.0 | 41 | MTREX | Control at 20 μM |
| 0.0 < AVE_{w} ≤ 0.2 | 42 | XPW-0661 | |
| | 43 | XPW-0665 | |
| | 44 | XPW-0674 | |
| | 45 | XPW-0678 | |
| | 46 | XPW-0686 | |
| | 47 | XPW-0700 | |
| | 48 | XPW-0706 | |
| | 49 | XPW-0714 | |
| | 50 | XPW-0728 | |
| | 51 | XPW-0734 | |
| | 52 | XPW-0742 | |
| | 53 | XPW-0770 | |
| | 54 | XPW-0776 | |
| | 55 | XPW-0784 | |
| | 56 | XPW-0924 | |
| | 57 | XPW-1750 | |
| | 58 | XPW-4584 | |
| | 59 | XPW-4586 | |
| | 60 | XPW-4587 | |
| | 61 | XPW-4590 | |
| | 62 | XPW-4593 | |
| | 63 | XPW-4613 | |
| | 64 | XPW-4614 | |
| | 65 | XPW-4616 | |
| | 66 | XPW-4617 | |
| | 67 | XPW-4618 | |
| | 68 | XPW-4619 | |
| | 69 | XPW-4620 | |
| | 70 | XPW-4621 | |
| | 71 | XPW-4622 | |
| | 72 | XPW-4623 | |
| | 73 | XPW-4625 | |
| | 74 | XPW-4630 | |
| | 75 | XPW-4631 | |
| | 76 | XPW-4632 | |
| | 77 | XPW-4633 | |
| | 78 | XPW-4637 | |
| | 79 | XPW-4638 | |
| | 80 | XPW-4639 | |
| | 81 | XPW-4640 | |
| | 82 | XPW-4644 | |
| | 83 | XPW-4646 | |
| | 84 | XPW-4647 | |

TABLE 78
Proliferation assay with CAL-27 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-3208 | |
| 0.8 ± 0.1 | 3 | RES | Control at 20 μM |
| 0.7 < AVE_{w} ≤ 0.8 | 4 | XPW-3232 | |
| 0.4 ± 0.3 | 5 | RES | Control at 40 μM |
| 0.1 ± 0.0 | 6 | MTREX | Control at 20 μM |
| 0.0 ≤ AVE_{w} ≤ 0.2 | 7 | XPW-0660 | |
| | 8 | XPW-2788 | |

The data in Table 77 relate to novel compounds, wherein the data in Table 78 relate to a novel medical use of compounds disclosed in PCT/EP2018/054686.

In one embodiment, several compounds of the invention were found to inhibit the growth of BHY cells (human oral squamous cell carcinoma cells) obtainable from the Deutsche Sammlung von Mikroorganismen und Zellkulturen GmbH (DSMZ) under the accession number ACC 404. BHY cells were cultivated in DMEM medium (Fisherscientific, #11584456) containing 10% fetal bovine serum (Fisherscientific, #15517589) at 37° C. and 5% CO₂.

A compound is considered as a growth inhibitor of BHY cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of BHY cells. The so far identified BHY growth inhibitors relate to the compounds listed in Table 79, Table 80 and Table 81. The entries of Table 79, Table 80 and Table 81 are categorized by the corresponding weighted arithmetic means of the compounds without consideration of the respective standard deviations, hence falling into the activity ranges as indicated.

TABLE 79
Proliferation assay with BHY cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0574 | |
| | 3 | XPW-0667 | |
| | 4 | XPW-0832 | |
| | 5 | XPW-2744 | |
| | 6 | XPW-2795 | |
| | 7 | XPW-2833 | |
| | 8 | XPW-2918 | |
| | 9 | XPW-4628 | |
| | 10 | XPW-4636 | |
| 0.7 < AVE_{w} ≤ 0.8 | 11 | XPW-0014 | |
| | 12 | XPW-0716 | |
| | 13 | XPW-4588 | |
| | 14 | XPW-4595 | |
| 0.6 < AVE_{w} ≤ 0.7 | 15 | XPW-0675 | |
| | 16 | XPW-0702 | |
| | 17 | XPW-2805 | |
| | 18 | XPW-2904 | |
| | 19 | XPW-4575 | |
| | 20 | XPW-4585 | |
| | 21 | XPW-4626 | |
| | 22 | XPW-4633 | |
| 0.5 ± 0.1 | 23 | RE'S | Control at 20 μM |
| 0.4 < AVE_{w} ≤ 0.6 | 24 | XPW-0720 | |
| | 25 | XPW-0728 | |
| | 26 | XPW-0916 | |
| | 27 | XPW-0930 | |
| | 28 | XPW-4583 | |
| | 29 | XPW-4589 | |
| | 30 | XPW-4594 | |
| | 31 | XPW-4637 | |
| 0.3 ± 0.0 | 32 | MTREX | Control at 20 μM |
| 0.3 ± 0.0 | 33 | RES | Control at 40 μM |
| 0.2 < AVE_{w} ≤ 0.4 | 34 | XPW-0539 | |
| | 35 | XPW-0661 | |
| | 36 | XPW-0679 | |
| | 37 | XPW-0706 | |
| | 38 | XPW-0714 | |
| | 39 | XPW-0762 | |
| | 40 | XPW-0770 | |
| | 41 | XPW-0776 | |
| | 42 | XPW-0784 | |
| | 43 | XPW-2806 | |
| | 44 | XPW-4580 | |
| | 45 | XPW-4586 | |
| | 46 | XPW-4638 | |
| 0.0 < AVE_{w} ≤ 0.2 | 47 | XPW-0665 | |
| | 48 | XPW-0674 | |
| | 49 | XPW-0678 | |
| | 50 | XPW-0686 | |
| | 51 | XPW-0700 | |
| | 52 | XPW-0734 | |
| | 53 | XPW-0742 | |
| | 54 | XPW-0924 | |
| | 55 | XPW-1750 | |
| | 56 | XPW-4584 | |
| | 57 | XPW-4587 | |
| | 58 | XPW-4590 | |
| | 59 | XPW-4593 | |
| | 60 | XPW-4613 | |
| | 61 | XPW-4614 | |
| | 62 | XPW-4616 | |
| | 63 | XPW-4617 | |
| | 64 | XPW-4618 | |
| | 65 | XPW-4619 | |
| | 66 | XPW-4620 | |
| | 67 | XPW-4621 | |
| | 68 | XPW-4622 | |
| | 69 | XPW-4623 | |
| | 70 | XPW-4625 | |
| | 71 | XPW-4630 | |
| | 72 | XPW-4631 | |
| | 73 | XPW-4632 | |
| | 74 | XPW-4639 | |
| | 75 | XPW-4640 | |
| | 76 | XPW-4644 | |
| | 77 | XPW-4646 | |
| | 78 | XPW-4647 | |

TABLE 80
Proliferation assay with BHY cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0518 | |
| | 3 | XPW-1582 | |
| | 4 | XPW-2665 | |
| | 5 | XPW-3199 | |
| | 6 | XPW-3211 | |
| | 7 | XPW-3216 | |
| 0.7 < AVE_{w} ≤ 0.8 | 8 | XPW-3223 | |
| 0.6 < AVE_{w} ≤ 0.7 | 9 | XPW-2658 | |
| | 10 | XPW-3217 | |
| | 11 | XPW-3232 | |
| 0.5 ± 0.1 | 12 | RES | Control at 20 μM |
| 0.4 < AVE_{w} ≤ 0.6 | 13 | XPW-0546 | |
| | 14 | XPW-1610 | |
| | 15 | XPW-2660 | |
| | 16 | XPW-3202 | |
| 0.3 ± 0.0 | 17 | MTREX | Control at 20 μM |
| 0.3 ± 0.0 | 18 | RES | Control at 40 μM |
| 0.2 < AVE_{w} ≤ 0.4 | 19 | XPW-0659 | |
| | 20 | XPW-1727 | |
| | 21 | XPW-3203 | |
| | 22 | XPW-3209 | |
| | 23 | XPW-3214 | |
| | 24 | XPW-3218 | |
| 0.0 ≤ AVE_{w} ≤ 0.2 | 25 | XPW-0660 | |
| | 26 | XPW-0663 | |
| | 27 | XPW-0669 | |
| | 28 | XPW-0670 | |
| | 29 | XPW-2787 | |
| | 30 | XPW-2788 | |
| | 31 | XPW-2791 | |
| | 32 | XPW-2797 | |
| | 33 | XPW-2798 | |
| | 34 | XPW-3194 | |
| | 35 | XPW-3197 | |
| | 36 | XPW-3200 | |
| | 37 | XPW-3206 | |
| | 38 | XPW-3212 | |
| | 39 | XPW-3213 | |
| | 40 | XPW-3225 | |
| | 41 | XPW-3226 | |
| | 42 | XPW-3227 | |
| | 43 | XPW-3231 | |
| | 44 | XPW-3233 | |

TABLE 81
Proliferation assay with BHY cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0520 | |
| | 3 | XPW-0524 | |
| | 4 | XPW-1596 | Measured at 10 μM |
| | 5 | XPW-2657 | |
| | 6 | XPW-2666 | |
| | 7 | XPW-2674 | |
| | 8 | XPW-3215 | |
| 0.7 < AVE_{w} ≤ 0.8 | 9 | XPW-0537 | |
| | 10 | XPW-0543 | |
| | 11 | XPW-2648 | |
| | 12 | XPW-4565 | |
| 0.6 < AVE_{w} ≤ 0.7 | 13 | XPW-0529 | |
| | 14 | XPW-0538 | |
| | 15 | XPW-4569 | |
| 0.5 ± 0.1 | 16 | RES | Control at 20 μM |
| 0.4 < AVE_{w} ≤ 0.6 | 17 | XPW-0530 | |
| | 18 | XPW-0544 | |
| 0.3 ± 0.0 | 19 | MTREX | Control at 20 μM |
| 0.3 ± 0.0 | 20 | RES | Control at 40 μM |

The data in Table 79 relate to novel compounds, wherein the data in Table 80 and Table 81 relate to a novel medical use of compounds disclosed in PCT/EP2018/054686.

In one embodiment, several compounds of the invention were found to inhibit the growth of SCC-25 cells (human tongue squamous cell carcinoma cells) obtainable from the Deutsche Sammlung von Mikroorganismen und Zellkulturen GmbH (DSMZ) under the accession number ACC 617. SCC-25 cells were cultivated in Ham's F-12/DMEM (1:1) medium (Fisherscientific, #11514436) containing 10% fetal bovine serum (Fisherscientific, #15517589) and 1 mM sodium pyruvate (Fisherscientific, #11501871) at 37° C. and 5% CO₂.

A compound is considered as a growth inhibitor of SCC-25 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of SCC-25 cells. The so far identified SCC-25 growth inhibitors relate to the compounds listed in Table 82 and Table 83. The entries of Table 82 and Table 83 are categorized by the corresponding weighted arithmetic means of the compounds without consideration of the respective standard deviations, hence falling into the activity ranges as indicated.

TABLE 82
Proliferation assay with SCC-25 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0014 | |
| | 3 | XPW-0182 | |
| | 4 | XPW-2795 | |
| | 5 | XPW-4578 | |
| | 6 | XPW-4585 | |
| | 7 | XPW-4610 | |
| | 8 | XPW-4612 | |
| | 9 | XPW-4637 | |
| | 10 | XPW-4638 | |
| | 11 | XPW-4643 | |
| 0.7 < AVE_{w} ≤ 0.8 | 12 | XPW-0667 | |
| | 13 | XPW-2805 | |
| 0.5 ± 0.1 | 14 | MTREX | Control at 20 μM |
| 0.4 < AVE_{w} ≤ 0.6 | 15 | XPW-0675 | |
| | 16 | XPW-0930 | |
| | 17 | XPW-2744 | |
| | 18 | XPW-4580 | |
| | 19 | XPW-4586 | |
| 0.4 ± 0.1 | 20 | RES | Control at 20 μM |
| 0.2 < AVE_{w} ≤ 0.4 | 21 | XPW-0665 | |
| | 22 | XPW-0679 | |
| | 23 | XPW-2806 | |
| | 24 | XPW-4633 | |
| | 25 | XPW-4636 | |
| 0.1 ± 0.0 | 26 | RES | Control at 40 μM |
| 0.0 < AVE_{w} ≤ 0.2 | 27 | XPW-0661 | |
| | 28 | XPW-0674 | |
| | 29 | XPW-0678 | |
| | 30 | XPW-0686 | |
| | 31 | XPW-0700 | |
| | 32 | XPW-0734 | |
| | 33 | XPW-0742 | |
| | 34 | XPW-0924 | |
| | 35 | XPW-1750 | |
| | 36 | XPW-4584 | |
| | 37 | XPW-4587 | |
| | 38 | XPW-4590 | |
| | 39 | XPW-4593 | |
| | 40 | XPW-4613 | |
| | 41 | XPW-4614 | |
| | 42 | XPW-4616 | |
| | 43 | XPW-4617 | |
| | 44 | XPW-4618 | |
| | 45 | XPW-4619 | |
| | 46 | XPW-4620 | |
| | 47 | XPW-4621 | |
| | 48 | XPW-4622 | |
| | 49 | XPW-4623 | |
| | 50 | XPW-4625 | |
| | 51 | XPW-4626 | |
| | 52 | XPW-4628 | |
| | 53 | XPW-4630 | |
| | 54 | XPW-4631 | |
| | 55 | XPW-4632 | |
| | 56 | XPW-4639 | |
| | 57 | XPW-4640 | |
| | 58 | XPW-4644 | |
| | 59 | XPW-4646 | |
| | 60 | XPW-4647 | |

TABLE 83
Proliferation assay with SCC-25 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0515 | |
| | 3 | XPW-1602 | |
| | 4 | XPW-2660 | |
| | 5 | XPW-2666 | |
| | 6 | XPW-2674 | |
| | 7 | XPW-3215 | |
| | 8 | XPW-3216 | |
| | 9 | XPW-3223 | |
| | 10 | XPW-3230 | |
| | 11 | XPW-4548 | |
| | 12 | XPW-4550 | |
| | 13 | XPW-4565 | |
| | 14 | XPW-4567 | |
| 0.7 < AVE_{w} ≤ 0.8 | 15 | XPW-0529 | |
| | 16 | XPW-3217 | |
| 0.5 ± 0.1 | 17 | MTREX | Control at 20 μM |
| 0.4 < AVE_{w} ≤ 0.6 | 18 | XPW-3202 | |
| 0.4 ± 0.1 | 19 | RES | Control at 20 μM |
| 0.2 < AVE_{w} ≤ 0.4 | 20 | XPW-0659 | |
| | 21 | XPW-1727 | |
| | 22 | XPW-3203 | |
| | 23 | XPW-3214 | |
| 0.1 ± 0.0 | 24 | RES | Control at 40 μM |
| 0.0 < AVE_{w} ≤ 0.2 | 25 | XPW-0660 | |
| | 26 | XPW-0663 | |
| | 27 | XPW-0664 | |
| | 28 | XPW-0669 | |
| | 29 | XPW-0670 | |
| | 30 | XPW-0672 | |
| | 31 | XPW-1728 | |
| | 32 | XPW-1736 | |
| | 33 | XPW-2787 | |
| | 34 | XPW-2788 | |
| | 35 | XPW-2791 | |
| | 36 | XPW-2792 | |
| | 37 | XPW-2797 | |
| | 38 | XPW-2798 | |
| | 39 | XPW-2800 | |
| | 40 | XPW-3193 | |
| | 41 | XPW-3194 | |
| | 42 | XPW-3197 | |
| | 43 | XPW-3200 | |
| | 44 | XPW-3201 | |
| | 45 | XPW-3206 | |
| | 46 | XPW-3209 | |
| | 47 | XPW-3212 | |
| | 48 | XPW-3213 | |
| | 49 | XPW-3218 | |
| | 50 | XPW-3219 | |
| | 51 | XPW-3221 | |
| | 52 | XPW-3225 | |
| | 53 | XPW-3226 | |
| | 54 | XPW-3227 | |
| | 55 | XPW-3231 | |
| | 56 | XPW-3233 | |

The data in Table 82 relate to novel compounds, wherein the data in Table 83 relate to a novel medical use of compounds disclosed in PCT/EP2018/054686.

In one embodiment, several compounds of the invention were found to inhibit the growth of A-431 cells (human epidermoid squamous cell carcinoma cells) obtainable from the Cell Lines Service GmbH (CLS) under the accession number 300112. A-431 cells were cultivated in DMEM medium (Fisherscientific, #11584456) containing 10% fetal bovine serum (Fisherscientific, #15517589) at 37° C. and 5% CO₂.

A compound is considered as a growth inhibitor of A-431 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of A-431 cells. The so far identified A-431 growth inhibitors relate to the compounds listed in Table 84 and Table 85. The entries of Table 84 and Table 85 are categorized by the corresponding weighted arithmetic means of the compounds without consideration of the respective standard deviations, hence falling into the activity ranges as indicated.

TABLE 84
Proliferation assay with A-431 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0314 | |
| | 3 | XPW-0702 | |
| | 4 | XPW-2833 | |
| | 5 | XPW-4588 | |
| 0.7 < AVE_{w} ≤ 0.8 | 6 | XPW-0028 | Used as HCl salt |
| | 7 | XPW-0716 | |
| | 8 | XPW-0832 | |
| | 9 | XPW-2847 | |
| | 10 | XPW-4575 | |
| 0.6 < AVE_{w} ≤ 0.7 | 11 | XPW-0776 | |
| | 12 | XPW-0916 | |
| | 13 | XPW-4583 | |
| | 14 | XPW-4589 | |
| | 15 | XPW-4595 | |
| | 16 | XPW-4629 | |
| 0.6 ± 0.0 | 17 | RES | Control at 20 μM |
| 0.4 < AVE_{w} ≤ 0.6 | 18 | XPW-0574 | |
| | 19 | XPW-0667 | |
| | 20 | XPW-0675 | |
| | 21 | XPW-0679 | |
| | 22 | XPW-0784 | |
| | 23 | XPW-0930 | |
| | 24 | XPW-2744 | |
| | 25 | XPW-2805 | |
| | 26 | XPW-4585 | |
| | 27 | XPW-4594 | |
| | 28 | XPW-4612 | |
| | 29 | XPW-4628 | |
| | 30 | XPW-4641 | |
| 0.2 < AVE_{w} ≤ 0.4 | 31 | XPW-0014 | |
| | 32 | XPW-0706 | |
| | 33 | XPW-0720 | |
| | 34 | XPW-0728 | |
| | 35 | XPW-0770 | |
| | 36 | XPW-4580 | |
| | 37 | XPW-4586 | |
| | 38 | XPW-4626 | |
| | 39 | XPW-4636 | |
| | 40 | XPW-4637 | |
| | 41 | XPW-4638 | |
| 0.2 ± 0.1 | 42 | MTREX | Control at 20 μM |
| 0.2 ± 0.0 | 43 | RES | Control at 40 μM |
| 0.0 < AVE_{w} ≤ 0.2 | 44 | XPW-0661 | |
| | 45 | XPW-0665 | |
| | 46 | XPW-0674 | |
| | 47 | XPW-0678 | |
| | 48 | XPW-0686 | |
| | 49 | XPW-0700 | |
| | 50 | XPW-0714 | |
| | 51 | XPW-0734 | |
| | 52 | XPW-0742 | |
| | 53 | XPW-0924 | |
| | 54 | XPW-1750 | |
| | 55 | XPW-2806 | |
| | 56 | XPW-4584 | |
| | 57 | XPW-4587 | |
| | 58 | XPW-4590 | |
| | 59 | XPW-4593 | |
| | 60 | XPW-4613 | |
| | 61 | XPW-4614 | |
| | 62 | XPW-4616 | |
| | 63 | XPW-4617 | |
| | 64 | XPW-4618 | |
| | 65 | XPW-4619 | |
| | 66 | XPW-4620 | |
| | 67 | XPW-4621 | |
| | 68 | XPW-4622 | |
| | 69 | XPW-4623 | |
| | 70 | XPW-4625 | |
| | 71 | XPW-4630 | |
| | 72 | XPW-4631 | |
| | 73 | XPW-4632 | |
| | 74 | XPW-4633 | |
| | 75 | XPW-4639 | |
| | 76 | XPW-4640 | |
| | 77 | XPW-4644 | |
| | 78 | XPW-4646 | |
| | 79 | XPW-4647 | |

TABLE 85
Proliferation assay with A-431 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0516 | |
| | 3 | XPW-2648 | |
| | 4 | XPW-2657 | |
| | 5 | XPW-2661 | |
| | 6 | XPW-3215 | |
| | 7 | XPW-3224 | |
| | 8 | XPW-4567 | |
| 0.7 < AVE_{w} ≤ 0.8 | 9 | XPW-0538 | |
| | 10 | XPW-1596 | Measured at 10 μM |
| | 11 | XPW-3234 | |
| | 12 | XPW-4565 | |
| 0.6 < AVE_{w} ≤ 0.7 | 13 | XPW-0543 | |
| | 14 | XPW-0546 | |
| | 15 | XPW-1602 | |
| | 16 | XPW-3202 | |
| | 17 | XPW-4569 | |
| 0.6 ± 0.0 | 18 | RES | Control at 20 μM |
| 0.4 < AVE_{w} ≤ 0.6 | 19 | XPW-0529 | |
| | 20 | XPW-1610 | |
| 0.2 < AVE_{w} ≤ 0.4 | 21 | XPW-0530 | |
| | 22 | XPW-0544 | |
| | 23 | XPW-3232 | |
| 0.2 ± 0.1 | 24 | MTREX | Control at 20 μM |
| 0.2 ± 0.0 | 25 | RES | Control at 40 μM |
| 0.0 < AVE_{w} ≤ 0.2 | 26 | XPW-0659 | |
| | 27 | XPW-0660 | |
| | 28 | XPW-0663 | |
| | 29 | XPW-0664 | |
| | 30 | XPW-0669 | |
| | 31 | XPW-0670 | |
| | 32 | XPW-0672 | |
| | 33 | XPW-1727 | |
| | 34 | XPW-1728 | |
| | 35 | XPW-1736 | |
| | 36 | XPW-2787 | |
| | 37 | XPW-2788 | |
| | 38 | XPW-2791 | |
| | 39 | XPW-2792 | |
| | 40 | XPW-2797 | |
| | 41 | XPW-2798 | |
| | 42 | XPW-2800 | |
| | 43 | XPW-3193 | |
| | 44 | XPW-3194 | |
| | 45 | XPW-3197 | |
| | 46 | XPW-3200 | |
| | 47 | XPW-3201 | |
| | 48 | XPW-3203 | |
| | 49 | XPW-3206 | |
| | 50 | XPW-3209 | |
| | 51 | XPW-3212 | |
| | 52 | XPW-3213 | |
| | 53 | XPW-3214 | |
| | 54 | XPW-3218 | |
| | 55 | XPW-3219 | |
| | 56 | XPW-3221 | |
| | 57 | XPW-3225 | |
| | 58 | XPW-3226 | |
| | 59 | XPW-3227 | |
| | 60 | XPW-3231 | |
| | 61 | XPW-3233 | |

The data in Table 84 relate to novel compounds, wherein the data in Table 85 relate to a novel medical use of compounds disclosed in PCT/EP2018/054686.

In one embodiment, several compounds of the invention were found to inhibit the growth of human epidermal keratinocyte progenitors, (HPEKp, pooled), obtainable from CELLnTEC Advanced Cell Systems AG under the accession number HPEKp. HPEKp cells were cultivated in CnT-Prime epithelial culture medium (CELLnTEC, #CnT-PR, a fully defined, low calcium formulation, completely free of animal or human-derived components) without addition of further components at 37° C. and 5% CO₂.

A compound is considered as a growth inhibitor of HPEKp cells, if—at a reference concentration of 10 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of HPEKp cells. The so far identified HPEKp growth inhibitors relate to the compounds listed in Table 86, Table 87 and Table 88. The entries of Table 86, Table 87 and Table 88 are categorized by the corresponding weighted arithmetic means of the compounds without consideration of the respective standard deviations, hence falling into the activity ranges as indicated.

TABLE 86
Proliferation assay with HPEKp cells at 10 μM

| Activity Range | Entry | Compound No. | Specification |
|---|---|---|---|
| 1.0 ± 0.0 | 1 | DMSO | Baseline control |
| 0.9 ± 0.0 | 2 | MTREX | Control at 20 μM |
| 0.8 < AVE_{w} ≤ 0.9 | 3 | XPW-0539 | |
| | 4 | XPW-0674 | |
| | 5 | XPW-0703 | |
| | 6 | XPW-0717 | |
| | 7 | XPW-2675 | |
| | 8 | XPW-2676 | |
| | 9 | XPW-2848 | |
| | 10 | XPW-2890 | |
| | 11 | XPW-4581 | |
| | 12 | XPW-4627 | |
| 0.7 < AVE_{w} ≤ 0.8 | 13 | XPW-0547 | |
| | 14 | XPW-0552 | |
| | 15 | XPW-0702 | |
| | 16 | XPW-0716 | |
| | 17 | XPW-0832 | |
| | 18 | XPW-0924 | |
| | 19 | XPW-2732 | |
| | 20 | XPW-2795 | |
| | 21 | XPW-2834 | |
| | 22 | XPW-2847 | |
| | 23 | XPW-4578 | |
| | 24 | XPW-4580 | |
| | 25 | XPW-4605 | |
| 0.6 < AVE_{w} ≤ 0.7 | 26 | XPW-0042 | |
| | 27 | XPW-0548 | |
| | 28 | XPW-0667 | |
| | 29 | XPW-0718 | |
| | 30 | XPW-0776 | |
| | 31 | XPW-0818 | |
| | 32 | XPW-2805 | |
| | 33 | XPW-2806 | |
| | 34 | XPW-2833 | |
| | 35 | XPW-4584 | |
| 0.4 < AVE_{w} ≤ 0.6 | 36 | XPW-0314 | |
| | 37 | XPW-0566 | |
| | 38 | XPW-0603 | |
| | 39 | XPW-0604 | |
| | 40 | XPW-0675 | |
| | 41 | XPW-0679 | |
| | 42 | XPW-0790 | |
| | 43 | XPW-0916 | |
| | 44 | XPW-2904 | |
| | 45 | XPW-2918 | |
| | 46 | XPW-4574 | |
| | 47 | XPW-4579 | |
| | 48 | XPW-4583 | |
| | 49 | XPW-4590 | |
| | 50 | XPW-4625 | |
| | 51 | XPW-4634 | |
| | 52 | XPW-4638 | |
| | 53 | XPW-4640 | |
| | 54 | XPW-4642 | |
| | 55 | XPW-4643 | |
| 0.2 < AVE_{w} ≤ 0.4 | 56 | XPW-2688 | |
| | 57 | XPW-4610 | |
| 0.2 ± 0.0 | 58 | RES | Control at 20 μM |
| 0.2 ± 0.0 | 59 | RES | Control at 40 μM |
| 0.0 < AVE_{w} ≤ 0.2 | 60 | XPW-0014 | |
| | 61 | XPW-0020 | Used as HCl salt |
| | 62 | XPW-0028 | Used as HCl salt |
| | 63 | XPW-0182 | |
| | 64 | XPW-0560 | |
| | 65 | XPW-0574 | |
| | 66 | XPW-0575 | |
| | 67 | XPW-0576 | |
| | 68 | XPW-0580 | |
| | 69 | XPW-0588 | |
| | 70 | XPW-0608 | |
| | 71 | XPW-0616 | |
| | 72 | XPW-0636 | |
| | 73 | XPW-0661 | |
| | 74 | XPW-0665 | |
| | 75 | XPW-0686 | |
| | 76 | XPW-0700 | |
| | 77 | XPW-0714 | |
| | 78 | XPW-0728 | |
| | 79 | XPW-0734 | |
| | 80 | XPW-0742 | |
| | 81 | XPW-0770 | |
| | 82 | XPW-0784 | |
| | 83 | XPW-0798 | |
| | 84 | XPW-0930 | |
| | 85 | XPW-1750 | |
| | 86 | XPW-2703 | |
| | 87 | XPW-2704 | |
| | 88 | XPW-2708 | |
| | 89 | XPW-2716 | |
| | 90 | XPW-2744 | |
| | 91 | XPW-2926 | |
| | 92 | XPW-3038 | |
| | 93 | XPW-4585 | |
| | 94 | XPW-4586 | |
| | 95 | XPW-4587 | |
| | 96 | XPW-4588 | |
| | 97 | XPW-4589 | |
| | 98 | XPW-4591 | |
| | 99 | XPW-4592 | |
| | 100 | XPW-4593 | |
| | 101 | XPW-4612 | |
| | 102 | XPW-4613 | |
| | 103 | XPW-4614 | |
| | 104 | XPW-4616 | |
| | 105 | XPW-4617 | |
| | 106 | XPW-4618 | |
| | 107 | XPW-4619 | |
| | 108 | XPW-4620 | |
| | 109 | XPW-4621 | |
| | 110 | XPW-4622 | |
| | 111 | XPW-4623 | |
| | 112 | XPW-4626 | |
| | 113 | XPW-4628 | |
| | 114 | XPW-4630 | |
| | 115 | XPW-4631 | |
| | 116 | XPW-4632 | |
| | 117 | XPW-4633 | |
| | 118 | XPW-4636 | |
| | 119 | XPW-4637 | |
| | 120 | XPW-4639 | |
| | 121 | XPW-4644 | |
| | 122 | XPW-4646 | |
| | 123 | XPW-4647 | |

TABLE 87
Proliferation assay with HPEKp cells at 10 μM

| Activity Range | Entry | Compound No. | Specification |
|---|---|---|---|
| 1.0 ± 0.0 | 1 | DMSO | Baseline control |
| 0.9 ± 0.0 | 2 | MTREX | Control 20 μM |
| 0.8 < AVE_{w} ≤ 0.9 | 3 | XPW-3199 | |
| | 4 | XPW-3232 | |
| 0.7 < AVE_{w} ≤ 0.8 | 5 | XPW-2651 | |
| | 6 | XPW-3210 | |
| | 7 | XPW-3230 | |
| | 8 | XPW-3234 | |
| 0.6 < AVE_{w} ≤ 0.7 | 9 | XPW-2643 | |
| | 10 | XPW-2665 | |
| | 11 | XPW-2672 | |
| | 12 | XPW-3196 | |
| 0.4 < AVE_{w} ≤ 0.6 | 13 | XPW-1596 | |
| | 14 | XPW-3211 | |
| 0.2 < AVE_{w} ≤ 0.4 | 15 | XPW-0532 | |
| | 16 | XPW-0659 | |
| | 17 | XPW-2657 | |
| | 18 | XPW-3202 | |
| | 19 | XPW-3216 | |
| | 20 | XPW-3217 | |
| 0.2 ± 0.0 | 21 | RES | Control 20 μM |
| 0.2 ± 0.0 | 22 | RES | Control 40 μM |
| 0.0 ≤ AVE_{w} ≤ 0.2 | 23 | XPW-0516 | |
| | 24 | XPW-0518 | |
| | 25 | XPW-0529 | |
| | 26 | XPW-0530 | |
| | 27 | XPW-0544 | |
| | 28 | XPW-0546 | |
| | 29 | XPW-0660 | |
| | 30 | XPW-0663 | |
| | 31 | XPW-0669 | |
| | 32 | XPW-0672 | |
| | 33 | XPW-1582 | |
| | 34 | XPW-1610 | |
| | 35 | XPW-1727 | |
| | 36 | XPW-1736 | |
| | 37 | XPW-2646 | |
| | 38 | XPW-2660 | |
| | 39 | XPW-2671 | |
| | 40 | XPW-2787 | |
| | 41 | XPW-2788 | |
| | 42 | XPW-2791 | |
| | 43 | XPW-2792 | |
| | 44 | XPW-2797 | |
| | 45 | XPW-2800 | |
| | 46 | XPW-3193 | |
| | 47 | XPW-3194 | |
| | 48 | XPW-3200 | |
| | 49 | XPW-3201 | |
| | 50 | XPW-3203 | |
| | 51 | XPW-3206 | |
| | 52 | XPW-3209 | |
| | 53 | XPW-3212 | |
| | 54 | XPW-3213 | |
| | 55 | XPW-3214 | |
| | 56 | XPW-3218 | |
| | 57 | XPW-3219 | |
| | 58 | XPW-3223 | |
| | 59 | XPW-3224 | |
| | 60 | XPW-3225 | |
| | 61 | XPW-3226 | |
| | 62 | XPW-3231 | |
| | 63 | XPW-3233 | |

TABLE 88
Proliferation assay with HPEKp cells at 10 μM

| Activity Range | Entry | Compound No. | Specification |
|---|---|---|---|
| 1.0 ± 0.0 | 1 | DMSO | Baseline control |
| 0.9 ± 0.0 | 2 | MTREX | Control at 20 μM |
| 0.8 < AVE_{w} ≤ 0.9 | 3 | XPW-0509 | |
| | 4 | XPW-0510 | |
| | 5 | XPW-2633 | |
| | 6 | XPW-2652 | |
| | 7 | XPW-4544 | |
| | 8 | XPW-4546 | |
| | 9 | XPW-4558 | |
| | 10 | XPW-4718 | |
| | 11 | XPW-4723 | |
| | 12 | XPW-4843 | |
| 0.7 < AVE_{w} ≤ 0.8 | 13 | XPW-0523 | |
| | 14 | XPW-0538 | |
| | 15 | XPW-1601 | |
| | 16 | XPW-2661 | |
| | 17 | XPW-2662 | |
| | 18 | XPW-3195 | |
| | 19 | XPW-4543 | |
| | 20 | XPW-4547 | |
| | 21 | XPW-4548 | |
| | 22 | XPW-4549 | |
| | 23 | XPW-4551 | |
| | 24 | XPW-4555 | |
| | 25 | XPW-4645 | |
| 0.6 < AVE_{w} ≤ 0.7 | 26 | XPW-0533 | |
| | 27 | XPW-0534 | |
| | 28 | XPW-0537 | |
| | 29 | XPW-2634 | |
| | 30 | XPW-2637 | |
| | 31 | XPW-4556 | |
| | 32 | XPW-4560 | |
| | 33 | XPW-4563 | |
| | 34 | XPW-4564 | |
| | 35 | XPW-4566 | |
| | 36 | XPW-4567 | |
| | 37 | XPW-4715 | |
| 0.4 < AVE_{w} ≤ 0.6 | 38 | XPW-0506 | |
| | 39 | XPW-0520 | |
| | 40 | XPW-2648 | |
| | 41 | XPW-3208 | |
| | 42 | XPW-4559 | |
| | 43 | XPW-4562 | |
| | 44 | XPW-4568 | |
| | 45 | XPW-4714 | |
| 0.2 < AVE_{w} ≤ 0.4 | 46 | XPW-4565 | |
| | 47 | XPW-4569 | |
| 0.2 ± 0.0 | 48 | RES | Control at 20 μM |
| 0.2 ± 0.0 | 49 | RES | Control at 40 μM |

The data in Table 86 relate to novel compounds, wherein the data in Table 87 and Table 88 relate to a novel medical use of compounds disclosed in PCT/EP2018/054686.

In one embodiment, several compounds of the invention were found to inhibit the growth of C2C12 cells (murine myoblast cells) obtainable from the Deutsche Sammlung von Mikroorganismen und Zellkulturen GmbH (DSMZ) under the accession number ACC 565. C2C12 cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of C2C12 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of C2C12 cells. The so far identified C2C12 growth inhibitors relate to the compounds listed in Table 89, Table 90 and Table 91. The entries of Table 89, Table 90 and Table 91 are categorized by the corresponding weighted arithmetic means of the compounds without consideration of the respective standard deviations, hence falling into the activity ranges as indicated.

TABLE 89
Proliferation assay with C2C12 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0539 | |
| | 3 | XPW-0703 | |
| | 4 | XPW-0720 | |
| | 5 | XPW-0762 | |
| | 6 | XPW-3052 | |
| | 7 | XPW-4578 | |
| 0.8 ± 0.0 | 8 | RES | Control at 20 μM |
| 0.7 < AVE_{w} ≤ 0.8 | 9 | XPW-0784 | |
| | 10 | XPW-0916 | |
| | 11 | XPW-2806 | |
| | 12 | XPW-2833 | |
| | 13 | XPW-4575 | |
| | 14 | XPW-4591 | |
| | 15 | XPW-4638 | |
| 0.6 < AVE_{w} ≤ 0.7 | 16 | XPW-0574 | |
| | 17 | XPW-0667 | |
| | 18 | XPW-0728 | |
| | 19 | XPW-4580 | |
| | 20 | XPW-4583 | |
| | 21 | XPW-4592 | |
| | 22 | XPW-4612 | |
| 0.4 < AVE_{w} ≤ 0.6 | 23 | XPW-0790 | |
| | 24 | XPW-0832 | |
| | 25 | XPW-4585 | |
| | 26 | XPW-4586 | |
| | 27 | XPW-4588 | |
| | 28 | XPW-4589 | |
| 0.2 < AVE_{w} ≤ 0.4 | 29 | XPW-0770 | |
| | 30 | XPW-0798 | |
| | 31 | XPW-4594 | |
| | 32 | XPW-4595 | |
| | 33 | XPW-4637 | |
| 0.2 ± 0.1 | 34 | RES | Control at 40 μM |
| 0.1 ± 0.0 | 35 | MTREX | Control at 20 μM |
| 0.0 < AVE_{w} ≤ 0.2 | 36 | XPW-0661 | |
| | 37 | XPW-0665 | |
| | 38 | XPW-0674 | |
| | 39 | XPW-0675 | |
| | 40 | XPW-0678 | |
| | 41 | XPW-0679 | |
| | 42 | XPW-0686 | |
| | 43 | XPW-0700 | |
| | 44 | XPW-0714 | |
| | 45 | XPW-0734 | |
| | 46 | XPW-0742 | |
| | 47 | XPW-0924 | |
| | 48 | XPW-0930 | |
| | 49 | XPW-1750 | |
| | 50 | XPW-2805 | |
| | 51 | XPW-4584 | |
| | 52 | XPW-4587 | |
| | 53 | XPW-4590 | |
| | 54 | XPW-4593 | |
| | 55 | XPW-4613 | |
| | 56 | XPW-4614 | |
| | 57 | XPW-4616 | |
| | 58 | XPW-4617 | |
| | 59 | XPW-4618 | |
| | 60 | XPW-4619 | |
| | 61 | XPW-4620 | |
| | 62 | XPW-4621 | |
| | 63 | XPW-4622 | |
| | 64 | XPW-4623 | |
| | 65 | XPW-4625 | |
| | 66 | XPW-4626 | |
| | 67 | XPW-4628 | |
| | 68 | XPW-4630 | |
| | 69 | XPW-4631 | |
| | 70 | XPW-4632 | |
| | 71 | XPW-4633 | |
| | 72 | XPW-4636 | |
| | 73 | XPW-4639 | |
| | 74 | XPW-4640 | |
| | 75 | XPW-4644 | |
| | 76 | XPW-4646 | |
| | 77 | XPW-4647 | |

TABLE 90
Proliferation assay with C2C12 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0529 | |
| | 3 | XPW-0546 | |
| | 4 | XPW-3223 | |
| 0.8 ± 0.0 | 5 | RES | Control 20 μM |
| 0.7 < AVE_{w} ≤ 0.8 | 6 | XPW-0544 | |
| | 7 | XPW-2658 | |
| | 8 | XPW-2660 | |
| | 9 | XPW-3217 | |
| 0.6 < AVE_{w} ≤ 0.7 | 10 | XPW-0530 | |
| 0.4 < AVE_{w} ≤ 0.6 | 11 | XPW-3202 | |
| | 12 | XPW-3216 | |
| 0.2 ± 0.1 | 13 | RES | Control 40 μM |
| 0.1 ± 0.0 | 14 | MTREX | Control 20 μM |
| 0.0 ≤ AVE_{w} ≤ 0.2 | 15 | XPW-0659 | |
| | 16 | XPW-0660 | |
| | 17 | XPW-0663 | |
| | 18 | XPW-0664 | |
| | 19 | XPW-0669 | |
| | 20 | XPW-0670 | |
| | 21 | XPW-0672 | |
| | 22 | XPW-1727 | |
| | 23 | XPW-1728 | |
| | 24 | XPW-1736 | |
| | 25 | XPW-2787 | |
| | 26 | XPW-2788 | |
| | 27 | XPW-2791 | |
| | 28 | XPW-2792 | |
| | 29 | XPW-2797 | |
| | 30 | XPW-2798 | |
| | 31 | XPW-3193 | |
| | 32 | XPW-3194 | |
| | 33 | XPW-3197 | |
| | 34 | XPW-3200 | |
| | 35 | XPW-3201 | |
| | 36 | XPW-3203 | |
| | 37 | XPW-3206 | |
| | 38 | XPW-3209 | |
| | 39 | XPW-3212 | |
| | 40 | XPW-3213 | |
| | 41 | XPW-3214 | |
| | 42 | XPW-3218 | |
| | 43 | XPW-3219 | |
| | 44 | XPW-3221 | |
| | 45 | XPW-3225 | |
| | 46 | XPW-3226 | |
| | 47 | XPW-3227 | |
| | 48 | XPW-3231 | |
| | 49 | XPW-3233 | |

TABLE 91
Proliferation assay with C2C12 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPW-0520 | |
| | 3 | XPW-0524 | |
| | 4 | XPW-0534 | |
| | 5 | XPW-0538 | |
| | 6 | XPW-1602 | |
| | 7 | XPW-2648 | |
| | 8 | XPW-3232 | |
| | 9 | XPW-3234 | |
| | 10 | XPW-4545 | |
| | 11 | XPW-4562 | |
| | 12 | XPW-4565 | |
| | 13 | XPW-4567 | |
| | 14 | XPW-4569 | |
| 0.8 ± 0.0 | 15 | RES | Control at 20 μM |
| 0.7 < AVE_{w} ≤ 0.8 | 16 | XPW-4645 | |
| 0.4 < AVE_{w} ≤ 0.6 | 17 | XPW-0533 | |
| 0.2 ± 0.1 | 18 | RES | Control at 40 μM |
| 0.1 ± 0.0 | 19 | MTREX | Control at 20 μM |

The data in Table 89 relate to novel compounds, wherein the data in Table 90 and Table 91 relate to a novel medical use of compounds disclosed in PCT/EP2018/054686.

In certain embodiments, compounds of the present invention may be modulators, in particular enhancers of Notch signalling.

The communication between cells via Notch signaling (reviewed in Kopan et al., Cell 2009, 137, 216-233; Bray, Nat. Rev. Mol. Cell Biol. 2016, 17, 722-735) is in the first step mediated by two types of transmembrane proteins: The Notch receptors being distributed across the cell membrane of the signal-receiving cell and the Notch ligands covering the membrane of the signal-sending cell. Mechanistically, Notch signaling is activated by receptor-ligand interaction, which leads to the proteolytic release of the intracellular domain (NICD) of the membrane bound Notch receptor into the inside of the signal-receiving cell. Subsequent translocation of NICD into the nucleus in turn leads to the transcriptional activation of certain and cell type specific genes. The Notch-mediated alteration of the previous gene-expression program of a cell is manifested in according cellular changes, which represent the response of the cell to a Notch signal.

The activation level of Notch signaling can be quantified in vitro reliably by measuring the expression levels of Notch specific target genes. This can be accomplished by the quantification of corresponding mRNA or protein of a particular Notch target gene. Alternatively, cells can be genetically modified to carry a luciferase gene as an artificial Notch target gene, which is expressed in dependence of Notch activity. In this setting, Notch signaling levels can be quantified by measuring the luciferase-derived bioluminescence values.

An according Notch-reporter assay, i.e. a luciferase-based luminescence readout, was used here to quantify the ability of the claimed compounds to augment Notch signaling in a cellular system. For this purpose, HeLa cells, obtainable from the American Type Culture Collection (ATCC) under the accession number ATCC-CCL-2, were transiently transfected for 24 hours using FuGENE® HD (Promega, #E2311) as transfection reagent with expression vectors of a membrane-tethered form of the constitutively active intracellular domain of the human Notch1 receptor (hNotch1ΔE) to activate the Notch signaling cascade (BPS Bioscience, customized human analogue to Notch Pathway Reporter Kit #60509 component C), a Firefly luciferase being expressed under the control of a Notch-responsive promoter to monitor Notch signaling (BPS Bioscience, Notch Pathway Reporter Kit #60509, CSL luciferase reporter vector from component A not premixed with Renilla luciferase vector), and a Renilla luciferase being constitutively expressed in a Notch signaling independent manner to include a measure for the cell number per sample (Promega, pRL-SV40, #E2231). HeLa cells were cultivated in DMEM medium (Fisherscientific, #11584456) containing 10% fetal bovine serum (Fisherscientific, #15517589). The transfection was carried out in a 100 mm-culture dish (StarLab, #CC7682-3394) with cells being properly attached to the plate at a cell confluency of 80-90% in a total volume of 7 mL culture medium. Per dish to be transfected, a transfection mix was prepared by adding to 238 μL Opti-MEM (Fisherscientific, #10149832) 40 μL of the hNotch1ΔE expression vector (100 ng/μL), 80 μL of the CSL luciferase reporter vector (40 ng/μL), 4 μL of the pRL-SV40-Renilla luciferase vector (10 ng/μL), and in the last step 18.1 μL of FuGENE® HD. After addition of FuGENE® HD the transfection mix was let stand for 15 min at room temperature and hereafter equally distributed into the culture dish. After 24 hours of transfection, the transfected cells were carefully detached from the dish using 0.5 mM EDTA in PBS and seeded into 96-well plates suitable for luminescence readouts (CORNING, #3610) at 10′000 cells per well. The cells were then incubated with the test-compounds at a final concentration of 10 μM (diluted from 10 mM stock-solutions in DMSO to a final DMSO concentration of 0.1% v/v in H₂O (Water For Injection, WFI, Fisherscientific #10378939)) or with the empty carrier DMSO at 0.1% v/v as control for 20 hours. Hereafter, the cells were washed once with PBS and then lysed with 30 μL per well of Passive Lysis Buffer (Promega, #E194A, component of Dual-Luciferase® Reporter Assay System, #E1910) by gently shaking the plates for 20 min at room temperature with an orbital plate shaker. Directly after the lysis, first the Firefly and then the Renilla luciferase values were measured consecutively from the same well with a luminescence reader immediately after applying 15 μL per well each of the corresponding enzyme substrates needed to create the luminescence signals (Promega, Dual-Luciferase® Reporter Assay System, #E1910).

The suitability of the assays for monitoring Notch signaling was controlled by additionally including a generally accepted commercial Notch inhibitor, i.e. DAPT, as negative control, as well as the reported Notch enhancer resveratrol (RES) as positive control (Pinchot et al., Cancer 2011, 117, 1386-1398; Truong et al., Ann. Surg. Oncol. 2011, 18, 1506-1511; Yu et al., Mol. Cancer Ther. 2013, 12, 1276-1287). Both control compounds were likewise tested at 10 μM.

Per single experiment the measurement was performed in six replicates per compound. For every compound, this experiment was repeated in three or more independent replicates. The values of the Notch-reporter luciferase were normalized by division through the corresponding individual Notch-independent Renilla values in order to eliminate the impact of variation in the absolute cell numbers in between the samples. For every individual plate, a second normalization was performed against the equally weighted arithmetic mean (here abbreviated as AVE) of the six associated Renilla-normalized DMSO-control values within a single experiment in order to obtain the relative values to a baseline level of 1.0. The statistical calculations were performed in analogy to the proliferation assay as described above. To this end, two independent outlier analyses were performed according to the methods by Peirce and Chauvenet (Ross, Journal of Engineering Technology 2003, 1-12). Outliers confirmed by at least one of the methods were excluded from the calculations but not more than one value out of six per compound within a single experiment. The weighted arithmetic mean AVE_{w} for each compound was calculated from the double-normalized values over all independent replicates of the single experiments comprising the six replicates each. The corresponding standard deviation for the weighted arithmetic mean was calculated according to the method described by Bronstein et al. (Bronstein, Semendjajew, Musiol, Mühlig, Taschenbuch der Mathematik, 5^{th} edition 2001 (German), publisher: Verlag Harri Deutsch, Frankfurt am Main and Thun) and was combined with the Gauß' error propagation associated with the performed calculation for the normalization. The resulting standard deviation is herein referred to as “combined standard deviation”.

In cases with considerable variation in the double-normalized equally weighted arithmetic means derived from three independent replicates, the number of independent replicates was increased to four or more. In the cases of four or more independent replicates, a second-line outlier analysis was applied on all double-normalized equally weighted arithmetic means according to the methods by Peirce and Chauvenet as described above.

A compound is considered as a Notch signaling augmenting molecule, i.e. an enhancer of Notch signaling, if the weighted arithmetic mean of the luminescence values after subtraction of the corresponding combined standard deviation amounts to 1.1 or higher, in particular to 1.2 or higher, 1.3 or higher, 1.4 or higher, 1.5 or higher, 1.7 or higher, and 2.0 or higher relative to the overall basis level of 1.0. The overall basis level was calculated as the weighted arithmetic mean of all double-normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ib) and (Ic), respectively, have been identified as enhancers of Notch signaling. The so far identified Notch enhancers relate to the compounds listed in Table 92. The entries of Table 92 are categorized by the corresponding weighted arithmetic mean of the compounds without consideration of the respective standard deviations, hence falling into the activity ranges as indicated.

TABLE 92
Notch reporter assay

| Activity Range | Entry | Compound No. | Specification |
|---|---|---|---|
| 2.0 ≤ AVEw | 1 | XPW-0314 | |
| | 2 | XPW-0566 | |
| | 3 | XPW-0574 | |
| | 4 | XPW-0665 | |
| | 5 | XPW-0686 | |
| | 6 | XPW-0700 | |
| | 7 | XPW-0702 | |
| | 8 | XPW-0716 | |
| | 9 | XPW-0717 | |
| | 10 | XPW-0734 | |
| | 11 | XPW-0742 | |
| | 12 | XPW-2904 | |
| | 13 | XPW-2918 | |
| | 14 | XPW-4587 | |
| | 15 | XPW-4593 | |
| | 16 | XPW-4612 | |
| | 17 | XPW-4614 | |
| | 18 | XPW-4617 | |
| | 19 | XPW-4618 | |
| | 20 | XPW-4619 | |
| | 21 | XPW-4621 | |
| | 22 | XPW-4622 | |
| | 23 | XPW-4623 | |
| | 24 | XPW-4631 | |
| | 25 | XPW-4632 | |
| | 26 | XPW-4637 | |
| | 27 | XPW-4639 | |
| | 28 | XPW-4646 | |
| | 29 | XPW-4647 | |
| 1.9 ± 0.5 | 30 | RES | Control at 10 μM |
| 1.7 ≤ AVEw < 2.0 | 31 | XPW-0679 | |
| 1.4 ≤ AVEw < 1.7 | 32 | XPW-0703 | |
| | 33 | XPW-0706 | |
| | 34 | XPW-0720 | |
| | 35 | XPW-0728 | |
| | 36 | XPW-0790 | |
| | 37 | XPW-2806 | |
| | 38 | XPW-2848 | |
| | 39 | XPW-4603 | |
| | 40 | XPW-4605 | |
| | 41 | XPW-4616 | |
| | 42 | XPW-4638 | |
| | 43 | XPW-0535 | |
| | 44 | XPW-0674 | |
| | 45 | XPW-0675 | |
| | 46 | XPW-0798 | |
| | 47 | XPW-2847 | |
| | 48 | XPW-4628 | |
| 1.3 ≤ AVEw < 1.4 | 49 | XPW-0608 | |
| | 50 | XPW-0661 | |
| | 51 | XPW-0916 | |
| | 52 | XPW-0930 | |
| | 53 | XPW-2805 | |
| | 54 | XPW-4640 | |
| | 55 | XPW-4644 | |
| 1.2 ≤ AVEw < 1.3 | 56 | XPW-0616 | |
| | 57 | XPW-0667 | |
| | 58 | XPW-0770 | |
| | 59 | XPW-0776 | |
| | 60 | XPW-0784 | |
| | 61 | XPW-0924 | |
| | 62 | XPW-2834 | |
| | 63 | XPW-4594 | |
| | 64 | XPW-4630 | |
| 1.0 ± 0.0 | 65 | DMSO | Baseline control |
| 0.1 ± 0.0 | 66 | DAPT | Control at 10 μM |

Several other molecules have not been identified as enhancers of Notch signaling according to the above method.

In some cases, the growth inhibiting properties correlate with Notch enhancing properties, in other cases the growth inhibiting properties do not correlate with Notch enhancing properties.

The biological activity of the claimed compounds can be attributed to but may not be limited to Notch signaling enhancing activity. The Notch regulating properties of the claimed compounds can be used alternatively or in combination with the mechanisms leading to antiproliferative effects in medicinal treatments, preferably in the treatment of hyperproliferative disorders including cancer and non-malignant hyperproliferative disorders.

In one aspect, the present invention relates to the treatment of skin, skin appendages, mucosa, mucosal appendages, cornea, and all kinds of epithelial tissue. The term “skin” relates to tissue including epidermis and dermis. The term “mucosa” relates to mucous and submucous tissues including oral mucosa, nasal mucosa, ocular mucosa, mucosa of the ear, respiratory mucosa, genital mucosa, urothelial mucosa, anal mucosa and rectal mucosa. The term “appendages” relates to tissue including hair follicles, hair, fingernails, toenails and glands including sebaceous glands, sweat glands, e.g. apocrine or eccrine sweat glands and mammary glands.

In one embodiment, the present invention relates to treatment of non-melanoma skin cancer and pre-cancerous lesions, such as basal cell carcinoma (BCC), squamous cell carcinoma (SCC), sebaceous gland carcinoma, Merkel cell carcinoma, angiosarcoma, cutaneous B-cell lymphoma, cutaneous T-cell lymphoma, dermatofibrosarcoma, actinic keratosis (AK) or Bowen's disease (BD), and cancer and pre-cancerous lesions of other squamous epithelia e.g. cutaneous SCC, lung SCC, head and neck SCC, oral SCC, tongue SCC, esophageal SCC, cervical SCC, periocular SCC, SCC of the thyroid, SCC of the penis, SCC of the vagina, SCC of the prostate and SCC of the bladder.

In a further embodiment, the present invention relates to the treatment of skin and mucosal disorders with cornification defects (keratoses) and/or abnormal keratinocyte proliferation, such as Psoriasis, Darier's disease, Lichen planus, Lupus erythematosus, Ichthyosis or Verruca vulgaris (senilis).

In a further embodiment, the invention relates to the treatment of skin and mucosal diseases, and skin and mucosal cancer each related to and/or caused by viral infections, such as warts, and warts related to HPV (human papilloma virus), papillomas, HPV-related papillomas, papillomatoses and HPV-related papillomatoses, e.g. Verruca (plantar warts), Verruca plana (flat warts/plane warts), Verruca filiformis (filiform warts), mosaic warts, periungual warts, subungual warts, oral warts, genital warts, fibroepithelial papilloma, intracanalicular papilloma, intraductal papilloma, inverted papilloma, basal cell papilloma, squamous papilloma, cutaneous papilloma, fibrovasular papilloma, plexus papilloma, nasal papilloma, pharyngeal papilloma, Papillomatosis cutis carcinoides, Papillomatosis cutis lymphostatica, Papillomatosis confluens et reticularis or laryngeal papillomatosis (respiratory papillomatosis), Herpes-related diseases, e.g. Herpes labialis, Herpes genitalis, Herpes zoster, Herpes corneae or Kaposi's sarcoma and HPV-related cancer of the cervix, vulva, penis, vagina, anus, oropharynx, tongue and oral cavity.

In a further embodiment, the invention relates to the treatment of atopic dermatitis.

In a further embodiment, the invention relates to the treatment of acne.

In a further embodiment, the invention relates to the treatment of wounds of the skin, wherein the process of wound healing is accelerated.

In a further embodiment, the invention relates to the treatment of cancer related to and/or caused by viral infections, i.e. oncoviral infections, e.g. cancer related to HBV- and HCV (hepatitis virus B and C) such as liver cancer, cancer related to EBV (Epstein-Barr virus) such as Burkitt lymphoma, Hodgkin's and non-Hodgkin's lymphoma and stomach cancer, cancer related to HPV (human papilloma virus) such as cervical cancer, cancer related to HHV (human herpes virus) such as Kaposi's sarcoma, and cancer related to HTLV (human T-lymphotrophic virus) such as T-cell leukemia and T-cell lymphoma.

A further aspect of the present invention relates to the treatment of immune system-related disorders. The term “immune system-related disorders” as used herein applies to a pathological condition of the haematopoietic system including the haematologic system, in particular a pathological condition of immune cells belonging to the innate or adaptive immune system.

A further aspect of the present invention relates to the therapeutic use in immune system-related applications. The term “immune system-related application” as used herein applies to the intervention into proliferation, differentiation and/or activation of cell lineages of the haematopoietic system including the haematologic system in order to modulate an immune response (immune modulation). The term “immune system-related application” as used herein also applies to the intervention into the cellular and non-cellular microenvironment of sites of action of immune cells in order to support and/or enable immune cells in their performance. In particular, the interventions as here defined with the term “immune system-related application” relate to immune cells belonging to the innate or adaptive immune system.

Thus, the compounds of the invention may be used in immunotherapy, alone or together with other immunotherapeutic methods or compounds, as immunologic adjuvant, e.g. as vaccine adjuvant, or as adjuvant for immunotherapy. The term “immunotherapy” as used herein applies to activation-immunotherapy in patients without immune deficiency or with acquired or congenital immune deficiency, and as immune recovery to enhance the functionality of the immune system in the response against pathogens or pathologically transformed endogenous cells, such as cancer cells.

The term “other immunotherapy methods” as used herein applies to vaccinations, antibody treatment, cytokine therapy, the use of immune checkpoint inhibitors and immune response-stimulating drugs, as well as to autologous transplantations of genetically modified or non-modified immune cells, which may be stimulated with intercellular signals, or signaling molecules, or antigens, or antibodies, i.e. adoptive immune-cell transfer.

The method of use of the present invention in immune system-related applications and other immunotherapy methods relates to the use in vivo, in vitro, and ex vivo, respectively.

Specific examples are activation and/or enhancement of activation of peripheral T-lymphocytes, including T-helper cells and cytotoxic T-cells, in order to amplify an immune response, particularly the stimulation of proliferation and/or production and/or secretion of cytokines and/or cytotoxic agents upon antigen recognition in order to amplify an immune response, such as the activation and/or enhancement of activation of B-lymphocytes in order to amplify an immune response, particularly the stimulation of proliferation and/or antibody production and/or secretion, such as the enhancement of an immune response through augmentation of the number of specific immune-cell subtypes, by regulation of differentiation and/or cell fate decision during immune-cell development, as for example to regulate, particularly to augment the number of immune cells belonging to the T- and B-cell lineage, including marginal zone B-cells, cytotoxic T-cells or T-helper (Th) subsets in particular Th1, Th2, Th17 and regulatory T-cells; or the use as immunologic adjuvant such as vaccine adjuvant.

A still further aspect of the invention relates to the treatment of muscular diseases including diseases of skeletal muscle, cardiac muscle and smooth muscle.

In one embodiment, the invention relates to the treatment of muscular dystrophies (MD).

Specific examples are Duchenne MD, Becker MD, congenital MD, Limb-Girdle MD, facioscapulohumeral MD, Emery-Dreifuss MD, distal MD, myotonic MD or oculopharyngeal MD.

In a further embodiment, the invention relates to the treatment of hyperproliferative disorders of the muscle, including myoblastoma, rhabdomyoma, and rhabdomyosarcoma, as well as muscle hyperplasia and muscle hypertrophy.

In a further embodiment, the compounds of the invention may be used for muscle regeneration after pathologic muscle degeneration or atrophy, e.g. caused by traumata, caused by muscle ischemia or caused by inflammation, in aging-related muscle-atrophy or in disease-related muscle atrophy such as myositis and fibromyositis or poliomyelitis.

A still further aspect relates to the treatment of disorders of the neuroendocrine system such as cancer of the neuroendocrine system, comprising neuroendocrine small cell carcinomas, neuroendocrine large cell carcinomas and carcinoid tumors, e.g. of the brain, thyroid, pancreas, gastrointestinal tract, liver, esophagus, and lung, such as neuroendocrine tumor of the pituitary gland, neuroendocrine tumor of the adrenal gland, medullary thyroid cancer (MTC), C-cell hyperplasia, anaplastic thyroid cancer (ATC), parathyroid adenoma, intrathyroidal nodules, insular carcinoma, hyalinizing trabecular neoplasm, paraganglioma, lung carcinoid tumors, neuroblastoma, gastrointestinal carcinoid, Goblet-cell carcinoid, pancreatic carcinoid, gastrinoma, glucagenoma, somatostatinoma, VIPoma, insulinoma, non-functional islet cell tumor, multiple endocrine neoplasia type-1, or pulmonary carcinoid.

A still further aspect relates to the treatment of disorders of the lung such as cancer of the lung, comprising small-cell lung cancer (SCLC) and non-small-cell lung cancer (NSCLC), including lung squamous cell carcinoma, lung adenocarcinoma and lung large cell carcinoma.

A still further aspect relates to the treatment of cancers or precancerous lesions of the brain, pancreas, breast, ovaries, liver, thyroid, genitourinary tract, gastrointestinal tract, and endothelial tissue, including glioma, mixed glioma, glioblastoma multiforme, astrocytoma, anaplastic astrocytoma, glioblastoma, oligodendroglioma, anaplastic oligodendroglioma, anaplastic oligoastrocytoma, ependymoma, anaplastic ependymoma, myxopapillary ependymoma, subependymoma, brain stem glioma, optic nerve glioma, and forebrain tumors, pancreatic adenocarcinoma, pancreatic ductal adenocarcinoma, pancreatic acinar cell carcinoma, pancreatic pseudopapillary neoplasm, pancreatic intraductal papillary-mucinous neoplasm, pancreatic mucinous cystadenocarcinoma, pancreatoblastoma and pancreatic intraepithelial neoplesia, hepatocellular carcinoma, fibrolamellar hepatocellular carcinoma, papillary thyroid cancer and follicular thyroid cancer, cervical cancer, hormone receptor-positive breast cancer and hormone receptor-negative breast cancer, ovarian cancer, gastric cancer and angiosarcoma.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease. The term “treating” also encompasses post-treatment care.

In some embodiments, administration of a compound of the invention, or pharmaceutically acceptable salt thereof, is effective in preventing the disease; for example, preventing a disease, condition or disorder in an individual who may be predisposed to the disease, condition or disorder but does not yet experience or display the pathology or symptomatology of the disease.

The compounds of the invention may be used in human and veterinary medicine, which includes the treatment of companion animals, e.g. horses, dogs, cats, rabbits, guinea pigs, fishes e.g. koi, birds e.g. falcon; and livestock, e.g. cattle, poultry, pig, sheep, goat, donkey, yak and camel.

Pharmaceutical Compositions

The present invention further provides pharmaceutical compositions comprising a compound as described herein or a pharmaceutically acceptable salt thereof for use in medicine, e.g. in human or veterinary medicine. In some embodiments, the composition further comprises a pharmaceutically acceptable carrier.

An effective dose of the compounds according to the invention, or their salts, solvates or prodrugs thereof is used, in addition to physiologically acceptable carriers, diluents and/or adjuvants for producing a pharmaceutical composition. The dose of the active compounds can vary depending on the route of administration, the age and weight of the patient, the nature and severity of the diseases to be treated, and similar factors. The daily dose can be given as a single dose, which is to be administered once, or be subdivided into two or more daily doses, and is as a rule 0.001-2000 mg. Particular preference is given to administering daily doses of 0.1-500 mg, e.g. 0.1-100 mg.

Suitable administration forms are topical or systemical including enteral, oral, rectal, and parenteral, as infusion and injection, intravenous, intra-arterial, intraperitoneal, intramuscular, intracardial, epidural, intracerebral, intracerebroventricular, intraosseous, intra-articular, intraocular, intravitreal, intrathecal, intravaginal, intracavernous, intravesical, subcutaneous, intradermal, transdermal, transmucosal, inhalative, intranasal, buccal, sublingual and intralesional preparations. Particular preference is given to using oral, parenteral, e.g. intravenous or intramuscular, intranasal preparations, e.g. dry powder or sublingual, of the compounds according to the invention. The customary galenic preparation forms, such as tablets, sugar-coated tablets, capsules, dispersible powders, granulates, aqueous solutions, alcohol-containing aqueous solutions, aqueous or oily suspensions, gels, hydrogels, ointments, creams, lotions, shampoos, lip balms, mouthwashs, foams, pastes, tinctures, dermal patches and tapes, forms in occlusion or in combination with time release drug delivery systems, with electrophoretic dermal delivery systems including implants and devices, and with jet injectors, liposome and transfersome vesicles, vapors, sprays, syrups, juices or drops and eye drops, can be used.

Solid medicinal forms can comprise inert components and carrier substances, such as calcium carbonate, calcium phosphate, sodium phosphate, lactose, starch, mannitol, alginates, gelatine, guar gum, magnesium stearate, aluminium stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavourings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators. Examples of such additives are tartrate and citrate buffers, ethanol and sequestering agents (such as ethylenediaminetetraacetic acid and its non-toxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable, synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or water-miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Cellulose ethers which can dissolve or swell both in water or in organic solvents, such as hydroxypropylmethyl cellulose, methyl cellulose or ethyl cellulose, or soluble starches, can be used as film-forming agents.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na-N-lauryl-β-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

As indicated above, the compounds of the invention may be administered as a combination therapy, as sequence therapy or as simultaneous combination therapy, with further active agents, e.g. therapeutically active compounds useful in the treatment of the above indicated disorders. These therapeutically active compounds may include but are not limited to chemotherapeutic agents such as nucleoside and nucleobase analogs, e.g. Cytarabin, Gemcitabine, Azathioprine, Mercaptopurine, Fluorouracil, Thioguanine, Azacitidine, Capecitabine, Doxifluridine; such as platinum-based drugs, e.g. Cisplatin, Oxaliplatin, Carboplatin and Nedaplatin; such as anthracyclines, e.g. Doxorubicin, Epirubicin, Valrubicin, Idarubicin, Daunorubicin, Sabarubicin, Pixantrone and Mitoxantrone; such as peptide antibiotics, e.g. Actinomycin and Bleomycin; such as alkylating agents e.g. Mechlorethamine, Chlorambucil, Melphalan, Nitrosoureas, Dacarbazine, Temozolomide and Cyclophosphamide; such as antimitotic agents including taxanes and vinca alkaloids, e.g. Docetaxel, Paclitaxel, Abraxane, Cabazitaxel, Vinblastine, Vindesine, Vinorelbine and Vincristine; such as topoisomerase inhibitors, e.g. Irinotecan, Topotecan, Teniposide and Etoposide; such as other cytostatic agents e.g. Hydroxyurea and Methotrexate; such as proteasome inhibitors, e.g Bortezomib, Ixazomib; and other targeted therapeutic agents such as kinase inhibitors, cell cycle inhibitors, regulators i.e. inhibitors and activators of signaling pathways including growth factor signaling, cytokine signaling, NF-kappaB signaling, AP1 signaling, JAK/STAT signaling, EGFR signaling, TGF-beta signaling, Notch signaling, Wnt signaling, Hedgehog signaling, hormone and nuclear receptor signaling, e.g. Erlotinib, Lapatinib, Dasatinib, Imatinib, Afatinib, Vemurafenib, Dabrafenib, Nilotinib, Cetuximab, Trametinib, Palbociclib, Cobimetinib, Cabozantinib, Pegaptanib, Crizotinib, Olaparib, Panitumumab, Cabozantinib, Ponatinib, Regorafenib, Entrectinib, Ranibizumab, Ibrutinib, Trastuzumab, Rituximab, Alemtuzumab, Gefitinib, Bevacizumab, Lenvatinib, Bosutinib, Axitinib, Pazopanib, Everolimus, Temsirolimus, Ruxolitinib, Tofacitinib, Sorafenib, Sunitinib, Aflibercept, Vandetanib; Vismodegib and Sonidegib; retinoids such as retinol, tretinoin, isotretinoin, alitretinoin, bexarotene, tazarotene, acitretin, adapalene and etretinate; hormone signaling modulators including estrogen receptor modulators, androgen receptor modulators and aromatase inhibitors e.g. Raloxifene, Tamoxifen, Fulvestrant, Lasofoxifene, Toremifene, Bicalutamide, Flutamide, Anastrozole, Letrozole and Exemestane; histone deacetylase inhibitors, e.g. Vorinostat, Romidepsin, Panobinostat, Belinostat and Chidamide; and Ingenol mebutate; and other Notch enhancers not encompassed by the compounds of the present invention, e.g. Valproic acid, Resveratrol, hesperetin, chrysin, phenethyl isothiocyanate, thiocoraline, N-methylhemeanthidine chloride and Notch Signaling-activating peptides or antibodies; and immune response modulating agents including immune checkpoint inhibitors e.g. Imiquimod, Ipilimumab, Atezolizumab, Ofatumumab, Rituximab, Nivolumab and Pembrolizumab; and anti-inflammatory agents including glucocorticoids and non-steroidal anti-inflammatory drugs, e.g. cortisol-based preparations, Dexamethason, Betamethason, Prednisone, Prednisolone, Methylprednisolone, Triamcinolon-hexacetonid, Mometasonfuroat, Clobetasolpropionat, acetylsalicylic acid, salicylic acid and other salicylates, Diflunisal, Ibuprofen, Dexibuprofen, Naproxen, Fenoprofen, Ketoprofen, Dexketoprofen, Loxoprofen, Flurbiprofen, Oxaprozin, Indomethacin, Ketorolac, Tolmetin, Diclofenac, Etodolac, Aceclofenac, Nabumetone, Sulindac, Mefenamic acid, Meclofenamic acid, Flufenamic acid, Tolfenamic acid, Celecoxib, Parecoxib, Etoricoxib and Firocoxib; and ACE inhibitors; and beta-blockers; and myostatin inhibitors; and PDE-S inhibitors; and antihistamines. For a combination therapy, the active ingredients may be formulated as compositions containing several active ingredients in a single dose form and/or as kits containing individual active ingredients in separate dose forms. The active ingredients used in combination therapy may be co-administered or administered separately.

The compounds of the invention may be administered as antibody-drug conjugates.

The compounds of the invention may be administered in combination with surgery, cryotherapy, electrodessication, radiotherapy, photodynamic therapy, laser therapy, chemotherapy, targeted therapy, immunotherapy, gene therapy, antisense therapy, cell-based transplantation therapy, stem cell therapy, physical therapy and occupational therapy.

Chemical Synthesis

### Abbreviations

Ac Acetyl

aq Aqueous

Alk Alkyl

Bn Benzyl

BRSM Based on Recovered Starting Material (yield)

Bu Butyl

mCPBA meta chloroperoxybenzoic acid

NMR Nuclear Magnetic Resonance Spectroscopy

DCE 1,2-dichloroethane

DCM Dichloromethane

DIBAL-H Diisobutylaluminium hydride

DMF N,N-dimethylformamide

DMSO Dimethyl sulfoxide

DMAP Dimethylaminopyridine

Equiv Equivalent

ESI Electron Spray Ionization

EDC N-(3dimethylaminopropyl)-N′-ethylcarbodimide

HOBt 1-hydroxybenzotriazole

HATU Hexafluorophosphate azabenzotriazole tetramethyl uronium

Et Ethyl

LiHMDS Lithium bis(trimetylsilyl)amide

Me Methyl

Ms Methanesulfonyl

PE Petroleum Ether

PG Protecting group

PTSA p-Toluenesulfonic acid

sat Saturated

TBAF Tetrabutylammonium Fluoride

Tf Trifluoromethanesulfonyl

THF Tetrahydrofuran

TLC Thin Layer Chromatography

TMS Trimethylsilyl

Ts p-Toluenesulfonyl

UV Ultraviolet

General Considerations

The compounds listed in Table 93 and Table 94 have been identified by TLC using pre-coated silica TLC sheets and common organic solvents such as petroleum ether, ethyl acetate, dichloromethane, methanol, toluene, triethylamine or acetic acid as eluent, preferably as binary or tertiary solvent mixtures thereof. UV light at a wavelength of 254 or 366 nm, and/or common staining solutions such as phosphomolybdic acid, potassium permanganate, or ninhydrin were used to visualize the compounds. Reactions were also monitored for completion this way. Reactions were run under inert atmosphere unless otherwise stated. Dry solvents were used wherever required. All reactions were stirred using a stir plate and magnetic stir bar.

The compounds listed in Table 93 have furthermore been identified by mass spectrometry using formic acid in the mobile phase for detection of positive ions, while no additive was used for negative ions. Ammonium Carbonate was used if the molecule was difficult to ionize in negative mode. Representative compounds and those which showed poor ionization in mass spectrometry were also identified by nuclear magnetic resonance spectroscopy (Table 94). Chemical shifts (6) were reported in parts per million (ppm) relative to residual solvent peaks rounded to the nearest 0.01 ppm for proton and 0.1 ppm for carbon (ref.: CHCl₃ [¹H: 7.26 ppm, ¹³C: 77.2 ppm], DMSO [¹H: 2.50 ppm, ¹³C: 39.5 ppm]). Coupling constants (J) were reported in Hz to the nearest 0.1 Hz. Peak multiplicity was indicated as follows: s (singlet), d (doublet), t (triplet), q (quartet), hept (heptet), m (multiplet), and br (broad).

Synthesis of Described Compounds:

The aforementioned compounds of the invention falling under the scope of formula I can be synthesized and purified by those persons skilled in the art and are preferably synthesized according to the general procedures (A to R) mentioned herein as illustrated in Scheme 1.

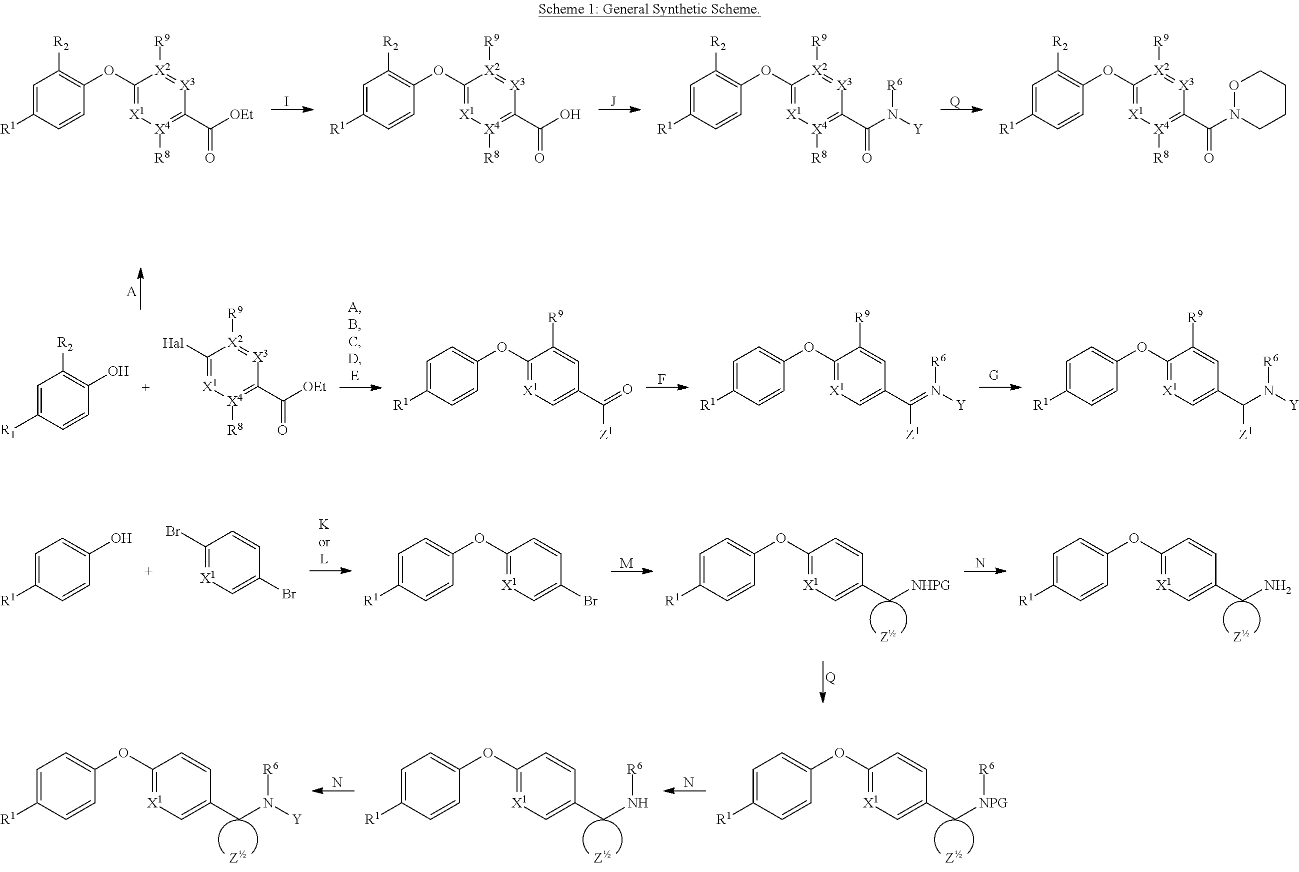

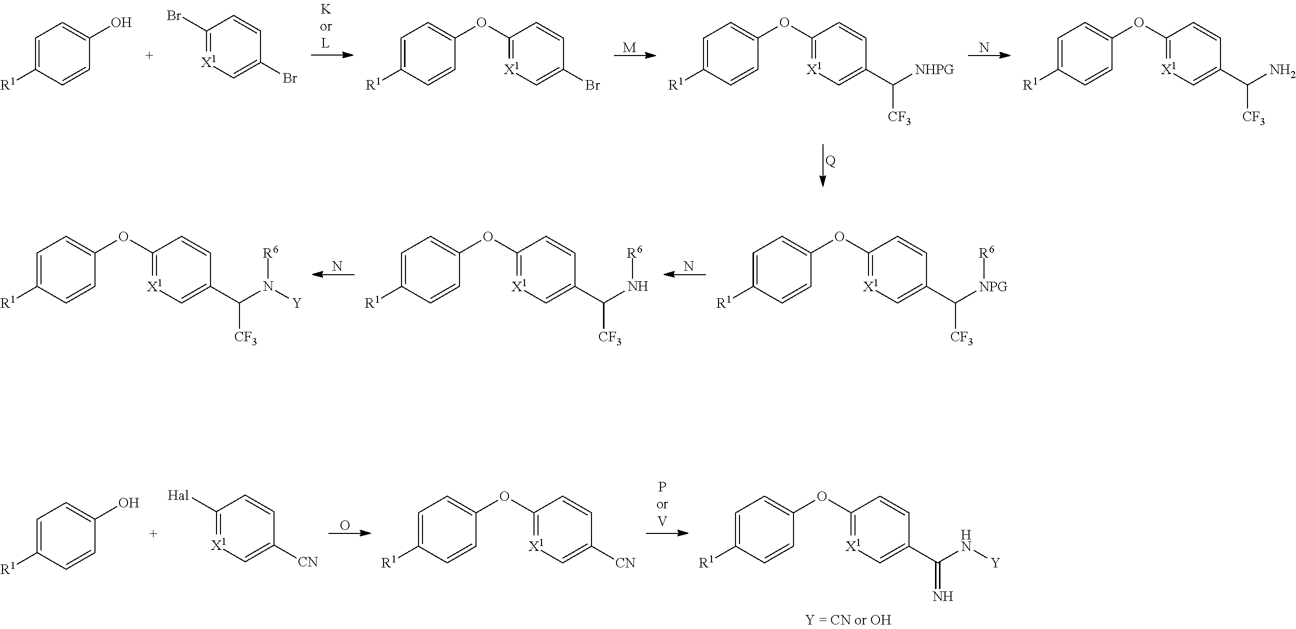

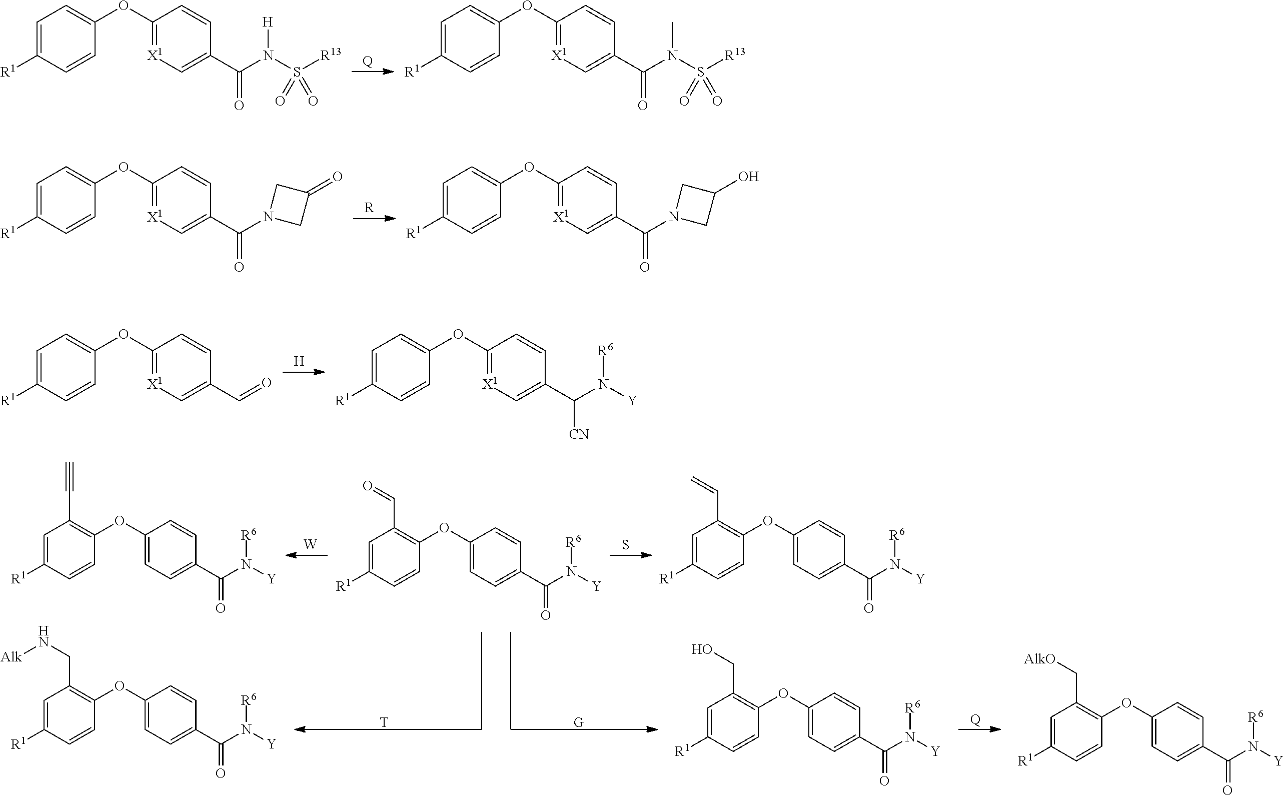

A) To the corresponding mono or bisubstituted phenol (1.0-1.5 equiv) and 4-alkyl ester halo(hetero)aryl (1 equiv), dissolved in DMSO (0.5 M) under argon and stirring, was added K₂CO₃ (1.5 equiv) and the mixture was either stirred at room temperature or heated between 40° C. and 160° C. until full conversion. The mixture was allowed to return to room temperature and was partitioned between an organic solvent, preferably petroleum ether and water. The aqueous layer was extracted twice more and the combined organic phases were then washed with NaOH (aq, 2M) followed by Brine, dried over Na₂SO₄, filtered and concentrated under vacuum. The residue was then purified by flash chromatography (SiO₂, gradient petroleum ether/AcOEt, DCM/MeOH or petroleum ether/AcOEt/NEt₃) to yield the desired bi(hetero)aryl ether ethyl ester.
B) The corresponding bis(hetero)aryl ether alkyl ester (1 equiv) was dissolved in dry THE (0.2 M) under argon and stirring and the resulting solution was cooled to 0° C. with an ice bath. DIBAL-H (2.5 equiv, 1.2 M in toluene) was then added dropwise and the mixture left to stir at that temperature till full conversion. The reaction was quenched via the Fieser method, filtered, concentrated under vacuum and the residue was then purified by flash chromatography (SiO₂, gradient petroleum ether/AcOEt) to yield the desired alcohol.
C) Depending on the scale and substrate, either of these procedures were used.
   To the corresponding alcohol (1 equiv), dissolved in DCM (0.2 M) under vigorous stirring, was added MnO₂ (2-4 equiv). The resulting suspension was stirred at room temperature or 40° C. till full conversion. The reaction was then diluted with AcOEt, filtered over celite and concentrated under vacuum. The residue was then purified by flash chromatography (SiO₂, gradient petroleum ether/AcOEt) to yield the desired aldehyde.
   To the corresponding alcohol (1 equiv), dissolved in DCM or DMSO (0.2 M) under vigorous stirring, was added Dess Martin Periodinane (1.2 equiv). The resulting suspension was stirred at room temperature till full conversion. The solution was diluted in AcOEt and quenched with aq. sat. NaHCO₃ and the phases separated. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired aldehyde.
   To a solution of oxalyl chloride (2 equiv) in DCM (0.2 M) at −78° C. was added dry DMSO (4 equiv) and the mixture was stirred for 30 min. A solution in DCM (0.2 M) of the corresponding alcohol (1 equiv) was then added followed by freshly distilled NEt₃ (8 equiv). The resulting solution was stirred for 1 hour before being slowly returned to room temperature. The solution was diluted in AcOEt and quenched with aq HCl 1M and the phases separated. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired aldehyde.
D) To the corresponding aldehyde (1 equiv), dissolved in dry THE (0.2 M) at 0° C. under argon and stirring, was added either TMSCF₃ (2 equiv) followed by TBAF (1 mol %) to obtain the corresponding CF₃ bearing secondary alcohol or a Grignard reagent (2 equiv) to obtain the corresponding secondary alkyl alcohol. In both cases, the resulting solution was left to stir at that temperature till full conversion. HCl aq (2.5 M) was then added and the reaction left to stir for a further hour. The reaction was then partitioned between AcOEt and water. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired secondary alcohol.
E) To a stirred solution of the corresponding secondary alcohol (1 equiv) in chloroform (0.2 M) at 0° C. was added Dess-Martin Periodinane (1.5 equiv). After completion of the reaction, it was partitioned between AcOEt and NaHCO₃ aq sat. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired ketone.
F) To a stirred solution of the corresponding ketone (1 equiv) in ethanol (0.2 M) was added the amine (2.5-40 equiv) followed by either a catalytic amount of PTSA in the case of aliphatic amines, or a base (2.5-40 equiv) in the case of hydroxylamines. The reaction was then refluxed for 24-72 h. After this time, Celite was added and the volatiles evaporated under vacuum. The residue was then purified by flash chromatography (SiO₂, gradient petroleum ether/AcOEt) to yield the desired imines.
G) To the corresponding aldehyde or crude imine solution, obtained before purification during procedure (F), in DCM (0.25 M) was added sodium borohydride (4 equiv) and the solution left to stir for a further 2 hours. The reaction was then partitioned between AcOEt and NaHCO₃ aq sat. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired product.
H) To a stirred solution under argon of the corresponding aldehyde (1 equiv) in toluene (0.2 M) was added the amine (2 equiv) followed by TMSCN (2 equiv) and the reaction was stirred for 16 h. The reaction was then partitioned between AcOEt and NaHCO₃ aq sat. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired cyanoamine.
I) To the corresponding bis(hetero)aryl ether alkyl ester (1 equiv), dissolved in EtOH or THE (0.5 M) was added NaOH aq 2 M (2 equiv) and the reaction was left to stir till completion. The reaction was then partitioned between AcOEt and HCl aq (1 M). The aqueous layer was extracted twice more and the combined organic phases were then washed with Brine, dried over Na₂SO₄, filtered and concentrated under vacuum. The residue was then purified either by flash chromatography (SiO₂, gradient petroleum ether/AcOEt) or by recrystallization (AcOEt) to yield the desired carboxylic acid.
J) Depending on the amine used, either of these procedures were employed. To the corresponding bis(hetero)aryl ether carboxylic acid (1 equiv), suspended in stirred toluene (0.2 M), under argon, was added first SOCl₂ (2.5 equiv) then DMF (1 mol %) and the mixture was heated to 80° C. for 3 hours. The reaction mixture was then evaporated to dryness and the resulting residue placed under argon again and redissolved in THE (0.2 M). To this was added in order, trimethylamine (2.5 equiv), DMAP (1 mol %) and the corresponding amine or amide (1.2-1.5 equiv) and the suspension was stirred for 16 hours. The reaction was then partitioned between AcOEt and HCl aq (1 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt or petroleum ether/AcOEt/AcOH or DCM/MeOH) to yield the desired amide.
   To the corresponding bis(hetero)aryl ether carboxylic acid (1 equiv) in DCM (0.2 M) was added in order NEt₃ (3 equiv) and HOBt/EDCI (1.5 equiv/1.5 equiv) or HATU (1.5 equiv). The reaction mixture was then stirred 5 to 60 min before the corresponding amine (1.25 equiv) was added and the mixture stirred till completion. The reaction was then partitioned between AcOEt and HCl aq (1 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt or petroleum ether/AcOEt/AcOH or DCM/MeOH) to yield the desired amide.
   To the corresponding bis(hetero)aryl ether carboxylic acid (1 equiv), suspended in stirred toluene (0.2 M), under argon, was added first SOCl₂ (2.5 equiv) then DMF (1 mol %) and the mixture was heated to 80° C. for 3 hours. The reaction mixture was then evaporated to dryness and the resulting residue placed under argon again and redissolved in toluene (0.2 M). This was added to a solution of the corresponding hydroxylamine in aq. sat. NaHCO₃ and the reaction mixture was stirred till completion. The reaction was then partitioned between AcOEt and aq HCl 1 M or water. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt or petroleum ether/AcOEt/AcOH or DCM/MeOH) to yield the desired amide.
K) To the corresponding 4-substituted phenol (1 equiv) and 1,4-dibromoaryl (2.5 equiv), dissolved in DMF (0.2 M), was added Cs₂CO₃ (2 equiv), CuI (10 mol %) and tBuXPos (20 mol %). The mixture was degassed using the freeze-pump-thaw method, placed under argon, vigorously stirred and refluxed (165° C.) for 72 h. The mixture was allowed to return to room temperature and was partitioned between petroleum ether and NaOH aq 2 M. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired bisaryl ether bromide.
L) To the corresponding 4-substituted phenol (1.2-1.5 equiv) and 1,4-diromo(hetero)aryl (1 equiv), dissolved in DMSO (0.5 M) under argon and stirring, was added K₂CO₃ (1.5 equiv) and the mixture was heated between 80° C. and 160° C. until full conversion. The mixture was allowed to return to room temperature and was partitioned between petroleum ether and NaOH aq 2M. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired bis(hetero)aryl ether bromide.
M) The corresponding bi(hetero)aryl ether bromide (1 equiv) was dissolved in dry THF (0.2 M) under argon and stirring and the resulting solution was cooled to −78° C. with a dry ice/acetone bath. n- or t-BuLi (1.1-2.2 equiv, 1.9-2.5 M in hexane or pentane) was then added dropwise and the mixture left to stir at that temperature for 30 min then at −50° C. till full consumption of the starting material (monitored by TLC in pentane). The mixture was then cooled back down to −78° C., a solution in dry THE of the corresponding electrophile (2 equiv, 0.5 M) was added, and the reaction was allowed to return to room temperature slowly over 16 h. The reaction was then partitioned between AcOEt and NH₄Cl aq. sat., the aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt/NEt₃) to yield the desired compound.
N) To the corresponding protected amine compound (1 equiv), dissolved in THE (0.1-0.2 M) was added HCl (0.5 M in MeOH, 2-6 equiv) and the reaction was left to stir till completion. Then either the reaction was evaporated to dryness to yield the desired amine as HCl salt or the reaction was partitioned between AcOEt and NaHCO₃ aq sat. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt/MeOH or DCM/MeOH) to yield the desired free amine.
O) To the corresponding 4-substituted phenol (1 equiv) and 4-cyano(hetero)haloaryl (2.5 equiv), dissolved in DMSO (0.5 M) under argon and stirring, was added K₂CO₃ (1.5 equiv) and the mixture was heated between 80° C. and 160° C. until full conversion. The mixture was allowed to return to room temperature and was partitioned between petroleum ether and NaOH aq 2M. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired bis(hetero)aryl ether cyanide.
P) To the corresponding bis(hetero)aryl ether cyanide (1 equiv), dissolved in THF/MeOH (1:1, 0.1 M), at 0° C. under argon and stirring, was added NaH (1.1 equiv). After 4 h, the ice bath was removed and cyanamide (1.5 equiv) was added and the mixture stirred for a further 16 h. The reaction was then partitioned between AcOEt and water. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt/MeOH) to yield the desired amine.
Q) To the corresponding bis(hetero)aryl ether alcohol, hydroxamic acid or amide (1 equiv), dissolved in THF/DMF (1:0 to 2:8 mixture, 0.2 M), under argon and stirring, was added NaH, NaOAc or Cs₂CO₃ (1.2-2 equiv). After 30 min, the alkyl (di)halide or acyl chloride (1.2-2 equiv) was added, with KI (1.2 equiv) in the cases of alkyl (di)bromides. The mixture then stirred for a further 16 h at room temperature or 50° C. in the cases of alkyl (di)bromides. The reaction was then partitioned between AcOEt and HCl aq (1 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt/AcOH) to yield the desired amide.
R) To the corresponding bis(hetero)aryl ether amide (1 equiv), dissolved in THF (0.2 M), at 0° C. under argon and stirring, was added NaBH₄ (1.1 equiv). After 1 h, The reaction was then partitioned between AcOEt and NaHCO₃ aq sat. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired amide.
S) To the corresponding aldehyde (1 equiv) in dry THF (0.2 M) was added the corresponding Wittig reagent (1.5 equiv) at 0° C. To this stirred mixture was added dropwise LiHMDS (1.3 equiv, 1 M in THF). The reaction was stirred until completion before being partitioned between AcOEt and HCl aq (1 M). The aqueous layer was extracted twice more and the combined organic phases were then washed with aq. sat. NaHCO₃, Brine, dried over Na₂SO₄, filtered and concentrated under vacuum. The residue was then purified by flash chromatography (SiO₂, gradient petroleum ether/AcOEt) to yield the desired olefin.
T) For adding methyl groups to the amine:
   To the corresponding free amine (1 equiv) in acetonitrile (0.2 M) was added formaldehyde (6 equiv, 37% w/w in water) followed by NaBH₃CN (2 equiv). The reaction mixture was stirred till completion before being partitioned between AcOEt and aq. sat. NaHCO₃, the aqueous layer was extracted twice more and the combined organic phases were then washed with Brine, dried over Na₂SO₄, filtered and concentrated under vacuum. The residue was then purified by flash chromatography (SiO₂, gradient DCM/MeOH/NEt₃) to yield the desired compound.
   For adding isopropyl groups to the amine:
   To the corresponding free amine (1 equiv) in acetone (0.2 M) was added NaBH₃CN (10 equiv) in five portions every 15 min while keeping the pH at approximatively 5 with acetic acid. The reaction mixture was then partitioned between AcOEt and aq. sat. NaHCO₃, the aqueous layer was extracted twice more and the combined organic phases were then washed with Brine, dried over Na₂SO₄, filtered and concentrated under vacuum. The residue was then purified by flash chromatography (SiO₂, gradient DCM/MeOH/NEt₃) to yield the desired compound.
U) To the corresponding amine (1 equiv) in DCM (0.1 M) was added mCPBA (1.2 equiv) and the mixture was stirred at room temperature till full conversion. The reaction mixture was then partitioned between AcOEt and aq. sat. NaHCO₃, the aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired compound.
V) To the corresponding nitrile (1 equiv) in ethanol (0.2 M) was added hydroxylamine hydrochloride (2.5 equiv) and sodium hydroxide (2.5 equiv). The reaction was heated to 80° C. overnight before being filtered over celite, concentrated under vacuum and the residue purified by flash chromatography (SiO₂, gradient petroleum ether/AcOEt) to yield the desired compound.
W) To the corresponding aldehyde (1 equiv) in methanol (0.1 M) was added K₂CO₃ (2 equiv) followed by the Ohira-Bestmann reagent (1.1 equiv) and the reaction stirred till completion. The reaction was then partitioned between AcOEt and NaHCO₃ aq sat. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired alkyne.

Analytical Data

The following compounds were synthetized according to the aforementioned protocols and characterized via mass spectrometry (Table 93) or NMR (Table 94).

TABLE 93

| Compound | | m/z | Ion | m/z | Ion | |
|---|---|---|---|---|---|---|
| No. | Formula | [ESI⁺] | [ESI⁺] | [ESI⁻] | [ESI⁻] | Procedure |
| XPW-0014 | C₂₄H₂₆F₃NO | 385.6 | [M + NH2]⁺ | | | N |
| XPW-0020 | C₂₁H₂₄F₃NO—HCl | 364 | [M + H]⁺ | | | G |
| XPW-0028 | C₂₅H₂₈F₃NO—HCl | 416.7 | [M + H]⁺ | | | N |
| XPW-0042 | C₂₆H₃₀F₃NO | 430.7 | [M + H]⁺ | | | T |
| XPW-0314 | C₂₂H₂₆N₂O | 335.31 | [M + H]⁺ (minor) | | | H |
| | | 308.24 | [M − CN]⁺ (major) | | | |
| | | 290.14 | [M − NMe₂]⁺ (major) | | | |
| XPW-0506 | C₁₇H₁₉NO₂ | 270.1 | [M + H]⁺ | | | J |
| XPW-0509 | C₁₇H₁₄F₃NO₂ | 322.2 | [M + H]⁺ | | | J |
| XPW-0510 | C₁₉H₂₁NO₂ | 296.2 | [M + H]⁺ | | | J |
| XPW-0515 | C₂₀H₂₁NO₂ | 308.2 | [M + H]⁺ | | | J |
| XPW-0516 | C₂₁H₂₃NO₂ | 322.3 | [M + H]⁺ | | | J |
| XPW-0518 | C₂₃H₂₅NO₂ | 348.3 | [M + H]⁺ | | | J |
| XPW-0520 | C₁₈H₂₁NO₂ | 284.2 | [M + H]⁺ | | | J |
| XPW-0523 | C₁₈H₁₆F₃NO₂ | 336.2 | [M + H]⁺ | | | J |
| XPW-0524 | C₂₀H₂₃NO₂ | 310.2 | [M + H]⁺ | | | I |
| XPW-0529 | C₂₁H₂₃NO₂ | 322.3 | [M + H]⁺ | | | I |
| XPW-0530 | C₂₂H₂₅NO₂ | 336.3 | [M + H]⁺ | | | I |
| XPW-0532 | C₂₄H₂₇NO₂ | 362.3 | [M + H]⁺ | | | J |
| XPW-0533 | C₁₉H₂₃NO₂ | 298.2 | [M + H]⁺ | | | J |
| XPW-0534 | C₁₉H₂₃NO₂ | 298.2 | [M + H]⁺ | | | J |
| XPW-0535 | C₁₈H₂₁NO₃ | 300.2 | [M + H]⁺ | | | J |
| XPW-0536 | C₁₉H₂₄N₂O₂ | 313.3 | [M + H]⁺ | | | J |
| XPW-0537 | C₁₉H₁₈F₃NO₂ | 350.2 | [M + H]⁺ | | | I |
| XPW-0538 | C₂₁H₂₅NO₂ | 324.3 | [M + H]⁺ | | | I |
| XPW-0539 | C₂₀H₂₃NO₃ | 326.2 | [M + H]⁺ | | | I |
| XPW-0541 | C₁₉H₂₂N₂O₃ | 327.5 | [M + H]⁺ | | | J |
| XPW-0543 | C₂₂H₂₅NO₂ | 336.3 | [M + H]⁺ | | | J |
| XPW-0544 | C₂₃H₂₇NO₂ | 350.3 | [M + H]⁺ | | | J |
| XPW-0546 | C₂₅H₂₉NO₂ | 376.3 | [M + H]⁺ | | | J |
| XPW-0547 | C₁₈H₂₁NO₄S | 348.2 | [M + H]⁺ | 346.24 | [M − H]⁻ | J |
| XPW-0548 | C₁₈H₂₁NO₄S | 348.2 | [M + H]⁺ | 346.23 | [M − H]⁻ | J |
| XPW-0552 | C₂₀H₂₃NO₄S | 374.3 | [M + H]⁺ | 372.27 | [M − H]⁻ | J |
| XPW-0560 | C₂₄H₂₇NO₄S | 426.3 | [M + H]⁺ | 424.3 | [M − H]⁻ | J |
| XPW-0566 | C₂₁H₂₅NO₄S | 388.3 | [M + H]⁺ | | | Q |
| XPW-0574 | C₂₅H₂₉NO₄S | 440.3 | [M + H]⁺ | | | Q |
| XPW-0575 | C₁₈H₁₈F₃NO₄S | 402.3 | [M + H]⁺ | 400.21 | [M − H]⁻ | J |
| XPW-0576 | C₁₈H₁₈F₃NO₄S | | [M + H]⁺ | 400.23 | [M − H]⁻ | J |
| XPW-0580 | C₂₀H₂₀F₃NO₄S | | [M + H]⁺ | 426.24 | [M − H]⁻ | J |
| XPW-0588 | C₂₄H₂₄F₃NO₄S | | [M + H]⁺ | 478.24 | [M − H]⁻ | J |
| XPW-0603 | C₂₄H₂₅NO₄S | 424.3 | [M + H]⁺ | 422.27 | [M − H]⁻ | J |
| XPW-0604 | C₂₄H₂₅NO₄S | 424.3 | [M + H]⁺ | 422.28 | [M − H]⁻ | O |
| XPW-0608 | C₂₆H₂₇NO₄S | 450.3 | [M + H]⁺ | 448.32 | [M − H]⁻ | J |
| XPW-0616 | C₃₀H₃₁NO₄S | 502.2 | [M + H]⁺ | 500.25 | [M − H]⁻ | J |
| XPW-0636 | C₂₀H₂₀N₂O₂ | | [M + H]⁺ | 319.28 | [M − H]⁻ | J |
| XPW-0659 | C₁₇H₁₉NO₃ | 286.2 | [M + H]⁺ | | | J |
| XPW-0660 | C₁₇H₁₉NO₃ | 286.2 | [M + H]⁺ | | | J |
| XPW-0661 | C₁₆H₁₇NO₄ | 288.2 | [M + H]⁺ | 286.14 | [M − H]⁻ | A |
| XPW-0663 | C₁₇H₁₄F₃NO₃ | 338.2 | [M + H]⁺ | | | J |
| XPW-0664 | C₁₉H₂₁NO₃ | 312.2 | [M + H]⁺ | | | J |
| XPW-0665 | C₁₈H₁₉NO₄ | 314.2 | [M + H]⁺ | 312.19 | [M − H]⁻ | J |
| XPW-0667 | C₁₇H₁₈N₂O₄ | 315.5 | [M + H]⁺ | | | J |
| XPW-0669 | C₂₀H₂₁NO₃ | 324.2 | [M + H]⁺ | | | J |
| XPW-0670 | C₂₁H₂₃NO₃ | 338.3 | [M + H]⁺ | | | J |
| XPW-0672 | C₂₃H₂₅NO₃ | 364.4 | [M + H]⁺ | | | J |
| XPW-0674 | C₁₈H₂₁NO₃ | 300.2 | [M + H]⁺ | | | J |
| XPW-0675 | C₁₇H₁₉NO₄ | 302.2 | [M + H]⁺ | | | J |
| XPW-0678 | C₂₀H₂₃NO₃ | 326.3 | [M + H]⁺ | | | J |
| XPW-0679 | C₁₉H₂₁NO₄ | 328.2 | [M + H]⁺ | | | J |
| XPW-0686 | C₂₄H₂₇NO₃ | 378.3 | [M + H]⁺ | | | J |
| XPW-0700 | C₂₆H₃₁NO₃ | 406.6 | [M + H]⁺ | | | J |
| XPW-0702 | C₁₈H₂₁NO₃ | 300.2 | [M + H]⁺ | | | J |
| XPW-0703 | C₁₇H₁₉NO₄ | 302.2 | [M + H]⁺ | | | A |
| XPW-0704 | C₁₈H₂₂N₂O₃—HCl | 315.2 | [M + H]⁺ | | | J |
| XPW-0706 | C₂₀H₂₃NO₃ | 326.3 | [M + H]⁺ | | | J |
| XPW-0714 | C₂₄H₂₇NO₃ | 378.3 | [M + H]⁺ | | | J |
| XPW-0716 | C₁₉H₂₃NO₃ | 314.2 | [M + H]⁺ | | | J |
| XPW-0717 | C₁₈H₂₁NO₄ | 316.2 | [M + H]⁺ | | | J |
| XPW-0718 | C₁₉H₂₄N₂O₃—HCl | 329.3 | [M + H]⁺ | | | J |
| XPW-0720 | C₂₁H₂₅NO₃ | 340.3 | [M + H]⁺ | | | J |
| XPW-0728 | C₂₄H₂₉NO₃ | 392.4 | [M + H]⁺ | | | J |
| XPW-0734 | C₂₆H₂₇NO₃ | 402.4 | [M + H]⁺ | | | J |
| XPW-0742 | C₃₀H₃₁NO₃ | 454.3 | [M + H]⁺ | | | J |
| XPW-0762 | C₂₃H₂₇NO₃ | 366.3 | [M + H]⁺ | | | J |
| XPW-0770 | C₂₇H₃₁NO₃ | 418.4 | [M + H]⁺ | | | J |
| XPW-0776 | C₂₂H₂₅NO₃ | 352.3 | [M + H]⁺ | | | R |
| XPW-0784 | C₂₆H₂₉NO₃ | 404.4 | [M + H]⁺ | | | R |
| XPW-0790 | C₂₂H₂₃NO₃ | 350.3 | [M + H]⁺ | | | J |
| XPW-0798 | C₂₆H₂₇NO₃ | 402.4 | [M + H]⁺ | | | J |
| XPW-0818 | C₂₄H₂₇NO₃ | 387.7 | [M + H]⁺ | | | J |
| XPW-0832 | C₂₀H₂₁N₃O | 320.3 | [M + H]⁺ | | | P |
| XPW-0902 | C₂₅H₃₃NO₃S | 428.4 | [M + H]⁺ | | | M |
| XPW-0916 | C₂₁H₂₅NO₂ | 324.3 | [M + H]⁺ | | | N |
| XPW-0924 | C₂₅H₂₉NO₂ | 379.32 min | [M + 1]⁺ (minor) | | | M |
| | | 359.28 maj | [M − NH₂]⁺ (major) | | | |
| XPW-0930 | C₂₂H₂₇NO₂ | 338.64 | [M + H]⁺ | | | N |
| | | 307.53 | [M − NHMe]⁺ | | | |
| XPW-1582 | C₂₃H₂₄FNO₂ | 366.3 | [M + H]⁺ | | | J |
| XPW-1587 | C₁₈H₁₅F₄NO₂ | 354.2 | [M + H]⁺ | | | J |
| XPW-1588 | C₂₀H₂₂FNO₂ | 328.3 | [M + H]⁺ | | | J |
| XPW-1596 | C₂₄H₂₆FNO₂ | 380.3 | [M + H]⁺ | | | J |
| XPW-1601 | C₁₉H₁₇F₄NO₂ | 368.2 | [M + H]⁺ | | | J |
| XPW-1602 | C₂₁H₂₄FNO₂ | 342.3 | [M + H]⁺ | | | J |
| XPW-1610 | C₂₅H₂₈FNO₂ | 394.3 | [M + H]⁺ | | | J |
| XPW-1727 | C₁₇H₁₃F₄NO₃ | 356.2 | [M + H]⁺ | | | J |
| XPW-1728 | C₁₉H₂₀FNO₃ | 330.2 | [M + H]⁺ | | | J |
| XPW-1736 | C₂₃H₂₄FNO₃ | 382.3 | [M + H]⁺ | | | J |
| XPW-1750 | C₂₄H₂₆FNO₃ | 396.3 | [M + H]⁺ | | | J |
| XPW-2633 | C₁₆H₁₈N₂O₂ | 271.1 | [M + H]⁺ | | | J |
| XPW-2634 | C₁₆H₁₈N₂O₂ | 271.1 | [M + H]⁺ | | | J |
| XPW-2637 | C₁₆H₁₃F₃N₂O₂ | 323.2 | [M + H]⁺ | | | J |
| XPW-2643 | C₁₉H₂₀N₂O₂ | 309.2 | [M + H]⁺ | | | J |
| XPW-2644 | C₂₀H₂₂N₂O₂ | 323.3 | [M + H]⁺ | | | J |
| XPW-2646 | C₂₂H₂₄N₂O₂ | 349.3 | [M + H]⁺ | | | J |
| XPW-2648 | C₁₇H₂₀N₂O₂ | 285.2 | [M + H]⁺ | | | J |
| XPW-2651 | C₁₇H₁₅F₃N₂O₂ | 337.2 | [M + H]⁺ | | | J |
| XPW-2652 | C₁₉H₂₂N₂O₂ | 311.2 | [M + H]⁺ | | | J |
| XPW-2657 | C₂₀H₂₂N₂O₂ | 323.3 | [M + H]⁺ | | | J |
| XPW-2658 | C₂₁H₂₄N₂O₂ | 337.3 | [M + H]⁺ | | | J |
| XPW-2660 | C₂₃H₂₆N₂O₂ | 363.3 | [M + H]⁺ | | | J |
| XPW-2661 | C₁₈H₂₂N₂O₂ | 299.2 | [M + H]⁺ | | | J |
| XPW-2662 | C₁₈H₂₂N₂O₂ | 299.2 | [M + H]⁺ | | | J |
| XPW-2665 | C₁₈H₁₇F₃N₂O₂ | 351.2 | [M + H]⁺ | | | J |
| XPW-2666 | C₂₀H₂₄N₂O₂ | 325.3 | [M + H]⁺ | | | J |
| XPW-2671 | C₂₁H₂₄N₂O₂ | 337.3 | [M + H]⁺ | | | J |
| XPW-2672 | C₂₂H₂₆N₂O₂ | 351.3 | [M + H]⁺ | | | J |
| XPW-2674 | C₂₄H₂₈N₂O₂ | 377.3 | [M + H]⁺ | | | J |
| XPW-2675 | C₁₇H₂₀N₂O₄S | 349.2 | [M + H]⁺ | 347.22 | [M − H]⁻ | J |
| XPW-2676 | C₁₇H₂₀N₂O₄S | 349.2 | [M + H]⁺ | 347.24 | [M − H]⁻ | J |
| XPW-2688 | C₂₃H₂₆N₂O₄S | 427.3 | [M + H]⁺ | 425.29 | [M − H]⁻ | J |
| XPW-2703 | C₁₇H₁₇F₃N₂O₄S | 403.2 | [M + H]⁺ | 401.21 | [M − H]⁻ | J |
| XPW-2704 | C₁₇H₁₇F₃N₂O₄S | 403.2 | [M + H]⁺ | 401.23 | [M − H]⁻ | J |
| XPW-2708 | C₁₉H₁₉F₃N₂O₄S | 429.2 | [M + H]⁺ | 427.24 | [M − H]⁻ | J |
| XPW-2716 | C₂₃H₂₃F₃N₂O₄S | 480.5 | [M + H]⁺ | 479.23 | [M − H]⁻ | J |
| XPW-2731 | C₂₃H₂₄N₂O₄S | 425.3 | [M + H]⁺ | 423.33 | [M − H]⁻ | J |
| XPW-2732 | C₂₃H₂₄N₂O₄S | 425.3 | [M + H]⁺ | 423.28 | [M − H]⁻ | J |
| XPW-2744 | C₂₉H₃₀N₂O₄S | 503.2 | [M + H]⁺ | 501.26 | [M − H]⁻ | J |
| XPW-2787 | C₁₆H₁₈N₂O₃ | 287.1 | [M + H]⁺ | | | J |
| XPW-2788 | C₁₆H₁₈N₂O₃ | 287.1 | [M + H]⁺ | | | J |
| XPW-2791 | C₁₆H₁₃F₃N₂O₃ | 339.2 | [M + H]⁺ | 337.22 | [M − H]⁻ | J |
| XPW-2792 | C₁₈H₂₀N₂O₃ | 313.2 | [M + H]⁺ | | | J |
| XPW-2795 | C₁₆H₁₇N₃O₄ | 316.5 | [M + H]⁺ | | | J |
| XPW-2797 | C₁₉H₂₀N₂O₃ | 325.2 | [M + H]⁺ | 323.29 | [M − H]⁻ | J |
| XPW-2798 | C₂₀H₂₂N₂O₃ | 339.2 | [M + H]⁺ | 337.31 | [M − H]⁻ | J |
| XPW-2800 | C₂₂H₂₄N₂O₃ | 365.3 | [M + H]⁺ | | | J |
| XPW-2805 | C₁₇H₁₅F₃N₂O₃ | 353.2 | [M + H]⁺ | 351.2 | [M − H]⁻ | J |
| XPW-2806 | C₁₉H₂₂N₂O₃ | 327.3 | [M + H]⁺ | | | J |
| XPW-2833 | C₁₇H₁₅F₃N₂O₃ | 353.2 | [M + H]⁺ | | | J |
| XPW-2834 | C₁₉H₂₂N₂O₃ | 327.3 | [M + H]⁺ | | | J |
| XPW-2847 | C₁₈H₁₇F₃N₂O₃ | 367.2 | [M + H]⁺ | | | J |
| XPW-2848 | C₂₀H₂₄N₂O₃ | 341.3 | [M + H]⁺ | | | J |
| XPW-2890 | C₂₂H₂₆N₂O₃ | 367.3 | [M + H]⁺ | | | J |
| XPW-2898 | C₂₆H₃₀N₂O₃ | 419.4 | [M + H]⁺ | | | J |
| XPW-2904 | C₂₁H₂₄N₂O₃ | 353.3 | [M + H]⁺ | | | R |
| XPW-2912 | C₂₅H₂₈N₂O₃ | 405.4 | [M + H]⁺ | | | R |
| XPW-2918 | C₂₁H₂₂N₂O₃ | 351.3 | [M + H]⁺ | | | J |
| XPW-2926 | C₂₅H₂₆N₂O₃ | 403.3 | [M + H]⁺ | | | J |
| XPW-3038 | C₂₈H₃₆N₂O₃S | 481.3 | [M + H]⁺ | | | M |
| XPW-3052 | C₂₄H₂₈N₂O₂ | 377.3 | [M + H]⁺ | | | N |
| XPW-3193 | C₁₈H₂₁NO₃ | 300.2 | [M + H]⁺ | | | J |
| XPW-3194 | C₁₇H₁₃ClF₃NO₃ | 372.2 | [M + H]⁺ | 370.29 | [M − H]⁻ | J |
| XPW-3195 | C₁₇H₂₀N₂O₂ | 285.2 | [M + H]⁺ | | | J |
| XPW-3196 | C₁₈H₂₂N₂O₂ | 299.2 | [M + H]⁺ | | | J |
| XPW-3197 | C₁₇H₂₀N₂O₃ | 301.2 | [M + H]⁺ | 299.23 | [M − H]⁻ | J |
| XPW-3199 | C₁₉H₂₂FNO₂ | 316.2 | [M + H]⁺ | | | J |
| XPW-3200 | C₁₈H₂₀FNO₃ | 318.2 | [M + H]⁺ | 316.26 | [M − H]⁻ | J |
| XPW-3201 | C₁₅H₁₆N₂O₃ | 273.1 | [M + H]⁺ | | | J |
| XPW-3202 | C₁₃H₁₂N₂O₃ | 245.1 | [M + H]⁺ | | | J |
| XPW-3203 | C₁₄H₁₄N₂O₃ | 259.1 | [M + H]⁺ | 257.17 | [M − H]⁻ | J |
| XPW-3205 | C₁₇H₂₀N₂O₂ | 285.2 | [M + H]⁺ | | | J |
| XPW-3206 | C₁₅H₁₆N₂O₃ | 273.1 | [M + H]⁺ | | | J |
| XPW-3207 | C₁₈H₂₂N₂O₂ | 299.2 | [M + H]⁺ | | | J |
| XPW-3208 | C₁₉H₂₄N₂O₂ | 313.2 | [M + H]⁺ | | | J |
| XPW-3209 | C₁₇H₂₀N₂O₃ | 301.2 | [M + H]⁺ | | | J |
| XPW-3210 | C₁₆H₁₇NO₂ | 256.1 | [M + H]⁺ | | | J |
| XPW-3211 | C₁₇H₁₉NO₂ | 270.1 | [M + H]⁺ | | | J |
| XPW-3212 | C₁₆H₁₇NO₃ | 272.1 | [M + H]⁺ | | | J |
| XPW-3213 | C₁₈H₂₁NO₃ | 300.2 | [M + H]⁺ | | | J |
| XPW-3214 | C₁₆H₁₇NO₃ | 272.1 | [M + H]⁺ | | | J |
| XPW-3215 | C₁₆H₁₇NO₂ | 256.1 | [M + H]⁺ | | | J |
| XPW-3216 | C₁₅H₁₅NO₃ | 258.1 | [M + H]⁺ | | | J |
| XPW-3217 | C₁₄H₁₃NO₃ | 244.1 | [M + H]⁺ | | | J |
| XPW-3218 | C₁₃H₉F₃N₂O₃ | 299.1 | [M + H]⁺ | 297.13 | [M − H]⁻ | J |
| XPW-3219 | C₁₇H₁₉FN₂O₃ | 319.2 | [M + H]⁺ | | | J |
| XPW-3221 | C₁₈H₁₉FN₂O₃ | 331.2 | [M + H]⁺ | | | J |
| XPW-3222 | C₂₂H₂₃FN₂O₂ | 367.4 | [M + H]⁺ | | | J |
| XPW-3223 | C₂₃H₂₅FN₂O₂ | 381.3 | [M + H]⁺ | | | J |
| XPW-3224 | C₂₄H₂₇FN₂O₂ | 395.3 | [M + H]⁺ | | | J |
| XPW-3225 | C₂₂H₂₃FN₂O₃ | 383.3 | [M + H]⁺ | 381.34 | [M − H]⁻ | J |
| XPW-3226 | C₁₆H₁₂F₄N₂O₃ | 357.2 | [M + H]⁺ | 355.24 | [M − H]⁻ | J |
| XPW-3227 | C₁₆H₁₂ClF₃N₂O₃ | 373.2 | [M + H]⁺ | 371.27 | [M − H]⁻ | J |
| XPW-3228 | C₁₇H₁₃F₅N₂O₂ | 373.2 | [M + H]⁺ | | | J |
| XPW-3229 | C₁₈H₁₅F₅N₂O₂ | 387.2 | [M + H]⁺ | | | J |
| XPW-3230 | C₁₉H₁₇F₅N₂O₂ | 401.3 | [M + H]⁺ | | | J |
| XPW-3231 | C₁₇H₁₃F₅N₂O₃ | 389.2 | [M + H]⁺ | 387.3 | [M − H]⁻ | J |
| XPW-3232 | C₂₀H₁₈F₅NO₂ | 400.3 | [M + H]⁺ | | | J |
| XPW-3233 | C₁₈H₁₄F₅NO₃ | 388.2 | [M + H]⁺ | 386.31 | [M − H]⁻ | J |
| XPW-3234 | C₁₉H₁₇ClF₃NO₂ | 384.2 | [M + H]⁺ | | | J |
| XPW-4543 | C₂₀H₂₅NO₂ | 312.2 | [M + H]⁺ | | | J |
| XPW-4544 | C₁₉H₂₃NO₂ | 298.2 | [M + H]⁺ | | | J |
| XPW-4545 | C₁₉H₂₄N₂O₂ | 313.3 | [M + H]⁺ | | | J |
| XPW-4546 | C₁₉H₂₃FN₂O₂ | 331.3 | [M + H]⁺ | | | J |
| XPW-4547 | C₁₅H₁₆N₂O₂ | 257.1 | [M + H]⁺ | | | J |
| XPW-4548 | C₁₆H₁₈N₂O₂ | 271.1 | [M + H]⁺ | | | J |
| XPW-4549 | C₁₇H₂₀N₂O₂ | 285.2 | [M + H]⁺ | | | J |
| XPW-4550 | C₁₉H₂₁FN₂O₂ | 329.3 | [M + H]⁺ | | | J |
| XPW-4551 | C₂₀H₂₃FN₂O₂ | 343.3 | [M + H]⁺ | | | J |
| XPW-4552 | C₁₃H₁₂N₂O₂ | 229.1 | [M + H]⁺ | | | J |
| XPW-4553 | C₁₄H₁₄N₂O₂ | 243.1 | [M + H]⁺ | | | J |
| XPW-4554 | C₁₅H₁₆N₂O₂ | 257.1 | [M + H]⁺ | | | J |
| XPW-4555 | C₁₄H₁₄N₂O₂ | 243.1 | [M + H]⁺ | | | J |
| XPW-4556 | C₁₅H₁₆N₂O₂ | 257.1 | [M + H]⁺ | | | J |
| XPW-4557 | C₁₆H₁₈N₂O₂ | 271.1 | [M + H]⁺ | | | J |
| XPW-4558 | C₁₅H₁₆N₂O₂ | 257.1 | [M + H]⁺ | | | J |
| XPW-4559 | C₁₇H₂₀N₂O₂ | 285.2 | [M + H]⁺ | | | J |
| XPW-4560 | C₁₈H₂₁NO₂ | 284.2 | [M + H]⁺ | | | J |
| XPW-4561 | C₁₉H₂₃NO₂ | 298.2 | [M + H]⁺ | | | J |
| XPW-4562 | C₂₀H₂₅NO₂ | 312.2 | [M + H]⁺ | | | J |
| XPW-4563 | C₁₇H₁₉NO₂ | 270.1 | [M + H]⁺ | | | J |
| XPW-4564 | C₁₈H₂₁NO₂ | 284.2 | [M + H]⁺ | | | J |
| XPW-4565 | C₁₅H₁₅NO₂ | 242.1 | [M + H]⁺ | | | J |
| XPW-4566 | C₁₆H₁₂F₄N₂O₂ | 341.2 | [M + H]⁺ | | | J |
| XPW-4567 | C₁₈H₁₆F₄N₂O₂ | 369.2 | [M + H]⁺ | | | J |
| XPW-4568 | C₁₃H₉F₃N₂O₂ | 283 | [M + H]⁺ | | | J |
| XPW-4569 | C₁₄H₁₁F₃N₂O₂ | 297.1 | [M + H]⁺ | | | J |
| XPW-4570 | C₁₅H₁₃F₃N₂O₂ | 311.1 | [M + H]⁺ | | | J |
| XPW-4571 | C₁₆H₁₂ClF₃N₂O₂ | 357.1 | [M + H]⁺ | | | J |
| XPW-4572 | C₁₇H₁₄ClF₃N₂O₂ | 371.2 | [M + H]⁺ | | | J |
| XPW-4573 | C₁₈H₁₆ClF₃N₂O₂ | 385.2 | [M + H]⁺ | | | J |
| XPW-4574 | C₁₉H₂₂N₂O₂ | 311.5 | [M + H]⁺ | | | V |
| XPW-4575 | C₂₁H₂₄ClNO₂ | 358.59/360.59 | [M + H]⁺ | | | J |
| XPW-4576 | C₂₃H₂₅NO₄ | 380.6 | [M + H]⁺ | | | J |
| XPW-4577 | C₂₅H₂₇NO₃ | 390.7 | [M + H]⁺ | | | R |
| XPW-4578 | C₂₄H₂₉NO₄ | 396.7 | [M + H]⁺ | | | G |
| XPW-4579 | C₂₈H₃₆N₂O₄ | 465.7 | [M + H]⁺ | | | G |
| XPW-4580 | C₂₅H₃₂N₂O₃ | 409.7 | [M + H]⁺ | | | G |
| XPW-4581 | C₂₅H₃₁NO₄ | 410.7 | [M + H]⁺ | | | Q |
| XPW-4583 | C₂₃H₂₆ClNO₃ | 400.7 | [M + H]⁺ | | | J |
| XPW-4584 | C₁₉H₂₀ClNO₃ | 346.5 | [M + H]⁺ | | | J |
| XPW-4585 | C₂₅H₂₈ClNO₂ | 410.7 | [M + H]⁺ | | | J |
| XPW-4586 | C₂₇H₃₀ClNO₃ | 452.7 | [M + H]⁺ | | | J |
| XPW-4587 | C₂₃H₂₄ClNO₃ | 398.6 | [M + H]⁺ | | | J |
| XPW-4588 | C₂₁H₂₄BrNO₂ | 402.57/404.58 | [M + H]⁺ | | | J |
| XPW-4589 | C₂₃H₂₆BrNO₃ | 444.57/446.57 | [M + H]⁺ | | | J |
| XPW-4590 | C₁₉H₂₀BrNO₃ | 390.52/392.52 | [M + H]⁺ | | | J |
| XPW-4591 | C₂₅H₂₈BrNO₂ | 454.59/456.58 | [M + H]⁺ | | | J |
| XPW-4592 | C₂₇H₃₀BrNO₃ | 496.62/498.59 | [M + H]⁺ | | | J |
| XPW-4593 | C₂₃H₂₄BrNO₃ | 442.54/444.54 | [M + H]⁺ | | | J |
| XPW-4594 | C₂₄H₂₉NO₃ | 380.6 | [M + H]⁺ | | | J |
| XPW-4595 | C₂₂H₂₇NO₂ | 338.6 | [M + H]⁺ | | | J |
| XPW-4603 | C₁₉H₂₁NO₃ | 312.5 | [M + H]⁺ | | | J |
| XPW-4605 | C₁₇H₁₉NO₃ | 286.4 | [M + H]⁺ | | | J |
| XPW-4612 | C₂₇H₃₃NO₃ | 420.7 | [M + H]⁺ | | | J |
| XPW-4613 | C₂₃H₂₅NO₄ | 380.6 | [M + H]⁺ | | | J |
| XPW-4614 | C₃₀H₃₁NO₄ | 470.7 | [M + H]⁺ | | | J |
| XPW-4616 | C₂₉H₂₉NO₃ | 440.7 | [M + H]⁺ | | | J |
| XPW-4617 | C₂₉H₃₅NO₃ | 446.7 | [M + H]⁺ | | | I |
| XPW-4618 | C₂₅H₂₉NO₃ | 392.7 | [M + H]⁺ | | | J |
| XPW-4619 | C₃₁H₃₃NO₃ | 468.7 | [M + H]⁺ | | | J |
| XPW-4620 | C₃₄H₃₉NO₃ | 510.7 | [M + H]⁺ | | | J |
| XPW-4622 | C₃₁H₃₃NO₄ | 484.6 | [M + H]⁺ | | | J |
| XPW-4623 | C₂₁H₂₃N₃O₃ | 366.6 | [M + H]⁺ | | | J |
| XPW-4624 | C₂₃H₂₇N₃O₂ | 378.7 | [M + H]⁺ | | | J |
| XPW-4625 | C₂₁H₂₃N₃O₃ | 366.6 | [M + H]⁺ | 364.54 | [M − H]⁻ | J |
| XPW-4626 | C₂₈H₂₉N₃O₃ | 456.7 | [M + H]⁺ | | | I |
| XPW-4627 | C₂₃H₂₇N₃O₂ | 378.7 | [M + H]⁺ | | | J |
| XPW-4628 | C₂₁H₂₃N₃O₃ | 366.6 | [M + H]⁺ | | | J |
| XPW-4629 | C₂₃H₂₇N₃O₂ | 378.7 | [M + H]⁺ | | | J |
| XPW-4630 | C₂₂H₂₄N₂O₃ | 365.6 | [M + H]⁺ | | | J |
| XPW-4631 | C₂₉H₃₀N₂O₃ | 455.7 | [M + H]⁺ | | | J |
| XPW-4632 | C₂₈H₂₉N₃O₃ | 456.6 | [M + H]⁺ | | | J |
| XPW-4633 | C₂₈H₃₄F₃NO₂S | 506.7 | [M + H]⁺ | | | M |
| XPW-4634 | C₂₃H₂₇N₃O₂ | 378.6 | [M + H]⁺ | | | J |
| XPW-4635 | C₂₄H₂₈N₂O₂ | 377.6 | [M + H]⁺ | | | J |
| XPW-4636 | C₂₁H₂₃N₃O₃ | 366.6 | [M + H]⁺ | | | J |
| XPW-4637 | C₂₇H₃₁NO₃ | 418.7 | [M + H]⁺ | | | Q |
| XPW-4638 | C₂₂H₂₅NO₃ | 352.6 | [M + H]⁺ | | | Q |
| XPW-4639 | C₂₃H₂₆N₂O₃ | 379.6 | [M + H]⁺ | | | J |
| XPW-4640 | C₃₀H₃₂N₂O₃ | 469.7 | [M + H]⁺ | | | J |
| XPW-4641 | C₂₅H₃₀N₂O₂ | 391.7 | [M + H]⁺ | | | J |
| XPW-4642 | C₂₉H₃₆F₃NO₂S | 520.7 | [M + H]⁺ | | | Q |
| XPW-4644 | C₂₈H₂₉N₃O₃ | 356.7 | [M + H]⁺ | | | J |
| XPW-4645 | C₁₇H₁₉NO₂ | 270.1 | [M + H]⁺ | | | J |
| XPW-4646 | C₃₁H₃₀F₃NO₃ | | | 520.72 | [M − H]⁻ | J |
| XPW-4647 | C₃₀H₂₉NO₄ | | | 466.69 | [M − H]⁻ | Q |
| XPW-4714 | C₁₄H₁₃NO₂ | 228.1 | [M + H]⁺ | | | J |
| XPW-4715 | C₁₅H₁₅NO₂ | 242.1 | [M + H]⁺ | | | J |
| XPW-4718 | C₁₈H₂₁NO₂ | 284.2 | [M + H]⁺ | | | J |
| XPW-4723 | C₁₆H₁₇NO₂ | 256.1 | [M + H]⁺ | | | J |
| XPW-4843 | C₁₇H₁₉FN₂O₂ | 303.2 | [M + H]⁺ | | | I |
| XPW-I-0001 | C₁₉H₂₂O₃ | 299.2 | [M + H]⁺ | | | A |
| XPW-I-0002 | C₂₁H₂₄O₃ | 325.3 | [M + H]⁺ | | | A |
| XPW-I-0003 | C₁₉H₂₂O₃ | 299.2 | [M + H]⁺ | | | A |
| XPW-I-0004 | C₂₅H₂₈O₃ | 377.4 | [M + H]⁺ | | | A |
| XPW-I-0005 | C₂₃H₂₃FO₃ | | | 365.39 | [M − H]⁻ | I |
| XPW-I-0006 | C₁₇H₁₂F₄O₃ | | | 339.22 | [M − H]⁻ | I |
| XPW-I-0008 | C₁₉H₂₂O₂ | 265.1 | [M − OH]⁺ | | | B |
| XPW-I-0009 | C₁₉H₂₀O₂ | 281.2 | [M + H]⁺ | | | C |
| XPW-I-0010 | C₂₀H₂₁F₃O₂ | 333.2 | [M − OH]⁺ | | | D |
| XPW-I-0011 | C₁₈H₂₀O₄ | 301.2 | [M + H]⁺ | | | A |
| XPW-I-0012 | C₁₉H₂₃NO₃ | 314.3 | [M + H]⁺ | | | A |
| XPW-I-0013 | C₁₇H₁₉NO₄ | 302.2 | [M + H]⁺ | | | A |
| XPW-I-0014 | C₁₆H₁₆O₄ | 273.1 | [M + H]⁺ | 271.13 | [M − H]⁻ | I |
| XPW-I-0015 | C₁₇H₁₉NO₃—HCl | 286.1 | [M + H]⁺ | | | I |
| XPW-I-0016 | C₁₅H₁₅NO₄ | 274.1 | [M + H]⁺ | 272.12 | [M − H]⁻ | I |
| XPW-I-0017 | C₂₀H₁₉F₃O₂ | | | 374.13 | [M − H]⁻ | E |
| XPW-I-0019 | C₂₀H₂₂O₄ | 327.2 | [M + H]⁺ | | | J |
| XPW-I-0020 | C₂₁H₂₂BrNO | 384.21/386.20 | [M + H]⁺ | | | L |
| XPW-I-0021 | C₁₈H₁₉BrO | | | 329.06 | [M − H]⁻ | K |
| XPW-I-0022 | C₁₈H₁₈O₄ | 299.2 | [M + H]⁺ | 297.13 | [M − H]⁻ | I |
| XPW-I-0023 | C₁₇H₁₈O₃ | | | 269.18 | [M − H]⁻ | I |
| XPW-I-0024 | C₁₇H₁₈O₃ | | | 269.18 | [M − H]⁻ | I |
| XPW-I-0025 | C₁₉H₂₀O₃ | | | 295.27 | [M − H]⁻ | I |
| XPW-I-0026 | C₂₂H₂₄O₃ | 337.3 | [M + H]⁺ | | | A |
| XPW-I-0027 | C₂₀H₂₀O₃ | | | 307.28 | [M − H]⁻ | I |
| XPW-I-0028 | C₂₃H₂₆O₃ | 351.3 | [M + H]⁺ | | | A |
| XPW-I-0029 | C₂₁H₂₂O₃ | | | 321.34 | [M − H]⁻ | I |
| XPW-I-0030 | C₂₃H₂₄O₃ | | | 347.37 | [M − H]⁻ | I |
| XPW-I-0031 | C₁₇H₁₃F₃O₃ | | | 321.22 | [M − H]⁻ | I |
| XPW-I-0032 | C₁₉H₁₇F₃O₃ | 351.2 | [M + H]⁺ | | | A |
| XPW-I-0033 | C₁₉H₂₁FO₃ | 317.2 | [M + H]⁺ | | | A |
| XPW-I-0034 | C₁₇H₁₇FO₃ | | | 287.18 | [M − H]⁻ | I |
| XPW-I-0035 | C₁₈H₂₁NO₃ | 300.2 | [M + H]⁺ | | | A |
| XPW-I-0036 | C₁₆H₁₇NO₃ | 272.1 | [M + H]⁺ | 270.18 | [M − H]⁻ | I |
| XPW-I-0037 | C₁₈H₂₁NO₃ | 300.2 | [M + H]⁺ | | | A |
| XPW-I-0038 | C₁₆H₁₇NO₃ | 272.1 | [M + H]⁺ | 270.18 | [M − H]⁻ | I |
| XPW-I-0039 | C₂₀H₂₃NO₃ | 326.3 | [M + H]⁺ | | | A |
| XPW-I-0040 | C₁₈H₁₉NO₃ | 298.2 | [M + H]⁺ | 296.22 | [M − H]⁻ | I |
| XPW-I-0041 | C₂₁H₂₃NO₃ | 338.2 | [M + H]⁺ | | | A |
| XPW-I-0042 | C₁₉H₁₉NO₃ | | | 308.25 | [M − H]⁻ | I |
| XPW-I-0043 | C₂₂H₂₅NO₃ | 352.3 | [M + H]⁺ | | | A |
| XPW-I-0044 | C₂₀H₂₁NO₃ | | | 322.3 | [M − H]⁻ | I |
| XPW-I-0045 | C₂₄H₂₇NO₃ | 378.3 | [M + H]⁺ | | | A |
| XPW-I-0046 | C₂₂H₂₃NO₃ | | | 348.37 | [M − H]⁻ | I |
| XPW-I-0047 | C₁₈H₁₆F₃NO₃ | 352.2 | [M + H]⁺ | | | A |
| XPW-I-0048 | C₁₆H₁₂F₃NO₃ | 324.1 | [M + H]⁺ | 322.22 | [M − H]⁻ | I |
| XPW-I-0049 | C₁₉H₂₁FO₃ | 317.2 | [M + H]⁺ | | | A |
| XPW-I-0050 | C₁₇H₁₇FO₃ | | | 287.18 | [M − H]⁻ | I |
| XPW-I-0051 | C₂₁H₂₃FO₃ | 343.3 | [M + H]⁺ | | | A |
| XPW-I-0052 | C₁₉H₁₉FO₃ | | | 313.24 | [M − H]⁻ | I |
| XPW-I-0053 | C₂₅H₂₇FO₃ | 395.3 | [M + H]⁺ | | | A |
| XPW-I-0054 | C₁₉H₁₆F₄O₃ | 369.2 | [M + H]⁺ | | | A |
| XPW-I-0055 | C₁₈H₁₉FO₃ | | | 301.24 | [M − H]⁻ | I |
| XPW-I-0056 | C₁₅H₁₃FO₃ | | | 259.15 | [M − H]⁻ | I |
| XPW-I-0057 | C₁₄H₁₁FO₃ | | | 245.13 | [M − H]⁻ | I |
| XPW-I-0058 | C₁₆H₁₅FO₃ | | | 273.15 | [M − H]⁻ | I |
| XPW-I-0059 | C₁₄H₈F₄O₃ | | | 299.12 | [M − H]⁻ | I |
| XPW-I-0060 | C₁₇H₁₉NO₃ | 286.2 | [M + H]⁺ | 284.2 | [M − H]⁻ | I |
| XPW-I-0062 | C₂₀H₂₄O₃ | 313.3 | [M + H]⁺ | | | A |
| XPW-I-0063 | C₁₈H₂₀O₃ | | | 283.21 | [M − H]⁻ | I |
| XPW-I-0064 | C₁₆H₁₆O₃ | 257.1 | [M + H]⁺ | | | A |
| XPW-I-0065 | C₁₇H₁₈O₃ | 271.1 | [M + H]⁺ | | | A |
| XPW-I-0066 | C₁₅H₁₄O₃ | | | 241.17 | [M − H]⁻ | I |
| XPW-I-0067 | C₁₈H₂₀O₃ | 285.2 | [M + H]⁺ | | | A |
| XPW-I-0068 | C₁₆H₁₆O₃ | | | 255.18 | [M − H]⁻ | I |
| XPW-I-0069 | C₁₈H₂₀O₃ | 285.2 | [M + H]⁺ | | | A |
| XPW-I-0070 | C₁₆H₁₆O₃ | | | 255.18 | [M − H]⁻ | I |
| XPW-I-0071 | C₂₀H₂₄O₃ | 313.3 | [M + H]⁺ | | | A |
| XPW-I-0072 | C₁₈H₂₀O₃ | | | 283.21 | [M − H]⁻ | I |
| XPW-I-0073 | C₁₅H₁₅NO₃ | 258.1 | [M + H]⁺ | | | A |
| XPW-I-0074 | C₁₃H₁₁NO₃ | 230.1 | [M + H]⁺ | 228.15 | [M − H]⁻ | I |
| XPW-I-0075 | C₁₆H₁₇NO₃ | 272.1 | [M + H]⁺ | | | A |
| XPW-I-0076 | C₁₄H₁₃NO₃ | 244 | [M + H]⁺ | 242.15 | [M − H]⁻ | I |
| XPW-I-0077 | C₁₇H₁₉NO₃ | 286.2 | [M + H]⁺ | | | A |
| XPW-I-0078 | C₁₅H₁₅NO₃ | 258.1 | [M + H]⁺ | 256.17 | [M − H]⁻ | I |
| XPW-I-0079 | C₁₉H₂₃NO₃ | 314.2 | [M + H]⁺ | | | A |
| XPW-I-0080 | C₁₇H₁₉NO₃ | 286.2 | [M + H]⁺ | 284.2 | [M − H]⁻ | I |
| XPW-I-0081 | C₁₇H₁₉NO₃ | 286.2 | [M + H]⁺ | | | A |
| XPW-I-0082 | C₁₅H₁₅NO₃ | 258.1 | [M + H]⁺ | 256.17 | [M − H]⁻ | I |
| XPW-I-0083 | C₁₉H₂₃NO₃ | 314.2 | [M + H]⁺ | | | A |
| XPW-I-0084 | C₁₅H₁₂F₃NO₃ | 312.1 | [M + H]⁺ | | | A |
| XPW-I-0085 | C₁₃H₈F₃NO₃ | 284 | [M + H]⁺ | 282.1 | [M − H]− | I |
| XPW-I-0086 | C₂₀H₂₃FO₃ | 331.3 | [M + H]⁺ | | | A |
| XPW-I-0087 | C₁₄H₁₂O₃ | | | 227.16 | [M − H]⁻ | I |
| XPW-I-0088 | C₁₉H₂₂FNO₃ | 332.2 | [M + H]⁺ | | | A |
| XPW-I-0089 | C₁₇H₁₈FNO₃ | 304.2 | [M + H]⁺ | 302.22 | [M − H]⁻ | I |
| XPW-I-0090 | C₂₀H₂₂FNO₃ | 344.2 | [M + H]⁺ | | | A |
| XPW-I-0091 | C₁₈H₁₈FNO₃ | | | 314.24 | [M − H]⁻ | I |
| XPW-I-0092 | C₂₄H₂₆FNO₃ | 396.3 | [M + H]⁺ | | | A |
| XPW-I-0093 | C₂₂H₂₂FNO₃ | 368.3 | [M + H]⁺ | 366.37 | [M − H]⁻ | I |
| XPW-I-0094 | C₁₈H₁₅F₄NO₃ | 370.2 | [M + H]⁺ | | | A |
| XPW-I-0095 | C₁₆H₁₁F₄NO₃ | | | 340.21 | [M − H]⁻ | I |
| XPW-I-0096 | C₁₇H₁₂ClF₃O₃ | | | 355.21 | [M − H]⁻ | I |
| XPW-I-0097 | C₁₉H₁₆ClF₃O₃ | 385.2 | [M + H]⁺ | | | A |
| XPW-I-0098 | C₁₈H₁₅ClF₃NO₃ | 386.2 | [M + H]⁺ | | | A |
| XPW-I-0099 | C₁₆H₁₁ClF₃NO₃ | 358.1 | [M + H]⁺ | 356.2 | [M − H]⁻ | I |
| XPW-I-0100 | C₂₀H₁₇F₅O₃ | 401.3 | [M + H]⁺ | | | A |
| XPW-I-0101 | C₁₉H₁₆F₅NO₃ | 402.3 | [M + H]⁺ | | | A |
| XPW-I-0102 | C₁₈H₁₃F₅O₃ | n.a | [M + H]⁺ | 371.3 | [M − H]⁻ | I |
| XPW-I-0103 | C₁₇H₁₂F₅NO₃ | 374.2 | [M + H]⁺ | 372.3 | [M − H]⁻ | I |
| XPW-I-0104 | C₁₆H₁₅FO₃ | 275.1 | [M + H]⁺ | | | A |
| XPW-I-0105 | C₁₇H₁₇FO₃ | 289.1 | [M + H]⁺ | | | A |
| XPW-I-0106 | C₁₈H₁₉FO₃ | 303.2 | [M + H]⁺ | | | A |
| XPW-I-0107 | C₁₆H₁₂F₄O₃ | 329.2 | [M + H]⁺ | | | A |
| XPW-I-0108 | C₂₀H₂₃FO₃ | 331.2 | [M + H]⁺ | | | A |
| XPW-I-0109 | C₁₈H₁₉FO₃ | 303.2 | [M + H]⁺ | | | A |
| XPW-I-0110 | C₁₈H₁₉FO₃ | | | 301.21 | [M − H]⁻ | I |
| XPW-I-0111 | C₁₆H₁₅FO₃ | | | 273.17 | [M − H]⁻ | I |
| XPW-I-0112 | C₁₇H₁₈FNO₃ | 304.2 | [M + H]⁺ | | | A |
| XPW-I-0113 | C₁₈H₂₀FNO₃ | 318.2 | [M + H]⁺ | | | A |
| XPW-I-0114 | C₁₅H₁₄FNO₃ | 276.1 | [M + H]⁺ | | | A |
| XPW-I-0115 | C₁₅H₁₁F₄NO₃ | 330.1 | [M + H]⁺ | | | A |
| XPW-I-0116 | C₁₅H₁₄FNO₃ | | | 274.15 | [M − H]⁻ | I |
| XPW-I-0117 | C₁₆H₁₆FNO₃ | | | 288.17 | [M − H]⁻ | I |
| XPW-I-0118 | C₁₃H₁₀FNO₃ | | | 246.12 | [M − H]⁻ | I |
| XPW-I-0119 | C₁₃H₇F₄NO₃ | | | 300.1 | [M − H]⁻ | I |
| XPW-I-0120 | C₂₁H₂₂O₄ | 339.6 | [M + H]⁺ | | | A |
| XPW-I-0121 | C₁₉H₁₈O₄ | | | 309.45 | [M − H]⁻ | I |
| XPW-I-0123 | C₂₀H₂₀O₄ | 325.5 | [M + H]⁺ | 323.48 | [M − H]⁻ | I |
| XPW-I-0124 | C₂₁H₂₃ClO₃ | 359.6 | [M + H]⁺ | | | A |
| XPW-I-0125 | C₂₅H₂₇ClO₃ | 411.6 | [M + H]⁺ | | | A |
| XPW-I-0126 | C₂₁H₂₃BrO₃ | 403.56/405.55 | [M + H]⁺ | | | A |
| XPW-I-0128 | C₁₉H₂₁NO₄ | 328.5 | [M + H]⁺ | | | A |
| XPW-I-0129 | C₁₈H₂₀N₂O₄ | 329.5 | [M + H]⁺ | | | A |
| XPW-I-0130 | C₁₉H₂₁NO₆ | 300.4 | [M + H]⁺ | | | I |
| XPW-I-0131 | C₁₉H₁₉ClO₃ | | | 329.44 | [M − H]⁻ | I |
| XPW-I-0132 | C₂₃H₂₃ClO₃ | | | 381.57 | [M − H]⁻ | I |
| XPW-I-0133 | C₁₉H₁₉BrO₃ | | | 373.49 | [M − H]⁻ | I |
| XPW-I-0134 | C₂₃H₂₃BrO₃ | | | 425.55 | [M − H]⁻ | I |
| XPW-I-0136 | C₂₃H₂₄O₄ | | | 363.61 | [M − H]⁻ | I |
| XPW-I-0138 | C₂₀H₂₂O₃ | | | 309.48 | [M − H]⁻ | I |
| XPW-I-0139 | C₂₁H₂₂N₂O₃ | 351.6 | [M + H]⁺ | | | A |
| XPW-I-0140 | C₂₂H₂₄N₂O₃ | 365.6 | [M + H]⁺ | | | A |
| XPW-I-0141 | C₂₂H₂₄N₂O₃ | 365.6 | [M + H]⁺ | | | A |
| XPW-I-0142 | C₂₄H₂₇NO₃ | 378.7 | [M + H]⁺ | | | A |
| XPW-I-0143 | C₂₃H₂₆N₂O₃ | 379.6 | [M + H]⁺ | | | A |
| XPW-I-0144 | C₂₁H₂₂N₂O₃ | 351.6 | [M + H]⁺ | | | I |
| XPW-I-0145 | C₂₁H₂₂N₂O₃ | 351.6 | [M + H]⁺ | | | I |
| XPW-I-0146 | C₂₂H₂₃NO₃ | 350.6 | [M + H]⁺ | | | I |
| XPW-I-0147 | C₂₁H₂₂N₂O₃ | 351.6 | [M + H]⁺ | 349.54 | [M − H]⁻ | I |
| XPW-I-0148 | C₂₄H₂₇NO₃ | 378.6 | [M + H]⁺ | | | A |
| XPW-I-0149 | C₂₃H₂₅NO₃ | 364.6 | [M + H]⁺ | | | I |
| XPW-I-0150 | C₂₄H₂₇NO₄ | 394.7 | [M + H]⁺ | | | J |

TABLE 94

| Compound No. | Formula | ¹H-NMR | Procedure |
|---|---|---|---|
| XPW-0182 | C₂₅H₂₈F₃NO₂ | ¹H NMR (400 MHz, CDCl₃) δ 7.41-7.33 (m, 4H), 7.05-6.96 (m, 4H), 4.32 | U |
| | | (q, J = 7.6 Hz, 1H), 2.87 (s, 3H), 2.16-2.07 (m, 3H), 1.92 (d, J = 2.9 Hz, 6H), | |
| | | 1.85-1.67 (m, 6H). | |
| XPW-4621 | C₃₄H₃₃NO₃ | ¹H NMR (400 MHz, CDCl₃) δ 7.93-7.76 (m, 3H), 7.62-7.46 (m, 6H), 7.32 | J |
| | | (d, J = 8.7 Hz, 2H), 7.27-7.13 (m, 1H), 6.97-6.88 (m, 3H), 5.40 (s, 2H), | |
| | | 2.09 (s, 3H), 1.94-1.87 (m, 6H), 1.76 (q, J = 12.5 Hz, 6H). | |
| XPW-4643 | C₂₈H₃₄F₃NO | ¹H NMR (400 MHz, CDCl₃) δ 7.38-7.30 (m, 4H), 7.02-6.91 (m, 4H), 4.24 | T |
| | | (q, J = 8.8 Hz, 1H), 3.04 (h, J = 6.5 Hz, 1H), 2.30 (d, J = 1.5 Hz, 3H), 2.11 (p, J = | |
| | | 3.2 Hz, 3H), 1.92 (d, J = 2.9 Hz, 6H), 1.86-1.70 (m, 6H), 1.06 (d, J = 6.6 | |
| | | Hz, 3H), 0.97 (d, J = 6.5 Hz, 3H). | |
| XPW-I-0007 | C₁₉H₁₉NO₃ | ¹H NMR (400 MHz, CDCl₃) δ 7.62-7.55 (m, 2H), 7.25-7.21 (m, 2H), 7.02- | J |
| | | 6.95 (m, 4H), 2.60-2.42 (m, 1H), 1.95-1.70 (m, 5H), 1.42 (dt, J = 12.1, | |
| | | 10.4 Hz, 4H), 1.33-1.19 (m, 1H). | |
| XPW-I-0018 | C₂₂H₂₃BrO₃ | ¹H NMR (400 MHz, CDCl₃) δ 7.43-7.38 (m, 2H), 7.35-7.30 (m, 2H), 6.97- | K |
| | | 6.91 (m, 2H), 6.90-6.84 (m, 2H), 2.10 (s, 3H), 1.90 (d, J = 2.9 Hz, 6H), 1.84- | |
| | | 1.69 (m, 6H). | |
| XPW-I-0122 | C₂₂H₂₄O₄ | ¹H NMR (400 MHz, CDCl₃) δ 10.36 (s, 1H), 8.12-7.98 (m, 2H), 7.80 (d, J = | A |
| | | 2.4 Hz, 1H), 7.43 (dd, J = 8.5, 2.4 Hz, 1H), 7.07-6.99 (m, 2H), 6.93 (d, J = | |
| | | 8.5 Hz, 1H), 4.37 (q, J = 7.1 Hz, 2H), 2.56 (s, 1H), 1.97-1.81 (m, 4H), 1.77 | |
| | | (d, J = 13.1 Hz, 1H), 1.51-1.20 (m, 8H). | |
| XPW-I-0127 | C₂₅H₂₇BrO₃ | ¹H NMR (400 MHz, CDCl₃) δ 8.05-7.95 (m, 2H), 7.61 (d, J = 2.3 Hz, 1H), | A |
| | | 7.31 (dd, J = 8.5, 2.3 Hz, 1H), 7.02 (d, J = 8.5 Hz, 1H), 6.95-6.87 (m, 2H), | |
| | | 4.35 (q, J = 7.1 Hz, 2H), 2.12 (s, 3H), 1.91 (d, J = 2.9 Hz, 6H), 1.78 (q, J = 12.5 | |
| | | Hz, 6H), 1.38 (d, J = 7.1 Hz, 2H). | |
| XPW-I-0135 | C₂₅H₂₈O₄ | ¹H NMR (400 MHz, CDCl₃) δ 8.02-7.91 (m, 2H), 6.99-6.89 (m, 6H), 4.32 | A |
| | | (q, J = 7.1 Hz, 2H), 2.16 (s, 3H), 1.84 (d, J = 3.0 Hz, 6H), 1.61 (t, J = 10.8 Hz, | |
| | | 6H), 1.34 (t, J = 7.1 Hz, 3H). | |
| XPW-I-0137 | C₂₂H₂₆O₃ | ¹H NMR (400 MHz, CDCl₃) δ 8.00-7.92 (m, 2H), 7.10 (d, J = 2.2 Hz, 1H), | A |
| | | 7.04 (dd, J = 8.3, 2.3 Hz, 1H), 6.91-6.83 (m, 3H), 4.35 (q, J = 7.1 Hz, 2H), | |
| | | 2.54-2.40 (m, 1H), 2.15 (s, 3H), 1.95-1.70 (m, 5H), 1.46-1.18 (m, 8H). | |

For illustrative purposes the synthesis and characterisation of the following examples are described in detail.

### XPW-0547 4-(4-butylphenoxy)-N-(methylsulfonyl)benzamide

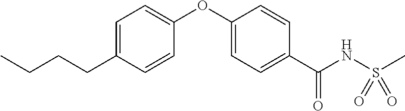 [SMILES: CCCCc1ccc(Oc2ccc(C(=O)NS(C)(=O)=O)cc2)cc1]

To 4-(4-butylphenoxy)benzoic acid (104 mg, 0.38 mmol, 1 equiv), suspended in stirred toluene (1.85 mL, 0.2 M), under argon, was added first SOCl₂ (67 μL, 0.93 mmol, 2.5 equiv) then DMF (0.3 μL, 3.7 μmol, 1 mol %) and the mixture was heated to 80° C. for 3 h. The reaction mixture was then evaporated to dryness and the resulting residue placed under argon again and redissolved in THF (1.85 mL, 0.2 M). To this was added in order, trimethylamine (0.13 mL, 0.93 mmol, 2.5 equiv), DMAP (0.45 mg, 3.7 μmol, 1 mol %) and methansulfonamide (42.3 mg, 0.45 mmol, 1.2 equiv) and the suspension was stirred for 16 h. The reaction was then partitioned between AcOEt and HCl aq (1 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 118 mg of 4-(4-butylphenoxy)-N-(methylsulfonyl)benzamide (88%).

MS: m/z [M−H]⁻, calc for [C₁₈H₂₀NO₄S]⁻=346.11; found 346.24.

¹H-NMR (300 MHz DMSO-d₆) δ 12.03 (s, 1H), 7.97 (d, J=8.9 Hz, 2H), 7.32-7.24 (m, 2H), 7.08-6.97 (m, 4H), 3.36 (s, 3H), 2.66-2.56 (m, 2H), 1.58 (tt, J=8.8, 6.8 Hz, 2H), 1.41-1.24 (m, 2H), 0.92 (t, J=7.3 Hz, 3H).

¹³C-NMR (75 MHz, DMSO-d₆) δ 166.1, 162.1, 153.2, 139.4, 131.4, 130.5, 126.3, 120.4, 117.2, 41.8, 34.6, 33.6, 22.2, 14.3.

### XPW-2890 (6-(4-cyclohexylphenoxy)pyridin-3-yl)(morpholino)methanone

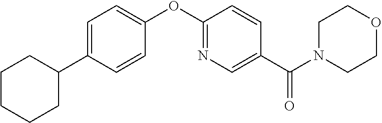 [SMILES: O=C(c1ccc(Oc2ccc(C3CCCCC3)cc2)nc1)N1CCOCC1]

To 6-(4-cyclohexylphenoxy)nicotinic acid (50 mg, 0.17 mmol, 1 equiv), suspended in stirred toluene (0.85 mL, 0.2 M), under argon, was added first SOCl₂ (31 μL, 0.43 mmol, 2.5 equiv) then DMF (0.14 μL, 1.7 μmol, 1 mol %) and the mixture was heated to 80° C. for 3 h. The reaction mixture was then evaporated to dryness and the resulting residue placed under argon again and redissolved in THE (0.85 mL, 0.2 M). To this was added in order, trimethylamine (9.4 μL, 0.68 mmol, 2.5 equiv), DMAP (0.2 mg, 1.7 μmol, 1 mol %) and morpholine (20 μL, 0.23 mmol, 1.5 equiv) and the suspension was stirred for 16 hours. The reaction was then partitioned between AcOEt and HCl aq (1 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 45 mg of (6-(4-cyclohexylphenoxy)pyridin-3-yl)(morpholino)methanone (71%).

MS: m/z [M+H]⁺, calc for [C₂₂H₂₇N₂O₃]⁺=367.20; found 367.32.

¹H-NMR (300 MHz, CDCl₃) δ 8.20 (d, J=2.3 Hz, 1H), 7.73 (dd, J=8.6, 2.2 Hz, 1H), 7.18 (d, J=7.7 Hz, 2H), 7.06-6.92 (m, 2H), 6.87 (d, J=8.5 Hz, 1H), 3.63 (s, 8H), 2.50-2.37 (m, 1H), 1.89-1.62 (m, 5H), 1.38-1.08 (m, 5H).

¹³C-NMR (75 MHz, CDCl3) δ 167.7, 164.7, 151.2, 146.6, 145.1, 139.5, 128.1, 125.5, 121.0, 111.3, 66.8, 53.4, 44.0, 34.5, 26.9, 26.1.

### XPW-0636 N-cyano-4-(4-cyclohexylphenoxy)benzamide

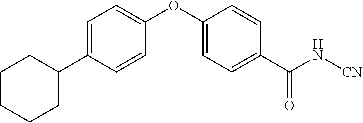 [SMILES: N#CNC(=O)c1ccc(Oc2ccc(C3CCCCC3)cc2)cc1]

To 4-(4-cyclohexylphenoxy)benzoic acid (60 mg, 0.2 mmol, 1 equiv), suspended in stirred toluene (0.8 mL, 0.2 M), under argon, was added first SOCl₂ (37 μL, 0.0.5 mmol, 2.5 equiv) then DMF (0.15 μL, 2.0 μmol, 1 mol %) and the mixture was heated to 80° C. for 3 h. The reaction mixture was then evaporated to dryness and the resulting residue placed under argon again and redissolved in THF (0.8 mL, 0.2 M). To this was added in order, trimethylamine (57 μL, 0.5 mmol, 2.5 equiv), DMAP (0.24 mg, 2.0 μmol, 1 mol %) and cyanamide (12.6 mg, 0.3 mmol, 1.5 equiv) and the suspension was stirred for 16 h. The reaction was then partitioned between AcOEt and HCl aq (1 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 15.4 mg of N-cyano-4-(4-cyclohexylphenoxy)benzamide (24%).

MS: m/z [M−H]⁻, calc for [C₂₀H₁₉N₂O₂]⁻=319.15; found 319.28.

¹H-NMR, (300 MHz, CDCl₃) δ 7.93 (d, J=8.8 Hz, 2H), 7.31 (d, J=8.5 Hz, 2H), 7.23-6.85 (m, 4H), 3.37 (brs, 1H), 2.59-2.51 (m, 1H) 1.91-1.63 (m, 5H), 1.57-1.12 (m, 5H).

¹³C-NMR, (75 MHz, CDCl₃) δ 166.5, 161.8, 152.7, 144.2, 130.8, 128.4, 124.8, 119.9, 117.1, 110.0, 43.1, 34.0, 26.3, 25.5.

### XPW-0675 N-hydroxy-4-(4-(2-methoxyethyl)phenoxy)-N-methylbenzamide

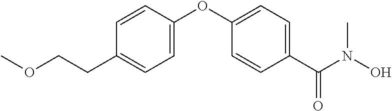 [SMILES: COCCc1ccc(Oc2ccc(C(=O)N(C)O)cc2)cc1]

To 4-(4-(2-methoxyethyl)phenoxy)benzoic acid (125 mg, 0.46 mmol, 1 equiv), suspended in stirred toluene (2.3 mL, 0.2 M), under argon, was added first SOCl₂ (84 μL, 1.15 mmol, 2.5 equiv) then DMF (0.35 μL, 4.6 μmol, 1 mol %) and the mixture was heated to 80° C. for 3 h. The reaction mixture was then evaporated to dryness and the resulting residue placed under argon again and redissolved in THE (2.3 mL, 0.2 M). To this was added in order, trimethylamine (144 μL, 1.13 mmol, 2.5 equiv), DMAP (0.56 mg, 4.6 μmol, 1 mol %) and N-methylhydroxylamine hydrochloride (58 mg, 0.69 mmol, 1.5 equiv) and the suspension was stirred for 16 h. The reaction was then partitioned between AcOEt and HCl aq (1 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 103.1 mg of N-hydroxy-4-(4-(2-methoxyethyl)phenoxy)-N-methylbenzamide (74%).

MS: m/z [M+H]⁺, calc for [C₁₇H₂₀NO₄]⁺=302.14; found 302.17.

¹H-NMR, (300 MHz, CDCl₃) δ 10.00 (s, 1H), 7.73-7.61 (m, 2H), 7.37-7.20 (m, 2H), 7.07-6.82 (m, 4H), 3.55 (t, J=6.8 Hz, 2H), 3.26 (s, 3H), 3.25 (s, 3H), 2.82 (t, J=6.8 Hz, 2H).

¹³C-NMR, (75 MHz, CDCl₃) δ 168.6, 159.2, 154.3, 135.6, 131.2, 130.9, 129.5, 119.9, 117.1, 73.2, 58.3, 37.9, 35.1.

### XPW-0832 N′-cyano-6-(4-cyclohexylphenoxy)nicotinimidamide

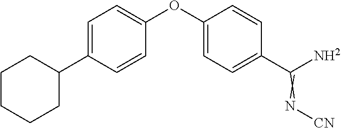 [SMILES: N#CN=C(N)c1ccc(Oc2ccc(C3CCCCC3)cc2)cc1]

To 6-(4-cyclohexylphenoxy)nicotinonitrile (41 mg, 0.15 mmol, 1 equiv), dissolved in THF/MeOH (1:1, 1.5 mL, 0.1 M), at 0° C. under argon and stirring, was added NaH (6.6 mg, 0.17 mmol, 1.1 equiv, 60% in oil). After 4 h, the ice bath was removed and cyanamide (9.5 mg, 0.23 mmol, 1.5 equiv) was added and the mixture stirred for a further 16 h. The reaction was then partitioned between AcOEt and water. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt/MeOH) to yield 32 mg of N′-cyano-6-(4-cyclohexylphenoxy)nicotinimidamide (67%).

MS: m/z [M+H]⁺, calc for [C₂₀H₂₂N₃O]⁺=320.18; found 320.27.

¹H-NMR (300 MHz, DMSO-d₆) δ 7.79 (d, J=8.9 Hz, 2H), 7.25-7.17 (m, 2H), 7.07-6.94 (m, 4H), 3.83 (s, 2H), 2.63-2.41 (m, 1H), 1.94-1.55 (m, 5H), 1.48-1.16 (m, 5H).

¹³C-NMR (75 MHz, DMSO-d₆) δ 152.9, 144.9, 129.3, 129.3, 128.4, 124.4, 120.1, 117.4, 116.4, 113.3, 77.5, 44.0, 34.6, 26.9, 26.1.

### XPW-0902: N-(1-(4-(4-((3r,5r,7r)-adamantan-1-yl)phenoxy)phenyl)-2,2,2-trifluoroethyl)-2-methylpropane-2-sulfinamide

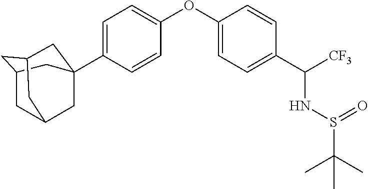 [SMILES: CC(C)(C)S(=O)NC(c1ccc(Oc2ccc(C34CC5CC(CC(C5)C3)C4)cc2)cc1)C(F)(F)F]

(3r,5r,7r)-1-(4-(4-bromophenoxy)phenyl)adamantane (100 mg, 0.26 mmol, 1 equiv) was dissolved in dry THF (1.3 mL, 0.2 M) under argon and stirring and the resulting solution was cooled to −78° C. with a dry ice/acetone bath. ⁿBuLi (0.11 mL, 0.26 mmol, 1.0 equiv, 2.3 M in pentane) was then added dropwise and the mixture left to stir at that temperature for 30 min then at −50° C. for another 30 min. The mixture was then cooled back down to −78° C. A solution of 2-methyl-N-(2,2,2-trifluoroethylidene)propane-2-sulfinamide (79 mg, 0.39 mmol, 1.5 equiv) in THF (0.39 mL, 1 M) was added dropwise and the reaction was stirred 1 hour before allowed to return to room temperature slowly overnight. The reaction was then partitioned between AcOEt and NH₄Cl aq sat., the aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 81 mg of N-(1-(4-(4-((3r,5r,7r)-adamantan-1-yl)phenoxy)phenyl)-2,2,2-trifluoroethyl)-2-methylpropane-2-sulfinamide (62%).

MS: m/z [M+H]⁺, calc for [C₂₈H₃₅F₃NO₂S]⁺=506.23; found 506.70.

¹H NMR (400 MHz, CDCl₃) δ 7.38-7.32 (m, 4H), 7.03-6.95 (m, 4H), 4.83 (qd, J=7.1, 3.5 Hz, 1H), 3.88 (d, J=3.5 Hz, 1H), 2.10 (p, J=3.5 Hz, 3H), 1.91 (d, J=2.9 Hz, 6H), 1.83-1.68 (m, 6H), 1.23 (s, 9H).

¹³C NMR (101 MHz, CDCl₃) δ 159.29, 153.59, 147.36, 130.78, 126.31, 125.24, 124.53 (q, J=281.3 Hz), 119.39, 117.93, 59.87 (q, J=30.4 Hz), 56.31, 43.33, 36.76, 35.91, 28.95, 22.41.

¹⁹F NMR (376 MHz, CDCl₃) δ −74.56 (d, J=7.2 Hz).

### XPW-3052 3-(6-(4-((adamantan-1-yl)phenoxy)pyridin-3-yl)oxetan-3-amine

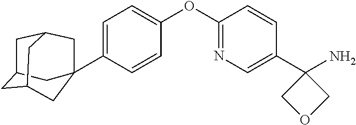 [SMILES: NC1(c2ccc(Oc3ccc(C45CC6CC(CC(C6)C4)C5)cc3)nc2)COC1]

To a solution of N-(3-(4-(4-((3r,5r,7r)-adamantan-1-yl)phenoxy)phenyl)oxetan-3-yl)-2-methylpropane-2-sulfinamide (31 mg, 0.065 mmol, 1 equiv) in THF (0.32 mL, 0.2 M) was added HCl (0.8 mL, 0.5 M in MeOH, 6 equiv) and the reaction was left to stir till completion. The reaction was then partitioned between AcOEt and NaHCO₃ aq (1 M). The aqueous layer was extracted twice more and the combined organic phases were then washed with Brine, dried over Na₂SO₄, filtered and concentrated under vacuum. The residue was then purified by flash chromatography (SiO₂, gradient DCM/MeOH) to yield 17.4 mg of 3-(6-(4-((adamantan-1-yl)phenoxy)pyridin-3-yl)oxetan-3-amine (71%).

MS: m/z [M+H]⁺, calc for [C₂₄H₂₉N₂O₂]⁺=377.22; found 377.34.

¹H-NMR, (300 MHz, DMSO-d₆) δ 8.33 (dd, J=2.6, 0.7 Hz, 1H), 8.02 (dd, J=8.6, 2.6 Hz, 1H), 7.50-7.36 (m, 2H), 7.14-6.96 (m, 3H), 4.70 (d, J=6.3 Hz, 2H), 4.64 (d, J=6.3 Hz, 2H), 2.65 (brs, 2H), 2.18-1.99 (m, 3H), 1.96-1.83 (m, 6H), 1.75 (s, 6H).

¹³C-NMR, (75 MHz, DMSO-d₆) δ 162.6, 152.3, 147.4, 144.9, 138.0, 136.4, 126.4, 121.0, 111.3, 85.7, 57.6, 43.2, 36.6, 35.9, 28.8.

### XPW-4642 N-(1-(4-(4-((3r,5r,7r)-adamantan-1-yl)phenoxy)phenyl)-2,2,2-trifluoroethyl)-N,2-dimethylpropane-2-sulfinamide

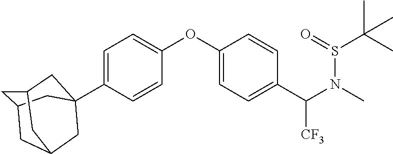 [SMILES: CN(C(c1ccc(Oc2ccc(C34CC5CC(CC(C5)C3)C4)cc2)cc1)C(F)(F)F)S(=O)C(C)(C)C]

To a solution of N-(1-(4-(4-((3r,5r,7r)-adamantan-1-yl)phenoxy)phenyl)-2,2,2-trifluoroethyl)-2-methylpropane-2-sulfinamide (360 mg, 0.71 mmol, 1 equiv) in THE (7.2 mL, 0.1 M) was added Cs₂CO₃ (464 mg, 1.42 mmol, 2 equiv). After 30 min, iodomethane (0.089 mL, 1.42 mmol, 2 equiv) was added. The mixture then heated for a further 16 h at 50° C. The reaction was then partitioned between AcOEt and water. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 300 mg of N-(1-(4-(4-((3r,5r,7r)-adamantan-1-yl)phenoxy)phenyl)-2,2,2-trifluoroethyl)-N,2-dimethylpropane-2-sulfinamide (81%).

MS: m/z [M+H]⁺, calc for [C₂₉H₃₇F₃NO₂S]⁺=520.25; found 520.69.

¹H NMR (400 MHz, CDCl₃) δ 7.42 (d, J=8.6 Hz, 2H), 7.37-7.32 (m, 2H), 7.02-6.96 (m, 4H), 4.92 (q, J=8.6 Hz, 1H), 2.71 (s, 3H), 2.11 (s, 3H), 1.91 (d, J=2.9 Hz, 6H), 1.85-1.71 (m, 6H), 1.20 (s, 9H).

¹⁹F NMR (376 MHz, CDCl₃) δ −67.23 (d, J=8.2 Hz).

### XPW-0028: 1-(4-(4-((3r,5r,7r)-adamantan-1-yl)phenoxy)phenyl)-2,2,2-trifluoro-N-methylethan-1-amine hydrochloride

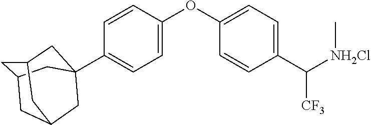

To N-(1-(4-(4-((3r,5r,7r)-adamantan-1-yl)phenoxy)phenyl)-2,2,2-trifluoroethyl)-N,2-dimethylpropane-2-sulfinamide (300 mg, 0.58 mmol, 1 equiv), dissolved in THE (5.8 mL, 0.1 M) was added HCl (2.3 mL, 1.15 mmol, 0.5 M in MeOH, 2 equiv) and the reaction was left to stir till completion. Then either the reaction was evaporated to dryness to yield 255 mg of 1-(4-(4-((3r,5r,7r)-adamantan-1-yl)phenoxy)phenyl)-2,2,2-trifluoro-N-methylethan-1-amine as HCl salt (Quantitative).

MS: m/z [M+H]⁺, calc for [C₂₅H₂₉F₃NO]⁺=416.22; found 416.69.

¹H NMR (400 MHz, CDCl₃) δ 11.04 (brs, 2H), 7.62 (d, J=8.5 Hz, 2H), 7.43-7.33 (m, 2H), 7.08 (d, J=8.4 Hz, 2H), 7.05-6.96 (m, 2H), 4.71-4.36 (m, 1H), 2.69 (s, 3H), 2.14 (s, 3H), 1.94 (d, J=2.9 Hz, 6H), 1.88-1.73 (m, 6H).

### XPW-0182: N-(1-(4-(4-((3r,5r,7r)-adamantan-1-yl)phenoxy)phenyl)-2,2,2-trifluoroethyl)-N-methyl hydroxylamine

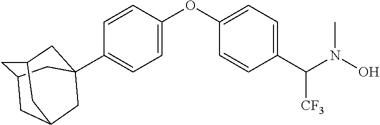 [SMILES: CN(O)C(c1ccc(Oc2ccc(C34CC5CC(CC(C5)C3)C4)cc2)cc1)C(F)(F)F]

To 1-(4-(4-((3r,5r,7r)-adamantan-1-yl)phenoxy)phenyl)-2,2,2-trifluoro-N-methylethan-1-amine hydrochloride (50 mg, 0.12 mmol, 1 equiv) in DCM (1.2 mL, 0.1 M) was added mCPBA (35 mg, 0.144 mmol, 70%, 1.2 equiv) and the mixture was stirred at room temperature for 30 min. The reaction mixture was then partitioned between AcOEt and aq. sat. NaHCO₃, the aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 44.5 mg of N-(1-(4-(4-((3r,5r,7r)-adamantan-1-yl)phenoxy)phenyl)-2,2,2-trifluoroethyl)-N-methylhydroxylamine (86%).

¹H NMR (400 MHz, CDCl₃) δ 7.41-7.33 (m, 4H), 7.05-6.96 (m, 4H), 4.32 (q, J=7.6 Hz, 1H), 2.87 (s, 3H), 2.16-2.07 (m, 3H), 1.92 (d, J=2.9 Hz, 6H), 1.85-1.67 (m, 6H).

¹⁹F NMR (376 MHz, CDCl₃) δ −67.82 (d, J=7.5 Hz).

¹³C NMR (101 MHz, CDCl₃) δ 159.24, 153.61, 147.38, 131.35, 126.33, 124.40, 124.18 (q, J=282.5 Hz), 119.37, 117.73, 74.70 (q, J=29.0 Hz), 50.31, 43.34, 36.76, 35.92, 28.96.

### XPW-0042: 1-(4-(4-((3r,5r,7r)-adamantan-1-yl)phenoxy)phenyl)-2,2,2-trifluoro-N,N-dimethylethan-1-amine

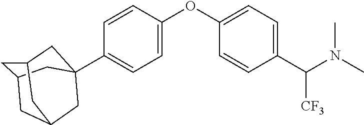 [SMILES: CN(C)C(c1ccc(Oc2ccc(C34CC5CC(CC(C5)C3)C4)cc2)cc1)C(F)(F)F]

To 1-(4-(4-((3r,5r,7r)-adamantan-1-yl)phenoxy)phenyl)-2,2,2-trifluoro-N-methylethan-1-amine hydrochloride (20 mg, 0.05 mmol, 1 equiv) in acetonitrile (0.25 mL, 0.2 M) was added formaldehyde (0.025 mL, 0.29 mmol, 6 equiv, 37% w/w in water) followed by NaBH₃CN (6.1 mg, 0.10 mmol, 2 equiv). The reaction mixture was stirred till completion before being partitioned between AcOEt and aq. sat. NaHCO₃, the aqueous layer was extracted twice more and the combined organic phases were then washed with Brine, dried over Na₂SO₄, filtered and concentrated under vacuum. The residue was then purified by flash chromatography (SiO₂, gradient DCM/MeOH) to yield 15 mg of 1-(4-(4-((3r,5r,7r)-adamantan-1-yl)phenoxy)phenyl)-2,2,2-trifluoro-N,N-dimethylethan-1-amine (75%).

MS: m/z [M+H]⁺, calc for [C₂₆H₃₁F₃NO]⁺=430.27; found 430.71.

¹H NMR (400 MHz, CDCl₃) δ 7.39-7.28 (m, 4H), 7.04-6.93 (m, 4H), 3.95 (q, J=8.7 Hz, 1H), 2.36 (s, 6H), 2.10 (s, 3H), 1.91 (d, J=2.9 Hz, 6H), 1.85-1.69 (m, 6H).

¹⁹F NMR (376 MHz, CDCl₃) δ −67.30.

### XPW-0314 2-(4-(4-cyclohexylphenoxy)phenyl)-2-(dimethylamino)acetonitrile

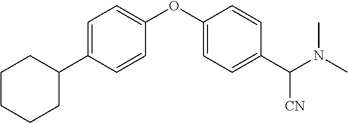 [SMILES: CN(C)C(C#N)c1ccc(Oc2ccc(C3CCCCC3)cc2)cc1]

To a stirred solution under argon of 4-(4-cyclohexylphenoxy)benzaldehyde (50 mg, 0.18 mmol, 1 equiv) in toluene (0.9 mL, 0.2 M) was added dimethylamine (0.18 mL, 0.36 mmol, 2 equiv) followed by TMSCN (0.05 mL, 0.36 mmol, 2 equiv) and the reaction was stirred for 16 h. The reaction was then partitioned between AcOEt and NaHCO₃ aq sat. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 29 mg of 2-(4-(4-cyclohexylphenoxy)phenyl)-2-(dimethylamino)acetonitrile (48%).

MS: m/z [M+H]⁺, calc for [C₂₂H₂₇N₂O]⁺=335.21; found 335.31.

¹H-NMR, (300 MHz, CDCl₃) δ 7.46-7.32 (m, 2H), 7.16-7.07 (m, 2H), 6.99-6.82 (m, 4H), 4.75 (s, 1H), 2.43 (ddt, J=11.7, 8.2, 5.0 Hz, 1H), 2.27 (s, 6H), 1.87-1.61 (m, 5H), 1.42-1.24 (m, 5H).

¹³C NMR (75 MHz, CDCl₃) δ 158.5, 154.2, 143.8, 129.3, 128.1, 119.3, 118.3, 115.0, 62.5, 43.9, 41.7, 34.6, 26.9, 26.1.

1. A compound according to formula (I) as defined herein or a salt or solvate thereof: 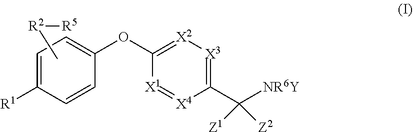
R¹=C₁-C₁₂ preferably C₄-C₁₂ alkyl, C₂-C₁₂ preferably C₄-C₁₂ alkenyl, C₂-C₁₂ preferably C₄-C₁₂ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, —OC₁-C₁₂ preferably —OC₃-C₁₂ alkyl, —OC₂-C₁₂ preferably —OC₃-C₁₂ alkenyl, —OC₂-C₁₂ preferably —OC₃-C₁₂ alkynyl, —OC₃-C₈ cycloalkyl, —OC₅-C₈ cycloalkenyl, —OC₅-C₁₂ bicycloalkyl, —OC₇-C₁₂ bicycloalkenyl, —OC₈-C₁₄ tricycloalkyl, —SC₁-C₁₂ preferably —SC₃-C₁₂ alkyl, —SC₂-C₁₂ preferably —SC₃-C₁₂ alkenyl, —SC₂-C₁₂ preferably —SC₃-C₁₂ alkynyl, —SC₃-C₈ cycloalkyl, —SC₅-C₈ cycloalkenyl, —SC₅-C₁₂ bicycloalkyl, —SC₇-C₁₂ bicycloalkenyl, —SC₈-C₁₄ tricycloalkyl, —NHR⁷ or —NR⁷R⁸ wherein R⁷ and R⁸ are independently from each other selected from: C₁-C₁₂ preferably C₃-C₁₂ alkyl, C₂-C₁₂ preferably C₃-C₁₂ alkenyl, C₂-C₁₂ preferably C₃-C₁₂ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, or wherein R⁷ can form a ring structure together with R⁸ wherein the said ring structure including the N-atom is selected from three to eight membered cyclic structures or five to twelve membered bicyclic structures and wherein all said ring structures can additionally contain one or more heteroatoms independently selected from O, S and N in replacement of a carbon atom contained in the ring structure, and particularly wherein such a replacement results in residues that contain at least twice the number of C atoms than heteroatoms independently selected from O, S and N;
wherein all alkyl, alkenyl and alkynyl residues contained in the definitions of R¹, R⁷ and R⁸ are linear or branched, and are unsubstituted or substituted with one or more substituents independently selected from: —F, —Cl, —Br, —I, —CN, —NCO, —NCS, —OH, —NH₂, —NO₂, ═O, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, linear or branched —OC₁-C₅ alkyl such as —OCH₃, —OC₃-C₅ cycloalkyl such as —O(cyclopropyl), linear or branched —NH(C₁-C₅ alkyl), linear or branched —N(C₁-C₅ alkyl)(C₁-C₅ alkyl), —NH(C₃-C₅ cycloalkyl) such as —NH(cyclopropyl), —N(C₃-C₅ cycloalkyl)(C₃-C₅ cycloalkyl), linear or branched —N(C₁-C₅ alkyl)(C₃-C₅ cycloalkyl);
wherein when an alkyl, alkenyl and alkynyl residue contained in the definitions of R¹, R⁷ and R⁸ is substituted with one or more substituents being ═O, such substitution with ═O cannot result in one of the groups selected from C═O, S═O and N═O directly bound to an aromatic ring;
wherein all cyclic structures, bicyclic structures and tricyclic structures including cycloalkyl, cycloalkenyl, bicycloalkyl, bicycloalkenyl and tricycloalkyl residues contained in the definitions of R¹, R⁷ and R⁸ are unsubstituted or substituted with one or more substituents independently selected from: —F, —Cl, —Br, —I, —CN, —NCO, —NCS, —OH, —NH₂, —NO₂, ═O, linear or branched C₁-C₅ alkyl such as —CH₃, linear or branched —OC₁-C₅ alkyl such as —OCH₃, linear or branched —NH(C₁-C₅ alkyl), linear or branched —N(C₁-C₅ alkyl)(C₁-C₅ alkyl), —NH(C₃-C₅ cycloalkyl) such as —NH(cyclopropyl), —N(C₃-C₅ cycloalkyl)(C₃-C₅ cycloalkyl), linear or branched —N(C₁-C₅ alkyl)(C₃-C₅ cycloalkyl);
wherein all alkyl, alkenyl and alkynyl residues contained in the definitions of R¹, R⁷ and R⁸ can contain one or more heteroatoms independently selected from O, S and N in replacement of a carbon atom, and wherein such a replacement results in residues that contain at least twice the number of C atoms than heteroatoms independently selected from O, S and N, and wherein such replacement additionally cannot result in one of the groups selected from C═O, S═O and N═O directly bound to an aromatic ring;
wherein all cycloalkyl, cycloalkenyl, bicycloalkyl, bicycloalkenyl and tricycloalkyl residues contained in the definitions of R¹, R⁷ and R⁸ can contain one or more heteroatoms independently selected from O, S and N in replacement of a carbon atom, and wherein such a replacement results in residues that contain at least the same number of C atoms than heteroatoms independently selected from O, S and N;
wherein all alkyl, alkenyl, alkynyl, cycloalkyl, cycloalkenyl, bicycloalkyl, bicycloalkenyl and tricycloalkyl residues contained in the definitions of R¹, R⁷ and R⁸ can be partially or fully halogenated, particularly fluorinated, more particularly perfluorinated;
wherein bicyclic and tricyclic residues include fused, bridged and spiro systems;
R²-R⁵ are independently from each other selected from —H, —F, —Cl, —Br, —I, —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
wherein all alkyl, alkenyl, alkynyl and cycloalkyl residues contained in the definitions of R²-R⁵ are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂;
wherein all alkyl, alkenyl, alkynyl and cycloalkyl residues contained in the definitions of R²-R⁵ can contain one or more heteroatoms independently selected from O, S and N in replacement of a carbon atom, and wherein such a replacement cannot result in one of the groups selected from C═O and S═O directly bound to an aromatic ring;
X¹-X⁴ are independently from each other selected from N, CR⁹, CR¹⁰, CR¹¹, CR¹²;
R⁹-R¹² are independently from each other selected from —H, —F, —Cl, —Br, —I, —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
wherein all alkyl, alkenyl, alkynyl and cycloalkyl residues contained in the definitions of R⁹-R¹² are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂;
wherein all alkyl, alkenyl, alkynyl and cycloalkyl residues contained in the definitions of R⁹-R¹² can contain one or more heteroatoms independently selected from O, S and N in replacement of a carbon atom, and wherein such a replacement cannot result in one of the groups selected from C═O and S═O directly bound to an aromatic ring;
wherein R⁹-R¹² are preferably selected from —H, —F, —Cl, —Br, —CH₃, —CF₃, —OH, —OCH₃, —OCF₃, cyclopropyl, oxiranyl, —C(CH₃)₃, —N(CH₃)₂, —NH₂, —CN, —CH₂OCH₃, —OCH(CH₃)₂, —CH₂NH₂, —CH₂N(CH₃)₂, —CH₂OH, —NO₂, —CH₂-N-morpholinyl;
R⁶=—H, C₁-C₈ preferably C₁-C₄ alkyl, C₂-C₈ preferably C₂-C₄ alkenyl, C₂-C₈ preferably C₂-C₄ alkynyl, C₃-C₆ cycloalkyl, C₅-C₆ cycloalkenyl, C₅-C₁₂ bicycloalkyl, C₇-C₁₂ bicycloalkenyl, C₈-C₁₄ tricycloalkyl, and aromatic and heteroaromatic residues preferably six-membered aromatic cycles and five to six membered heteroaromatic cycles;
and wherein bicyclic and tricyclic residues include fused, bridged and spiro systems;
wherein said cycloalkyl, cycloalkenyl bicycloalkyl, bicycloalkenyl, tricycloalkyl, aromatic and heteroaromatic residues contained in the definition of R⁶ can optionally be linked through a C₁ alkylene or a C₂ alkylene or a C₃ alkylene linker to the N to which R⁶ is bound;
wherein all aromatic and heteroaromatic residues contained in the definition of R⁶ are unsubstituted or substituted with one or more substituents independently selected from: —F, —Cl, —Br, —I, —CN, —NCO, —NCS, —OH, —NH₂, —NO₂, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
wherein all alkyl, alkenyl, alkynyl, cycloalkyl, cycloalkenyl, bicycloalkyl, bicycloalkenyl and tricycloalkyl residues, and alkylene linkers contained in the definition of R⁶ are linear or branched, and are unsubstituted or substituted with one or more substituents independently selected from: —F, —Cl, —Br, —I, —CN, —NCO, —NCS, —OH, —NH₂, ═O, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
wherein all alkyl, alkenyl, alkynyl, cycloalkyl, cycloalkenyl, bicycloalkyl, bicycloalkenyl, tricycloalkyl and heteroaromatic residues, and alkylene linkers contained in the definition of R⁶ can contain one or more heteroatoms independently selected from O, S and N in replacement of a carbon atom;
wherein R⁶ is preferably —H, —CH₃, —CH₂CH₃, n-propyl, isopropyl, cyclopropyl, —CF₃ and —CF₂CF₃, benzyl, tert-butyl, phenyl, cyclohexyl, 1-phenylethyl, 2,2-dimethyl-1-phenylpropyl, (1-naphtyl)-methyl, 4-methoxybenzyl, 4-trifluoromethylbenzyl, tetrahydropyranyl;
wherein all alkyl, alkenyl, alkynyl, cycloalkyl, cycloalkenyl, bicycloalkyl, bicycloalkenyl, tricycloalkyl, aromatic and heteroaromatic residues contained in the definitions of R²-R⁶ and R⁹-R¹² can be partially or fully halogenated, particularly fluorinated, more particularly perfluorinated;
Y=—H, linear or branched C₁-C₆ alkyl, linear or branched C₂-C₆ alkenyl, linear or branched C₂-C₆ alkynyl, C₃-C₆ cycloalkyl, C₅-C₆ cycloalkenyl, —OH, linear or branched —OC₁-C₆ alkyl, linear or branched —OC₂-C₆ alkenyl, linear or branched —OC₂-C₆ alkynyl, —OC₃-C₆ cycloalkyl, —OC₅-C₆ cycloalkenyl, —CN, aromatic and heteroaromatic residues preferably six-membered aromatic cycles and five- to six-membered heteroaromatic cycles, —S(O)R¹³ and —S(O)₂R¹³ wherein R¹³ is selected from linear or branched C₁-C₆ alkyl, linear or branched C₂-C₆ alkenyl, linear or branched C₂-C₆ alkynyl, C₃-C₆ cycloalkyl, C₅-C₆ cycloalkenyl, —CF₃, and —C₆H₄CH₃;
wherein all cycloalkyl, cycloalkenyl, aromatic and heteroaromatic residues contained in the definition of Y can optionally be linked through a C₁ alkylene, or a C₂ alkylene, or a C₃ alkylene, or an —O—, or an —O—CH₂—, or an —O—CH₂—CH₂— linker to the N to which Y is bound;
wherein all aromatic and heteroaromatic residues contained in the definition of Y are unsubstituted or substituted with one or more substituents independently selected from: —F, —Cl, —Br, —I, —CN, —NCO, —NCS, —OH, —NH₂, —NO₂, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
wherein all alkyl, alkenyl, alkynyl, cycloalkyl, and cycloalkenyl residues, and alkylene linkers contained in the definition of Y are linear or branched, and are unsubstituted or substituted with one or more substituents independently selected from: —F, —Cl, —Br, —I, —CN, —NCO, —NCS, —OH, —NH₂, ═O, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);
wherein all alkyl, alkenyl, alkynyl, cycloalkyl, cycloalkenyl and heteroaromatic residues, and alkylene linkers contained in the definition of Y can contain one or more heteroatoms independently selected from O, S and N in replacement of a carbon atom;
wherein all alkyl, alkenyl, alkynyl, cycloalkyl, cycloalkenyl, aromatic and heteroaromatic residues and alkylene linkers contained in the definition of Y can be partially or fully halogenated, particularly fluorinated, more particularly perfluorinated;
wherein Y can form a ring structure together with R⁶, wherein the said ring structure including the N-atom of formula I is selected from three-membered rings, four-membered rings, five-membered rings, six-membered rings, from five- to twelve-membered bicyclic residues, from eight- to fourteen-membered tricyclic residues, and from heteroaromatic residues, wherein all rings, bicyclic, tricyclic and heteroaromatic residues can additionally contain one or more heteroatoms independently selected from O, S and N in replacement of a carbon atom contained in the ring structure, and wherein all rings, bicyclic, tricyclic and heteroaromatic residues are unsubstituted or substituted with one or more substituents independently selected from: —F, —Cl, —Br, —I, —CN, —NCO, —NCS, —OH, —OCH₃, —NH₂, —NHCH₃, —N(CH₃)₂, ═O, —CH₃, —CF₃, morpholinyl;
and wherein bicyclic and tricyclic residues include fused, bridged and spiro systems;
Z¹ and Z² are selected from the following groups: 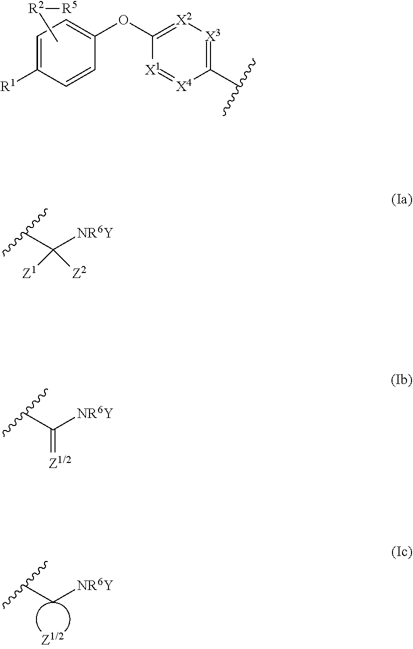
wherein Z¹ is selected from linear or branched C₁-C₃ alkyl preferably —CH₃, cyclopropyl, oxiranyl, N-methyl-aziridinyl, thiiranyl, —CN, —N₃, —CF₃, —CF₂CF₃, and wherein Z² is independently selected from —H and linear or branched C₁-C₃ alkyl preferably —CH₃, —CF₃, —CF₂CF₃ (la);
or wherein Z¹ and Z² are together ═O, ═S, ═NR¹⁴ (Ib); wherein R¹⁴ is selected from —H, —OH, —OCH₃, —CN, —S(O)C(CH₃)₃, —S(O)₂CH₃, —S(O)₂CF₃, linear or branched C₁-C₃ alkyl preferably —CH₃, cyclopropyl, —CF₃, —CF₂CF₃, —CH₂CF₃, —C₆H₅, —CH₂C₆H₅;
or wherein Z¹ and Z² form together a cyclic residue including the carbon atom to which they are bound (Ic); wherein the cyclic residue is selected from three-membered rings, four-membered rings, five-membered rings and six-membered rings, wherein all rings optionally can contain one or more heteroatoms independently selected from O, S and N in replacement of a carbon atom contained in the ring structure; wherein all rings are unsubstituted or substituted with one or more substituents independently selected from: —F, —Cl, —Br, —I, —CN, —NCO, —NCS, —OH, —OCH₃, —NH₂, —NHCH₃, —N(CH₃)₂, ═O, —CH₃ and —CF₃;
wherein all alkyl and cyclic residues contained in the definitions of Z¹ and Z² can be partially or fully halogenated, particularly fluorinated, more particularly perfluorinated.

2. The compound of claim 1 according to formula (Ia) or a salt or solvate thereof.

3. The compound of claim 1 according to formula (Ib) or a salt or solvate thereof.

4. The compound of claim 1 according to formula (Ic) or a salt or solvate thereof.

5. The compound of claim 1 with the proviso that
(i) compounds as indicated in Table 1 are excluded,
(ii) compounds as indicated in Table 2 are excluded and/or
(iii) the compound as indicated in Table 3 are excluded.

6. The compound of claim 1
wherein R¹ is selected from methyl, ethyl, n-propyl, n-butyl, n-pentyl, n-hexyl, iso-propyl, sec-butyl, tert-butyl, tert-pentyl, tert-octyl, 3-pentyl, —CF₃, —CF₂CF₃, —(CF₂)₂CF₃, —CH(CF₃)₂, —CH₂SCH₃, —CH₂CH₂SCH₃, —CH₂SCH₂CH₃, —CH₂CH₂SCH₂CH₃, methoxymethyl, methoxyethyl, methoxypropyl, ethoxymethyl, ethoxyethyl, propoxymethyl, dimethyl-aminomethyl, dimethyl-aminoethyl, diethyl-aminomethyl, ethyl-methyl-aminomethyl, cyclopropyl, methyl-cyclopropyl, ethyl-cyclopropyl, trifluoromethyl-cyclopropyl, perfluoroethyl-cyclopropyl, cyclobutyl, cyclopentyl, cyclohexyl, cycloheptyl, bicyclopentyl, bicyclohexyl, bicycloheptyl preferably norbornyl, bicyclooctyl, bicyclooctenyl, bicyclononyl, methylbicyclononyl, adamantyl, tricyclodecyl, oxiranyl, oxetanyl, tetrahydrofuranyl, methyltetrahydrofuranyl, trimethyltetrahydrofuranyl, tetrahydropyranyl, aziridinyl, N-methylaziridinyl, azetidinyl, N-methylazetidinyl, difluoroazetidinyl, pyrrolidinyl, N-methylpyrrolidinyl, piperidinyl, N-methylpiperidinyl, difluoropiperidinyl, thiiranyl, thietanyl, tetrahydrothiophenyl, tetrahydrothiopyranyl, dioxanyl, piperazinyl, dimethylpiperazinyl, dithianly, morpholinyl, N-methylmorpholinyl, thiomorpholinyl, N-methylthiomorpholinyl, oxa-azaspiroheptyl, N-methyloxa-azaspiroheptyl, azaspiroheptyl, N-methylazaspiroheptyl, thia-azaspiroheptyl, N-methylthia-azaspiroheptyl, difluorothia-azaspiroheptyl, azaspirooctyl, N-methylazaspirooctyl, oxa-azaspirooctyl, N-methyloxa-azaspirooctyl, oxa-azaspirononyl, N-methyloxa-azaspirononyl, azaspirononyl, N-methylazaspirononyl, oxa-azaspirodecyl, N-methyloxa-azaspirodecyl, azaspirodecyl, N-methylazaspirodecyl, dihydro-oxazinyl, N-methyldihydro-oxazinyl, oxazolidinyl, N-methyloxazolidinyl, dioxolanyl, imidazolidinyl, N-methylimidazolidinyl, N,N-dimethylimidazolidinyl, azepanyl, N-methylazepanyl, azaspirohexyl, N-methylazaspirohexyl, oxa-azadispirodecyl, N-methyloxa-azadispirodecyl, azadispirodecyl, N-methylazadispirodecyl, oxa-azabicyclooctyl, N-methyloxa-azabicyclooctyl, azabicyclooctyl, N-methylazabicyclooctyl, azabicycloheptyl, N-methylazabicycloheptyl, azabicyclononyl, N-methylazabicyclononyl, azaadamantyl, —O(adamantyl), oxa-azabicyclononyl, N-methyloxa-azabicyclononyl, oxa-azabicycloheptyl, N-methyloxa-azabicycloheptyl, diazabicyclooctyl, N-methyldiazabicyclooctyl, N,N-dimethyldiazabicyclooctyl, diazabicycloheptyl, N-methyldiazabicycloheptyl, N,N-dimethyldiazabicycloheptyl; 4-oxocyclohexyl; 3-oxocyclopentyl; 2-oxocyclobutyl, 4-oxobicyclo[4.1.0]heptan-1-yl.

7. The compound of claim 1
wherein R¹ is selected from C₄-C₁₂ alkyl, C₄-C₁₂ alkenyl, C₄-C₁₂ alkynyl, cyclic, bicyclic and tricyclic residues, wherein the alkyl, alkenyl and alkynyl residues are preferably branched, including:

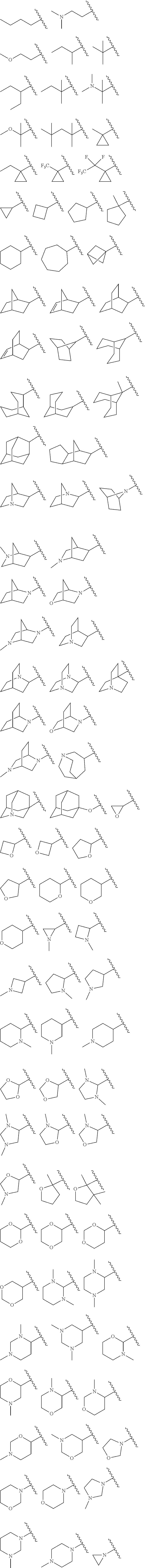

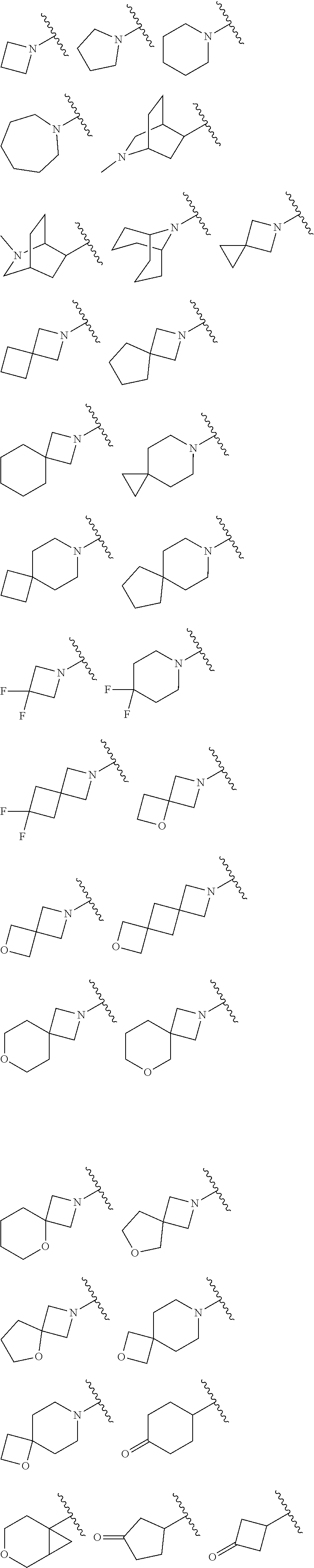

8. The compound of claim 1
wherein R²-R³ each are —H, R⁴ is preferably —H or —F, and/or R⁵ is —H, —F, —Cl, —Br, —CH₃, —CF₃, —CH═CH₂, —C≡CH, —CH₂OH, —CH₂NHCH₃, —OH, —OCH₃, —OCF₃, cyclopropyl, oxiranyl, —CH₂—N-morpholinyl, —C(CH₃)₃, —CH₂OCH₃, —NO₂, —CN, —NH₂, —N(CH₃)₂, —OCH(CH₃)₂, —CH₂NH₂, —CH₂N(CH₃)₂.

9. The compound of claim 1
wherein the six-membered aromatic ring, to which substituents R¹ to R⁵ are bound as defined in general formula (I), is selected from:

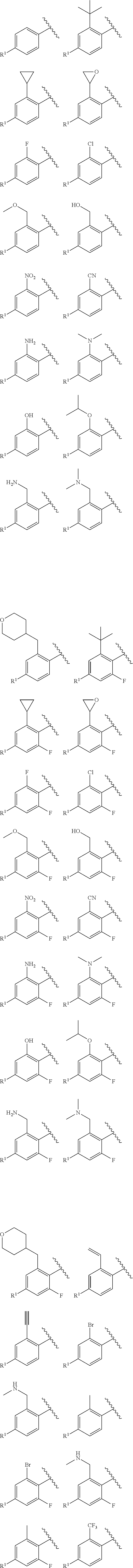

10. The compound of claim 1
wherein the six-membered aromatic ring containing X¹-X⁴ as defined in general formula (I) is selected from:

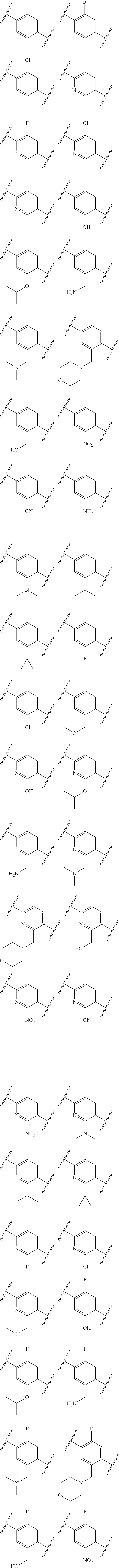

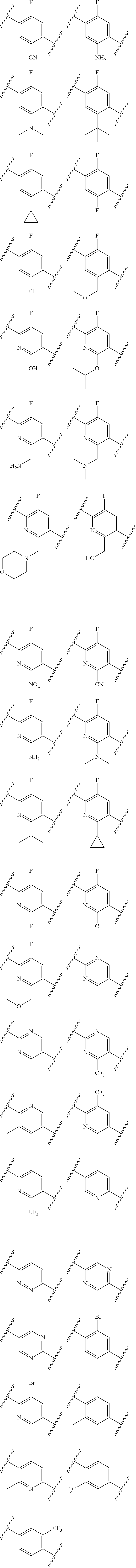

11. The compound of claim 1
wherein Y is —H, —CH₃, —CH₂CH₃, n-propyl, isopropyl, cyclopropyl, cyclohexyl, tetrahydropyranyl, —CF₃, —CF₂CF₃, —OH, —OCH₃, —OCH₂CH₃, —OCH₂(cyclopropyl), —CN, —S(O)C(CH₃)₃, —S(O)₂CH₃, —S(O)₂CF₃, —S(O)₂C₆H₄CH₃, —OCH₂C₆H₅ and —OC₆H₅; and for R⁶=—H or —CH₃ or benzyl, then Y is preferably —OH, —OCH₃, —OCH₂CH₃, —OCH₂(cyclopropyl).

12. The compound of claim 1
wherein the ring structure of Y together with R⁶ including the N-atom of formula I is selected from aziridinyl, azetidinyl, pyrrolidinyl, piperidinyl, difluoropiperidinyl, morpholinyl, morpholinylazetidinyl, hydroxyazetidinyl, azetidinonyl, azetidinyl, difluoroazetidinyl, azaspirohexyl, azaspiroheptyl, difluoroazaspiroheptyl, hydroxyazaspiroheptyl, methylhydroxyazaspiroheptyl, trifluoromethylhydroxyazaspiroheptyl, azaspirooctyl, azaspirononyl, oxa-azaspiroheptyl, oxa-azaspirooctyl, oxa-azaspirononyl, thia-azaspiroheptyl, oxazolidinyl, tetrahydro-oxazinyl, isoxazolidinyl, oxazinane, isoxazolidine, piperazine.

13. The compound of claim 1
wherein the ring structure of Y together with R⁶ including the N-atom of formula I is selected from:

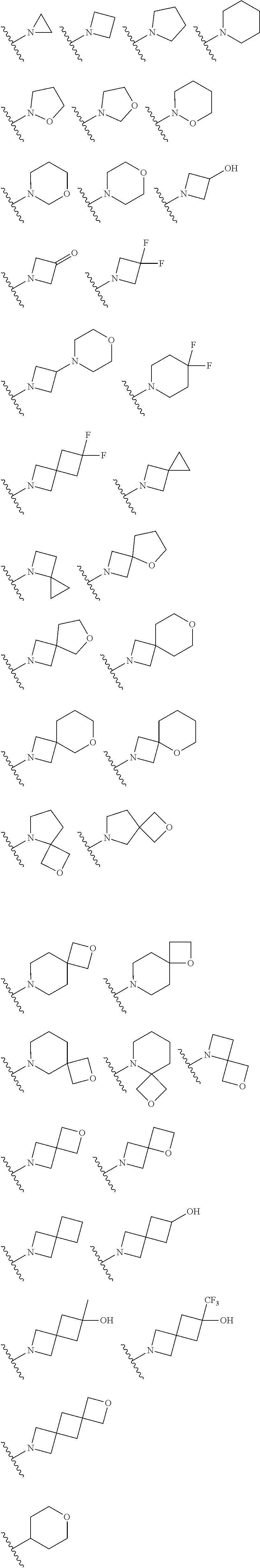

14. The compound of claim 1
wherein Z¹ is —CH₃, —CF₃, —CN, cyclopropyl; and/or Z² is preferably —H, —CH₃ and —CF₃; e.g.:

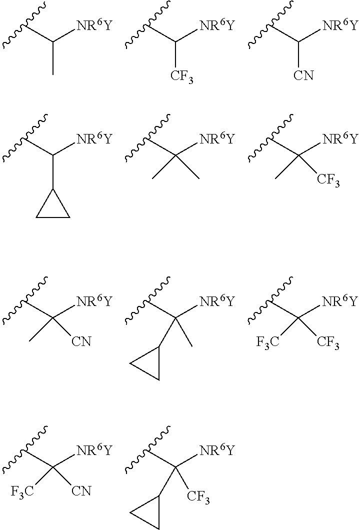

15. The compound of claim 1
wherein Z¹ and Z² are together preferably ═O, ═NR¹⁴; wherein R¹⁴ is preferably selected from —H, —CH₃, cyclopropyl, —OH, —OCH₃, —CN:

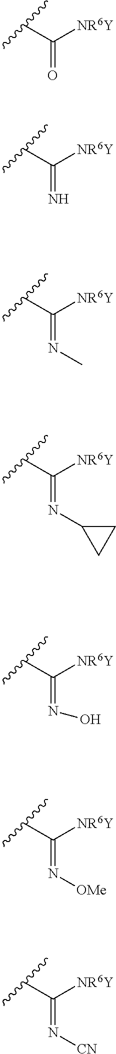

16. The compound of claim 1
wherein Z¹ and Z² form together a three membered or four membered cyclic residue including the carbon atom to which they are bound; wherein this cyclic residue is preferably selected from cyclopropyl, cyclobutyl, oxiranyl, oxetanyl, aziridinyl, azetidinyl and thietanyl; and wherein this cyclic residue is optionally substituted preferably with —F, —OH, —OCH₃, —NH₂, —NHCH₃, —N(CH₃)₂, ═O, —CH₃ and —CF₃;
and wherein this cyclic residue is even more preferably selected from:

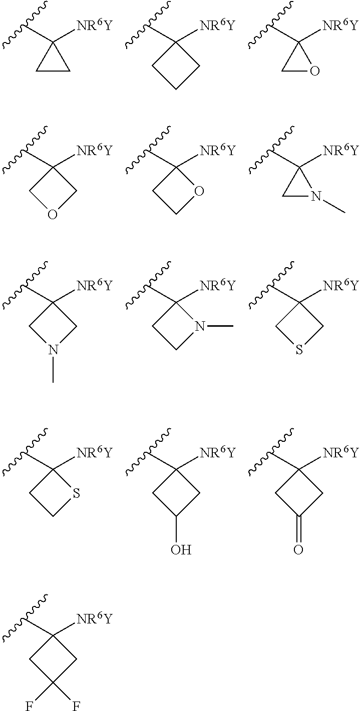

17. The compound of claim 1
wherein Y is selected from residues as contained in the general definition of Y, which are bound with an oxygen atom to the N, to which Y is bound.

18. The compound of claim 1
wherein R¹ is selected from residues as contained in the general definition of R¹, which contain four or more, preferably six or more and even more preferably seven or more carbon atoms, and wherein R¹ contains no heteroatom.

19. The compound of claim 18
wherein R¹ is selected from cyclic, bicyclic and tricyclic structures.

20. The compound of claim 18
wherein R¹ is selected from cyclohexyl, norbornyl, bicyclooctyl, bicyclononyl, methylbicyclononyl, tricyclodecyl and adamantyl.

21. The compound of claim 1
wherein R¹ is selected from residues as contained in the general definition of R¹, which contain four or more, preferably six or more and even more preferably seven or more carbon atoms, and wherein R¹ contains one or more preferably one to two heteroatoms independently selected from O, S and N in replacement of a carbon atom contained in R¹.

22. The compound of claim 21 wherein R¹ is selected from tetrahydropyranyl, N-methylpiperidinyl, morpholinyl, 4-oxocyclohexyl, azabicycloheptyl, N-methylazabicycloheptyl, oxa-azabicycloheptyl, N-methyldiazabicycloheptyl, azabicyclooctyl, diazabicyclooctyl, N-methyldiazabicyclooctyl, oxa-azabicyclooctyl, azabicyclononyl, azaadamantyl and —O(adamantyl).

23. The compound of claim 1
wherein the compound has the following structure (1-1): 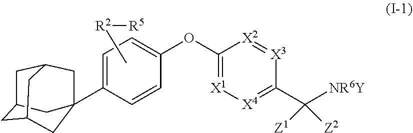
wherein Z¹ and Z² are defined as in general formula (I), including general formula (Ia), general formula (Ib) and general formula (Ic), including the substitutions and preferred definitions, optionally with the proviso that in the case of general formula (Ib) Z¹ and Z² are together different from ═O,
and wherein R¹⁴ is defined as in general formula (Ib) including the substitutions and preferred definitions,
and wherein Y, R²-R⁶, R⁹-R¹³ and X¹-X⁴ are defined as in general formula (I) including the substitutions and preferred definitions.

24. The compound of claim 1
wherein the compound has the following structure (1-4): 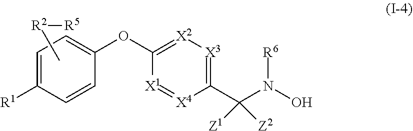
wherein R⁶ is defined as in general formula (I) including the substitutions and preferred definitions, with the proviso that R⁶ is different from —H,
and wherein Z¹ and Z² are defined as in general formula (I), including general formula (Ia), general formula (Ib) and general formula (Ic), including the substitutions and preferred definitions,
and wherein R¹⁴ is defined as in general formula (Ib) including the substitutions and preferred definitions,
and wherein R¹-R⁵, R⁷-R¹² and X¹-X⁴ are defined as in general formula (I) including the substitutions and preferred definitions.

25. The compound of claim 1
wherein the compound has the following structure (Ib-1): 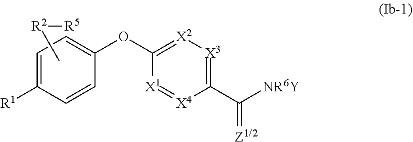
wherein R¹ is defined as in general formula (I) including the substitutions and preferred definitions, wherein R¹ contains six or more carbon atoms, which are optionally independently replaced by a heteroatom selected from O, S and N as defined in general formula (I), and wherein R¹ is selected from cyclic, bicyclic and tricyclic structures,
and wherein R⁵ is defined as in general formula (I) including the substitutions and preferred definitions, with the proviso that R⁵ is different from —H,
and wherein Z¹, Z² and R¹⁴ are defined as in general formula (Ib), including the substitutions and preferred definitions,
and wherein R²-R⁴, R⁶-R¹³ and X¹-X⁴ and Y are defined as in general formula (I) including the substitutions and preferred definitions.

26. The compound of claim 1
wherein the compound has the following structure (Ib-2): 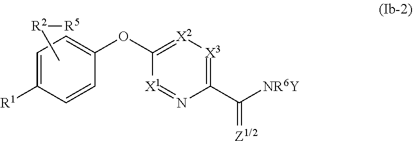
wherein R¹ is defined as in general formula (I) including the substitutions and preferred definitions, wherein R¹ contains six or more carbon atoms, which are optionally independently replaced by a heteroatom selected from O, S and N as defined in general formula (I), and wherein R¹ is selected from cyclic, bicyclic and tricyclic structures,
and wherein Z¹, Z² and R¹⁴ are defined as in general formula (Ib), including the substitutions and preferred definitions,
and wherein R²-R¹³, X¹-X³ and Y are defined as in general formula (I) including the substitutions and preferred definitions.

27. A compound as shown in any one of Table 6 to Table 54 a salt or solvate thereof.

28. A pharmaceutical composition comprising the compound of claim 1 in combination with a pharmaceutical carrier suitable for in human medicine or veterinary medicine.

29. (canceled)

30. A method for enhancing Notch signaling, comprising administering a compound according to claim 1.

31. (canceled)

32. A method for treating diseases and malignant, non-malignant and hyperproliferative disorders of the skin, mucosa, skin and mucosal appendages, cornea, and epithelial tissues, including cancer such as non-melanoma skin cancer including squamous and basal cell carcinoma and precancerous lesions including actinic keratosis, skin and/or mucosal disorders with cornification defects and/or abnormal keratinocyte proliferation, skin and/or mucosal diseases associated with, accompanied by and/or caused by viral infections, atopic dermatitis and acne and in the promotion of wound healing of the skin and mucosa, comprising administering a compound according to claim 1 to a patient in need of such treatment.

33. A method for treating hyperproliferative disorders, cancers or precancerous lesions of the skin, oral mucosa, tongue, lung, stomach, breast, cancer of the neuroendocrine system, such as medullary thyroid cancer, brain, pancreas, liver, thyroid, and genitourinary tract, including cancer of the cervix and ovaries, comprising administering the compound according to claim 1 to a patient in need of such treatment.

34. A method for treating malignant and non-malignant muscular diseases including muscular dystrophies, or in muscle regeneration, or in hyperproliferative disorders of the muscle, such as muscle hyperplasia and muscle hypertrophy, comprising administering the compound according to claim 1 to a patient in need of such treatment.

35. A method for treating immune system-related disorders, including disorders of the haematopoietic system including the haematologic system, such as cancer of the haematopoietic and haematologic system such as leukemias and lymphomas, such as malignancies of the myeloid lineage e.g. acute and chronic myeloid leukemia and acute and chronic promyelocytic leukemia, and malignancies of the lymphoid lineage, e.g. acute and chronic T-cell leukemia and acute and chronic B-cell leukemia, and cutaneous T-cell lymphoma, comprising administering the compound according to claim 1 to a patient in need of such treatment.

36. A method for improving therapeutic immune system-related applications including immunotherapy and other immunotherapy methods comprising administering an immunologic adjuvant or vaccine adjuvant comprising a compound according to claim 1.

37. A method of treating a hyperproliferative disorder comprising administering a subject in need thereof, particularly a human subject, a therapeutically effective amount of a compound according to claim 1.

38. A method of treating a disorder associated with, accompanied by and/or caused by dysfunctional Notch signaling, comprising administering to a subject in need thereof, particularly a human subject, a therapeutically effective amount of a compound according to claim 1.
